(19) United States
(12) Patent Application Publication
(10) Pub. No.: US 2023/0332164 A1
(43) Pub. Date: Oct. 19, 2023

(54) BACTERIA ENGINEERED TO SECRETE ACTIVE PROTEINS

(71) Applicant: Synlogic Operating Company, Inc., Cambridge, MA (US)

(72) Inventors: Dean Falb, Sherborn, MA (US); Vincent M. Isabella, Medford, MA (US); Paul F. Miller, Salem, CT (US); Adam B. Fisher, Cambridge, MA (US); Ning Li, Winchester, MA (US); Jose M. Lora, Boston, MA (US)

(21) Appl. No.: 18/028,785

(22) PCT Filed: Sep. 30, 2021
(86) PCT No.: PCT/US2021/052882

§ 371 Date: Mar. 28, 2023

Related U.S. Application Data
(60) Provisional application No. 63/164,815, filed on Mar. 23, 2021.
(60) Provisional application No. 63/085,363, filed on Sep. 30, 2020.

(51) Int. Cl. C12N 15/70; C07K 14/54

(52) U.S. Cl. CPC C12N 15/70; C07K 14/54; C12R 2001/19

(57) ABSTRACT

Recombinant bacteria capable of producing effector molecules, which are secreted as therapeutically active polypeptides, pharmaceutical compositions thereof, and methods of treating or preventing autoimmune disorders, cancer and/or metabolic diseases, are disclosed.

### RELATED APPLICATIONS

The instant application claims priority to U.S. Provisional Application No. 63/164,815 filed Mar. 23, 2021 and U.S. Provisional Application No. 63/085,363 filed Sep. 30, 2020, the entire contents of each of which are expressly incorporated by reference herein in their entireties.

### SEQUENCE LISTING

The instant application contains a Sequence Listing which has been submitted electronically in ASCII format and is hereby incorporated by reference in its entirety. Said ASCII copy, created on Sep. 28, 2021, is named 126046-03020_SL.txt and is 1,086,656 bytes in size.

### BACKGROUND

A growing body of scientific evidence suggests that probiotic bacteria are beneficial in the treatment or prevention of various diseases or disorders associated with the gut, including, for example, gastrointestinal disorders such as Crohn's disease and inflammatory bowel syndrome. More recently, recombinant bacteria have emerged as a potential new therapeutic treatment modality for gastrointestinal diseases and have also opened the field of bacterial therapies to a large number of other indications, including metabolic diseases, inflammatory diseases, and cancer. One benefit of recombinant bacteria is the ability to specifically target one or more disease mechanisms. For example, for gastrointestinal disorders, bacteria can be engineered to contain genes for the expression of anti-inflammatory agents or agents that aid in the healing of a disrupted gut-barrier, such as the short chain fatty acid butyrate, e.g., as described in International Patent Publication WO2016141108.

Additionally, bacterial therapies have the additional advantage that the size of the bacterial chromosome(s) allows for the insertion of gene(s) for the production and secretion of multiple effectors. Potential secreted polypeptides include signaling molecules, such as cytokines and growth factors, their receptors, and single chain antibodies directed against cell surface molecules, many of which have been proposed as are promising candidates for therapeutic interference in a wide range of indications.

A certain level of technical understanding of approaches to the secretion of heterologous proteins from bacteria can be gained from recombinant production strategies for therapeutic or other proteins. However, effective protocols for generation of recombinant bacteria which produce and secrete biologically active polypeptides in vivo have yet to be established.

Multiple conditions must be met for the successful secretion of effective amounts of biologically active polypeptides. In Gram-negative bacteria, secreted polypeptides have to cross the two membranes and a thin layer of peptidoglycan in the periplasmic space between the inner and outer lipid membranes. Type I, II, III, IV, and V secretion pathways are common among Gram-negative bacteria, and all of these pathways have been exploited for the secretion of recombinant proteins. However, given that secretion of a polypeptide across the inner and outer membranes of a gram-negative bacterium is complex, involving the execution of several steps and the use of different biological factors, a number of complications can arise. For example, problems include incomplete translocation across the inner membrane, insufficient capacity of the export machinery, and proteolytic degradation (Mergulhao et al., Biotechnology Advances 23 (2005) 177-202). In addition, the ability of a polypeptide to be secreted from a Gram-negative bacteria, such as E. coli, depends on the specific polypeptide to be secreted and the biochemical properties thereof, such as formation of correct disulfide bonds, size of protein or levels of expression.

Given the number of factors involved in secreting polypeptides from Gram-negative bacteria, in combination with factors arising from the different biological properties and characteristics of individual polypeptides, e.g., size, dimer formation, secondary and tertiary protein folding, and polypeptide expression levels, secretion of polypeptides from Gram-negative bacteria remains challenging. Further, even if successful secretion is achieved, the polypeptide is not always secreted in a biologically active form.

In view of the difficulties outlined here as well as others, there remains a need for engineering and methods for the successful secretion of biologically active polypeptides.

### SUMMARY

The instant disclosure relates to compositions of recombinant bacteria and methods for secreting therapeutically active proteins from recombinant bacteria for treatment of diseases or disorders. The recombinant bacteria disclosed herein are capable of high yield production of functionally active effector molecules, which are secreted as therapeutically active polypeptides.

In some embodiments, the recombinant bacteria are functionally silent until they reach an inducing environment, e.g., a mammalian gut, or the tumor microenvironment wherein expression of the therapeutic molecule is induced. In certain embodiments, the recombinant bacteria are naturally non-pathogenic and may be introduced into the gut in order to reduce gut inflammation and/or enhance gut barrier function and may thereby further ameliorate or prevent an autoimmune disorder. In some embodiments, the recombinant bacteria are tumor targeting and may be introduced into the tumor to stimulate the immune system, combat immune suppression or otherwise fight the cancer. In certain embodiments, the secreted effector molecule is stably produced by the recombinant bacteria, and/or the recombinant bacteria are stably maintained in vivo and/or in vitro. The disclosure also provides pharmaceutical compositions comprising the recombinant bacteria. Methods of treating diseases are also provided.

In some embodiments, the recombinant bacteria produce one or more therapeutic molecule(s) under the control of one or more promoters induced by an environmental condition, e.g., an environmental condition found in the mammalian gut, such as an inflammatory condition or a low oxygen condition. In some embodiments, the recombinant bacteria produce one or more therapeutic molecule(s) under the control of one or more promoters induced the tumor microenvironment. In non-limiting exemplary embodiments, the recombinant bacteria produce one or more therapeutic molecule(s) under the control of an oxygen level-dependent promoter, a reactive oxygen species (ROS)-dependent promoter, or a reactive nitrogen species (RNS)-dependent promoter, and a corresponding transcription factor.

In some embodiments, the recombinant bacterium comprises a gene sequence encoding one or more effector polypeptides and one or more secretion tags, wherein the gene sequence is operably linked to a directly or indirectly inducible promoter that is not associated with the gene sequence in nature and wherein the encoded effector polypeptide is secreted in a biologically active form, and comprising one or more mutations or deletions in an outer membrane protein selected from a group consisting of lpp, nlP, tolA, and PAL.

In some embodiments, the recombinant bacterium comprise one or more one gene(s) encoding one or more effector polypeptides for secretion of an active polypeptide in vivo, wherein the one or more gene sequence(s) for producing the effector polypeptide is operably linked to a directly or indirectly inducible promoter that is not associated with the gene(s) in nature. In some embodiments, the secretion tags are N terminally or C terminally fused to the effector polypeptides. For example, the secretion tag may be covalently linked to the N terminus of the polypeptide through a peptide bond or polypeptide linker. Alternatively, the secretion tag may be covalently linked to the C terminus of the polypeptide through a peptide bond or polypeptide linker.

In some embodiments, the effector is selected from a group consisting of GLP-2, IL-22, IL-10, IL-27, IL-19, IL-20, IL-24, IL-15, IL-2, GMCSF, TNF-alpha, IFN-gamma, CXCL10, CXCL9 and hyaluronidase. In some embodiments, the effector include hIL-12a (SEQ ID NO: 1131), hIL-1213 (SEQ ID NO: 1132); mIL-12 (SEQ ID NO: 1131); mIL-1213 (SEQ ID NO: 1133); hIL-15 (SEQ ID NO: 1134); GMCSF (SEQ ID NO: 1135); TNF-alpha (extracellular portion) (SEQ ID NO: 1136); IFN-gamma (SEQ ID NO: 1137); CXCL10 (SEQ ID NO: 1138); and CXCL9 (SEQ ID NO: 1139). In some embodiments, recombinant bacteria comprise a nucleic acid sequence that encodes a polypeptide which is at least about 80%, 85%, 90%, 95%, or 99% homologous to the DNA sequence of any one of SEQ ID NOs: 1131-1139. In some embodiments, the gene encoding the effector include hIL-12a (SEQ ID NO: 1140), hIL-1213 (SEQ ID NO: 1141); mIL-12 (SEQ ID NO: 1142); mIL-1213 (SEQ ID NO: 1143); hIL-15 (SEQ ID NO: 1144); GMCSF (SEQ ID NO: 1145); TNF-alpha (extracellular portion) (SEQ ID NO: 1146); IFN-gamma (SEQ ID NO: 1147); and CXCL10 (SEQ ID NO: 1148). In some embodiments, recombinant bacteria comprise a nucleic acid sequence that is at least about 80%, 85%, 90%, 95%, or 99% homologous to the DNA sequence of any one of SEQ ID NOs: 1140-1148.

Non-limiting examples of contemplated secretion tags include PhoA, OmpF, ompA, cvaC, TorA, fdnG, dmsA, PelB, tolB, torT, dsbA, GltI, GspD, HdeB, MalE, mglB, OppA, PpiA, lamb, ECOLIN_05715, ECOLIN_16495, ECOLIN_19410, and ECOLIN_19880 secretion signals. In some embodiments, the secretion tag is cleaved after secretion into the extracellular environment. In some embodiments, the secretion tag is PhoA. In some embodiments, the secretion tag is ECOLIN 19410 secretion tag. In some embodiments, the secretion tag is GspD secretion tag. In some embodiments, the secretion tag is HdeB secretion tag. In some embodiments, the secretion tag is torT secretion tag. In some embodiments, the secretion tags and/or FliC components are selected from the group consisting of fliC-FliC20 (SEQ ID NO: 894); flic-RBS (SEQ ID NO: 895-897); RBS-phoA (SEQ ID NO: 898); phoA (SEQ ID NO: 889); RBS-ompF (SEQ ID NO: 900); ompF (SEQ ID NO: 901); RBS-cvaC (SEQ ID NO: 902); cvaC (SEQ ID NO: 903); RBS-phoA (Optimized) (SEQ ID NO: 904); optimized phoA (SEQ ID NO: 905); RBS-TorA (SEQ ID NO: 906); TorA (SEQ ID NO: 907); RBS-TorA alternate (SEQ ID NO: 908); TorA (alternate) (SEQ ID NO: 909); RBS-fdnG (SEQ ID NO: 910); fdnG (SEQ ID NO: 911); RBS-dmsA (SEQ ID NO: 912); dmsA (SEQ ID NO: 913) and PelB. In some embodiments, recombinant bacteria comprise a nucleic acid sequence that is at least about 80%, 85%, 90%, 95%, or 99% homologous to the DNA sequence of any one of SEQ ID NOs: 894-913.

In some embodiments, the recombinant bacteria further have one or more mutations or deletions in an outer membrane protein selected from lpp, nlP, tolA, and PAL. In some embodiments, the fully or partially deleted or mutated outer membrane protein is PAL. In some embodiments, the recombinant bacteria further encode a stabilizing polypeptide. In some embodiments, the effector polypeptide is covalently fused to the stabilizing polypeptide through a peptide linker or a peptide bond.

In some embodiments, the C terminus of the effector polypeptide is covalently fused to the N terminus of the stabilizing polypeptide through the peptide linker or peptide bond. In some embodiments, the N terminus of the effector polypeptide is covalently fused to the C terminus of the stabilizing polypeptide through the peptide linker or peptide bond. In some embodiments, the stabilizing polypeptide comprises an immunoglobulin Fc polypeptide. In some embodiments, the immunoglobulin Fc polypeptide comprises at least a portion of an immunoglobulin heavy chain CH2 constant region. In some embodiments, the immunoglobulin Fc polypeptide comprises at least a portion of an immunoglobulin heavy chain CH3 constant region. In some embodiments, the immunoglobulin Fc polypeptide comprises at least a portion of an immunoglobulin heavy chain CH1 constant region. In some embodiments, the immunoglobulin Fc polypeptide comprises at least a portion of an immunoglobulin variable hinge region. In some embodiments, the immunoglobulin Fc polypeptide comprises at least a portion of an immunoglobulin variable hinge region, immunoglobulin heavy chain CH2 constant region and an immunoglobulin heavy chain CH3 constant region. In some embodiments, the immunoglobulin Fc polypeptide is a human IgA or human IgG Fc polypeptide. In some embodiments, the immunoglobulin Fc polypeptide is a human IgG Fc polypeptide. In some embodiments, the immunoglobulin Fc polypeptide is a human IgA polypeptide. In some embodiments, the linker comprises a glycine rich peptide. In some embodiments, the glycine rich peptide comprises the sequence [GlyGlyGlyGlySer]n where n is 1, 2, 3, 4, 5 or 6 (SEQ ID NO: 1053). In some embodiments, the glycine rich peptide comprises the sequence GGGGSGGGGSGGGGSGGGGS (SEQ ID NO: 509). In some embodiments, the one or more effector polypeptides for secretion of an active polypeptide in vivo require multimerization for the effector polypeptide to be active in vivo. In some embodiments, the one or more effector polypeptides for secretion of an active polypeptide in vivo require dimerization for the effector polypeptide to be active in vivo. In some embodiments, the active polypeptide is a homodimer. In some embodiments, the active polypeptide is a heterodimer. In some embodiments, the gene sequence(s) encoding the more effector polypeptides encode a first monomer polypeptide and a second monomer polypeptide and wherein the first and second monomer polypeptides are covalently to each other through a peptide linker or a peptide bond. In some embodiments, the linker comprises GGGGSGGGS (SEQ ID NO: 1054). In some embodiments, the stabilizing polypeptide has the ability to perform an effector function. In some embodiments, the stabilizing polypeptide is able to perform an anti-inflammatory effector function. In some embodiments, the stabilizing polypeptide is able to perform an pro-inflammatory effector function. In some embodiments, the stabilizing polypeptide is a cytokine.

In some embodiments, the stabilizing polypeptide is a multimer. In some embodiments, the stabilizing polypeptide is a dimer. In some embodiments, the gene sequences encoding the stabilizing polypeptide comprise a monomer and a second monomer, wherein the first and second monomer are covalently linked to each other through a peptide bond or a peptide linker.

In some embodiments, the gene sequences are located on a chromosome in the bacterium. In some embodiments, the gene sequences are located on a plasmid in the bacterium. In some embodiments, the bacterium is a probiotic bacterium. In some embodiments, the bacterium is a tumor targeting bacterium. In some embodiments, the bacterium is selected from the group consisting of Bacteroides, Bifidobacterium, Clostridium, Escherichia, Lactobacillus, and Lactococcus. In some embodiments, the bacterium is selected from Clostridium novyi NT, and Clostridium butyricum, and Bifidobacterium longum. In some embodiments, the bacterium is Escherichia coli strain Nissle. In some embodiments, the bacterium is an auxotroph in a gene that is complemented when the bacterium is present in a mammalian gut, e.g., an auxotroph in diaminopimelic acid or an enzyme in the thymine biosynthetic pathway.

In some embodiments, the recombinant bacterium is capable of producing about 25 pg/1E9 cells/hr to about 4500 pg/1E9 cells/hr of IL-2 in vitro. In some embodiments, the bacterium is capable of producing at least 25 pg/1E9 cells/hr, at least 50 pg/1E9 cells/hr, at least 100 pg/1E9 cells/hr, at least 150 pg/1E9 cells/hr, at least 200 pg/1E9 cells/hr, at least 250 pg/1E9 cells/hr, at least 300 pg/1E9 cells/hr, at least 400 pg/1E9 cells/hr, at least 500 pg/1E9 cells/hr, at least 750 pg/1E9 cells/hr, at least 1000 pg/1E9 cells/hr, at least 1500 pg/1E9 cells/hr, at least 2000 pg/1E9 cells/hr, at least 2500 pg/1E9 cells/hr, at least 3000 pg/1E9 cells/hr, at least 3500 pg/1E9 cells/hr, at least 4000 pg/1E9 cells/hr, or at least 4500 pg/1E9 cells/hr of IL-2 in vitro. In some embodiments, the bacterium is capable of producing about 25 pg/1E9 cells/hr to about 4500 pg/1E9 cells/hr, about 500 pg/1E9 cells/hr to about 4500 pg/1E9 cells/hr, about 1000 pg/1E9 cells/hr to about 4500 pg/1E9 cells/hr, about 2000 pg/1E9 cells/hr to about 4500 pg/1E9 cells/hr, about 3000 to about 4500 pg/1E9 cells/hr, about 25 pg/1E9 cells/hr to about 3000 pg/1E9 cells/hr, about 500 pg/1E9 cells/hr to about 3000 pg/1E9 cells/hr, about 1000 pg/1E9 cells/hr to about 3000 pg/1E9 cells/hr about 2000 pg/1E9 cells/hr to about 3000 pg/1E9 cells/hr, about 25 pg/1E9 cells/hr to about 2000 pg/1E9 cells/hr, about 500 to about 2000 pg/1E9 cells/hr, about 1000 pg/1E9 cells/hr to about 2000 pg/1E9 cells/hr, or about 25 pg/1E9 cells/hr to about 1000 pg/1E9 cells/hr of IL-2 in vitro.

In some embodiments, the recombinant bacterium is capable of producing about 1.5 ng/1E9 cells/hr to about 3 ng/1E9 cells/hr of IL15 in vitro. In some embodiments, the bacterium is capable of producing at least 1 ng/1E9 cells/hr, at least 1.5 ng/1E9 cells/hr, at least 2 ng/1E9 cells/hr, at least 2.5 ng/1E9 cells/hr, at least 3 ng/1E9 cells/hr, at least 3.5 ng/1E9 cells/hr of IL15 in vitro. In some embodiments, the recombinant bacterium is capable of producing about 1 ng/1E9 cells/hr to about 3.5 ng/1E9 cells/hr, about 1.5 ng/1E9 cells/hr to about 3.5 ng/1E9 cells/hr, about 2 ng/1E9 cells/hr to about 3.5 ng/1E9 cells/hr, about 2.5 ng/1E9 cells/hr to about 3.5 ng/1E9 cells/hr, about 3 ng/1E9 cells/hr to about 3.5 ng/1E9 cells/hr, 1 ng/1E9 cells/hr to about 3 ng/1E9 cells/hr, about 1.5 ng/1E9 cells/hr to about 3 ng/1E9 cells/hr, about 2 ng/1E9 cells/hr to about 3 ng/1E9 cells/hr, about 2.5 ng/1E9 cells/hr to about 3 ng/1E9 cells/hr, 1 ng/1E9 cells/hr to about 2.5 ng/1E9 cells/hr, about 1.5 ng/1E9 cells/hr to about 2.5 ng/1E9 cells/hr, about 2 ng/1E9 cells/hr to about 2.5 ng/1E9 cells/hr, 1 ng/1E9 cells/hr to about 2 ng/1E9 cells/hr, about 1.5 ng/1E9 cells/hr to about 2 ng/1E9 cells/hr, or 1 ng/1E9 cells/hr to about 1.5 ng/1E9 cells/hr of IL15 in vitro.

In another aspect, the recombinant bacterium comprises 1) a gene sequence encoding IL-22 and one or more secretion tags, wherein the gene sequence is operably linked to an FNR-responsive promoter; 2) one or more mutations or deletions in an outer membrane protein PAL; and wherein IL-22 is secreted in a biologically active form. In some embodiments, the secretion tag is selected from a group consisting of PhoA, OmpF, ompA, cvaC, TorA, fdnG, dmsA, PelB, tolB, torT, dsbA, GltI, GspD, HdeB, MalE, mglB, OppA, PpiA, lamb, ECOLIN_05715, ECOLIN_16495, ECOLIN_19410, and ECOLIN_19880 secretion signals.

In some embodiments, the bacterium is capable of producing about 40 ng/1E8 cells/hr of IL-22 in vitro. In some embodiments, the bacterium is capable of producing about 5 ng/1E8 cells/hr to about 50 ng/1E8 cells/hr, about 10 ng/1E8 cells/hr to about 50 ng/1E8 cells/hr, about 15 ng/1E8 cells/hr to about 50 ng/1E8 cells/hr, about 20 ng/1E8 cells/hr to about 50 ng/1E8 cells/hr, about 25 ng/1E8 cells/hr to about 50 ng/1E8 cells/hr, about 30 ng/1E8 cells/hr to about 50 ng/1E8 cells/hr, about 35 ng/1E8 cells/hr to about 50 ng/1E8 cells/hr, about 40 ng/1E8 cells/hr to about 50 ng/1E8 cells/hr, about 45 ng/1E8 cells/hr to about 50 ng/1E8 cells/hr, 5 ng/1E8 cells/hr to about 45 ng/1E8 cells/hr, about 10 ng/1E8 cells/hr to about 45 ng/1E8 cells/hr, about 15 ng/1E8 cells/hr to about 45 ng/1E8 cells/hr, about 20 ng/1E8 cells/hr to about 45 ng/1E8 cells/hr, about 25 ng/1E8 cells/hr to about 45 ng/1E8 cells/hr, about 30 ng/1E8 cells/hr to about 45 ng/1E8 cells/hr, about 35 ng/1E8 cells/hr to about 50 ng/1E8 cells/hr, about 40 ng/1E8 cells/hr to about 45 ng/1E8 cells/hr, 5 ng/1E8 cells/hr to about 40 ng/1E8 cells/hr, about 10 ng/1E8 cells/hr to about 40 ng/1E8 cells/hr, about 15 ng/1E8 cells/hr to about 40 ng/1E8 cells/hr, about 20 ng/1E8 cells/hr to about 40 ng/1E8 cells/hr, about 25 ng/1E8 cells/hr to about 40 ng/1E8 cells/hr, about 30 ng/1E8 cells/hr to about 40 ng/1E8 cells/hr, about 35 ng/1E8 cells/hr to about 50 ng/1E8 cells/hr, 5 ng/1E8 cells/hr to about 35 ng/1E8 cells/hr, about 10 ng/1E8 cells/hr to about 35 ng/1E8 cells/hr, about 15 ng/1E8 cells/hr to about 35 ng/1E8 cells/hr, about 20 ng/1E8 cells/hr to about 35 ng/1E8 cells/hr, about 25 ng/1E8 cells/hr to about 35 ng/1E8 cells/hr, about 30 ng/1E8 cells/hr to about 35 ng/1E8 cells/hr, 5 ng/1E8 cells/hr to about 30 ng/1E8 cells/hr, about 10 ng/1E8 cells/hr to about 30 ng/1E8 cells/hr, about 15 ng/1E8 cells/hr to about 30 ng/1E8 cells/hr, about 20 ng/1E8 cells/hr to about 30 ng/1E8 cells/hr, about 25 ng/1E8 cells/hr to about 30 ng/1E8 cells/hr, 5 ng/1E8 cells/hr to about 25 ng/1E8 cells/hr, about 10 ng/1E8 cells/hr to about 25 ng/1E8 cells/hr, about 15 ng/1E8 cells/hr to about 25 ng/1E8 cells/hr, about 20 ng/1E8 cells/hr to about 25 ng/1E8 cells/hr, 5 ng/1E8 cells/hr to about 20 ng/1E8 cells/hr, about 10 ng/1E8 cells/hr to about 20 ng/1E8 cells/hr, about 15 ng/1E8 cells/hr to about 20 ng/1E8 cells/hr, 5 ng/1E8 cells/hr to about 15 ng/1E8 cells/hr, about 10 ng/1E8 cells/hr to about 15 ng/1E8 cells/hr, or 5 ng/1E8 cells/hr to about 10 ng/1E8 cells/hr of IL-22 in vitro.

Pharmaceutically acceptable compositions comprising the bacteria and methods of treating or preventing disorders are also provided. In some embodiments, the composition is formulated for oral or rectal administration.

In another aspect, the disclosure provides for a method of treating or preventing a disorder comprising the step of administering to a patient in need thereof, the composition provided herein.

In some embodiments, the disorder is selected from a group consisting of autoimmune disorders, cancer, metabolic diseases, diseases relating to inborn errors of metabolism, and neurological or neurodegenerative diseases.

In some embodiments, the autoimmune disorder is selected from the group consisting of acute disseminated encephalomyelitis (ADEM), acute necrotizing hemorrhagic leukoencephalitis, Addison's disease, agammaglobulinemia, alopecia areata, amyloidosis, ankylosing spondylitis, anti-GBM/anti-TBM nephritis, antiphospholipid syndrome (APS), autoimmune angioedema, autoimmune aplastic anemia, autoimmune dysautonomia, autoimmune hemolytic anemia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenic purpura (ATP), autoimmune thyroid disease, autoimmune urticarial, Axonal & neuronal neuropathies, Balo disease, Behcet's disease, Bullous pemphigoid, Cardiomyopathy, Castleman disease, Celiac disease, Chagas disease, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogan syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibrosing alveolitis, Giant cell arteritis (temporal arteritis), Giant cell myocarditis, Glomerulonephritis, Goodpasture's syndrome, Granulomatosis with Polyangiitis (GPA), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile idiopathic arthritis, Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (Systemic Lupus Erythematosus), chronic Lyme disease, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatic, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, subacute bacterial endocarditis (SBE), Susac's syndrome, sympathetic ophthalmia, Takayasu's arteritis, temporal arteritis/giant cell arteritis, thrombocytopenic purpura (TTP), Tolosa-Hunt syndrome, transverse myelitis, type 1 diabetes, asthma, ulcerative colitis, undifferentiated connective tissue disease (UCTD), uveitis, vasculitis, vesiculobullous dermatosis, vitiligo, and Wegener's granulomatosis.

In some embodiments, the cancer is selected from adrenal cancer, adrenocortical carcinoma, anal cancer, appendix cancer, bile duct cancer, bladder cancer, bone cancer (e.g., Ewing sarcoma tumors, osteosarcoma, malignant fibrous histiocytoma), brain cancer (e.g., astrocytomas, brain stem glioma, craniopharyngioma, ependymoma), bronchial tumors, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, gestational trophoblastic disease, heart cancer, Kaposi sarcoma, kidney cancer, largyngeal cancer, hypopharyngeal cancer, leukemia (e.g., acute lymphoblastic leukemia, acute myeloid leukemia, chronic lymphocytic leukemia, chronic myelogenous leukemia), liver cancer, lung cancer, lymphoma (e.g., AIDS-related lymphoma, Burkitt lymphoma, cutaneous T cell lymphoma, Hodgkin lymphoma, Non-Hodgkin lymphoma, primary central nervous system lymphoma), malignant mesothelioma, multiple myeloma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma, skin cancer (e.g., basal cell carcinoma, melanoma), small intestine cancer, stomach cancer, teratoid tumor, testicular cancer, throat cancer, thymus cancer, thyroid cancer, unusual childhood cancers, urethral cancer, uterine cancer, uterine sarcoma, vaginal cancer, vulvar cancer, Waldenström macrogloblulinemia, and Wilms tumor.

In some embodiments, the metabolic disorder or condition is selected from the group consisting of: type 1 diabetes; type 2 diabetes; metabolic syndrome; Bardet-Biedel syndrome; Prader-Willi syndrome; non-alcoholic fatty liver disease; tuberous sclerosis; Albright hereditary osteodystrophy; brain-derived neurotrophic factor (BDNF) deficiency; Single-minded 1 (SIM1) deficiency; leptin deficiency; leptin receptor deficiency; pro-opiomelanocortin (POMC) defects; proprotein convertase subtilisin/kexin type 1 (PCSK1) deficiency; Src homology 2B1 (SH2B1) deficiency; pro-hormone convertase 1/3 deficiency; melanocortin-4-receptor (MC4R) deficiency; Wilms tumor, aniridia, genitourinary anomalies, and mental retardation (WAGR) syndrome; pseudohypoparathyroidism type 1A; Fragile X syndrome; Borjeson-Forsmann-Lehmann syndrome; Alstrom syndrome; Cohen syndrome; and ulnar-mammary syndrome.

### BRIEF DESCRIPTION OF THE FIGURES

FIGS. 1A-1C depict schematics of the gene organization of exemplary circuits of the disclosure for the expression of therapeutic polypeptides, which are secreted using components of the flagellar type III secretion system. A therapeutic polypeptide of interest, such as, GLP-2, IL-10, and IL-22, is assembled behind a fliC-5′UTR, and is driven by the native fliC and/or fliD promoter (FIG. 1A and FIG. 1B) or a tet-inducible promoter (FIG. 1C). The therapeutic polypeptide of interest is either expressed from a plasmid (e.g., a medium copy plasmid) or integrated into fliC loci (thereby deleting all or a portion of fliC and/or fliD). Optionally, an N terminal part of FliC is included in the construct, as shown in FIG. 1B and FIG. 1C.

FIG. 2A and FIG. 2B depict schematics of the gene organization of exemplary circuits of the disclosure for the expression of therapeutic polypeptides, which are secreted via a diffusible outer membrane (DOM) system. The therapeutic polypeptide of interest is fused to a prototypical N-terminal Sec-dependent secretion signal or Tat-dependent secretion signal, which is cleaved upon secretion into the periplasmic space. Exemplary secretion tags include sec-dependent PhoA, OmpF, OmpA, cvaC, and Tat-dependent tags (TorA, FdnG, DmsA). In certain embodiments, the recombinant bacteria comprise deletions in one or more of lpp, pal, tolA, and/or nlpI. Optionally, periplasmic proteases are also deleted, including, but not limited to, degP and ompT, e.g., to increase stability of the polypeptide in the periplasm. A FRT-KanR-FRT cassette is used for downstream integration. Expression is driven by a tet promoter (FIG. 2A) or an inducible promoter, such as oxygen level-dependent promoters (e.g., FNR-inducible promoter, FIG. 2B), promoters induced by inflammation or an inflammatory response (RNS, ROS promoters), and promoters induced by a metabolite that may or may not be naturally present (e.g., can be exogenously added) in the gut or the tumor microenvironment, e.g., arabinose.

FIG. 3 depicts a construct for the expression of GLP2-FcIgA-HIS fusion protein. The human glucagon-like peptide-2 (GLP-2) is fused to the N-terminus of the Fc portion of mouse IgA protein linked by a 20 amino acid linker. The Fc portion of mouse IgA used herein contains the hinge, CH2 and CH3 regions. An 8×HIS tag (SEQ ID NO: 1056) is fused to the C-terminus of FcIgA connected with a linker.

FIGS. 4A-4D depict schematics of non-limiting examples of recombinant bacteria of the disclosure which comprises one or more gene sequence(s) and/or gene cassette(s) as described herein.

FIG. 5 depicts a map of integration sites within the E. coli Nissle chromosome. These sites indicate regions where circuit components may be inserted into the chromosome without interfering with essential gene expression. Backslashes (/) are used to show that the insertion will occur between divergently or convergently expressed genes. Insertions within biosynthetic genes, such as thyA, can be useful for creating nutrient auxotrophies. In some embodiments, an individual circuit component is inserted into more than one of the indicated sites.

FIG. 6 depicts an exemplary schematic of the E. coli 1917 Nissle chromosome comprising multiple mechanisms of action (MoAs).

FIG. 7 depicts a schematic of a secretion system based on the flagellar type III secretion in which an incomplete flagellum is used to secrete a therapeutic peptide of interest (star) by recombinantly fusing the peptide to an N-terminal flagellar secretion signal of a native flagellar component so that the intracellularly expressed chimeric peptide can be mobilized across the inner and outer membranes into the surrounding host environment.

FIG. 8 depicts a schematic of a type V secretion system for the extracellular production of recombinant proteins in which a therapeutic peptide (star) can be fused to an N-terminal secretion signal, a linker and the beta-domain of an autotransporter.

FIG. 9 depicts a schematic of a type I secretion system, which translocates a passenger peptide directly from the cytoplasm to the extracellular space using HlyB (an ATP-binding cassette transporter); HlyD (a membrane fusion protein); and TolC (an outer membrane protein) which form a channel through both the inner and outer membranes. The secretion signal-containing C-terminal portion of HlyA is fused to the C-terminal portion of a therapeutic peptide (star) to mediate secretion of this peptide.

FIG. 10 depicts a schematic of the outer and inner membranes of a gram-negative bacterium, and several deletion targets for generating a leaky or destabilized outer membrane, thereby facilitating the translocation of a therapeutic polypeptides to the extracellular space, e.g., therapeutic polypeptides of eukaryotic origin containing disulphide bonds. Deactivating mutations of one or more genes encoding a protein that tethers the outer membrane to the peptidoglycan skeleton, e.g., lpp, ompC, ompA, ompF, tolA, tolB, pal, and/or one or more genes encoding a periplasmic protease, e.g., degS, degP, nlpl, generates a leaky phenotype. Combinations of mutations may synergistically enhance the leaky phenotype.

FIG. 11 depicts a modified type 3 secretion system (T3SS) to allow the bacteria to inject secreted therapeutic proteins into the gut lumen. An inducible promoter (small arrow, top), e.g. a FNR-inducible promoter, drives expression of the T3 secretion system gene cassette (3 large arrows, top) that produces the apparatus that secretes tagged peptides out of the cell. An inducible promoter (small arrow, bottom), e.g. a FNR-inducible promoter, drives expression of a regulatory factor, e.g. T7 polymerase, that then activates the expression of the tagged therapeutic peptide (hexagons).

FIG. 12 depicts the use of an antibiotic independent plasmid system (AIPS) as an engineered safety component. All engineered DNA is present on a plasmid which can be conditionally destroyed (Wright et al., ACS Synthetic Biology (2015) 4: 307-316).

FIGS. 13A-13D depict schematics of non-limiting examples of the gene organization of plasmids, which function as a component of a biosafety system (FIG. 13A and FIG. 13B), which also contains a chromosomal component (shown in FIG. 13C and FIG. 13D). The biosafety plasmid system vector comprises Kid Toxin and R6K minimal ori, dapA (FIG. 13A) and thyA (FIG. 13B) and promoter elements driving expression of these components. In some embodiments, bla is knocked out and replaced with one or more constructs described herein, in which a first protein of interest (POI1) and/or a second protein of interest, e.g., a transporter (POI2), and/or a third protein of interest (POI3) are expressed from an inducible or constitutive promoter. FIG. 13C and FIG. 13D depict schematics of the gene organization of the chromosomal component of a biosafety system. FIG. 13C depicts a construct comprising low copy Rep (Pi) and Kis antitoxin, in which transcription of Pi (Rep), which is required for the replication of the plasmid component of the system, is driven by a low copy RBS containing promoter. FIG. 13D depicts a construct comprising a medium-copy Rep (Pi) and Kis antitoxin, in which transcription of Pi (Rep), which is required for the replication of the plasmid component of the system, is driven by a medium copy RBS containing promoter. If the plasmid containing the functional DapA is used (as shown in FIG. 13A), then the chromosomal constructs shown in FIG. 13C and FIG. 13D are knocked into the DapA locus. If the plasmid containing the functional ThyA is used (as shown in FIG. 13B), then the chromosomal constructs shown in FIG. 13C and FIG. 13D are knocked into the ThyA locus. In this system, the bacteria comprising the chromosomal construct and a knocked out dapA or thyA gene can grow in the absence of dap or thymidine only in the presence of the plasmid.

FIG. 14 depicts a schematic of a polypeptide of interest displayed on the surface of the bacterium. A non-limiting example of such a therapeutic protein is a scFv. The polypeptide is expressed as a fusion protein, which comprises a outer membrane anchor from another protein, which was developed as part of a display system. Non-limiting examples of such anchors are described herein and include LppOmpA, NGIgAsig-NGIgAP, InaQ, Intimin, Invasin, pelB-PAL, and blcA/BAN. In a nonlimiting example, a bacterial strain has one or more diffusible outer membrane phenotype (“leaky membrane”) mutation, e.g., as described herein.

FIG. 15A depicts a schematic of the gene organization of a PssB promoter. The ssB gene product protects ssDNA from degradation; SSB interacts directly with numerous enzymes of DNA metabolism and is believed to have a central role in organizing the nucleoprotein complexes and processes involved in DNA replication (and replication restart), recombination and repair. The PssB promoter was cloned in front of a LacZ reporter and beta-galactosidase activity was measured. FIG. 15B depicts the reporter gene activity for the PssB promoter under aerobic and anaerobic conditions.

FIG. 16 depicts Western Blot analysis of total cytosolic extracts of a wild type E. coli (lane 1) and of a strain expressing anti-PD1 scFv (lane 2).

FIG. 17 depicts a diagram of a flow cytometric analysis of PD1 expressing EL4 cells which were incubated with extracts from a strain expressing tet inducible anti-PD1-scFv, and showing that anti-PD1-scFv expressed in E. coli binds to PD1 on mouse EL4 cells.

FIG. 18 depicts Western Blot analysis of total cytosolic extracts of various strain secreting anti-PD1 scFv. A single band was detected around 34 kDa in lane 1-6 corresponding to extracts from SYN2767, SYN2769, SYN2771, SYN2773, SYN2775 and SYN2777, respectively.

FIG. 19 depicts a diagram of a flow cytometric analysis of PD1 expressing EL4 cells, which were incubated with extracts from a E coli Nissle strain secreting tet-inducible anti-PD1-scFv, showing that anti-PD1-scFv secreted from E. coli Nissle binds to PD1 on mouse EL4 cells.

FIG. 20 depicts a diagram of a flow cytometric analysis of PD1 expressing EL4 cells, which were incubated with various amounts of extracts (0, 2, 5, and 15 ul) from an E. coli Nissle strain secreting tet-inducible anti-PD1-scFv, showing that anti-PD1-scFv secreted from E. coli Nissle binds to PD1 on mouse EL4 cells, in a dose dependent manner.

FIGS. 21A-21B depict diagrams of flow cytometric analysis of EL4 cells. A competition assay was conducted, in which extracts from a E coli Nissle strain secreting tet-inducible anti-PD1-scFv was incubated with various amounts of soluble PDL1 (0, 5, 10, and 30 ug) showing that PDL1 can dose-dependently compete with the binding of anti-PD1-scFv secreted from E. coli Nissle to PD1 on mouse EL4 cells.

FIG. 22 depicts Western blot analysis of bacterial supernatants showing murine CD40L1 (47-260) and CD40L2 (112-260) secreted by E. coli strains SYN3366 and SYN3367 are detected by mCD40 antibody.

FIG. 23 depicts Western blot analysis of bacterial supernatants from SYN2996 (lane 1), SYN3159 (lane 2), SYN3160 (lane 3), SYN3021 (lane 4), SYN3020 (lane 5), and SYN3161 (lane 6) showing that WT mSIRPα, mCV1SIRPα, mFD6x2SIRPα, mCV1SIRPα-IgG4, mFD6SIRPα-IgG4, and anti-mCD47 scFv are secreted from these strains, respectively.

FIG. 24 depicts a diagram of a flow cytometric analysis of CD47 expressing CT26 cells which were incubated with supernatants from a SYN1557 (1; delta PAL parental strain), SYN2996 (2; expressing tet inducible mSIRPα), SYN3021 (3; expressing tet inducible anti-mCD47scFv), SYN3161 (4; expressing tet inducible mCV1SIRPα-hIgG fusion) and showing that secreted products expressed in E. coli can bind to CD47 on mouse CT26 cells.

FIG. 25 depicts a diagram of a flow cytometric analysis of CD47 expressing CT26 cells which were incubated with supernatants from a SYN1557 (1; delta PAL parental strain), SYN3020 (2; expressing tet inducible mFD6SIRPα-hIgG fusion), SYN3160 (3; expressing tet inducible FD1x2SIRPα), SYN3159 (4; expressing tet inducible mCV1SIRPα), SYN3021 (5; expressing tet inducible mCV1SIRPα-hIgG fusion) and showing that secreted products expressed in E. coli can bind to CD47 on mouse CT26 cells.

FIG. 26 depicts a diagram of a flow cytometric analysis of CT26 cells. A competition assay was conducted, in which extracts from a E coli Nissle strain secreting tet-inducible murine SIRPalpha was incubated with recombinant SIRPalpha showing that recombinant SIRPalpha can compete with the binding of SIRPalpha secreted from E. coli Nissle to CD47 on CT26 cells.

FIG. 27 depicts a diagram of a flow cytometric analysis of CT26 cells. A competition assay was conducted, in which extracts from a E coli Nissle strain secreting tet-inducible murine SIRPalpha was incubated with an anti-CD47 antibody showing that the antibody can compete with the binding of SIRPalpha secreted from E. coli Nissle to CD47 on CT26 cells.

FIG. 28A depicts the circuitry for the secretion of mouse and human hyaluronidases expressed in SYN2997 and SYN2998. FIG. 28B depicts a Western blot analysis of bacterial supernatants from SYN2997 (lane 1) and SYN2998 (lane 2), showing that mouse and human hyaluronidases are secreted from these strains, respectively.

FIG. 29 depicts a bar graph showing hyaluronidase activity of SYN1557 (parental strain delta PAL), SYN2997 and SYN2998 as a measure of hyluronan degradation in an ELISA assay.

FIG. 30A depicts Western blot analysis of bacterial supernatants from SYN3369 expressing tetracycline inducible leech hyaluronidase (lane 1) and SYN1557 (parental strain delta PAL) (lane 2), showing that leech hyaluronidase is secreted from SYN3369. M=Marker. FIG. 30B and FIG. 30C depict bar graphs showing hyaluronidase activity as a measure of hyluronan degradation in an ELISA assay. FIG. 30B shows a positive control with recombinant hyaluronidase. FIG. 30C shows hyaluronidase activity of SYN1557 (parental strain delta PAL), and SYN3369 expressing tetracycline inducible leech hyaluronidase.

FIG. 31 depicts a bar graph showing levels of CXCL10 secreted from various strains into culture supernatant.

FIG. 32 depicts a bar graph showing levels of IL-15-Sushi fusion protein secreted from various strains into culture supernatant.

FIG. 33 depicts a schematic of a construct of the disclosure comprising a GLP2-linker-IL10-linker-IL-10 fusion protein.

FIG. 34 depicts a schematic of a construct of the disclosure comprising a GLP2-IL-22 fusion protein.

FIG. 35 depicts IL-22 secreting strains overview.

FIG. 36 depicts in vitro production and biological activity of secreted IL-12.

FIG. 37 depicts in vivo production and biological activity of secreted IL-12.

FIG. 38 depicts kinetics of IL-22 production in vivo during inflammation.

FIG. 39A depicts a schematic of IL-2 secreting strains. FIG. 39B depicts a schematic of DOM mutation. FIG. 39C depicts in vitro production and secretion of hIL-2 (pg/1E9 cells/hr).

FIG. 40A depicts a schematic of IL-2 secreting strains with Apal deletion. FIG. 40B depicts in vitro production and secretion of hIL-2 (ng/1E9 cells/hr). FIG. 40C depicts bioactive IL-2 concentration from IL-2 secreting E. coli compared to a control strain.

FIG. 41A depicts a schematic of IL15Rα-IL15 secreting strains. FIG. 41B depicts in vitro production and secretion of IL15Rα-IL15 (ng/1E9 cells/hr). FIG. 41C depicts bioactive IL15Rα-IL15 concentration from IL15Rα-IL15 secreting E. coli compared to a control strain.

FIG. 42A depicts a schematic of the protein-producing bacterial strain (hIL-22) containing ΔPAL mutation (parental strain SYN1557), FnR promoter, optimized RBS and signaling peptide (phoA). FIG. 42B depicts bar graph measurement of IL-22 production in vitro from the engineered E. coli Nissle strain (EcN-IL22) compared to the control strains with incomplete secretion machinery: Secretion depends on the presence of signaling peptide phoA and significantly increases with engineering DOM (ΔPAL) mutant as compared to WT strain (parental strain SYN094).

### DETAILED DESCRIPTION

The present disclosure relates to compositions of recombinant bacteria and methods for secreting therapeutically active proteins from recombinant bacteria for treatment of diseases or disorders. The recombinant bacteria disclosed herein are capable of high yield production of functionally active effector molecules, which are secreted as therapeutically active polypeptides.

### Definitions

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, “effector molecules” or “therapeutic molecules” or “therapeutic polypeptides” include, but are not limited to, anti-inflammation molecules and/or gut barrier function enhancer molecules, such as GLP-2, IL-22, IL-10, IL-27, IL-19, IL-20, IL-25, IL-15, IL-2, IL-1beta, IL-6, IL-8, UL-17, GMCSF, TNFalpha, IFNgamma, CXCL-8, CCL2, CXCL10, CXCL9, hyaluronidase, CLTA-4, PD1, PDL1, CD47, and CD40L. Such molecules may also include compounds that inhibit pro-inflammatory molecules, e.g., a single-chain variable fragment (scFv), antisense RNA, siA, or shRNA that neutralizes TNF-α, IFN-γ, IL-113, IL-6, IL-8, IL-17, and/or chemokines, e.g., CXCL-8 and CCL2. A molecule may be primarily anti-inflammatory, e.g., IL-10, or primarily gut barrier function enhancing, e.g., GLP-2. A molecule may be both anti-inflammatory and gut barrier function enhancing. An anti-inflammation and/or gut barrier function enhancer molecule may be encoded by a single gene, e.g., elafin is encoded by the PI3 gene. Alternatively, an anti-inflammation and/or gut barrier function enhancer molecule may be synthesized by a biosynthetic pathway requiring multiple genes, e.g., butyrate. In some instances, the effector molecules” or “therapeutic molecules” or “therapeutic polypeptides” are referred to herein as “anti-inflammation molecules” and/or “gut barrier function enhancer molecules”.

As used herein, the term “recombinant microorganism” refers to a microorganism, e.g., bacterial, yeast, or viral cell, or bacteria, yeast, or virus, that has been genetically modified from its native state. Thus, a “recombinant bacterial cell” or “recombinant bacteria” refers to a bacterial cell or bacteria that have been genetically modified from their native state. For instance, a recombinant bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Recombinant bacterial cells disclosed herein may comprise exogenous nucleotide sequences on plasmids. Alternatively, recombinant bacterial cells may comprise exogenous nucleotide sequences stably incorporated into their chromosome.

A “programmed or engineered microorganism” refers to a microorganism, e.g., bacterial or viral cell, or bacteria or virus, that has been genetically modified from its native state to perform a specific function. Thus, a “programmed or engineered bacterial cell” or “programmed or engineered bacteria” refers to a bacterial cell or bacteria that has been genetically modified from its native state to perform a specific function. In certain embodiments, the programmed or engineered bacterial cell has been modified to express one or more proteins, for example, one or more proteins that have a therapeutic activity or serve a therapeutic purpose. The programmed or engineered bacterial cell may additionally have the ability to stop growing or to destroy itself once the protein(s) of interest have been expressed.

As used herein, the term “gene” refers to a nucleic acid fragment that encodes a protein or fragment thereof, optionally including regulatory sequences preceding (5′ non-coding sequences) and following (3′ non-coding sequences) the coding sequence. In one embodiment, a “gene” does not include regulatory sequences preceding and following the coding sequence. A “native gene” refers to a gene as found in nature, optionally with its own regulatory sequences preceding and following the coding sequence. A “chimeric gene” refers to any gene that is not a native gene, optionally comprising regulatory sequences preceding and following the coding sequence, wherein the coding sequences and/or the regulatory sequences, in whole or in part, are not found together in nature. Thus, a chimeric gene may comprise regulatory sequences and coding sequences that are derived from different sources, or regulatory and coding sequences that are derived from the same source, but arranged differently than is found in nature.

As used herein, the term “gene sequence” is meant to refer to a genetic sequence, e.g., a nucleic acid sequence. The gene sequence or genetic sequence is meant to include a complete gene sequence or a partial gene sequence. The gene sequence or genetic sequence is meant to include sequence that encodes a protein or polypeptide and is also meant to include genetic sequence that does not encode a protein or polypeptide, e.g., a regulatory sequence, leader sequence, signal sequence, or other non-protein coding sequence.

In some embodiments, the term “gene” or “gene sequence” is meant to refer to a nucleic acid sequence encoding any of the effector molecules described herein, e.g., gut barrier enhancers, anti or proinflammatory, anti-cancer molecules and others. The nucleic acid sequence may comprise the entire gene sequence or a partial gene sequence encoding a functional molecule. The nucleic acid sequence may be a natural sequence or a synthetic sequence. The nucleic acid sequence may comprise a native or wild-type sequence or may comprise a modified sequence having one or more insertions, deletions, substitutions, or other modifications, for example, the nucleic acid sequence may be codon-optimized.

As used herein, a “heterologous” gene or “heterologous sequence” refers to a nucleotide sequence that is not normally found in a given cell in nature. As used herein, a heterologous sequence encompasses a nucleic acid sequence that is exogenously introduced into a given cell and can be a native sequence (naturally found or expressed in the cell) or non-native sequence (not naturally found or expressed in the cell) and can be a natural or wild-type sequence or a variant, non-natural, or synthetic sequence. “Heterologous gene” includes a native gene, or fragment thereof, that has been introduced into the host cell in a form that is different from the corresponding native gene. For example, a heterologous gene may include a native coding sequence that is a portion of a chimeric gene to include non-native regulatory regions that is reintroduced into the host cell. A heterologous gene may also include a native gene, or fragment thereof, introduced into a non-native host cell. Thus, a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature. As used herein, the term “endogenous gene” refers to a native gene in its natural location in the genome of an organism. As used herein, the term “transgene” refers to a gene that has been introduced into the host organism, e.g., host bacterial cell, genome.

As used herein, a “non-native” nucleic acid sequence refers to a nucleic acid sequence not normally present in a microorganism, e.g., an extra copy of an endogenous sequence, or a heterologous sequence such as a sequence from a different species, strain, or substrain of bacteria or virus, or a sequence that is modified and/or mutated as compared to the unmodified sequence from bacteria or virus of the same subtype. In some embodiments, the non-native nucleic acid sequence is a synthetic, non-naturally occurring sequence (see, e.g., Purcell et al., 2013). The non-native nucleic acid sequence may be a regulatory region, a promoter, a gene, and/or one or more genes in gene cassette. In some embodiments, “non-native” refers to two or more nucleic acid sequences that are not found in the same relationship to each other in nature. The non-native nucleic acid sequence may be present on a plasmid or chromosome. In some embodiments, the genetically engineered microorganism of the disclosure comprises a gene that is operably linked to a promoter that is not associated with said gene in nature. For example, in some embodiments, the recombinant bacteria disclosed herein comprise a gene that is operably linked to a directly or indirectly inducible promoter that is not associated with said gene in nature, e.g., an FNR responsive promoter (or other promoter disclosed herein) operably linked to an effector molecule, e.g., an anti-inflammatory or gut barrier enhancer molecule, or a pro-inflammatory or an anti-cancer molecule.

As used herein, the term “coding region” refers to a nucleotide sequence that codes for a specific amino acid sequence. The term “regulatory sequence” refers to a nucleotide sequence located upstream (5′ non-coding sequences), within, or downstream (3′ non-coding sequences) of a coding sequence, and which influences the transcription, RNA processing, RNA stability, or translation of the associated coding sequence. Examples of regulatory sequences include, but are not limited to, promoters, translation leader sequences, effector binding sites, signal sequences, and stem-loop structures. In one embodiment, the regulatory sequence comprises a promoter, e.g., an FNR responsive promoter or other promoter disclosed herein.

As used herein, a “gene cassette” or “operon” encoding a biosynthetic pathway refers to the two or more genes that are required to produce an effector molecule, e.g., an anti-inflammatory or gut barrier enhancer molecule or a pro-inflammatory or an anti-cancer molecule or another effector molecule. In addition to encoding a set of genes capable of producing said molecule, the gene cassette or operon may also comprise additional transcription and translation elements, e.g., a ribosome binding site.

A regulatory region “operably linked” refers to the association of nucleic acid sequences on a single nucleic acid fragment so that the function of one is affected by the other. A regulatory element is operably linked with a coding sequence when it is capable of affecting the expression of the gene coding sequence, regardless of the distance between the regulatory element and the coding sequence. More specifically, operably linked refers to a nucleic acid sequence, e.g., a gene encoding an effector molecule, that is joined to a regulatory sequence in a manner which allows expression of the nucleic acid sequence, e.g., the gene encoding the effector molecule described herein. In other words, the regulatory sequence acts in cis. In one embodiment, a gene may be “directly linked” to a regulatory sequence in a manner which allows expression of the gene. In another embodiment, a gene may be “indirectly linked” to a regulatory sequence in a manner which allows expression of the gene. In one embodiment, two or more genes may be directly or indirectly linked to a regulatory sequence in a manner which allows expression of the two or more genes. A regulatory region or sequence is a nucleic acid that can direct transcription of a gene of interest and may comprise promoter sequences, enhancer sequences, response elements, protein recognition sites, inducible elements, promoter control elements, protein binding sequences, 5′ and 3′-untranslated regions, transcriptional start sites, termination sequences, polyadenylation sequences and introns.

A “promoter” as used herein, refers to a nucleotide sequence that is capable of controlling the expression of a coding sequence or gene. Promoters are generally located 5′ of the sequence that they regulate. Promoters may be derived in their entirety from a native gene, or be composed of different elements derived from promoters found in nature, and/or comprise synthetic nucleotide segments. Those skilled in the art will readily ascertain that different promoters may regulate expression of a coding sequence or gene in response to a particular stimulus, e.g., in a cell- or tissue-specific manner, in response to different environmental or physiological conditions, or in response to specific compounds. Prokaryotic promoters are typically classified into two classes: inducible and constitutive. A “constitutive promoter” refers to a promoter that allows for continual transcription of the coding sequence or gene under its control.

“Constitutive promoter” refers to a promoter that is capable of facilitating continuous transcription of a coding sequence or gene under its control and/or to which it is operably linked. Constitutive promoters and variants are well known in the art and include, but are not limited to, Ptac promoter, BBa_J23100, a constitutive Escherichia coli σS promoter (e.g., an osmY promoter (International Genetically Engineered Machine (iGEM) Registry of Standard Biological Parts Name BBa_J45992; BBa_J45993)), a constitutive Escherichia coli σ32 promoter (e.g., htpG heat shock promoter (BBa_J45504)), a constitutive Escherichia coli σ70 promoter (e.g., lacq promoter (BBa_J54200; BBa_J56015), E. coli CreABCD phosphate sensing operon promoter (BBa_J64951), GlnRS promoter (BBa_K088007), lacZ promoter (BBa_K119000; BBa_K119001); M13K07 gene I promoter (BBa_M13101); M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), M13110 (BBa_M13110)), a constitutive Bacillus subtilis σA promoter (e.g., promoter veg (BBa_K143013), promoter 43 (BBa_K143013), PliaG (BBa_K823000), PlepA (BBa_K823002), Pveg (BBa_K823003)), a constitutive Bacillus subtilis σB promoter (e.g., promoter ctc (BBa_K143010), promoter gsiB (BBa_K143011)), a Salmonella promoter (e.g., Pspv2 from Salmonella (BBa_K112706), Pspv from Salmonella (BBa_K112707)), a bacteriophage T7 promoter (e.g., T7 promoter (BBa_I712074; BBa_I719005; BBa_J34814; BBa_J64997; BBa_K113010; BBa_K113011; BBa_K113012; BBa_R0085; BBa_R0180; BBa_R0181; BBa_R0182; BBa_R0183; BBa_Z0251; BBa_Z0252; BBa_Z0253)), and a bacteriophage SP6 promoter (e.g., SP6 promoter (BBa_J64998)).

An “inducible promoter” refers to a regulatory region that is operably linked to one or more genes, wherein expression of the gene(s) is increased in the presence of an inducer of said regulatory region. An “inducible promoter” refers to a promoter that initiates increased levels of transcription of the coding sequence or gene under its control in response to a stimulus or an exogenous environmental condition. A “directly inducible promoter” refers to a regulatory region, wherein the regulatory region is operably linked to a gene encoding a protein or polypeptide, where, in the presence of an inducer of said regulatory region, the protein or polypeptide is expressed. An “indirectly inducible promoter” refers to a regulatory system comprising two or more regulatory regions, for example, a first regulatory region that is operably linked to a first gene encoding a first protein, polypeptide, or factor, e.g., a transcriptional regulator, which is capable of regulating a second regulatory region that is operably linked to a second gene, the second regulatory region may be activated or repressed, thereby activating or repressing expression of the second gene. Both a directly inducible promoter and an indirectly inducible promoter are encompassed by “inducible promoter.”

As used herein, “stably maintained” or “stable” bacterium is used to refer to a bacterial host cell carrying non-native genetic material, e.g., a gene encoding one or more effector molecule(s), which is incorporated into the host genome or propagated on a self-replicating extra-chromosomal plasmid, such that the non-native genetic material is retained, expressed, and propagated. The stable bacterium is capable of survival and/or growth in vitro, e.g., in medium, and/or in vivo, e.g., in the gut. For example, the stable bacterium may be a recombinant bacterium comprising a gene encoding a encoding a payload, e.g., one or more effector molecule(s), in which the plasmid or chromosome carrying the gene is stably maintained in the bacterium, such that the payload can be expressed in the bacterium, and the bacterium is capable of survival and/or growth in vitro and/or in vivo. In some embodiments, copy number affects the stability of expression of the non-native genetic material. In some embodiments, copy number affects the level of expression of the non-native genetic material.

As used herein, the term “expression” refers to the transcription and stable accumulation of sense (mRNA) or anti-sense RNA derived from a nucleic acid, and/or to translation of an mRNA into a polypeptide.

As used herein, the term “plasmid” or “vector” refers to an extrachromosomal nucleic acid, e.g., DNA, construct that is not integrated into a bacterial cell's genome. Plasmids are usually circular and capable of autonomous replication. Plasmids may be low-copy, medium-copy, or high-copy, as is well known in the art. Plasmids may optionally comprise a selectable marker, such as an antibiotic resistance gene, which helps select for bacterial cells containing the plasmid and which ensures that the plasmid is retained in the bacterial cell. A plasmid disclosed herein may comprise a nucleic acid sequence encoding a heterologous gene, e.g., a gene encoding effector molecule.

As used herein, the term “transform” or “transformation” refers to the transfer of a nucleic acid fragment into a host bacterial cell, resulting in genetically-stable inheritance. Host bacterial cells comprising the transformed nucleic acid fragment are referred to as “recombinant” or “transgenic” or “transformed” organisms.

The term “genetic modification,” as used herein, refers to any genetic change. Exemplary genetic modifications include those that increase, decrease, or abolish the expression of a gene, including, for example, modifications of native chromosomal or extrachromosomal genetic material. Exemplary genetic modifications also include the introduction of at least one plasmid, modification, mutation, base deletion, base addition, base substitution, and/or codon modification of chromosomal or extrachromosomal genetic sequence(s), gene over-expression, gene amplification, gene suppression, promoter modification or substitution, gene addition (either single or multi-copy), antisense expression or suppression, or any other change to the genetic elements of a host cell, whether the change produces a change in phenotype or not. Genetic modification can include the introduction of a plasmid, e.g., a plasmid comprising an effector molecule operably linked to a promoter, into a bacterial cell. Genetic modification can also involve a targeted replacement in the chromosome, e.g., to replace a native gene promoter with an inducible promoter, regulated promoter, strong promoter, or constitutive promoter. Genetic modification can also involve gene amplification, e.g., introduction of at least one additional copy of a native gene into the chromosome of the cell. Alternatively, chromosomal genetic modification can involve a genetic mutation.

As used herein, the term “genetic mutation” refers to a change or changes in a nucleotide sequence of a gene or related regulatory region that alters the nucleotide sequence as compared to its native or wild-type sequence. Mutations include, for example, substitutions, additions, and deletions, in whole or in part, within the wild-type sequence. Such substitutions, additions, or deletions can be single nucleotide changes (e.g., one or more point mutations), or can be two or more nucleotide changes, which may result in substantial changes to the sequence. Mutations can occur within the coding region of the gene as well as within the non-coding and regulatory sequence of the gene. The term “genetic mutation” is intended to include silent and conservative mutations within a coding region as well as changes which alter the amino acid sequence of the polypeptide encoded by the gene. A genetic mutation in a gene coding sequence may, for example, increase, decrease, or otherwise alter the activity (e.g., enzymatic activity) of the gene's polypeptide product. A genetic mutation in a regulatory sequence may increase, decrease, or otherwise alter the expression of sequences operably linked to the altered regulatory sequence.

As used herein, the term “transporter” is meant to refer to a mechanism, e.g., protein, proteins, or protein complex, for importing a molecule, e g, amino acid, peptide (di-peptide, tri-peptide, polypeptide, etc), toxin, metabolite, substrate, as well as other biomolecules into the microorganism from the extracellular milieu.

As used herein, the phrase “exogenous environmental condition” or “exogenous environment signal” refers to settings, circumstances, stimuli, or biological molecules under which a promoter described herein is directly or indirectly induced. The phrase “exogenous environmental conditions” is meant to refer to the environmental conditions external to the engineered microorganism, but endogenous or native to the host subject environment. Thus, “exogenous” and “endogenous” may be used interchangeably to refer to environmental conditions in which the environmental conditions are endogenous to a mammalian body, but external or exogenous to an intact microorganism cell. In some embodiments, the exogenous environmental conditions are specific to the gut of a mammal. In some embodiments, the exogenous environmental conditions are specific to the upper gastrointestinal tract of a mammal. In some embodiments, the exogenous environmental conditions are specific to the lower gastrointestinal tract of a mammal. In some embodiments, the exogenous environmental conditions are specific to the small intestine of a mammal. In some embodiments, the exogenous environmental conditions are specific to the tumor microenvironment. In some embodiments, the exogenous environmental conditions are low-oxygen, microaerobic, or anaerobic conditions, such as the environment of the mammalian gut or the tumor microenvironment. In some embodiments, exogenous environmental conditions are molecules or metabolites that are specific to the mammalian gut, e.g., propionate. In some embodiments, the exogenous environmental condition is a tissue-specific or disease-specific metabolite or molecule(s). In some embodiments, the exogenous environmental condition is specific to an inflammatory disease. In some embodiments, the exogenous environmental condition is a low-pH environment. In some embodiments, the genetically engineered microorganism of the disclosure comprises a pH-dependent promoter. In some embodiments, the genetically engineered microorganism of the disclosure comprise an oxygen level-dependent promoter. In some aspects, bacteria have evolved transcription factors that are capable of sensing oxygen levels. Different signaling pathways may be triggered by different oxygen levels and occur with different kinetics. An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression.

Examples of oxygen level-dependent transcription factors include, but are not limited to, FNR (fumarate and nitrate reductase), ANR (anaerobic nitrate respiration), and DNR (dissimilatory nitrate respiration regulator). Corresponding FNR-responsive promoters, ANR-responsive promoters, and DNR-responsive promoters are known in the art (see, e.g., Castiglione et al., 2009; Eiglmeier et al., 1989; Galimand et al., 1991; Hasegawa et al., 1998; Hoeren et al., 1993; Salmon et al., 2003), and non-limiting examples are shown in Table 1A.

In a non-limiting example, a promoter (PfnrS) was derived from the E. coli Nissle fumarate and nitrate reductase gene S (fnrS) that is known to be highly expressed under conditions of low or no environmental oxygen (Durand and Storz, 2010; Boysen et al, 2010). The PfnrS promoter is activated under anaerobic conditions by the global transcriptional regulator FNR that is naturally found in Nissle. Under anaerobic conditions, FNR forms a dimer and binds to specific sequences in the promoters of specific genes under its control, thereby activating their expression. However, under aerobic conditions, oxygen reacts with iron-sulfur clusters in FNR dimers and converts them to an inactive form. In this way, the PfnrS inducible promoter is adopted to modulate the expression of proteins or RNA. PfnrS is used interchangeably in this application as FNRS, fnrs, FNR, P-FNRS promoter and other such related designations to indicate the promoter PfnrS.

TABLE 1A
Examples of transcription factors and
responsive genes and regulatory regions

| | Examples of responsive genes, promoters, |
|---|---|
| Transcription Factor | and/or regulatory regions: |
| FNR | nirB, ydfZ, pdhR, focA, ndH, hlyE, narK, |
| | narX, narG, yfiD, tdcD |
| ANR | arcDABC |
| DNR | norb, norC |

As used herein, a “tunable regulatory region” refers to a nucleic acid sequence under direct or indirect control of a transcription factor and which is capable of activating, repressing, derepressing, or otherwise controlling gene expression relative to levels of an inducer. In some embodiments, the tunable regulatory region comprises a promoter sequence. The inducer may be RNS, or other inducer described herein, and the tunable regulatory region may be a RNS-responsive regulatory region or other responsive regulatory region described herein. The tunable regulatory region may be operatively linked to a gene sequence(s) or gene cassette for the production of one or more payloads, e.g., an effector molecule gene cassette or gene sequence(s). For example, in one specific embodiment, the tunable regulatory region is a RNS-derepressible regulatory region, and when RNS is present, a RNS-sensing transcription factor no longer binds to and/or represses the regulatory region, thereby permitting expression of the operatively linked gene or gene cassette. In this instance, the tunable regulatory region derepresses gene or gene cassette expression relative to RNS levels. Each gene or gene cassette may be operatively linked to a tunable regulatory region that is directly or indirectly controlled by a transcription factor that is capable of sensing at least one RNS.

In some embodiments, the exogenous environmental conditions are the presence or absence of reactive oxygen species (ROS). In other embodiments, the exogenous environmental conditions are the presence or absence of reactive nitrogen species (RNS). In some embodiments, exogenous environmental conditions are biological molecules that are involved in the inflammatory response, for example, molecules present in an inflammatory disorder of the gut. In some embodiments, the exogenous environmental conditions or signals exist naturally or are naturally absent in the environment in which the recombinant bacterial cell resides. In some embodiments, the exogenous environmental conditions or signals are artificially created, for example, by the creation or removal of biological conditions and/or the administration or removal of biological molecules.

In some embodiments, the exogenous environmental condition(s) and/or signal(s) stimulates the activity of an inducible promoter. In some embodiments, the exogenous environmental condition(s) and/or signal(s) that serves to activate the inducible promoter is not naturally present within the gut or any other organ of a mammal. In some embodiments, the inducible promoter is stimulated by a molecule or metabolite that is administered in combination with the pharmaceutical composition of the disclosure, for example, tetracycline, arabinose, or any biological molecule that serves to activate an inducible promoter. In some embodiments, the exogenous environmental condition(s) and/or signal(s) is added to culture media comprising a recombinant bacterial cell of the disclosure. In some embodiments, the exogenous environmental condition that serves to activate the inducible promoter is naturally present within the gut of a mammal (for example, low oxygen or anaerobic conditions, or biological molecules involved in an inflammatory response). In some embodiments, the molecule that activates the inducible promoter is present in the tumor microenvironment. In some embodiments, the loss of exposure to an exogenous environmental condition (for example, in vivo) inhibits the activity of an inducible promoter, as the exogenous environmental condition is not present to induce the promoter (for example, an aerobic environment outside the gut). “Gut” refers to the organs, glands, tracts, and systems that are responsible for the transfer and digestion of food, absorption of nutrients, and excretion of waste. In humans, the gut comprises the gastrointestinal (GI) tract, which starts at the mouth and ends at the anus, and additionally comprises the esophagus, stomach, small intestine, and large intestine. The gut also comprises accessory organs and glands, such as the spleen, liver, gallbladder, and pancreas. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines.

“Hypoxia” is used to refer to reduced oxygen supply to a tissue as compared to physiological levels, thereby creating an oxygen-deficient environment. “Normoxia” refers to a physiological level of oxygen supply to a tissue. Hypoxia is a hallmark of solid tumors and characterized by regions of low oxygen and necrosis due to insufficient perfusion.

As used herein, the term “low oxygen” is meant to refer to a level, amount, or concentration of oxygen (O₂) that is lower than the level, amount, or concentration of oxygen that is present in the atmosphere (e.g., <21% O₂; <160 torr O₂)). Thus, the term “low oxygen condition or conditions” or “low oxygen environment” refers to conditions or environments containing lower levels of oxygen than are present in the atmosphere. In some embodiments, the term “low oxygen” is meant to refer to the level, amount, or concentration of oxygen (O₂) found in a mammalian gut, e.g., lumen, stomach, small intestine, duodenum, jejunum, ileum, large intestine, cecum, colon, distal sigmoid colon, rectum, and anal canal. In some embodiments, the term “low oxygen” is meant to refer to a level, amount, or concentration of O₂ that is 0-60 mmHg O₂ (0-60 torr O₂) (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60 mmHg O₂), including any and all incremental fraction(s) thereof (e.g., 0.2 mmHg, 0.5 mmHg O₂, 0.75 mmHg O₂, 1.25 mmHg O₂, 2.175 mmHg O₂, 3.45 mmHg O₂, 3.75 mmHg O₂, 4.5 mmHg O₂, 6.8 mmHg O₂, 11.35 mmHg O₂, 46.3 mmHg O₂, 58.75 mmHg, etc., which exemplary fractions are listed here for illustrative purposes and not meant to be limiting in any way). In some embodiments, “low oxygen” refers to about 60 mmHg O₂ or less (e.g., 0 to about 60 mmHg O₂). The term “low oxygen” may also refer to a range of O₂ levels, amounts, or concentrations between 0-60 mmHg O₂ (inclusive), e.g., 0-5 mmHg O₂, <1.5 mmHg O₂, 6-10 mmHg, <8 mmHg, 47-60 mmHg, etc. which listed exemplary ranges are listed here for illustrative purposes and not meant to be limiting in any way. See, for example, Albenberg et al., Gastroenterology, 147(5): 1055-1063 (2014); Bergofsky et al., J Clin. Invest., 41(11): 1971-1980 (1962); Crompton et al., J Exp. Biol., 43: 473-478 (1965); He et al., PNAS (USA), 96: 4586-4591 (1999); McKeown, Br. J. Radiol., 87:20130676 (2014) (doi: 10.1259/brj.20130676), each of which discusses the oxygen levels found in the mammalian gut of various species and each of which are incorportated by reference herewith in their entireties. In some embodiments, the term “low oxygen” is meant to refer to the level, amount, or concentration of oxygen (O₂) found in a mammalian organ or tissue other than the gut, e.g., urogenital tract, tumor tissue, etc. in which oxygen is present at a reduced level, e.g., at a hypoxic or anoxic level. In some embodiments, “low oxygen” is meant to refer to the level, amount, or concentration of oxygen (O₂) present in partially aerobic, semi aerobic, microaerobic, nanoaerobic, microoxic, hypoxic, anoxic, and/or anaerobic conditions. For example, Table 1B summarizes the amount of oxygen present in various organs and tissues. In some embodiments, the level, amount, or concentration of oxygen (O₂) is expressed as the amount of dissolved oxygen (“DO”) which refers to the level of free, non-compound oxygen (O₂) present in liquids and is typically reported in milligrams per liter (mg/L), parts per million (ppm; 1 mg/L=1 ppm), or in micromoles (umole) (1 umole O₂=0.022391 mg/L O₂). Fondriest Environmental, Inc., “Dissolved Oxygen”, Fundamentals of Environmental Measurements, 19 Nov. 2013, www.fondriest.com/environmental-measurements/parameters/water-quality/dissolved-oxygen/>. In some embodiments, the term “low oxygen” is meant to refer to a level, amount, or concentration of oxygen (O₂) that is about 6.0 mg/L DO or less, e.g., 6.0 mg/L, 5.0 mg/L, 4.0 mg/L, 3.0 mg/L, 2.0 mg/L, 1.0 mg/L, or 0 mg/L, and any fraction therein, e.g., 3.25 mg/L, 2.5 mg/L, 1.75 mg/L, 1.5 mg/L, 1.25 mg/L, 0.9 mg/L, 0.8 mg/L, 0.7 mg/L, 0.6 mg/L, 0.5 mg/L, 0.4 mg/L, 0.3 mg/L, 0.2 mg/L and 0.1 mg/L DO, which exemplary fractions are listed here for illustrative purposes and not meant to be limiting in any way. The level of oxygen in a liquid or solution may also be reported as a percentage of air saturation or as a percentage of oxygen saturation (the ratio of the concentration of dissolved oxygen (O₂) in the solution to the maximum amount of oxygen that will dissolve in the solution at a certain temperature, pressure, and salinity under stable equilibrium). Well-aerated solutions (e.g., solutions subjected to mixing and/or stirring) without oxygen producers or consumers are 100% air saturated. In some embodiments, the term “low oxygen” is meant to refer to 40% air saturation or less, e.g., 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, and 0% air saturation, including any and all incremental fraction(s) thereof (e.g., 30.25%, 22.70%, 15.5%, 7.7%, 5.0%, 2.8%, 2.0%, 1.65%, 1.0%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of air saturation levels between 0-40%, inclusive (e.g., 0-5%, 0.05-0.1%, 0.1-0.2%, 0.1-0.5%, 0.5-2.0%, 0-10%, 5-10%, 10-15%, 15-20%, 20-25%, 25-30%, etc.). The exemplary fractions and ranges listed here are for illustrative purposes and not meant to be limiting in any way. In some embodiments, the term “low oxygen” is meant to refer to 9% O₂ saturation or less, e.g., 9%, 8%, 7%, 6%, 5%, 4%, 3%, 2%, 1%, 0%, O₂ saturation, including any and all incremental fraction(s) thereof (e.g., 6.5%, 5.0%, 2.2%, 1.7%, 1.4%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of O₂ saturation levels between 0-9%, inclusive (e.g., 0-5%, 0.05-0.1%, 0.1-0.2%, 0.1-0.5%, 0.5-2.0%, 0-8%, 5-7%, 0.3-4.2% O₂, etc.). The exemplary fractions and ranges listed here are for illustrative purposes and not meant to be limiting in any way.

TABLE 1B

| Compartment | Oxygen Tension |
|---|---|
| stomach | ~60 torr (e.g., 58 +/− 15 torr) |
| duodenum and first part of | ~30 torr (e.g., 32 +/− 8 torr); ~20% |
| jejunum | oxygen in ambient air |
| Ileum (mid- small intestine) | ~10 torr; ~6% oxygen in ambient |
| | air (e.g., 11 +/− 3 torr) |
| Distal sigmoid colon | ~3 torr (e.g., 3 +/− 1 torr) |
| colon | <2 torr |
| Lumen of cecum | <1 torr |
| tumor | <32 torr (most tumors are <15 torr) |

“Microorganism” refers to an organism or microbe of microscopic, submicroscopic, or ultramicroscopic size that typically consists of a single cell. Examples of microorganisms include bacteria, viruses, parasites, fungi, certain algae, yeast, e.g., Saccharomyces, and protozoa. In some aspects, the microorganism is engineered (“engineered microorganism”) to produce one or more therapeutic molecules, e.g., an antiinflammatory or barrier enhancer molecule. In certain embodiments, the engineered microorganism is an engineered bacterium.

“Non-pathogenic bacteria” refer to bacteria that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria do not contain lipopolysaccharides (LPS). In some embodiments, non-pathogenic bacteria are commensal bacteria. Examples of non-pathogenic bacteria include, but are not limited to certain strains belonging to the genus Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Escherichia coli Nissle, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis and Saccharomyces boulardii (Sonnenborn et al., 2009; Dinleyici et al., 2014; U.S. Pat. Nos. 6,835,376; 6,203,797; 5,589,168; 7,731,976). Non-pathogenic bacteria also include commensal bacteria, which are present in the indigenous microbiota of the gut. In one embodiment, the disclosure further includes non-pathogenic Saccharomyces, such as Saccharomyces boulardii. Naturally pathogenic bacteria may be genetically engineered to reduce or eliminate pathogenicity.

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. In some embodiments, the probiotic bacteria are Gram-negative bacteria. In some embodiments, the probiotic bacteria are Gram-positive bacteria. Some species, strains, and/or subtypes of non-pathogenic bacteria are currently recognized as probiotic bacteria. Examples of probiotic bacteria include, but are not limited to, certain strains belonging to the genus Bifidobacteria, Escherichia Coli, Lactobacillus, and Saccharomyces e.g., Bifidobacterium bifidum, Enterococcus faecium, Escherichia coli strain Nissle, Lactobacillus acidophilus, Lactobacillus bulgaricus, Lactobacillus paracasei, and Lactobacillus plantarum, and Saccharomyces boulardii (Dinleyici et al., 2014; U.S. Pat. Nos. 5,589,168; 6,203,797; 6,835,376).

The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, the term “auxotroph” or “auxotrophic” refers to an organism that requires a specific factor, e.g., an amino acid, a sugar, or other nutrient) to support its growth. An “auxotrophic modification” is a genetic modification that causes the organism to die in the absence of an exogenously added nutrient essential for survival or growth because it is unable to produce said nutrient. As used herein, the term “essential gene” refers to a gene which is necessary to for cell growth and/or survival. Essential genes are described in more detail infra and include, but are not limited to, DNA synthesis genes (such as thyA), cell wall synthesis genes (such as dapA), and amino acid genes (such as serA and metA).

As used herein, the term “modulate” and its cognates means to alter, regulate, or adjust positively or negatively a molecular or physiological readout, outcome, or process, to effect a change in said readout, outcome, or process as compared to a normal, average, wild-type, or baseline measurement. Thus, for example, “modulate” or “modulation” includes up-regulation and down-regulation. A non-limiting example of modulating a readout, outcome, or process is effecting a change or alteration in the normal or baseline functioning, activity, expression, or secretion of a biomolecule (e.g. a protein, enzyme, cytokine, growth factor, hormone, metabolite, short chain fatty acid, or other compound). Another non-limiting example of modulating a readout, outcome, or process is effecting a change in the amount or level of a biomolecule of interest, e.g. in the serum and/or the gut lumen. In another non-limiting example, modulating a readout, outcome, or process relates to a phenotypic change or alteration in one or more disease symptoms. Thus, “modulate” is used to refer to an increase, decrease, masking, altering, overriding or restoring the normal functioning, activity, or levels of a readout, outcome or process (e.g, biomolecule of interest, and/or molecular or physiological process, and/or a phenotypic change in one or more disease symptoms).

As used herein, the terms “modulate” and “treat” a disease and their cognates refer to an amelioration of a disease, disorder, and/or condition, or at least one discernible symptom thereof. In another embodiment, “modulate” and “treat” refer to an amelioration of at least one measurable physical parameter, not necessarily discernible by the patient. In another embodiment, “modulate” and “treat” refer to inhibiting the progression of a disease, disorder, and/or condition, either physically (e.g., stabilization of a discernible symptom), physiologically (e.g., stabilization of a physical parameter), or both. In another embodiment, “modulate” and “treat” refer to slowing the progression or reversing the progression of a disease, disorder, and/or condition. As used herein, “prevent” and its cognates refer to delaying the onset or reducing the risk of acquiring a given disease, disorder and/or condition or a symptom associated with such disease, disorder, and/or condition.

Those in need of treatment may include individuals already having a particular medical disorder, as well as those at risk of having, or who may ultimately acquire the disorder. The need for treatment is assessed, for example, by the presence of one or more risk factors associated with the development of a disorder, the presence or progression of a disorder, or likely receptiveness to treatment of a subject having the disorder. Treating autoimmune disorders and/or diseases and conditions associated with gut inflammation and/or compromised gut barrier function may encompass reducing or eliminating excess inflammation and/or associated symptoms, and does not necessarily encompass the elimination of the underlying disease. Treating the diseases described herein may encompass increasing levels of butyrate, increasing levels of acetate, increasing levels of butyrate and increasing GLP-2, IL-22, and/or IL-10, and/or modulating levels of tryptophan and/or its metabolites (e.g., kynurenine), and/or providing any other effector molecule and does not necessarily encompass the elimination of the underlying disease.

Those in need of treatment may include individuals already having a particular cancer, as well as those at risk of having, or who may ultimately acquire the cancer. The need for treatment is assessed, for example, by the presence of one or more risk factors associated with the development of a cancer (e.g., alcohol use, tobacco use, obesity, excessive exposure to ultraviolet radiation, high levels of estrogen, family history, genetic susceptibility), the presence or progression of a cancer, or likely receptiveness to treatment of a subject having the cancer. Cancer is caused by genomic instability and high mutation rates within affected cells. Treating cancer may encompass eliminating symptoms associated with the cancer and/or modulating the growth and/or volume of a subject's tumor, and does not necessarily encompass the elimination of the underlying cause of the cancer, e.g., an underlying genetic predisposition.

“Cancer” or “cancerous” is used to refer to a physiological condition that is characterized by unregulated cell growth. In some embodiments, cancer refers to a tumor. “Tumor” is used to refer to any neoplastic cell growth or proliferation or any pre-cancerous or cancerous cell or tissue. A tumor may be malignant or benign. Side effects of cancer treatment may include, but are not limited to, opportunistic autoimmune disorder(s), systemic toxicity, anemia, loss of appetite, irritation of bladder lining, bleeding and bruising (thrombocytopenia), changes in taste or smell, constipation, diarrhea, dry mouth, dysphagia, edema, fatigue, hair loss (alopecia), infection, infertility, lymphedema, mouth sores, nausea, pain, peripheral neuropathy, tooth decay, urinary tract infections, and/or problems with memory and concentration (National Cancer Institute). Those in need of treatment may include individuals already having a particular cancer, as well as those at risk of having, or who may ultimately acquire an autoimmune disorder.

As used herein, “diseases and conditions associated with gut inflammation and/or compromised gut barrier function” include, but are not limited to, inflammatory bowel diseases, diarrheal diseases, and related diseases. “Inflammatory bowel diseases” and “IBD” are used interchangeably herein to refer to a group of diseases associated with gut inflammation, which include, but are not limited to, Crohn's disease, ulcerative colitis, collagenous colitis, lymphocytic colitis, diversion colitis, Behcet's disease, and indeterminate colitis. As used herein, “diarrheal diseases” include, but are not limited to, acute watery diarrhea, e.g., cholera; acute bloody diarrhea, e.g., dysentery; and persistent diarrhea. As used herein, related diseases include, but are not limited to, short bowel syndrome, ulcerative proctitis, proctosigmoiditis, left-sided colitis, pancolitis, and fulminant colitis.

Symptoms associated with the aforementioned diseases and conditions include, but are not limited to, one or more of diarrhea, bloody stool, mouth sores, perianal disease, abdominal pain, abdominal cramping, fever, fatigue, weight loss, iron deficiency, anemia, appetite loss, weight loss, anorexia, delayed growth, delayed pubertal development, inflammation of the skin, inflammation of the eyes, inflammation of the joints, inflammation of the liver, and inflammation of the bile ducts.

As used herein, “metabolic diseases” include, but are not limited to, type 1 diabetes; type 2 diabetes; metabolic syndrome; Bardet-Biedel syndrome; Prader-Willi syndrome; non-alcoholic fatty liver disease; tuberous sclerosis; Albright hereditary osteodystrophy; brain-derived neurotrophic factor (BDNF) deficiency; Single-minded 1 (SIM1) deficiency; leptin deficiency; leptin receptor deficiency; pro-opiomelanocortin (POMC) defects; proprotein convertase subtilisin/kexin type 1 (PCSK1) deficiency; Src homology 2B1 (SH2B1) deficiency; pro-hormone convertase 1/3 deficiency; melanocortin-4-receptor (MC4R) deficiency; Wilms tumor, aniridia, genitourinary anomalies, and mental retardation (WAGR) syndrome; pseudohypoparathyroidism type 1A; Fragile X syndrome; Borjeson-Forsmann-Lehmann syndrome; Alstrom syndrome; Cohen syndrome; and ulnar-mammary syndrome.

Symptoms associated with the aforementioned diseases and conditions include, but are not limited to, one or more of weight gain, obesity, fatigue, hyperlipidemia, hyperphagia, hyperdipsia, polyphagia, polydipsia, polyuria, pain of the extremities, numbness of the extremities, blurry vision, nystagmus, hearing loss, cardiomyopathy, insulin resistance, light sensitivity, pulmonary disease, liver disease, liver cirrhosis, liver failure, kidney disease, kidney failure, seizures, hypogonadism, and infertility.

As used herein a “pharmaceutical composition” refers to a preparation of genetically engineered microorganism of the disclosure, e.g., recombinant bacteria, with other components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial or viral compound. An adjuvant is included under these phrases.

The term “excipient” refers to an inert substance added to a pharmaceutical composition to further facilitate administration of an active ingredient. Examples include, but are not limited to, calcium bicarbonate, sodium bicarbonate calcium phosphate, various sugars and types of starch, cellulose derivatives, gelatin, vegetable oils, polyethylene glycols, and surfactants, including, for example, polysorbate 20.

The terms “therapeutically effective dose” and “therapeutically effective amount” are used to refer to an amount of a compound that results in prevention, delay of onset of symptoms, or amelioration of symptoms of a condition, e.g., inflammation, diarrhea.an autoimmune disorder. A therapeutically effective amount may, for example, be sufficient to treat, prevent, reduce the severity, delay the onset, and/or reduce the risk of occurrence of one or more symptoms of an autoimmune a disorder and/or a disease or condition as described herein. A therapeutically effective amount, as well as a therapeutically effective frequency of administration, can be determined by methods known in the art and discussed below.

As used herein, the term “bacteriostatic” or “cytostatic” refers to a molecule or protein which is capable of arresting, retarding, or inhibiting the growth, division, multiplication or replication of recombinant bacterial cell of the disclosure.

As used herein, the term “bactericidal” refers to a molecule or protein which is capable of killing the recombinant bacterial cell of the disclosure.

As used herein, the term “toxin” refers to a protein, enzyme, or polypeptide fragment thereof, or other molecule which is capable of arresting, retarding, or inhibiting the growth, division, multiplication or replication of the recombinant bacterial cell of the disclosure, or which is capable of killing the recombinant bacterial cell of the disclosure. The term “toxin” is intended to include bacteriostatic proteins and bactericidal proteins. The term “toxin” is intended to include, but not limited to, lytic proteins, bacteriocins (e.g., microcins and colicins), gyrase inhibitors, polymerase inhibitors, transcription inhibitors, translation inhibitors, DNases, and RNases. The term “anti-toxin” or “antitoxin,” as used herein, refers to a protein or enzyme which is capable of inhibiting the activity of a toxin. The term anti-toxin is intended to include, but not limited to, immunity modulators, and inhibitors of toxin expression. Examples of toxins and antitoxins are known in the art and described in more detail infra.

As used herein, “payload” refers to one or more molecules of interest to be produced by a genetically engineered microorganism, such as a bacteria or a virus. In some embodiments, the payload is a therapeutic payload, e.g. an effector molecule, e.g. butyrate, acetate, propionate, GLP-2, IL-10, IL-22, IL-2, other interleukins, and/or tryptophan and/or one or more of its metabolites, as described herein. In some embodiments, the payload is a regulatory molecule, e.g., a transcriptional regulator such as FNR. In some embodiments, the payload comprises a regulatory element, such as a promoter or a repressor. In some embodiments, the payload comprises an inducible promoter, such as from FNRS. In some embodiments, the payload comprises a repressor element, such as a kill switch. In some embodiments, the payload comprises an antibiotic resistance gene or genes. In some embodiments, the payload is encoded by a gene, multiple genes, gene cassette, or an operon. In alternate embodiments, the payload is produced by a biosynthetic or biochemical pathway, wherein the biosynthetic or biochemical pathway may optionally be endogenous to the microorganism. In alternate embodiments, the payload is produced by a biosynthetic or biochemical pathway, wherein the biosynthetic or biochemical pathway is not endogenous to the microorganism. In some embodiments, the genetically engineered microorganism comprises two or more payloads.

As used herein, the term “conventional treatment” or “conventional therapy” refers to treatment or therapy that is currently accepted, considered current standard of care, and/or used by most healthcare professionals for treating a disease or disorder, e.g., cancer, autoimmune disorders, metabolic diseases, diseases relating to inborn errors of metabolism, neurological or neurodegenerative diseases, or diseases associated with inflammation and/or reduced gut barrier function. It is different from alternative or complementary therapies, which are not as widely used.

As used herein, the term “polypeptide” includes “polypeptide” as well as “polypeptides,” and refers to a molecule composed of amino acid monomers linearly linked by amide bonds (i.e., peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. A polypeptide may be derived from a natural biological source or produced by recombinant technology. In other embodiments, the polypeptide is produced by the recombinant bacteria of the current disclosure. A polypeptide may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides, which do not possess a defined three-dimensional structure, but rather can adopt a large number of different conformations, are referred to as unfolded. The term “peptide” or “polypeptide” may refer to an amino acid sequence that corresponds to a protein or a portion of a protein or may refer to an amino acid sequence that corresponds with non-protein sequence, e.g., a sequence selected from a regulatory peptide sequence, leader peptide sequence, signal peptide sequence, linker peptide sequence, and other peptide sequence.

An “isolated” polypeptide or a fragment, variant, or derivative thereof refers to a polypeptide that is not in its natural milieu. No particular level of purification is required. Recombinantly produced polypeptides and proteins expressed in host cells, including but not limited to bacterial or mammalian cells, are considered isolated for purposed of the invention, as are native or recombinant polypeptides which have been separated, fractionated, or partially or substantially purified by any suitable technique. Recombinant peptides, polypeptides or proteins refer to peptides, polypeptides or proteins produced by recombinant DNA techniques, i.e. produced from cells, microbial or mammalian, transformed by an exogenous recombinant DNA expression construct encoding the polypeptide. Proteins or peptides expressed in most bacterial cultures will typically be free of glycan. Fragments, derivatives, analogs or variants of the foregoing polypeptides, and any combination thereof are also included as polypeptides. The terms “fragment,” “variant,” “derivative” and “analog” include polypeptides having an amino acid sequence sufficiently similar to the amino acid sequence of the original peptide and include any polypeptides, which retain at least one or more properties of the corresponding original polypeptide. Fragments of polypeptides include proteolytic fragments, as well as deletion fragments. Fragments also include specific antibody or bioactive fragments or immunologically active fragments derived from any polypeptides described herein. Variants may occur naturally or be non-naturally occurring. Non-naturally occurring variants may be produced using mutagenesis methods known in the art. Variant polypeptides may comprise conservative or non-conservative amino acid substitutions, deletions or additions.

Polypeptides also include fusion proteins. As used herein, the term “variant” includes a fusion protein, which comprises a sequence of the original peptide or sufficiently similar to the original peptide. As used herein, the term “fusion protein” refers to a chimeric protein comprising amino acid sequences of two or more different proteins. Typically, fusion proteins result from well known in vitro recombination techniques. Fusion proteins may have a similar structural function (but not necessarily to the same extent), and/or similar regulatory function (but not necessarily to the same extent), and/or similar biochemical function (but not necessarily to the same extent) and/or immunological activity (but not necessarily to the same extent) as the individual original proteins which are the components of the fusion proteins. “Derivatives” include but are not limited to peptides, which contain one or more naturally occurring amino acid derivatives of the twenty standard amino acids. “Similarity” between two peptides is determined by comparing the amino acid sequence of one peptide to the sequence of a second peptide. An amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785. For example, amino acids belonging to one of the following groups represent conservative changes or substitutions: -Ala, Pro, Gly, Gln, Asn, Ser, Thr; -Cys, Ser, Tyr, Thr; -Val, Ile, Leu, Met, Ala, Phe; -Lys, Arg, His; -Phe, Tyr, Trp, His; and -Asp, Glu.

In some embodiments of the disclosure, the recombinant bacteria comprise one or more gene sequence(s) encoding one or more fusion proteins. In some embodiments, the recombinant bacteria express a fusion protein, in which a secretion tag polypeptide is fused to an effector polypeptide, i.e., the secretion tag is linked to the polypeptide through a peptide bond or a linker. In some embodiments, the recombinant bacteria express an effector polypeptide which is fused to a stabilizing polypeptide. As used herein “stabilizing polypeptide” extends the half-life of the effector polypeptide to which it is fused. Non-limiting examples of fusion proteins containing such stabilizing polypeptides include Fc fusion proteins, transferrin fusion proteins, and albumin fusion proteins (Strohl, BioDrugs. 2015; 29(4): 215-239). In some embodiments, the effector polypeptide is fused to an inert polypeptide to extend the half-life. A non-limiting example of such a polypeptide is XTEN (Schellenberger V, et al. Nat Biotechnol. 2009; 27:1186-1190). Another non-limiting example of a half-life extending polypeptide is CTP. CTP naturally extends protein's half-life in human serum, likely because the negatively charged, heavily sialylated CTP impairs renal clearance. Another non-limiting example of a polypeptide which can be fused to an effector to extend half-life is ELPs, which are repeating peptide units containing sequences commonly found in elastin (repeats of V-P-G-x-G, where x is any amino acid except proline (SEQ ID NO: 1057) (Strohl et al.). Another non-limiting examples of a polypeptide containing a polypeptide repeat sequence which can be fused to an effector are PAS (polymer using three repeating amino acids, proline, alanine and serine) and HAP (glycine-rich HAP). Finally, gelatin-like protein (GLK) polymer has also been used to extend effector half-life (Strohl, 2015).

An antibody generally refers to a polypeptide of the immunoglobulin family or a polypeptide comprising fragments of an immunoglobulin that is capable of noncovalently, reversibly, and in a specific manner binding a corresponding antigen. An exemplary antibody structural unit comprises a tetramer. Each tetramer is composed of two identical pairs of polypeptide chains, each pair having one “light” (about 25 kD) and one “heavy” chain (about 50-70 kD), connected through a disulfide bond. The recognized immunoglobulin genes include the κ, λ, γ, δ, ε, and μ constant region genes, as well as the myriad immunoglobulin variable region genes. Light chains are classified as either κ or λ. Heavy chains are classified as γ, μ, α, δ, or ε, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (VL) and variable heavy chain (VH) refer to these regions of light and heavy chains respectively.

As used herein, the term “antibody” or “antibodies” is meant to encompasses all variations of antibody and fragments thereof that possess one or more particular binding specificities. Thus, the term “antibody” or “antibodies” is meant to include full length antibodies, chimeric antibodies, humanized antibodies, single chain antibodies (ScFv, camelids), Fab, Fab′, multimeric versions of these fragments (e.g., F(ab′)2), single domain antibodies (sdAB, VHH fragments), heavy chain antibodies (HCAb), nanobodies, diabodies, and minibodies. Antibodies can have more than one binding specificity, e.g., be bispecific. The term “antibody” is also meant to include so-called antibody mimetics. Antibody mimetics refers to small molecules, e.g., 3-30 kDa, which can be single amino acid chain molecules, which can specifically bind antigens but do not have an antibody-related structure. Antibody mimetics, include, but are not limited to, Affibody molecules (Z domain of Protein A), Affilins (Gamma-B crystalline), Ubiquitin, Affimers (Cystatin), Affitins (Sac7d (from Sulfolobus acidocaldarius), Alphabodies (Triple helix coiled coil), Anticalins (Lipocalins), Avimers (domains of various membrane receptors), DARPins (Ankyrin repeat motif), Fynomers (SH3 domain of Fyn), Kunitz domain peptides Kunitz domains of various protease inhibitors), Ecallantide (Kalbitor), and Monobodies. In certain aspects, the term “antibody” or “antibodies” is meant to refer to a single chain antibody(ies), single domain antibody(ies), and camelid antibody(ies). Utility of antibodies in the treatment of cancer and additional anti-cancer antibodies can for example be found in Scott et al., Antibody Therapy for Cancer, Nature Reviews Cancer April 2012 Volume 12, incorporated by reference in its entirety.

A “single-chain antibody” or “single-chain antibodies” typically refers to a peptide comprising a heavy chain of an immunoglobulin, a light chain of an immunoglobulin, and optionally a linker or bond, such as a disulfide bond. The single-chain antibody lacks the constant Fc region found in traditional antibodies. In some embodiments, the single-chain antibody is a naturally occurring single-chain antibody, e.g., a camelid antibody. In some embodiments, the single-chain antibody is a synthetic, engineered, or modified single-chain antibody. In some embodiments, the single-chain antibody is capable of retaining substantially the same antigen specificity as compared to the original immunoglobulin despite the addition of a linker and the removal of the constant regions. In some aspects, the single chain antibody can be a “scFv antibody”, which refers to a fusion protein of the variable regions of the heavy (VH) and light chains (VL) of immunoglobulins (without any constant regions), optionally connected with a short linker peptide of ten to about 25 amino acids, as described, for example, in U.S. Pat. No. 4,946,778, the contents of which is herein incorporated by reference in its entirety. The Fv fragment is the smallest fragment that holds a binding site of an antibody, which binding site may, in some aspects, maintain the specificity of the original antibody. Techniques for the production of single chain antibodies are described in U.S. Pat. No. 4,946,778. The Vh and VL sequences of the scFv can be connected via the N-terminus of the VH connecting to the C-terminus of the VL or via the C-terminus of the VH connecting to the N-terminus of the VL. ScFv fragments are independent folding entities that can be fused indistinctively on either end to other epitope tags or protein domains. Linkers of varying length can be used to link the Vh and VL sequences, which the linkers can be glycine rich (provides flexibility) and serine or threonine rich (increases solubility). Short linkers may prevent association of the two domains and can result in multimers (diabodies, tribodies, etc.). Long linkers may result in proteolysis or weak domain association (described in Voelkel et al el., 2011). Linkers of length between 15 and 20 amino acids or 18 and 20 amino acids are most often used. Additional non-limiting examples of linkers, including other flexible linkers are described in Chen et al., 2013 (Adv Drug Deliv Rev. 2013; 65(10): 1357-1369.), the contents of which is herein incorporated by reference in its entirety. Flexible linkers are also rich in small or polar amino acids such as Glycine and Serine, but can contain additional amino acids such as Threonine and Alanine to maintain flexibility, as well as polar amino acids such as Lysine and Glutamate to improve solubility. Exemplary linkers include, but are not limited to, (Gly-Gly-Gly-Gly-Ser)n (SEQ ID NO: 1058), KESGSVSSEQLAQFRSLD (SEQ ID NO: 1059) and EGKSSGSGSESKST (SEQ ID NO: 1060), (Gly)8 (SEQ ID NO: 1061), and Gly and Ser rich flexible linker, GSAGSAAGSGEF (SEQ ID NO: 1062).

“Single chain antibodies” as used herein also include single-domain antibodies, which include camelid antibodies and other heavy chain antibodies, light chain antibodies, including nanobodies and single domains VH or VL domains derived from human, mouse or other species. Single domain antibodies may be derived from any species including, but not limited to mouse, human, camel, llama, fish, shark, goat, rabbit, and bovine. Single domain antibodies include domain antigen-binding units which have a camelid scaffold, derived from camels, llamas, or alpacas. Camelids produce functional antibodies devoid of light chains. The heavy chain variable (VH) domain folds autonomously and functions independently as an antigen-binding unit. Its binding surface involves only three CDRs as compared to the six CDRs in classical antigen-binding molecules (Fabs) or single chain variable fragments (scFvs). Camelid antibodies are capable of attaining binding affinities comparable to those of conventional antibodies. Camelid scaffold-based antibodies can be produced using methods well known in the art. Cartilaginous fishes also have heavy-chain antibodies (IgNAR, ‘immunoglobulin new antigen receptor’), from which single-domain antibodies called VNAR fragments can be obtained. Alternatively, the dimeric variable domains from IgG from humans or mice can be split into monomers. Nanobodies are single chain antibodies derived from light chains. The term “single chain antibody” also refers to antibody mimetics.

In some embodiments, the antibodies expressed by the engineered microorganisms are bispecific. In certain embodiments, a bispecific antibody molecule comprises a scFv, or fragment thereof, have binding specificity for a first epitope and a scFv, or fragment thereof, have binding specificity for a second epitope. Antigen-binding fragments or antibody portions include bivalent scFv (diabody), bispecific scFv antibodies where the antibody molecule recognizes two different epitopes, single binding domains (dAbs), and minibodies. Monomeric single-chain diabodies (scDb) are readily assembled in bacterial and mammalian cells and show improved stability under physiological conditions (Voelkel et al., 2001 and references therein; Protein Eng. (2001) 14 (10): 815-823).

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, 50%, 55%, 60%, 65%, 70%, 75%, 80%, 85%, 90%, 91%, 92%, 93%, 94%, 95%, 96%, 97%, 98%, 99%, or 100%, identical are defined herein as sufficiently similar. Preferably, variants will be sufficiently similar to the amino acid sequence of the peptides of the disclosure. Such variants generally retain the functional activity of the peptides of the present disclosure. Variants include peptides that differ in amino acid sequence from the native and wt peptide, respectively, by way of one or more amino acid deletion(s), addition(s), and/or substitution(s). These may be naturally occurring variants as well as artificially designed ones.

As used herein the term “linker”, “linker peptide” or “peptide linkers” or “linker” refers to synthetic or non-native or non-naturally-occurring amino acid sequences that connect or link two polypeptide sequences, e.g., that link two polypeptide domains. As used herein the term “synthetic” refers to amino acid sequences that are not naturally occurring. Exemplary linkers are described herein. Additional exemplary linkers are provided in US 20140079701 and in Chen et al, Adv Drug Deliv Rev. 2013; 65(10): 1357-1369, the contents of which are herein incorporated by reference in its entirety. Table 2 depicts non-limiting examples of linkers known in the art.

TABLE 2
Linkers

SEQ ID NO:

Increase Stability/Folding

| | | | |
|---|---|---|---|
| scFv + DB2:D24 | 1063 | (GGGGS)3 | 1063 |
| G-CSF-Tf | 1063 | (GGGGS)3 | 1063 |
| HBsAg preS1 | 1063 | (GGGGS)3 | 1063 |
| Myc- Est2p | 1061 | (Gly)8 | 1061 |
| albumin-ANF | 1064 | (Gly)6 | 1064 |
| virus coat | 1065 | (EAAAK)3 | 1065 |
| protein | | | |
| beta-glucanase- | 1066 | (EAAAK)n (n = 1-3) | 1066 |
| xylanase | | | |

Increase expression

| | | | |
|---|---|---|---|
| hGH-Tf and | 1067 | A(EAAAK)4ALEA(EAAAK)4A | 1067 |
| Tf-hGH | | | |
| G-CSF-Tf and | 1067 | A(EAAAK)4ALEA(EAAAK)4A | 1067 |
| Tf-G-CSF | rigid | | |

Improve biological activty

| | | | |
|---|---|---|---|
| G-CSF-Tf | 1063 | (GGGGS)3 | 1063 |
| G-CSF-Tf | 1067 | A(EAAAK)4ALEA(EAAAK)4A | 1067 |
| hGH-Tf | 1067 | A(EAAAK)4ALEA(EAAAK)4A | 1067 |
| HSA-IFN-α2b | 1058 | GGGGS | |
| HSA-IFN-α2b | 1068 | PAPAP | 1068 |
| HSA-IFN-α2b | 1069 | AEAAAKEAAAKA | 1069 |
| PGA-rTHS | 1070 | (GGGGS)n (n = 1, 2, 4) | 1070 |
| interferon-γ- | 1071 | (Ala-Pro)n (10-34 aa) | 1071 |
| gp120 | | | |
| GSF-S-S-Tf | | disulfide | |
| IFN-α2b-HSA | | disulfide | |

Enable targeting

| | | | |
|---|---|---|---|
| FIX-albumin | 1072 | VSQTSKLTR↓AETVFPDV | 1072 |
| LAP-IFN-β | 1073 | PLG↓LWA | 1073 |
| MazE-MazF | 1074; 1075 | RVL↓AEA; EDVVCC↓SMSY; | 1074; 1075 |
| | | GGIEGR↓GSC | 1076 |
| Immunotoxins | cleavable | TRHRQPR↓GWE; | 1077 |
| | cleavable | AGNRVRR↓SVG; | 1078 |
| | cleavable | RRRRRRR↓R↓R | 1079 |
| Immunotoxin | cleavable | GFLG↓ | 1080 |

Alter PK

| | | | |
|---|---|---|---|
| G-CSF-Tf and | dipeptide | LE | |
| hGH-Tf | rigid | A(EAAAK)4ALEA(EAAAK)4A | 1067 |
| | cleavable | Disulfide | |

As used herein the term “codon-optimized” refers to the modification of codons in the gene or coding regions of a nucleic acid molecule to reflect the typical codon usage of the host organism without altering the polypeptide encoded by the nucleic acid molecule. Such optimization includes replacing at least one, or more than one, or a significant number, of codons with one or more codons that are more frequently used in the genes of the host organism. A “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism. Many organisms display a bias or preference for use of particular codons to code for insertion of a particular amino acid in a growing polypeptide chain. Codon preference or codon bias, differences in codon usage between organisms, is allowed by the degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

As used herein, the terms “secretion system” or “secretion protein” refers to a native or non-native secretion mechanism capable of secreting or exporting a biomolecule, e.g., polypeptide from the microbial, e.g., bacterial cytoplasm. The secretion system may comprise a single protein or may comprise two or more proteins assembled in a complex e.g., HlyBD. Non-limiting examples of secretion systems for gram negative bacteria include the modified type III flagellar, type I (e.g., hemolysin secretion system), type II, type IV, type V, type VI, and type VII secretion systems, resistance-nodulation-division (RND) multi-drug efflux pumps, and various single membrane secretion systems. Non-liming examples of secretion systems for gram positive bacteria include Sec and TAT secretion systems. In some embodiments, the polypeptide to be secreted include a “secretion tag” of either RNA or peptide origin to direct the polypeptide to specific secretion systems. In some embodiments, the secretion system is able to remove this tag before secreting the polypeptide from the engineered bacteria. For example, in Type V auto-secretion-mediated secretion the N-terminal peptide secretion tag is removed upon translocation of the “passenger” peptide from the cytoplasm into the periplasmic compartment by the native Sec system. Further, once the auto-secretor is translocated across the outer membrane the C-terminal secretion tag can be removed by either an autocatalytic or protease-catalyzed e.g., OmpT cleavage thereby releasing the antinflammatory or barrier enhancer molecule(s) into the extracellular milieu. In some embodiments, the secretion system involves the generation of a “leaky” or de-stabilized outer membrane, which may be accomplished by deleting or mutagenizing genes responsible for tethering the outer membrane to the rigid peptidoglycan skeleton, including for example, lpp, ompC, ompA, ompF, tolA, tolB, pal, degS, degP, and nlpl. Lpp functions as the primary ‘staple’ of the bacterial cell wall to the peptidoglycan. TolA-PAL and OmpA complexes function similarly to Lpp and are other deletion targets to generate a leaky phenotype. Additionally, leaky phenotypes have been observed when periplasmic proteases, such as degS, degP or nlpl, are deactivated. Thus, in some embodiments, the engineered bacteria have one or more deleted or mutated membrane genes, e.g., selected from lpp, ompA, ompA, ompF, tolA, tolB, and pal genes. In some embodiments, the engineered bacteria have one or more deleted or mutated periplasmic protease genes, e.g., selected from degS, degP, and nlpl. In some embodiments, the engineered bacteria have one or more deleted or mutated gene(s), selected from lpp, ompA, ompA, ompF, tolA, tolB, pal, degS, degP, and nlpl genes.

The articles “a” and “an,” as used herein, should be understood to mean “at least one,” unless clearly indicated to the contrary.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Bacteria

The genetically engineered microorganisms, or programmed microorganisms, such as recombinant bacteria of the disclosure are capable of producing one or more non-native effector molecules, therapeutic polypeptides, anti-inflammation and/or gut barrier function enhancer molecules, as described herein. In certain embodiments, the recombinant bacteria are obligate anaerobic bacteria. In certain embodiments, the recombinant bacteria are facultative anaerobic bacteria. In certain embodiments, the recombinant bacteria are aerobic bacteria. In some embodiments, the recombinant bacteria are Gram-positive bacteria. In some embodiments, the recombinant bacteria are Gram-positive bacteria and lack LPS. In some embodiments, the recombinant bacteria are Gram-negative bacteria. In some embodiments, the recombinant bacteria are Gram-positive and obligate anaerobic bacteria. In some embodiments, the recombinant bacteria are Gram-positive and facultative anaerobic bacteria. In some embodiments, the recombinant bacteria are non-pathogenic bacteria. In some embodiments, the recombinant bacteria are commensal bacteria. In some embodiments, the recombinant bacteria are probiotic bacteria. In some embodiments, the recombinant bacteria are naturally pathogenic bacteria that are modified or mutated to reduce or eliminate pathogenicity. Exemplary bacteria include, but are not limited to, Bacillus, Bacteroides, Bifidobacterium, Brevibacteria, Caulobacter, Clostridium, Enterococcus, Escherichia coli, Lactobacillus, Lactococcus, Listeria, Mycobacterium, Saccharomyces, Salmonella, Staphylococcus, Streptococcus, Vibrio, Bacillus coagulans, Bacillus subtilis, Bacteroides fragilis, Bacteroides subtilis, Bacteroides thetaiotaomicron, Bifidobacterium adolescentis, Bifidobacterium bifidum, Bifidobacterium breve UCC2003, Bifidobacterium infantis, Bifidobacterium lactis, Bifidobacterium longum, Clostridium acetobutylicum, Clostridium butyricum, Clostridium butyricum M-55, Clostridium cochlearum, Clostridium felsineum, Clostridium histolyticum, Clostridium multifermentans, Clostridium novyi-NT, Clostridium paraputrificum, Clostridium pasteureanum, Clostridium pectinovorum, Clostridium perfringens, Clostridium roseum, Clostridium sporogenes, Clostridium tertium, Clostridium tetani, Clostridium tyrobutyricum, Corynebacterium parvum, Escherichia coli MG1655, Escherichia coli Nissle 1917, Listeria monocytogenes, Mycobacterium bovis, Salmonella choleraesuis, Salmonella typhimurium, and Vibrio cholera. In certain embodiments, the recombinant bacteria are selected from the group consisting of Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii, Clostridium clusters IV and XIVa of Firmicutes (including species of Eubacterium), Roseburia, Faecalibacterium, Enterobacter, Faecalibacterium prausnitzii, Clostridium difficile, Subdoligranulum, Clostridium sporogenes, Campylobacter jejuni, Clostridium saccharolyticum, Klebsiella, Citrobacter, Pseudobutyrivibrio, and Ruminococcus. In certain embodiments, the recombinant bacteria are selected from Bacteroides fragilis, Bacteroides thetaiotaomicron, Bacteroides subtilis, Bifidobacterium bifidum, Bifidobacterium infantis, Bifidobacterium lactis, Clostridium butyricum, Escherichia coli, Escherichia coli Nissle, Lactobacillus acidophilus, Lactobacillus plantarum, Lactobacillus reuteri, and Lactococcus lactis

In some embodiments, the recombinant bacterium is a Gram-positive bacterium, e.g., Clostridium, that is naturally capable of producing high levels of butyrate. In some embodiments, the recombinant bacterium is selected from the group consisting of C. butyricum ZJUCB, C. butyricum S21, C. thermobutyricum ATCC 49875, C. beijerinckii, C. populeti ATCC 35295, C. tyrobutyricum JM1, C. tyrobutyricum CIP 1-776, C. tyrobutyricum ATCC 25755, C. tyrobutyricum CNRZ 596, and C. tyrobutyricum ZJU 8235. In some embodiments, the recombinant bacterium is C. butyricum CBM588, a probiotic bacterium that is highly amenable to protein secretion and has demonstrated efficacy in treating IBD (Kanai et al., 2015). In some embodiments, the recombinant bacterium is Bacillus, a probiotic bacterium that is highly genetically tractable and has been a popular chassis for industrial protein production; in some embodiments, the bacterium has highly active secretion and/or no toxic byproducts (Cutting, 2011).

In one embodiment, the bacterial cell is a Bacteroides fragilis bacterial cell. In one embodiment, the bacterial cell is a Bacteroides thetaiotaomicron bacterial cell. In one embodiment, the bacterial cell is a Bacteroides subtilis bacterial cell. In one embodiment, the bacterial cell is a Bifidobacterium bifidum bacterial cell. In one embodiment, the bacterial cell is a Bifidobacterium infantis bacterial cell. In one embodiment, the bacterial cell is a Bifidobacterium lactis bacterial cell. In one embodiment, the bacterial cell is a Clostridium butyricum bacterial cell. In one embodiment, the bacterial cell is an Escherichia coli bacterial cell. In one embodiment, the bacterial cell is a Lactobacillus acidophilus bacterial cell. In one embodiment, the bacterial cell is a Lactobacillus plantarum bacterial cell. In one embodiment, the bacterial cell is a Lactobacillus reuteri bacterial cell. In one embodiment, the bacterial cell is a Lactococcus lactis bacterial cell.

In some embodiments, the recombinant bacteria are Escherichia coli strain Nissle 1917 (E. coli Nissle), a Gram-negative bacterium of the Enterobacteriaceae family that has evolved into one of the best characterized probiotics (Ukena et al., 2007). The strain is characterized by its complete harmlessness (Schultz, 2008), and has GRAS (generally recognized as safe) status (Reister et al., 2014, emphasis added). Genomic sequencing confirmed that E. coli Nissle lacks prominent virulence factors (e.g., E. coli α-hemolysin, P-fimbrial adhesins) (Schultz, 2008). In addition, it has been shown that E. coli Nissle does not carry pathogenic adhesion factors, does not produce any enterotoxins or cytotoxins, is not invasive, and not uropathogenic (Sonnenborn et al., 2009). As early as in 1917, E. coli Nissle was packaged into medicinal capsules, called Mutaflor, for therapeutic use. E. coli Nissle has since been used to treat ulcerative colitis in humans in vivo (Rembacken et al., 1999), to treat inflammatory bowel disease, Crohn's disease, and pouchitis in humans in vivo (Schultz, 2008), and to inhibit enteroinvasive Salmonella, Legionella, Yersinia, and Shigella in vitro (Altenhoefer et al., 2004). It is commonly accepted that E. coli Nissle's therapeutic efficacy and safety have convincingly been proven (Ukena et al., 2007). In some embodiments, the recombinant bacteria are E. coli Nissle and are naturally capable of promoting tight junctions and gut barrier function. In some embodiments, the recombinant bacteria are E. coli and are highly amenable to recombinant protein technologies.

One of ordinary skill in the art would appreciate that the genetic modifications disclosed herein may be adapted for other species, strains, and subtypes of bacteria. It is known, for example, that the clostridial butyrogenic pathway genes are widespread in the genome-sequenced clostridia and related species (Aboulnaga et al., 2013). Furthermore, genes from one or more different species of bacteria can be introduced into one another, e.g., the butyrogenic genes fromPeptoclostridium difficile have been expressed in Escherichia coli (Aboulnaga et al., 2013).

In one embodiment, the recombinant bacterial cell does not colonize the subject having the disorder. Unmodified E. coli Nissle and the recombinant bacteria may be destroyed, e.g., by defense factors in the gut or blood serum (Sonnenborn et al., 2009) or by activation of a kill switch, several hours or days after administration. Thus, the recombinant bacteria may require continued administration. Residence time in vivo may be calculated for the recombinant bacteria. In some embodiments, the residence time is calculated for a human subject. In some embodiments, residence time in vivo is calculated for the recombinant bacteria of the disclosure, e.g. as described herein.

In some embodiments, the bacterial cell is a recombinant bacterial cell. In another embodiment, the bacterial cell is a recombinant bacterial cell. In some embodiments, the disclosure comprises a colony of bacterial cells disclosed herein. In another aspect, the disclosure provides a recombinant bacterial culture which comprises bacterial cells disclosed herein.

In some embodiments, the recombinant bacteria comprising an effector molecule further comprise a kill-switch circuit, such as any of the kill-switch circuits provided herein. For example, in some embodiments, the recombinant bacteria further comprise one or more genes encoding one or more recombinase(s) under the control of an inducible promoter, and an inverted toxin sequence. In some embodiments, the recombinant bacteria further comprise one or more genes encoding an antitoxin. In some embodiments, the engineered bacteria further comprise one or more genes encoding one or more recombinase(s) under the control of an inducible promoter and one or more inverted excision genes, wherein the excision gene(s) encode an enzyme that deletes an essential gene. In some embodiments, the recombinant bacteria further comprise one or more genes encoding an antitoxin. In some embodiments, the engineered bacteria further comprise one or more genes encoding a toxin under the control of a promoter having a TetR repressor binding site and a gene encoding the TetR under the control of an inducible promoter that is induced by arabinose, such as ParaBAD. In some embodiments, the recombinant bacteria further comprise one or more genes encoding an antitoxin.

In some embodiments, the recombinant bacteria is an auxotroph comprising gene sequence encoding an effector molecule and further comprises a kill-switch circuit, such as any of the kill-switch circuits described herein.

In some embodiments of the above described recombinant bacteria, the gene encoding an effector molecule is present on a plasmid in the bacterium. In some embodiments, the gene sequence(s) encoding an effector molecule is present in the bacterial chromosome. In some embodiments, a gene sequence encoding a secretion protein or protein complex, such as any of the secretion systems disclosed herein, for secreting a biomolecule (e.g. an effector molecule), is present on a plasmid in the bacterium. In some embodiments, the gene sequence encoding a secretion protein or protein complex for secreting a biomolecule, such as any of the secretion systems disclosed herein, is present in the bacterial chromosome. In some embodiments, the gene sequence(s) encoding an antibiotic resistance gene is present on a plasmid in the bacterium. In some embodiments, the gene sequence(s) encoding an antibiotic resistance gene is present in the bacterial chromosome.

Secreted Polypeptides

IL-10

In some embodiments, the recombinant bacteria are capable of producing IL-10. Interleukin-10 (IL-10) is a class 2 cytokine, a category which includes cytokines, interferons, and interferon-like molecules, such as IL-19, IL-20, IL-22, IL-24, IL-26, IL-28A, IL-28B, IL-29, IFN-α, IFN-β, IFN-δ, IFN-ε, IFN-κ, IFN-τ, IFN-ω, and limitin. IL-10 is an anti-inflammatory cytokine that signals through two receptors, IL-10R1 and IL-10R2. Anti-inflammatory properties of human IL-10 include down-regulation of pro-inflammatory cytokines, inhibition of antigen presentation on dendritic cells or suppression of major histocompatibility complex expression. Deficiencies in IL-10 and/or its receptors are associated with IBD and intestinal sensitivity (Nielsen, 2014). Bacteria expressing IL-10 or protease inhibitors may ameliorate conditions such as Crohn's disease and ulcerative colitis (Simpson et al., 2014). The recombinant bacteria may comprise any suitable gene encoding IL-10, e.g., human IL-10. In some embodiments, the gene encoding IL-10 is modified and/or mutated, e.g., to enhance stability, increase IL-10 production, and/or increase anti-inflammatory potency under inducing conditions. In some embodiments, the recombinant bacteria are capable of producing IL-10 under inducing conditions, e.g., under a condition(s) associated with inflammation. In some embodiments, the recombinant bacteria are capable of producing IL-10 in low-oxygen conditions. In some embodiments, the recombinant bacteria comprise a nucleic acid sequence that encodes IL-10. In some embodiments, the recombinant bacteria comprise a nucleic acid sequence comprising SEQ ID NO: 141 or a functional fragment thereof. In some embodiments, recombinant bacteria comprise a nucleic acid sequence that is at least about 80%, at least about 85%, at least about 90%, at least about 95%, or at least about 99% homologous to a nucleic acid sequence comprising SEQ ID NO: 141 or a functional fragment thereof.

In some embodiments, the recombinant bacteria comprise a gene sequence encoding a monomerized form of IL-10. Wild type IL-10 (wtIL-10) is a domain swapped dimer whose structural integrity depends on the dimerization of two peptide chains. wtIL-10 was converted to a monomeric isomer by inserting 6 amino acids into the loop connecting the swapped secondary structural elements (see, e.g., Josephson, K. et al. J. Biol. Chem. 275, 13552-13557 (2000), and Yoon, S. I. et al. J. Biol. Chem. 287, 26586-26595 (2012). Monomoerized IL-10 therefore comnprises a small linker which deviates from the wild-type human IL-10 sequence. This linker causes the IL10 to become active as a monomer rather than a dimer.

Secretion of a monomeric protein may have advantages, avoiding the extra step of dimerization in the periplasmic space. Moreover, there is more flexibility in the selection of appropriate secretion systems. For example, the tat-dependent secretion system secretes polypeptides in a folded fashion. Dimers cannot fold correctly without the formation of disulfide bonds. Disulfide bonds, however, cannot form in the reducing intracellular environment and require the oxidizing environment of the periplasm to form. Therefore, the tat-dependent system may not be appropriate for the secretion of proteins which require dimerization to function properly.

In some embodiments, the recombinant bacteria are capable of producing viral IL-10. Exemplary viral IL-10 homologues encoded by the bacteria include human cytomegalo- (HCMV) and Epstein-Barr virus (EBV) IL-10. Apart from its anti-inflammatory effects, human IL-10 also possesses pro-inflammatory activity, e.g., stimulation of B-cell maturation and proliferation of natural killer cells (Foerster et al., BMC Biotechnol. 2013; 13: 82, and references therein). In contrast, viral IL-10 homologues share many biological activities of hIL-10 but, due to selective pressure during virus evolution and the need to escape the host immune system, also display unique traits, including increased stability and lack of immunostimulatory functions (Foerster et al, and references therein). As such, viral counterparts may be useful and possibly more effective than hIL-10 with respect to anti-inflammatory and/or immune suppressing effects.

In some embodiments, the recombinant bacteria are capable of producing viral IL-10 under inducing conditions, e.g., under a condition(s) associated with inflammation. In some embodiments, the recombinant bacteria are capable of producing viral IL-10 in low-oxygen conditions. In some embodiments, the recombinant bacteria comprise a nucleic acid sequence that encodes viral IL-10. In some embodiments, the viral d IL-10 expressed by the bacteria stimulates IL-10R1 and IL-10R2 and initiates signal transduction. Signaling includes Stat signaling, e.g. through the phosphorylation of Tyr705 and/or Ser727.

In some embodiments, the recombinant bacteria are capable of producing monomerized human IL-10. In some embodiments, the recombinant bacteria are capable of producing monomerized IL-10 under inducing conditions, e.g., under a condition(s) associated with inflammation. In some embodiments, the recombinant bacteria are capable of producing monomerized IL-10 in low-oxygen conditions. In some embodiments, the recombinant bacteria comprise a nucleic acid sequence that encodes monomerized IL-10. In some embodiments, the recombinant bacteria comprise a nucleic acid sequence comprising SEQ ID NO: 142 or a functional fragment thereof. In some embodiments, recombinant bacteria comprise a nucleic acid sequence that is at least about 80%, 85%, 90%, 95%, or 99% homologous to a nucleic acid sequence comprising SEQ ID NO: 142 or a functional fragment thereof. In some embodiments, the recombinant bacteria comprise a sequence which encodes the polypeptide encoded by SEQ ID NO: 142 or a fragment or functional variant thereof. In some embodiments, the monomerized IL-10 expressed by the bacteria stimulates IL-10R1 and IL-10R2 and initiates signal transduction. Signaling includes Stat signaling, e.g. through the phosphorylation of Tyr705 and/or Ser727.

In some embodiments, the recombinant bacteria encode a covalently linked human interleukin-10 fusion protein. In some embodiments, the recombinant bacteria comprise a gene sequences two interleukin-10 monomer subunits covalently linked by a linker. In some embodiments, the linker is a glycine-serine rich linker. In some embodiments, the linker is cysteine free. In some embodiments, the peptide linker is comprises one or more of (Gly3SerGly3)n (SEQ ID NO: 1081), (Gly2Ser1Gly2)n (SEQ ID NO: 1082), (GlySer1Gly)n (SEQ ID NO: 1083), and (Gly3Ser1Gly2)n (SEQ ID NO: 1084), wherein n is an integer independently selected from 0, 1, 2, 3 or 4. In some embodiments, the linker comprises GGGSGGGS (SEQ ID NO: 142). In some embodiments, the linker is covalently linked to the C-terminus of one interleukin-10 monomer subunit and the N-terminus of the second interleukin-10 monomer subunit.

A non-limiting example of an covalently linked human interleukin-10 fusion protein of the disclosure is provided in SEQ ID NO: 143. In some embodiments, the recombinant bacteria comprise one or more nucleic acid sequence(s) encoding a polypeptide of SEQ ID NO: 143 or a functional fragment thereof. In some embodiments, the recombinant bacteria comprise a nucleic acid sequence that encodes a polypeptide that is at least about 80%, 85%, 90%, 95%, or 99% homologous to the polypeptide sequence of SEQ ID NO: 143 or a functional fragment thereof.

In some embodiments, the recombinant bacteria comprise gene sequence encoding a IL-10 monomer fusion protein which comprises SEQ ID NO: 497 or a functional fragment thereof. In some embodiments, recombinant bacteria comprise a gene sequence encoding a IL-10 monomer polypeptide that is at least about 80%, 85%, 90%, 95%, or 99% identity to IL-10 monomer polypeptide comprising SEQ ID NO: 497 or a functional fragment thereof. In some embodiments, the IL-10 monomer polypeptide has at least about 85%, 86%, 87%, 88%, 89%, 90%, 91%, 92%, 93%, 94%, 95%, 96%, 97%, 98%, or 99% identity with SEQ ID NO: 497. In some specific embodiments, the IL-10 monomer polypeptide comprises SEQ ID NO: 497. In some embodiments, the recombinant bacteria comprise gene sequence encoding a IL-10 monomer fusion protein. In certain embodiments, the IL-10 monomer fusion protein gene sequence has at least about 80%, 85%, 90%, 95%, or 99% identity with SEQ ID NO: 493. In some embodiments, the IL-10 monomer gene sequence has at least about 80% 85%, 86%, 87%, 88%, 89%, 90%, 91%, 92%, 93%, 94%, 95%, 96%, 97%, 98%, or 99% identity with SEQ ID NO: 493. In some specific embodiments, the IL-10 monomer gene sequence comprises SEQ ID NO: 493.

In some embodiments, the recombinant bacteria comprise gene sequence encoding a IL-10-linker-IL-10 fusion protein which comprises SEQ ID NO: 143 or a functional fragment thereof. In some embodiments, recombinant bacteria comprise a gene sequence encoding a IL-10-linker-IL-10 polypeptide that is at least about 80%, 85%, 90%, 95%, or 99% identity to IL-10-linker-IL-10 polypeptide comprising SEQ ID NO: 143 or a functional fragment thereof. In some embodiments, the IL-10-linker-IL-10 polypeptide has at least about 85%, 86%, 87%, 88%, 89%, 90%, 91%, 92%, 93%, 94%, 95%, 96%, 97%, 98%, or 99% identity with SEQ ID NO: 143. In some specific embodiments, the IL-10-linker-IL-10 polypeptide comprises SEQ ID NO: 143. In some embodiments, the recombinant bacteria comprise gene sequence encoding a IL-10-linker-IL-10 protein. In certain embodiments, the IL-10-linker-IL-10 gene sequence has at least about 80%, 85%, 90%, 95%, or 99% identity with SEQ ID NO: 492. In some embodiments, the IL-10-linker-IL-10 gene sequence has at least about 85%, 86%, 87%, 88%, 89%, 90%, 91%, 92%, 93%, 94%, 95%, 96%, 97%, 98%, or 99% identity with SEQ ID NO: 492. In some specific embodiments, the IL-10-linker-IL-10 gene sequence comprises SEQ ID NO: 492.

In some embodiments, the recombinant bacteria comprise gene sequence encoding a linker fusion protein which comprises SEQ ID NO: 498 or a functional fragment thereof. In some embodiments, recombinant bacteria comprise a gene sequence encoding a linker polypeptide that is at least about 80%, 85%, 90%, 95%, or 99% identity to linker polypeptide comprising SEQ ID NO: 498 or a functional fragment thereof. In some embodiments, the linker polypeptide has at least about 85%, 86%, 87%, 88%, 89%, 90%, 91%, 92%, 93%, 94%, 95%, 96%, 97%, 98%, or 99% identity with SEQ ID NO: 498. In some specific embodiments, the linker polypeptide comprises SEQ ID NO: 498. In some embodiments, the gene sequence encoding ECOLIN 19410 secretion tag comprises SEQ ID NO: 498. In some embodiments, the recombinant bacteria comprise gene sequence encoding a linker fusion protein. In certain embodiments, the linker fusion protein gene sequence has at least about 80%, 85%, 90%, 95%, or 99% identity with SEQ ID NO: 494. In some embodiments, the linker gene sequence has at least about 85%, 86%, 87%, 88%, 89%, 90%, 91%, 92%, 93%, 94%, 95%, 96%, 97%, 98%, or 99% identity with SEQ ID NO: 494. In some specific embodiments, the linker gene sequence comprises SEQ ID NO: 494.

In any of these embodiments, the recombinant bacteria may further comprise gene sequences encoding a secretion tag. Non-limiting examples of such secretion tags are described herein and include PhoA, OmpF, cvaC, TorA, fdnG, dmsA, PelB, the ECOLIN_05715 secretion signal, ECOLIN_16495 secretion signal, ECOLIN_19410 secretion signal, and the ECOLIN_19880 secretion signal. In some embodiments, the secretion tag is PhoA. In some embodiments, the PhoA secretion tag comprises SEQ ID NO: 385. In some embodiments, the PhoA secretion tag comprises SEQ ID NO: 386.

In some embodiments, the recombinant bacteria comprise gene sequence encoding a PhoA-IL-10 fusion protein which comprises SEQ ID NO: 563 or a functional fragment thereof. In some embodiments, recombinant bacteria comprise a gene sequence encoding a PhoA-IL-10 polypeptide that is at least about 80%, 85%, 90%, 95%, or 99% identity to PhoA-IL-10 polypeptide comprising SEQ ID NO: 563 or a functional fragment thereof. In some embodiments, the PhoA-IL-10 polypeptide has at least about 85%, 86%, 87%, 88%, 89%, 90%, 91%, 92%, 93%, 94%, 95%, 96%, 97%, 98%, or 99% identity with SEQ ID NO: 563. In some specific embodiments, the PhoA-IL-10 polypeptide comprises SEQ ID NO: 563. In some embodiments, the recombinant bacteria comprise gene sequence encoding a PhoA-IL-10. In certain embodiments, the PhoA-IL-10 gene sequence has at least about 80%, 85%, 90%, 95%, or 99% identity with SEQ ID NO: 566. In some embodiments, the PhoA-IL-10 gene sequence has at least about 85%, 86%, 87%, 88%, 89%, 90%, 91%, 92%, 93%, 94%, 95%, 96%, 97%, 98%, or 99% identity with SEQ ID NO: 566. In some specific embodiments, the PhoA-IL-10 gene sequence comprises SEQ ID NO: 566.

In some embodiments, the recombinant bacteria comprise gene sequence encoding a PhoA-IL-10-linker-IL-10 fusion protein which comprises SEQ ID NO: 496 or a functional fragment thereof. In some embodiments, recombinant bacteria comprise a gene sequence encoding a PhoA-IL-10-linker-IL-10 polypeptide that is at least about 80%, 85%, 90%, 95%, or 99% identity to PhoA-IL-10-linker-IL-10 polypeptide comprising SEQ ID NO: 496 or a functional fragment thereof. In some embodiments, the PhoA-IL-10-linker-IL-10 polypeptide has at least about 85%, 86%, 87%, 88%, 89%, 90%, 91%, 92%, 93%, 94%, 95%, 96%, 97%, 98%, or 99% identity with SEQ ID NO: 496. In some specific embodiments, the PhoA-IL-10-linker-IL-10 polypeptide comprises SEQ ID NO: 496. In some embodiments, the recombinant bacteria comprise gene sequence encoding a PhoA-IL-10-linker-IL-10 protein. In certain embodiments, the PhoA-IL-10-linker-IL-10 fusion protein gene sequence has at least about 80%, 85%, 90%, 95%, or 99% identity with SEQ ID NO: 489. In some embodiments, the PhoA-IL-10-linker-IL-10 gene sequence has at least about 85%, 86%, 87%, 88%, 89%, 90%, 91%, 92%, 93%, 94%, 95%, 96%, 97%, 98%, or 99% identity with SEQ ID NO: 489. In some specific embodiments, the PhoA-IL-10-linker-IL-10 gene sequence comprises SEQ ID NO: 489.

In some embodiments, the secretion tag is ECOLIN 19410. In some embodiments, the ECOLIN 19410 secretion tag comprises SEQ ID NO: 397. In some embodiments, the ECOLIN 19410 secretion tag gene sequence comprises SEQ ID NO: 491. In some embodiments, the recombinant bacteria comprise gene sequence encoding a ECOLIN 19410-IL-10 fusion protein which comprises SEQ ID NO: 564 or a functional fragment thereof. In some embodiments, recombinant bacteria comprise a gene sequence encoding a ECOLIN 19410-IL-10 polypeptide that is at least about 80%, 85%, 90%, 95%, or 99% identity to ECOLIN 19410-IL-10 polypeptide comprising SEQ ID NO: 564 or a functional fragment thereof. In some embodiments, the ECOLIN 19410-IL-10 polypeptide has at least about 85%, 86%, 87%, 88%, 89%, 90%, 91%, 92%, 93%, 94%, 95%, 96%, 97%, 98%, or 99% identity with SEQ ID NO: 564. In some specific embodiments, the ECOLIN 19410-IL-10 polypeptide comprises SEQ ID NO: 564. In some embodiments, the recombinant bacteria comprise gene sequence encoding a ECOLIN 19410-IL-10 fusion protein. In certain embodiments, the ECOLIN 19410-IL-10 fusion protein gene sequence has at least about 80%, 85%, 90%, 95%, or 99% identity with SEQ ID NO: 566. In some embodiments, the ECOLIN 19410-IL-10 gene sequence has at least about 85%, 86%, 87%, 88%, 89%, 90%, 91%, 92%, 93%, 94%, 95%, 96%, 97%, 98%, or 99% identity with SEQ ID NO: 566. In some specific embodiments, the ECOLIN 19410-IL-10 gene sequence comprises SEQ ID NO: 566.

In some embodiments, the recombinant bacteria comprise gene sequence encoding a ECOLIN 19410-IL-10-linker-IL-10 fusion protein which comprises SEQ ID NO: 495 or a functional fragment thereof. In some embodiments, recombinant bacteria comprise a gene sequence encoding a ECOLIN 19410-IL-10-linker-IL-10 polypeptide that is at least about 80%, 85%, 90%, 95%, or 99% identity to ECOLIN 19410-IL-10-linker-IL-10 polypeptide comprising SEQ ID NO: 495 or a functional fragment thereof. In some embodiments, the ECOLIN 19410-IL-10-linker-IL-10 polypeptide has at least about 85%, 86%, 87%, 88%, 89%, 90%, 91%, 92%, 93%, 94%, 95%, 96%, 97%, 98%, or 99% identity with SEQ ID NO: 495. In some specific embodiments, the ECOLIN 19410-IL-10-linker-IL-10 polypeptide comprises SEQ ID NO: 495. In some embodiments, the recombinant bacteria comprise gene sequence encoding a ECOLIN 19410-IL-10-linker-IL-10 fusion protein. In certain embodiments, the ECOLIN 19410-IL-10-linker-IL-10 fusion protein gene sequence has at least about 80%, 85%, 90%, 95%, or 99% identity with SEQ ID NO: 488. In some embodiments, the ECOLIN 19410-IL-10-linker-IL-10 gene sequence has at least about 85%, 86%, 87%, 88%, 89%, 90%, 91%, 92%, 93%, 94%, 95%, 96%, 97%, 98%, or 99% identity with SEQ ID NO: 488. In some specific embodiments, the ECOLIN 19410-IL-10-linker-IL-10 gene sequence comprises SEQ ID NO: 488.

In any of these embodiments, the IL-10 constructs described herein may further comprise a secretion tag. Non-limiting examples of secretion tags are described herein. The secretion tag is at the N-terminus or at the C-terminus.

In some embodiments, the genetically engineered are capable of producing and secreting IL-10. In some embodiments, the IL-10 gene is functionally replaced, modified, and/or mutated in order to enhance stability and/or increase IL-10 production or secretion. In some embodiments, the recombinant bacteria are capable of expressing and secreting IL-10 in low-oxygen conditions, in the presence of certain molecules or metabolites, in the presence of molecules or metabolites associated with inflammation or an inflammatory response, or in the presence of some other metabolite that may or may not be present in the gut, such as arabinose. Exemplary chemical inducers are described herein.

In one embodiment, the IL-10 gene is directly operably linked to a first promoter. In another embodiment, the IL-10 gene is indirectly operably linked to a first promoter. In one embodiment, the promoter is not operably linked with the IL-10 gene in nature.

In some embodiments, the IL-10 gene is expressed under the control of a constitutive promoter. In another embodiment, the IL-10 gene is expressed under the control of an inducible promoter. In some embodiments, the IL-10 gene is expressed under the control of a promoter that is directly or indirectly induced by exogenous environmental conditions. In one embodiment, the IL-10 gene is expressed under the control of a promoter that is directly or indirectly induced by low-oxygen or anaerobic conditions, wherein expression of the IL-10 gene is activated under low-oxygen or anaerobic environments, such as the environment of the mammalian gut. Inducible promoters are described in more detail infra.

The IL-10 gene may be present on a plasmid or chromosome in the bacterial cell. In one embodiment, the IL-10 gene is located on a plasmid in the bacterial cell. In another embodiment, the IL-10 gene is located in the chromosome of the bacterial cell. In yet another embodiment, a native copy of the IL-10 gene is located in the chromosome of the bacterial cell. The IL-10 gene may be expressed on a low-copy plasmid or a high-copy plasmid. The high-copy plasmid may be useful for increasing expression of IL-10.

IL-2

In some embodiments, the recombinant bacteria are capable of producing IL-2. Interleukin 2 (IL-2) mediates autoimmunity by preserving health of regulatory T cells (Treg). Treg cells, including those expressing Foxp3, typically suppress effector T cells that are active against self-antigens, and in doing so, can dampen autoimmune activity. IL-2 functions as a cytokine to enhance Treg cell differentiation and activity while diminished IL-2 activity can promote autoimmunity events. IL-2 is generated by activated CD4+ T cells, and by other immune mediators including activated CD8+ T cells, activated dendritic cells, natural killer cells, and NK T cells. IL-2 binds to IL-2R, which is composed of three chains including CD25, CD122, and CD132. IL-2 promotes growth of Treg cells in the thymus, while preserving their function and activity in systemic circulation. Treg cell activity plays an intricate role in the IBD setting, with murine studies suggesting a protective role in disease pathogenesis. In some embodiments, the recombinant bacteria comprise a nucleic acid sequence encoding a polypeptide of SEQ ID NO: 144 or a functional fragment thereof. In some embodiments, recombinant bacteria comprise a nucleic acid sequence that is at least about 80%, 85%, 90%, 95%, or 99% homologous to a nucleic acid sequence encoding a polypeptide of SEQ ID NO: 144 or a functional fragment thereof. In some embodiments, the recombinant bacteria are capable of producing IL-2 under inducing conditions, e.g., under a condition(s) associated with inflammation. In some embodiments, the recombinant bacteria are capable of producing IL-2 in low-oxygen conditions.

In some embodiments, the recombinant bacteria comprise a nucleic acid sequence encoding an IL-2 mutein (IL-2 molecule with one or more mutations). Non-limiting examples of IL-2-muteins can be found at least in in WO2016/164937, U.S. Pat. Nos. 9,580,486, 7,105,653, 9,616,105, 9,428,567, US2017/0051029, US2014/0286898A1, WO2014153111A2, WO2010/085495, WO2016014428A2, WO2016025385A1, and US20060269515, each of which are incorporated by reference in its entirety.

In some embodiments, the alanine at position 2 of SEQ ID NO: 144 is deleted. In some embodiments, the IL-2 mutein molecule comprises a serine substituted for cysteine at position 126 of the IL-2 sequence (SEQ ID NO: 144). Other combinations of mutations and substitutions that are IL-2 mutein molecules are described in US20060269515, which is incorporated by reference in its entirety. In some embodiments, the cysteine at position 125 (position 126 in SEQ ID NO: 144) is also substituted with a valine or alanine. In some embodiments, the IL-2 mutein molecule comprises a V91K substitution (position 92 in SEQ ID NO: 144). In some embodiments, the IL-2 mutein molecule comprises a N88D substitution (position 89 in SEQ ID NO: 144). In some embodiments, the IL-2 mutein molecule comprises a N88R substitution (position 89 in SEQ ID NO: 144). In some embodiments, the IL-2 mutein molecule comprises a substitution of H16E, D84K, V91N, N88D, V91K, or V91R, any combinations thereof (position 17, 85, 92, 89, 92, or 92, respectively, in SEQ ID NO: 144). In some embodiments, the IL-2 mutein molecule comprises one or more substitutions selected from the group consisting of: T3N or T3A (position 4 in SEQ ID NO: 144); L12G, L12K, L12Q, or L12S (position 13 in SEQ ID NO: 144); Q13G (position 14 in SEQ ID NO: 144); E15A, E15G, or E15S (position 16 in SEQ ID NO: 144); H16A, H16D, H16G, H16K, H16M, H16N, H16R, H16S, H16T, H16V, or H16Y (position 17 in SEQ ID NO: 144); L19A, L19D, L19E, L19G, L19N, L19R, L19S, L19T, or L19V (position 20 in SEQ ID NO: 144); D20A, D20E, D20H, D201, D20Y, D20F, D20G, D20T, or D20W (position 21 in SEQ ID NO: 144); M23R (position 24 in SEQ ID NO: 144); R81A, R81G, R81S, or R81T (position 82 in SEQ ID NO: 144); D84A, D84E, D84G, D84I, D84M, D84Q D84R, D84S, or D84T (position 85 in SEQ ID NO: 144); S87R (position 88 in SEQ ID NO: 144); N88A, N88D, N88E, N88I, N88F, N88G, N88M, N88R, N88S, N88V, or N88W (position 89 in SEQ ID NO: 144); V91D, V91E, V91G, or V91S (position 92 in SEQ ID NO: 144); 192K, or I92R (position 93 in SEQ ID NO: 144); E95G (position 96 in SEQ ID NO: 144); and Q126 (position 127 in SEQ ID NO: 144). In some embodiments, the amino acid sequence of the IL-2 mutein molecule comprises a C125A or C125S substitution (position 126 in SEQ ID NO: 144) and with one substitution selected from T3N or T3A (position 4 in SEQ ID NO: 144); L12G, L12K, L12Q or L12S (position 13 in SEQ ID NO: 144); Q13G (position 14 in SEQ ID NO: 144); E15A, E15G, or E15S (position 16 in SEQ ID NO: 144); H16A, H16D, H16G, H16K, H16M, H16N, H16R, H165, H16T, H16V, or H16Y (position 17 in SEQ ID NO: 144); L19A, L19D, L19E, L19G, L19N, L19R, L19S, L19T, or L19V (position 20 in SEQ ID NO: 144); D20A, D20E, D20F, D20G, D20T, or D20W (position 21 in SEQ ID NO: 144); M23R (position 24 in SEQ ID NO: 144); R81A, R81G, R81S, or R81T (position 82 in SEQ ID NO: 144); D84A, D84E, D84G, D84I, D84M, D84Q, D84R, D84S, or D84T (position 85 in SEQ ID NO: 144); S87R (position 88 in SEQ ID NO: 144); N88A, N88D, N88E, N88F, N88I, N88G, N88M, N88R, N88S, N88V, or N88W (position 89 in SEQ ID NO: 144); V91D, V91E, V91G, or V91S (position 92 in SEQ ID NO: 144); I92K, or I92R (position 93 in SEQ ID NO: 144); E95G (position 96 in SEQ ID NO: 144); and Q1261, Q126L, or Q126F (position 127 in SEQ ID NO: 144). In some embodiments, the IL-2 mutein molecule comprises a C125A or C125S substitution (position 126 in SEQ ID NO: 144) and with one substitution selected from D20H, D201, D20Y, D20E, D20G, or D20W (position 21 in SEQ ID NO: 144); D84A, or D84S (position 85 in SEQ ID NO: 144); H16D, H16G, H16K, H16R, H16T, or H16V (position 17 in SEQ ID NO: 144); I92K, or I92R (position 93 in SEQ ID NO: 144); L12K (position 13 in SEQ ID NO: 144); L19D, L19N, or L19T (position 20 in SEQ ID NO: 144); N88D, N88R, or N88S (position 89 in SEQ ID NO: 144); and V91D, V91G, V91K, or V91S (position 92 in SEQ ID NO: 144). In some embodiments, the IL-2 mutein comprises N88R (position 89 in SEQ ID NO: 144) and/or D20H (position 21 in SEQ ID NO: 144) mutations.

In some embodiments, the IL-2 mutein comprises a mutation at one more of positions 30, 32, 36, 38, 49, 70, 72, 75, 89, and 126 that correspond to SEQ ID NO: 144. The substitutions can be used alone or in combination with one another. In some embodiments, the IL-2 mutein comprises substitutions at 3, 4, 5, 6, 7, 8, 9, 10 or each of positions 50, 52, 56, 58, 69, 90, 92, 95, 109, and 146. Non-limiting examples such combinations include, but are not limited to, a mutation at positions 50, 52, 56, 58, 69, 90, 92, 95, 109, and 146; 50, 52, 56, 58, 69, 90, 92, 95, and 109; 50, 52, 56, 58, 69, 90, 92, and 95; 50, 52, 56, 58, 69, 90, and 92; 50, 52, 56, 58, 69, and 90; 50, 52, 56, 58, and 69; 50, 52, 56, and 58; 50, 52, and 56; 50 and 52; 52, 56, 58, 69, 90, 92, 95, 109, and 146; 52, 56, 58, 69, 90, 92, 95, and 109; 52, 56, 58, 69, 90, 92, and 95; 52, 56, 58, 69, 90, and 92; 52, 56, 58, 69, and 90; 56, 58, and 69; 56 and 58; 56, 58, 69, 90, 92, 95, 109, and 146; 56, 58, 69, 90, 92, 95, and 109; 56, 58, 69, 90, 92, and 95; 56, 58, 69, 90, and 92; 56, 57, 69, and 90; 56, 58, and 69; 56 and 58; 58, 69, 90, 92, 95, 109, and 146; 58, 69, 90, 92, 95, and 109; 58, 69, 90, 92, and 95; 58, 69, 90, and 92; 58, 69, and 90; 58 and 69; 69, 90, 92, 95, 109, and 146; 69, 90, 92, 95, and 109; 69, 90, 92, and 95; 69, 90, and 92; 69 and 90; 90, 92, 95, 109, and 146; 90, 92, 95, and 109; 90, 92, and 95; 90 and 92; 92, 95, 109, and 146; 92, 95, and 109; 92, and 95; or 95 and 109. Each mutation can be combined with one another. The same substitutions can be made in SEQ ID NO: 144.

In some embodiments, the recombinant bacteria comprise one or more gene sequence(s) selected from PhoA-IL-2, OmpF-IL-2, and TorA-IL-2.

IL-22

In some embodiments, the recombinant bacteria are capable of producing IL-22. Interleukin 22 (IL-22) cytokine can be produced by dendritic cells, lymphoid tissue inducer-like cells, natural killer cells and expressed on adaptive lymphocytes. Through initiation of Jak-STAT signaling pathways, IL-22 expression can trigger expression of antimicrobial compounds as well as a range of cell growth related pathways, both of which enhance tissue repair mechanisms. IL-22 is critical in promoting intestinal barrier fidelity and healing, while modulating inflammatory states. Murine models have demonstrated improved intestinal inflammation states following administration of IL-22. Additionally, IL-22 activates STAT3 signaling to promote enhanced mucus production to preserve barrier function. IL-22's association with IBD susceptibility genes may modulate phenotypic expression of disease as well. In some embodiments, the recombinant bacteria comprise a nucleic acid sequence encoding SEQ ID NO: 145 or a functional fragment thereof. In some embodiments, recombinant bacteria comprise a nucleic acid sequence that is at least about 80%, 85%, 90%, 95%, or 99% homologous to a nucleic acid sequence encoding SEQ ID NO: 145 a functional fragment thereof. In some embodiments, the recombinant bacteria are capable of producing IL-22 under inducing conditions, e.g., under a condition(s) associated with inflammation. In some embodiments, the recombinant bacteria are capable of producing IL-22 in low-oxygen conditions.

In some embodiments, the recombinant bacteria comprise one or more gene sequence(s) selected from PhoA-IL-22, OmpF-IL-22, and TorA-IL-22.

IL-27

In some embodiments, the recombinant bacteria are capable of producing IL-27. Interleukin 27 (IL-27) cytokine is predominately expressed by activated antigen presenting cells, while IL-27 receptor is found on a range of cells including T cells, NK cells, among others. In particular, IL-27 suppresses development of pro-inflammatory T helper 17 (Th17) cells, which play a critical role in IBD pathogenesis. Further, IL-27 can promote differentiation of IL-10 producing Tr1 cells and enhance IL-10 output, both of which have anti-inflammatory effects. IL-27 has protective effects on epithelial barrier function via activation of MAPK and STAT signaling within intestinal epithelial cells. Additionally, IL-27 enhances production of antibacterial proteins that curb bacterial growth. Improvement in barrier function and reduction in bacterial growth suggest a favorable role for IL-27 in IBD pathogenesis. In some embodiments, the recombinant bacteria comprise a nucleic acid sequence encoding SEQ ID NO: 146 or a functional fragment thereof. In some embodiments, recombinant bacteria comprise a nucleic acid sequence that is at least about 80%, 85%, 90%, 95%, or 99% homologous to a nucleic acid sequence encoding SEQ ID NO: 146 or a functional fragment thereof. In some embodiments, the recombinant bacteria are capable of producing IL-27 under inducing conditions, e.g., under a condition(s) associated with inflammation. In some embodiments, the recombinant bacteria are capable of producing IL-27 in low-oxygen conditions.

In some embodiments, the recombinant bacteria comprise one or more gene sequence(s) selected from PhoA-IL-27, OmpF-IL-27, and TorA-IL-27.

SOD

In some embodiments, the recombinant bacteria are capable of producing superoxide dismutase (SOD). Increased ROS levels contribute to pathophysiology of inflammatory bowel disease. Increased ROS levels may lead to enhanced expression of vascular cell adhesion molecule 1 (VCAM-1), which can facilitate translocation of inflammatory mediators to disease affected tissue, and result in a greater degree of inflammatory burden. Antioxidant systems including superoxide dismutase (SOD) can function to mitigate overall ROS burden. However, studies indicate that the expression of SOD in the setting of IBD may be compromised, e.g., produced at lower levels in IBD, thus allowing disease pathology to proceed. Further studies have shown that supplementation with SOD to rats within a colitis model is associated with reduced colonic lipid peroxidation and endothelial VCAM-1 expression as well as overall improvement in inflammatory environment. Thus, in some embodiments, the recombinant bacteria comprise a nucleic acid sequence encoding a polypeptide of SEQ ID NO: 147 or a functional fragment thereof. In some embodiments, recombinant bacteria comprise a nucleic acid sequence that is at least about 80%, 85%, 90%, 95%, or 99% homologous to a nucleic acid sequence encoding a polypeptide of SEQ ID NO: 147 or a functional fragment thereof. In some embodiments, the recombinant bacteria are capable of producing SOD under inducing conditions, e.g., under a condition(s) associated with inflammation. In some embodiments, the recombinant bacteria are capable of producing SOD in low-oxygen conditions.

GLP2

In some embodiments, the recombinant bacteria are capable of producing GLP-2 or proglucagon. Glucagon-like peptide 2 (GLP-2) is produced by intestinal endocrine cells and stimulates intestinal growth and enhances gut barrier function. GLP-2 administration has therapeutic potential in treating IBD, short bowel syndrome, and small bowel enteritis (Yazbeck et al., 2009). The recombinant bacteria may comprise any suitable gene encoding GLP-2 or proglucagon, e.g., human GLP-2 or proglucagon. In some embodiments, a protease inhibitor, e.g., an inhibitor of dipeptidyl peptidase, is also administered to decrease GLP-2 degradation. In some embodiments, the recombinant bacteria express a degradation resistant GLP-2 analog, e.g., Teduglutide (Yazbeck et al., 2009). In some embodiments, the gene encoding GLP-2 or proglucagon is modified and/or mutated, e.g., to enhance stability, increase GLP-2 production, and/or increase gut barrier enhancing potency under inducing conditions. In some embodiments, the recombinant bacteria are capable of producing GLP-2 or proglucagon under inducing conditions. GLP-2 administration in a murine model of IBD is associated with reduced mucosal damage and inflammation, as well as a reduction in inflammatory mediators, such as TNF-α and IFN-gamma. Further, GLP-2 supplementation may also lead to reduced mucosal myeloperoxidase in colitis/ileitis models.

In some embodiments, the recombinant bacteria comprise a gene sequence encoding GLP-2. In some embodiments, the recombinant bacteria comprise a gene sequence encoding SEQ ID NO: 148 or a functional fragment thereof. In some embodiments, recombinant bacteria comprise a nucleic acid sequence that is at least about 80%, 85%, 90%, 95%, or 99% homologous to a nucleic acid sequence encoding SEQ ID NO: 148 or a functional fragment thereof. In certain embodiments, the GLP-2 gene sequence has at least about 80% identity with SEQ ID NO: 523. In certain embodiments, the GLP-2 gene sequence has at least about 90% identity with SEQ ID NO: 523. In certain embodiments, the GLP-2 gene sequence has at least about 95% identity with SEQ ID NO: 523. In some embodiments, the GLP-2 gene sequence has at least about 85%, 86%, 87%, 88%, 89%, 90%, 91%, 92%, 93%, 94%, 95%, 96%, 97%, 98%, or 99% identity with SEQ ID NO: 523. In some specific embodiments, the GLP-2 gene sequence comprises SEQ ID NO: 523. In other specific embodiments, the GLP-2 gene sequence consists of SEQ ID NO: 523.

In some embodiments, the recombinant bacteria are capable of producing GLP-2 under inducing conditions, e.g., under a condition(s) associated with inflammation. In some embodiments, the recombinant bacteria are capable of producing GLP-2 in low-oxygen conditions.

In some embodiments, the recombinant bacteria comprise gene sequence(s) for the production of GLP-2-Fc fusion proteins. In some embodiments, the recombinant bacteria comprise gene sequence(s) encoding GLP2-Fc fusion protein(s). In some embodiments, the recombinant bacteria comprise gene sequence(s) encoding GLP2-FcIgA fusion protein(s). In some embodiments, the GLP-2 polypeptide is located N terminally relative to the Fc polypeptide. In some embodiments, the GLP-2 polypeptide is located C-terminally relative to the polypeptide.

In some embodiments, the recombinant bacteria comprise gene sequence(s) encoding GLP2-FcIgG fusion protein(s). In some embodiments, the Fc portion of the human IgA used herein contains the hinge, CH2 and CH3 regions of the Fc protein. In some embodiments, the Fc portion of the human IgA used herein contains the hinge, CH1, CH2 and CH3 regions of the Fc protein. In some embodiments, the Fc portion of the human IgA used herein contains the hinge, and CH2 region of the Fc protein. In some embodiments, the Fc portion of the human IgA used herein contains the hinge, and CH3 region of the Fc protein. In some embodiments, the Fc portion of the human IgA used herein contains the hinge, and CH1 region of the Fc protein.

TABLE 3
Non-limiting Examples of GLP-2 Polypeptide Sequences

Construct comprising human GLP2 - linker - Fc (human IgA) (SEQ ID
NO: 537)
Construct comprising PhoA-GLP2 (SEQ ID NO: 538)
Construct comprising ECOLIN 19410-GLP2 (SEQ ID NO: 539)
Construct comprising mutated GLP2 - linker - Fc (human IgA) (SEQ ID
NO: 540)
Construct comprising PhoA-mutated GLP2 (SEQ ID NO: 541)
Construct comprising ECOLIN 19410-mutated GLP2 (SEQ ID NO: 542)
Construct comprising PhoA secretion tag -mutated human GLP2 -
linker - Fc (human IgA) - 8X His Tag (SEQ ID NO: 543)
Construct comprising PhoA secretion tag -mutated human GLP2 -
linker - Fc (human IgA) (SEQ ID NO: 544)
Construct comprising ECOLIN 19410 secretion tag -mutated human
GLP2 - linker - Fc (human IgA) - 8X His Tag (SEQ ID NO: 545)
Construct comprising ECOLIN 19410 secretion tag -mutated human
GLP2 - linker - Fc (human IgA) (SEQ ID NO: 546)

TABLE 4
Non-limiting Examples of GLP-2 fusion
protein Polynucleotide Sequences

Construct comprising human GLP2 - linker - Fc (human IgA) (SEQ ID
NO: 547)
Second construct comprising human GLP2 - linker - Fc (human IgA)
(SEQ ID NO: 548)
PhoA-GLP-2 (SEQ ID NO: 549)
ECOLIN 19410- GLP-2 (SEQ ID NO: 550)

TABLE 5
Non-limiting Examples of Mutant GLP-2
Fusion Protein Polynucleotide Sequences

Construct comprising PhoA secretion tag -mutated GLP2 - linker - Fc
(human IgA) - 8X His Tag (SEQ ID NO: 551)
Second construct comprising PhoA secretion tag - mutated GLP2 -
linker - Fc (human IgA) - 8X His Tag (SEQ ID NO: 552)
Construct comprising PhoA secretion tag - mutated GLP2 - linker - Fc
(human IgA) (SEQ ID NO: 553)
Second construct comprising PhoA secretion tag - mutated GLP2 -
linker - Fc (human IgA) (SEQ ID NO: 554)
Construct comprising secretion tag from ECOLIN 19410 - mutated
GLP2-linker-Fc (human IgA) - - 8X His Tag (SEQ ID NO: 555)
Second construct comprising secretion tag from ECOLIN 19410 -
mutated GLP2-linker-Fc (human IgA) - - 8X His Tag (SEQ ID NO: 556)
Construct comprising secretion tag from ECOLIN 19410 - mutated
GLP2-linker-Fc (human IgA) (SEQ ID NO: 557)
Second construct comprising secretion tag from ECOLIN 19410 -
mutated GLP2-linker-Fc (human IgA) (SEQ ID NO: 558)
Construct comprising mutated GLP2 - linker - Fc (human IgA) (SEQ ID
NO: 559)
Second construct comprising mutated GLP2 - linker - Fc (human IgA)
(SEQ ID NO: 560)
PhoA- mutated GLP-2 (SEQ ID NO: 561)
ECOLIN 19410- mutated GLP-2 (SEQ ID NO: 562)

In one embodiment, the 33 amino acid human GLP-2 sequence e.g., of SEQ ID NO: 148 is fused to the N-terminus of the Fc portion of human IgA protein, e.g., SEQ ID NO: 499 linked by a 20 amino acid linker, e.g., SEQ ID NO: 509.

In some embodiments, the recombinant bacteria comprise a Fc (IgA) gene sequence encoding SEQ ID NO: 499 or a functional fragment thereof. In some embodiments, recombinant bacteria comprise a Fc (IgA) gene sequence encoding a polypeptide that is at least about 80%, 85%, 90%, 95%, or 99% identity to a Fc (IgA) polypeptide comprising SEQ ID NO: 499 or a functional fragment thereof. In some embodiments, the Fc (IgA) polypeptide has at least about 85%, 86%, 87%, 88%, 89%, 90%, 91%, 92%, 93%, 94%, 95%, 96%, 97%, 98%, or 99% identity with SEQ ID NO: 499. In some specific embodiments, the Fc (IgA) polypeptide comprises SEQ ID NO: 499. In other specific embodiments, the Fc (IgA) polypeptide consists of SEQ ID NO: 499. In some embodiments, the recombinant bacteria comprise gene sequence encoding a Fc (IgA) fusion protein. In certain embodiments, the Fc (IgA) gene sequence has at least about 80%, 85%, 90%, 95%, or 99% identity with SEQ ID NO: 527. In some embodiments, the Fc (IgA) gene sequence has at least about 85%, 86%, 87%, 88%, 89%, 90%, 91%, 92%, 93%, 94%, 95%, 96%, 97%, 98%, or 99% identity with SEQ ID NO: 527. In some specific embodiments, the Fc (IgA) gene sequence comprises SEQ ID NO: 527. In other specific embodiments, the Fc (IgA) gene sequence consists of SEQ ID NO: 527. In certain embodiments, the Fc (IgA) gene sequence has at least about 80%, 85%, 90%, 95%, or 99% identity with SEQ ID NO: 528. In some embodiments, the Fc (IgA) gene sequence has at least about 85%, 86%, 87%, 88%, 89%, 90%, 91%, 92%, 93%, 94%, 95%, 96%, 97%, 98%, or 99% identity with SEQ ID NO: 528. In some specific embodiments, the Fc (IgA) gene sequence comprises SEQ ID NO: 528. In other specific embodiments, the Fc (IgA) gene sequence consists of SEQ ID NO: 528.

In some embodiments, the recombinant bacteria comprise a linker gene sequence encoding SEQ ID NO: 509 or a functional fragment thereof. In some embodiments, recombinant bacteria comprise a linker gene sequence encoding a linker that is at least about 80%, 85%, 90%, 95%, or 99% identity to a linker comprising SEQ ID NO: 509 or a functional fragment thereof. In some embodiments, the linker polypeptide has at least about 85%, 86%, 87%, 88%, 89%, 90%, 91%, 92%, 93%, 94%, 95%, 96%, 97%, 98%, or 99% identity with SEQ ID NO: 509. In some specific embodiments, the linker polypeptide comprises SEQ ID NO: 509. In other specific embodiments, the linker polypeptide consists of SEQ ID NO: 509.

In some embodiments, the recombinant bacteria comprise gene sequence encoding a glp2-linker Fc (IgA) fusion protein. In some embodiments, the recombinant bacteria comprise gene sequence(s) encoding the GLP-2 fusion protein of SEQ ID NO: 537. In some embodiments, the recombinant bacteria comprise a glp2-linker Fc (IgA) gene sequence encoding SEQ ID NO: 537 or a functional fragment thereof. In some embodiments, recombinant bacteria comprise a glp2-linker Fc (IgA) gene sequence encoding a polypeptide that is at least about 80%, 85%, 90%, 95%, or 99% identity to a glp2-linker Fc (IgA) polypeptide comprising SEQ ID NO: 537 or a functional fragment thereof. In some embodiments, the glp2-linker Fc (IgA) polypeptide has at least about 85%, 86%, 87%, 88%, 89%, 90%, 91%, 92%, 93%, 94%, 95%, 96%, 97%, 98%, or 99% identity with SEQ ID NO: 537. In some specific embodiments, the glp2-linker Fc (IgA) polypeptide comprises SEQ ID NO: 537. In other specific embodiments, the glp2-linker Fc (IgA) polypeptide consists of SEQ ID NO: 537. In certain embodiments, the glp2-linker Fc (IgA) gene sequence has at least about 80%, 85%, 90%, 95%, or 99% identity with SEQ ID NO: 547. In some embodiments, the glp2-linker Fc (IgA) gene sequence has at least about 85%, 86%, 87%, 88%, 89%, 90%, 91%, 92%, 93%, 94%, 95%, 96%, 97%, 98%, or 99% identity with SEQ ID NO: 547. In some specific embodiments, the glp2-linker Fc (IgA) gene sequence comprises SEQ ID NO: 547. In other specific embodiments, the glp2-linker Fc (IgA) gene sequence consists of SEQ ID NO: 547.

In certain embodiments, the glp2-linker Fc (IgA) gene sequence has at least about 80%, 85%, 90%, 95%, or 99% identity with SEQ ID NO: 548. In some embodiments, the glp2-linker Fc (IgA) gene sequence has at least about 85%, 86%, 87%, 88%, 89%, 90%, 91%, 92%, 93%, 94%, 95%, 96%, 97%, 98%, or 99% identity with SEQ ID NO: 548. In some specific embodiments, the glp2-linker Fc (IgA) gene sequence comprises SEQ ID NO: 548. In other specific embodiments, the glp2-linker Fc (IgA) gene sequence consists of SEQ ID NO: 548.

In any of these embodiments, the recombinant bacteria may further comprise gene sequences encoding a secretion tag. Non-limiting examples of such secretion tags are described herein and include PhoA, OmpF, cvaC, TorA, fdnG, dmsA, PelB, the ECOLIN_05715 secretion signal, ECOLIN_16495 secretion signal, ECOLIN_19410 secretion signal, and the ECOLIN_19880 secretion signal. In some embodiments, the secretion tag is PhoA. In some embodiments, the PhoA secretion tag comprises SEQ ID NO: 385. In some embodiments, the PhoA secretion tag comprises SEQ ID NO: 386. In some embodiments, the recombinant bacteria comprise PhoA tag gene sequence comprising SEQ ID NO: 490. In some embodiments, the recombinant bacteria comprise gene sequence encoding a PhoA-GLP-2 fusion protein. In some embodiments, the recombinant bacteria comprise a PhoA-GLP-2 fusion gene sequence encoding SEQ ID NO: 538 or a functional fragment thereof. In some embodiments, recombinant bacteria comprise a PhoA-GLP-2 fusion gene sequence encoding a polypeptide that is at least about 80%, 85%, 90%, 95%, or 99% homologous to PhoA-GLP-2 fusion polypeptide comprising SEQ ID NO: 538 or a functional fragment thereof. In some embodiments, the PhoA-GLP-2 fusion polypeptide has at least about 85%, 86%, 87%, 88%, 89%, 90%, 91%, 92%, 93%, 94%, 95%, 96%, 97%, 98%, or 99% identity with SEQ ID NO: 538. In some specific embodiments, the PhoA-GLP-2 fusion polypeptide comprises SEQ ID NO: 538. In other specific embodiments, the PhoA-GLP-2 fusion polypeptide consists of SEQ ID NO: 538. In certain embodiments, the PhoA-GLP-2 fusion gene sequence has at least about 80%, 85%, 90%, 95%, or 99% identity with SEQ ID NO: 549. In some embodiments, the PhoA-GLP-2 fusion gene sequence has at least about 85%, 86%, 87%, 88%, 89%, 90%, 91%, 92%, 93%, 94%, 95%, 96%, 97%, 98%, or 99% identity with SEQ ID NO: 549. In some specific embodiments, the PhoA-GLP-2 fusion gene sequence comprises SEQ ID NO: 549. In other specific embodiments, the PhoA-GLP-2 fusion gene sequence consists of SEQ ID NO: 549.

In some embodiments, the genetically engineered bacteria comprise a GLP-2 fusion protein encoding PhoA-GLP-2-Fc (hIgA). In some embodiments, the recombinant bacteria comprise gene sequence encoding a PhoA-GLP-2-Fc (hIgA) fusion protein which comprises SEQ ID NO: 511 or a functional fragment thereof. In some embodiments, recombinant bacteria comprise a gene sequence encoding a PhoA-GLP-2-Fc (hIgA) polypeptide that is at least about 80%, 85%, 90%, 95%, or 99% identity to PhoA-GLP-2-Fc (hIgA) polypeptide comprising SEQ ID NO: 511 or a functional fragment thereof. In some embodiments, the PhoA-GLP-2-Fc (hIgA) polypeptide has at least about 85%, 86%, 87%, 88%, 89%, 90%, 91%, 92%, 93%, 94%, 95%, 96%, 97%, 98%, or 99% identity with SEQ ID NO: 511. In some specific embodiments, the PhoA-GLP-2-Fc (hIgA) polypeptide comprises SEQ ID NO: 511. In other specific embodiments, the PhoA-GLP-2-Fc (hIgA) polypeptide consists of SEQ ID NO: 511. In some embodiments, the recombinant bacteria comprise gene sequence encoding a GLP-2 fusion protein which comprises SEQ ID NO: 512 or a functional fragment thereof. In some embodiments, recombinant bacteria comprise a gene sequence encoding a polypeptide that is at least about 80%, 85%, 90%, 95%, or 99% identity to polypeptide comprising SEQ ID NO: 512 or a functional fragment thereof. In some embodiments, the polypeptide has at least about 85%, 86%, 87%, 88%, 89%, 90%, 91%, 92%, 93%, 94%, 95%, 96%, 97%, 98%, or 99% identity with SEQ ID NO: 512. In some specific embodiments, the polypeptide comprises SEQ ID NO: 512. In other specific embodiments, the polypeptide consists of SEQ ID NO: 512.

In certain embodiments, the PhoA-GLP-2-Fc (hIgA) gene sequence has at least about 80%, 85%, 90%, 95%, or 99% identity with SEQ ID NO: 529. In some embodiments, the PhoA-GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 529. In some specific embodiments, the PhoA-GLP-2-Fc (hIgA) gene sequence comprises SEQ ID NO: 529. In other specific embodiments, the PhoA-GLP-2-Fc (hIgA) gene sequence consists of SEQ ID NO: 529.

In certain embodiments, the PhoA-GLP-2-Fc (hIgA) gene sequence has at least about 80%, 85%, 90%, 95%, or 99% identity with SEQ ID NO: 528. In some embodiments, the PhoA-GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 528. In some specific embodiments, the PhoA-GLP-2-Fc (hIgA) gene sequence comprises SEQ ID NO: 528. In other specific embodiments, the PhoA-GLP-2-Fc (hIgA) gene sequence consists of SEQ ID NO: 528.

In certain embodiments, the PhoA-GLP-2-Fc (hIgA) gene sequence has at least about 80%, 85%, 90%, 95%, or 99% identity with SEQ ID NO: 530. In some embodiments, the PhoA-GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 530. In some specific embodiments, the PhoA-GLP-2-Fc (hIgA) gene sequence comprises SEQ ID NO: 530. In other specific embodiments, the PhoA-GLP-2-Fc (hIgA) gene sequence consists of SEQ ID NO: 530.

In certain embodiments, the PhoA-GLP-2-Fc (hIgA) gene sequence has at least about 80%, 85%, 90%, 95%, or 99% identity with SEQ ID NO: 531. In some embodiments, the PhoA-GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 531. In some specific embodiments, the PhoA-GLP-2-Fc (hIgA) gene sequence comprises SEQ ID NO: 531. In other specific embodiments, the PhoA-GLP-2-Fc (hIgA) gene sequence consists of SEQ ID NO: 531.

In some embodiments, the secretion tag is ECOLIN 19410. In some embodiments, the ECOLIN 19410 secretion tag comprises SEQ ID NO: 397. In some embodiments, the gene sequence encoding ECOLIN 19410 secretion tag comprises SEQ ID NO: 491. In some embodiments, the recombinant bacteria comprise gene sequence encoding a ECOLIN 19410-GLP-2 fusion protein. In some embodiments, the recombinant bacteria comprise gene sequence encoding a ECOLIN 19410-GLP-2 fusion protein which comprises SEQ ID NO: 539 or a functional fragment thereof. In some embodiments, recombinant bacteria comprise a gene sequence encoding a ECOLIN 19410-GLP-2 polypeptide that is at least about 80%, 85%, 90%, 95%, or 99% identity to ECOLIN 19410-GLP-2 polypeptide comprising SEQ ID NO: 539 or a functional fragment thereof. In some embodiments, the ECOLIN 19410-GLP-2 polypeptide has at least about 85%, 86%, 87%, 88%, 89%, 90%, 91%, 92%, 93%, 94%, 95%, 96%, 97%, 98%, or 99% identity with SEQ ID NO: 539. In some specific embodiments, the ECOLIN 19410-GLP-2 polypeptide comprises SEQ ID NO: 539. In other specific embodiments, the ECOLIN 19410-GLP-2 polypeptide consists of SEQ ID NO: 539.

In certain embodiments, the ECOLIN 19410-GLP-2 fusion protein gene sequence has at least about 80%, 85%, 90%, 95%, or 99% identity with SEQ ID NO: 550. In some embodiments, the ECOLIN 19410-GLP-2 gene sequence has at least about 85%, 86%, 87%, 88%, 89%, 90%, 91%, 92%, 93%, 94%, 95%, 96%, 97%, 98%, or 99% identity with SEQ ID NO: 550. In some specific embodiments, the ECOLIN 19410-GLP-2 gene sequence comprises SEQ ID NO: 550. In other specific embodiments, the ECOLIN 19410-GLP-2 gene sequence consists of SEQ ID NO: 550.

In some embodiments, the recombinant bacteria comprise a GLP-2 fusion protein encoding ECOLIN 19410-GLP-2-Fc (hIgA). In some embodiments, the recombinant bacteria comprise gene sequence encoding a ECOLIN 19410-GLP-2-Fc (hIgA) protein which comprises SEQ ID NO: 513 or a functional fragment thereof. In some embodiments, recombinant bacteria comprise a gene sequence encoding a ECOLIN 19410-GLP-2-Fc (hIgA) polypeptide that is at least about 80%, 85%, 90%, 95%, or 99% identity to ECOLIN 19410-GLP-2-Fc (hIgA) polypeptide comprising SEQ ID NO: 513 or a functional fragment thereof. In some embodiments, the ECOLIN 19410-GLP-2-Fc (hIgA) polypeptide has at least about 85%, 86%, 87%, 88%, 89%, 90%, 91%, 92%, 93%, 94%, 95%, 96%, 97%, 98%, or 99% identity with SEQ ID NO: 513. In some specific embodiments, the ECOLIN 19410-GLP-2-Fc (hIgA) polypeptide comprises SEQ ID NO: 513. In other specific embodiments, the ECOLIN 19410-GLP-2-Fc (hIgA) polypeptide consists of SEQ ID NO: 513. In certain embodiments, the gene sequence has at least about 80%, 85%, 90%, 95%, or 99% identity with SEQ ID NO: 533. In some embodiments, the ECOLIN 19410-GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 533. In some specific embodiments, the ECOLIN 19410-GLP-2-Fc (hIgA) gene sequence comprises SEQ ID NO: 533. In other specific embodiments, the ECOLIN 19410-GLP-2-Fc (hIgA) gene sequence consists of SEQ ID NO: 533.

In certain embodiments, the ECOLIN 19410-GLP-2-Fc (hIgA) ECOLIN 19410-GLP-2-Fc (hIgA) gene sequence has at least about 80%, 85%, 90%, 95%, or 99% identity with SEQ ID NO: 534. In some embodiments, the ECOLIN 19410-GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 534. In some specific embodiments, the ECOLIN 19410-GLP-2-Fc (hIgA) gene sequence comprises SEQ ID NO: 534. In other specific embodiments, the ECOLIN 19410-GLP-2-Fc (hIgA) gene sequence consists of SEQ ID NO: 534.

In some embodiments, the recombinant bacteria comprise ECOLIN 19410-GLP-2-Fc (hIgA) gene sequence encoding a ECOLIN 19410-GLP-2-Fc (hIgA) fusion protein. In certain embodiments, the ECOLIN 19410-GLP-2-Fc (hIgA) gene sequence has at least about 80%, 85%, 90%, 95%, or 99% identity with SEQ ID NO: 535. In some embodiments, the ECOLIN 19410-GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 535. In some specific embodiments, the ECOLIN 19410-GLP-2-Fc (hIgA) gene sequence comprises SEQ ID NO: 535. In other specific embodiments, the ECOLIN 19410-GLP-2-Fc (hIgA) gene sequence consists of SEQ ID NO: 535.

In certain embodiments, the ECOLIN 19410-GLP-2-Fc (hIgA) gene sequence has at least about 80%, 85%, 90%, 95%, or 99% identity with SEQ ID NO: 536. In some embodiments, the ECOLIN 19410-GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 536. In some specific embodiments, the ECOLIN 19410-GLP-2-Fc (hIgA) gene sequence comprises SEQ ID NO: 536. In other specific embodiments, the ECOLIN 19410-GLP-2-Fc (hIgA) gene sequence consists of SEQ ID NO: 536.

In some embodiments, the recombinant bacteria comprise ECOLIN 19410-GLP-2-Fc (hIgA) gene sequence encoding a GLP-2 fusion protein which comprises SEQ ID NO: 514 or a functional fragment thereof. In some embodiments, recombinant bacteria comprise a ECOLIN 19410-GLP-2-Fc (hIgA) gene sequence encoding a polypeptide that is at least about 80%, 85%, 90%, 95%, or 99% identity to polypeptide comprising SEQ ID NO: 514 or a functional fragment thereof. In some embodiments, the polypeptide has at least about 85%, 86%, 87%, 88%, 89%, 90%, 91%, 92%, 93%, 94%, 95%, 96%, 97%, 98%, or 99% identity with SEQ ID NO: 514. In some specific embodiments, the polypeptide comprises SEQ ID NO: 514. In other specific embodiments, the polypeptide consists of SEQ ID NO: 514.

In some embodiments, the recombinant bacteria are capable of producing GLP-2 analogs, including but not limited to, Gattex and teduglutide. Teduglutide is a protease resistan analog of GLP-2. It is made up of 33 amino acids and differs from GLP-2 by one amino acid (alanine is substituted by glycine). The significance of this substitution is that teduglutide is longer acting than endogenous GLP-2 as it is more resistant to proteolysis from dipeptidyl peptidase-4.

In some embodiments, the recombinant bacteria comprise a gene sequence encoding SEQ ID NO: 149 or a functional fragment thereof. In some embodiments, recombinant bacteria comprise a nucleic acid sequence that is at least about 80%, 85%, 90%, 95%, or 99% homologous to a nucleic acid sequence encoding SEQ ID NO: 149 or a functional fragment thereof. In certain embodiments, the mutated GLP-2 gene sequence has at least about 80%, 85%, 90%, 95%, or 99% identity with SEQ ID NO: 526. In some embodiments, the mutated GLP-2 gene sequence has at least about 85%, 86%, 87%, 88%, 89%, 90%, 91%, 92%, 93%, 94%, 95%, 96%, 97%, 98%, or 99% identity with SEQ ID NO: 526. In some specific embodiments, the mutated GLP-2 gene sequence comprises SEQ ID NO: 526. In other specific embodiments, the mutated GLP-2 gene sequence consists of SEQ ID NO: 526.

In one embodiment, a mutated human GLP-2 sequence e.g., of SEQ ID NO: 149 is fused to the N-terminus of the Fc portion of human IgA protein, e.g., SEQ ID NO: 499 linked by a 20 amino acid linker, e.g., SEQ ID NO: 509. In some embodiments, the recombinant bacteria comprise a gene sequence encoding mutated GLP-2. In some embodiments, the recombinant bacteria comprise gene sequence encoding a mutated glp2-linker Fc (IgA) which comprises SEQ ID NO: 540 or a functional fragment thereof. In some embodiments, recombinant bacteria comprise a gene sequence encoding a mutated glp2-linker Fc (IgA) polypeptide that is at least about 80%, 85%, 90%, 95%, or 99% identity to mutated glp2-linker Fc (IgA) polypeptide comprising SEQ ID NO: 540 or a functional fragment thereof. In some embodiments, the mutated glp2-linker Fc (IgA) polypeptide has at least about 85%, 86%, 87%, 88%, 89%, 90%, 91%, 92%, 93%, 94%, 95%, 96%, 97%, 98%, or 99% identity with SEQ ID NO: 540. In some specific embodiments, the mutated glp2-linker Fc (IgA) polypeptide comprises SEQ ID NO: 540. In other specific embodiments, the mutated glp2-linker Fc (IgA) polypeptide consists of SEQ ID NO: 540. In certain embodiments, the mutated glp2-linker Fc (IgA) fusion protein mutated glp2-linker Fc (IgA) gene sequence has at least about 80%, 85%, 90%, 95%, or 99% identity with SEQ ID NO: 559. In some embodiments, the mutated glp2-linker Fc (IgA) gene sequence has at least about 85%, 86%, 87%, 88%, 89%, 90%, 91%, 92%, 93%, 94%, 95%, 96%, 97%, 98%, or 99% identity with SEQ ID NO: 559. In some specific embodiments, the mutated glp2-linker Fc (IgA) gene sequence comprises SEQ ID NO: 559. In other specific embodiments, the mutated glp2-linker Fc (IgA) gene sequence consists of SEQ ID NO: 559.

In certain embodiments, the mutated glp2-linker Fc (IgA) fusion protein mutated glp2-linker Fc (IgA) gene sequence has at least about 80%, 85%, 90%, 95%, or 99% identity with SEQ ID NO: 560. In some embodiments, the mutated glp2-linker Fc (IgA) gene sequence has at least about 85%, 86%, 87%, 88%, 89%, 90%, 91%, 92%, 93%, 94%, 95%, 96%, 97%, 98%, or 99% identity with SEQ ID NO: 560. In some specific embodiments, the mutated glp2-linker Fc (IgA) gene sequence comprises SEQ ID NO: 560. In other specific embodiments, the mutated glp2-linker Fc (IgA) gene sequence consists of SEQ ID NO: 560.

In any of these embodiments, the recombinant bacteria may further comprise gene sequences encoding a secretion tag. Non-limiting examples of such secretion tags are described herein and include PhoA, OmpF, cvaC, TorA, fdnG, dmsA, PelB, the ECOLIN_05715 secretion signal, ECOLIN_16495 secretion signal, ECOLIN_19410 secretion signal, and the ECOLIN_19880 secretion signal. In some embodiments, the secretion tag is PhoA. In some embodiments, the PhoA secretion tag comprises SEQ ID NO: 385. In some embodiments, the PhoA secretion tag comprises SEQ ID NO: 386. In some embodiments, the recombinant bacteria comprise gene sequence encoding a PhoA-mutated GLP-2 fusion protein. In some embodiments, the recombinant bacteria comprise gene sequence encoding a mutated GLP-2 fusion protein which comprises SEQ ID NO: 541 or a functional fragment thereof. In some embodiments, recombinant bacteria comprise a gene sequence encoding a PhoA-mutated GLP-2 polypeptide that is at least about 80%, 85%, 90%, 95%, or 99% identity to PhoA-mutated GLP-2 polypeptide comprising SEQ ID NO: 541 or a functional fragment thereof. In some embodiments, the PhoA-mutated GLP-2 polypeptide has at least about 85%, 86%, 87%, 88%, 89%, 90%, 91%, 92%, 93%, 94%, 95%, 96%, 97%, 98%, or 99% identity with SEQ ID NO: 541. In some specific embodiments, the PhoA-mutated GLP-2 polypeptide comprises SEQ ID NO: 541. In other specific embodiments, the PhoA-mutated GLP-2 polypeptide consists of SEQ ID NO: 541. In some embodiments, the recombinant bacteria comprise gene sequence encoding a PhoA-mutated GLP-2 fusion protein. In certain embodiments, the PhoA-mutated GLP-2 fusion protein gene sequence has at least about 80%, 85%, 90%, 95%, or 99% identity with SEQ ID NO: 561. In some embodiments, the PhoA-mutated GLP-2 gene sequence has at least about 85%, 86%, 87%, 88%, 89%, 90%, 91%, 92%, 93%, 94%, 95%, 96%, 97%, 98%, or 99% identity with SEQ ID NO: 561. In some specific embodiments, the PhoA-mutated GLP-2 gene sequence comprises SEQ ID NO: 561. In other specific embodiments, the PhoA-mutated GLP-2 gene sequence consists of SEQ ID NO: 561.

In some embodiments, the genetically engineered bacteria comprise a mutated GLP-2 fusion protein encoding PhoA-GLP-2-Fc (hIgA). In some embodiments, the recombinant bacteria comprise gene sequence encoding a mutated GLP-2 fusion protein which comprises SEQ ID NO: 543 or a functional fragment thereof. In some embodiments, recombinant bacteria comprise a gene sequence encoding a PhoA-GLP-2-Fc (hIgA) polypeptide that is at least about 80%, 85%, 90%, 95%, or 99% identity to PhoA-GLP-2-Fc (hIgA) polypeptide comprising SEQ ID NO: 543 or a functional fragment thereof. In some embodiments, the PhoA-GLP-2-Fc (hIgA) polypeptide has at least about 85%, 86%, 87%, 88%, 89%, 90%, 91%, 92%, 93%, 94%, 95%, 96%, 97%, 98%, or 99% identity with SEQ ID NO: 543. In some specific embodiments, the PhoA-GLP-2-Fc (hIgA) polypeptide comprises SEQ ID NO: 543. In other specific embodiments, the PhoA-GLP-2-Fc (hIgA) polypeptide consists of SEQ ID NO: 543.

In some embodiments, the recombinant bacteria comprise gene sequence encoding a PhoA-GLP-2-Fc (hIgA) fusion protein which comprises SEQ ID NO: 544 or a functional fragment thereof. In some embodiments, recombinant bacteria comprise a gene sequence encoding a PhoA-GLP-2-Fc (hIgA) polypeptide that is at least about 80%, 85%, 90%, 95%, or 99% identity to PhoA-GLP-2-Fc (hIgA) polypeptide comprising SEQ ID NO: 544 or a functional fragment thereof. In some embodiments, the PhoA-GLP-2-Fc (hIgA) polypeptide has at least about 85%, 86%, 87%, 88%, 89%, 90%, 91%, 92%, 93%, 94%, 95%, 96%, 97%, 98%, or 99% identity with SEQ ID NO: 544. In some specific embodiments, the PhoA-GLP-2-Fc (hIgA) polypeptide comprises SEQ ID NO: 544. In other specific embodiments, the PhoA-GLP-2-Fc (hIgA) polypeptide consists of SEQ ID NO: 544.

In certain embodiments, the a PhoA-mutated GLP-2-Fc (hIgA) fusion protein gene sequence has at least about 80%, 85%, 90%, 95%, or 99% identity with SEQ ID NO: 551. In some embodiments, the PhoA-mutated GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 551. In some specific embodiments, the PhoA-mutated GLP-2-Fc (hIgA) gene sequence comprises SEQ ID NO: 551. In other specific embodiments, the PhoA-mutated GLP-2-Fc (hIgA) gene sequence consists of SEQ ID NO: 551.

In certain embodiments, the PhoA-mutated GLP-2-Fc (hIgA) fusion protein gene sequence has at least about 80%, 85%, 90%, 95%, or 99% identity with SEQ ID NO: 552. In some embodiments, the PhoA-mutated GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 552. In some specific embodiments, the PhoA-mutated GLP-2-Fc (hIgA) gene sequence comprises SEQ ID NO: 552. In other specific embodiments, the PhoA-mutated GLP-2-Fc (hIgA) gene sequence consists of SEQ ID NO: 552.

In certain embodiments, the PhoA-mutated GLP-2-Fc (hIgA) fusion protein gene sequence has at least about 80%, 85%, 90%, 95%, or 99% identity with SEQ ID NO: 553. In some embodiments, the PhoA-mutated GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 553. In some specific embodiments, the PhoA-mutated GLP-2-Fc (hIgA) gene sequence comprises SEQ ID NO: 553. In other specific embodiments, the PhoA-mutated GLP-2-Fc (hIgA) gene sequence consists of SEQ ID NO: 553.

In certain embodiments, the PhoA-mutated GLP-2-Fc (hIgA) fusion protein gene sequence has at least about 80%, 85%, 90%, 95%, or 99% identity with SEQ ID NO: 554. In some embodiments, the PhoA-mutated GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 554. In some specific embodiments, the PhoA-mutated GLP-2-Fc (hIgA) gene sequence comprises SEQ ID NO: 554. In other specific embodiments, the PhoA-mutated GLP-2-Fc (hIgA) gene sequence consists of SEQ ID NO: 554.

In some embodiments, the secretion tag is ECOLIN 19410. In some embodiments, the ECOLIN 19410 secretion tag comprises SEQ ID NO: 397. In some embodiments, the ECOLIN 19410 secretion tag gene sequence comprises SEQ ID NO: 491.

In some embodiments, the recombinant bacteria comprise gene sequence encoding a ECOLIN 19410-mutated GLP-2 fusion protein which comprises SEQ ID NO: 542 or a functional fragment thereof. In some embodiments, recombinant bacteria comprise a gene sequence encoding a ECOLIN 19410-mutated GLP-2 polypeptide that is at least about 80%, 85%, 90%, 95%, or 99% identity to ECOLIN 19410-mutated GLP-2 polypeptide comprising SEQ ID NO: 542 or a functional fragment thereof. In some embodiments, the ECOLIN 19410-mutated GLP-2 polypeptide has at least about 85%, 86%, 87%, 88%, 89%, 90%, 91%, 92%, 93%, 94%, 95%, 96%, 97%, 98%, or 99% identity with SEQ ID NO: 542. In some specific embodiments, the ECOLIN 19410-mutated GLP-2 polypeptide comprises SEQ ID NO: 542. In other specific embodiments, the ECOLIN 19410-mutated GLP-2 polypeptide consists of SEQ ID NO: 542. In some embodiments, the gene sequence encoding ECOLIN 19410 secretion tag comprises SEQ ID NO: 491. In some embodiments, the recombinant bacteria comprise gene sequence encoding a ECOLIN 19410-mutated GLP-2 fusion protein. In certain embodiments, the ECOLIN 19410-mutated GLP-2 fusion protein gene sequence has at least about 80%, 85%, 90%, 95%, or 99% identity with SEQ ID NO: 562. In some embodiments, the ECOLIN 19410-mutated GLP-2 gene sequence has at least about 85%, 86%, 87%, 88%, 89%, 90%, 91%, 92%, 93%, 94%, 95%, 96%, 97%, 98%, or 99% identity with SEQ ID NO: 562. In some specific embodiments, the ECOLIN 19410-mutated GLP-2 gene sequence comprises SEQ ID NO: 562. In other specific embodiments, the ECOLIN 19410-mutated GLP-2 gene sequence consists of SEQ ID NO: 562.

In some embodiments, the recombinant bacteria comprise a GLP-2 fusion protein encoding ECOLIN 19410-GLP-2-Fc (hIgA). In some embodiments, the recombinant bacteria comprise gene sequence encoding a ECOLIN 19410-mutated GLP-2-Fc (hIgA) which comprises SEQ ID NO: 545 or a functional fragment thereof. In some embodiments, recombinant bacteria comprise a gene sequence encoding a ECOLIN 19410-mutated GLP-2-Fc (hIgA) polypeptide that is at least about 80%, 85%, 90%, 95%, or 99% identity to ECOLIN 19410-mutated GLP-2-Fc (hIgA) polypeptide comprising SEQ ID NO: 545 or a functional fragment thereof. In some embodiments, the ECOLIN 19410-mutated GLP-2-Fc (hIgA) polypeptide has at least about 85%, 86%, 87%, 88%, 89%, 90%, 91%, 92%, 93%, 94%, 95%, 96%, 97%, 98%, or 99% identity with SEQ ID NO: 545. In some specific embodiments, the ECOLIN 19410-mutated GLP-2-Fc (hIgA) polypeptide comprises SEQ ID NO: 545. In other specific embodiments, the ECOLIN 19410-mutated GLP-2-Fc (hIgA) polypeptide consists of SEQ ID NO: 545. In some embodiments, the recombinant bacteria comprise gene sequence encoding a GLP-2-Fc (hIgA) fusion protein which comprises SEQ ID NO: 546 or a functional fragment thereof. In some embodiments, recombinant bacteria comprise a gene sequence encoding a polypeptide that is at least about 80%, 85%, 90%, 95%, or 99% identity to -Fc (hIgA) polypeptide comprising SEQ ID NO: 546 or a functional fragment thereof. In some embodiments, the -Fc (hIgA) polypeptide has at least about 85%, 86%, 87%, 88%, 89%, 90%, 91%, 92%, 93%, 94%, 95%, 96%, 97%, 98%, or 99% identity with SEQ ID NO: 546. In some specific embodiments, the -Fc (hIgA) polypeptide comprises SEQ ID NO: 546. In other specific embodiments, the -Fc (hIgA) polypeptide consists of SEQ ID NO: 546.

In some embodiments, the recombinant bacteria comprise gene sequence encoding a ECOLIN 19410-mutated GLP-2-Fc (hIgA) fusion protein. In certain embodiments, the ECOLIN 19419-mutated GLP-2-Fc (hIgA) gene sequence has at least about 80%, 85%, 90%, 95%, or 99% identity with SEQ ID NO: 555. In some embodiments, the ECOLIN 19419-mutated GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 555. In some specific embodiments, the ECOLIN 19419-mutated GLP-2-Fc (hIgA) gene sequence comprises SEQ ID NO: 555. In other specific embodiments, the ECOLIN 19419-mutated GLP-2-Fc (hIgA) gene sequence consists of SEQ ID NO: 555.

In certain embodiments, the ECOLIN 19419-mutated GLP-2-Fc (hIgA) gene sequence has at least about 80%, 85%, 90%, 95%, or 99% identity with SEQ ID NO: 556. In some embodiments, the ECOLIN 19419-mutated GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 556. In some specific embodiments, the ECOLIN 19419-mutated GLP-2-Fc (hIgA) gene sequence comprises SEQ ID NO: 556. In other specific embodiments, the ECOLIN 19419-mutated GLP-2-Fc (hIgA) gene sequence consists of SEQ ID NO: 556.

In certain embodiments, the ECOLIN 19419-mutated GLP-2-Fc (hIgA) gene sequence has at least about 80%, 85%, 90%, 95%, or 99% identity with SEQ ID NO: 557. In some embodiments, the ECOLIN 19419-mutated GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 557. In some specific embodiments, the ECOLIN 19419-mutated GLP-2-Fc (hIgA) gene sequence comprises SEQ ID NO: 557. In other specific embodiments, the ECOLIN 19419-mutated GLP-2-Fc (hIgA) gene sequence consists of SEQ ID NO: 557.

In certain embodiments, the ECOLIN 19419-mutated GLP-2-Fc (hIgA) gene sequence has at least about 80%, 85%, 90%, 95%, or 99% identity with SEQ ID NO: 558. In some embodiments, the ECOLIN 19419-mutated GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 558. In some specific embodiments, the ECOLIN 19419-mutated GLP-2-Fc (hIgA) gene sequence comprises SEQ ID NO: 558. In other specific embodiments, the ECOLIN 19419-mutated GLP-2-Fc (hIgA) gene sequence consists of SEQ ID NO: 558

In any of these embodiments, the recombinant bacteria may further comprise gene sequences encoding a secretion tag. Non-limiting examples of such secretion tags are described herein and include PhoA, OmpF, cvaC, TorA, fdnG, dmsA, PelB, the ECOLIN_05715 secretion signal, ECOLIN_16495 secretion signal, ECOLIN_19410 secretion signal, and the ECOLIN_19880 secretion signal. In some embodiments, the secretion tag is PhoA. In some embodiments, the secretion tag is ECOLIN 19410. The recombinant bacteria may further comprise one or more mutations to outer membrane proteins, i.e., to generate a diffusible outer membrane phenotype (DOM). Non-limiting examples of such outer membrane proteins are described herein and include lpp, nlP, tolA, and PAL. In one embodiment, the recombinant bacteria comprise a deletion or mutation in PAL.

In some embodiments, the genetically engineered are capable of producing and secreting GLP-2. In some embodiments, the GLP-2 gene is functionally replaced, modified, and/or mutated in order to enhance stability and/or increase GLP-2 production or secretion. In some embodiments, the recombinant bacteria are capable of expressing and secreting GLP-2 in low-oxygen conditions, in the presence of certain molecules or metabolites, in the presence of molecules or metabolites associated with inflammation or an inflammatory response, or in the presence of some other metabolite that may or may not be present in the gut, such as arabinose. Exemplary chemical inducers are described herein.

In one embodiment, the GLP-2 gene is directly operably linked to a first promoter. In another embodiment, the GLP-2 gene is indirectly operably linked to a first promoter. In one embodiment, the promoter is not operably linked with the GLP-2 gene in nature.

In some embodiments, the GLP-2 gene is expressed under the control of a constitutive promoter. In another embodiment, the GLP-2 gene is expressed under the control of an inducible promoter. In some embodiments, the GLP-2 gene is expressed under the control of a promoter that is directly or indirectly induced by exogenous environmental conditions. In one embodiment, the GLP-2 gene is expressed under the control of a promoter that is directly or indirectly induced by low-oxygen or anaerobic conditions, wherein expression of the GLP-2 gene is activated under low-oxygen or anaerobic environments, such as the environment of the mammalian gut. Inducible promoters are described in more detail infra.

The GLP-2 gene may be present on a plasmid or chromosome in the bacterial cell. In one embodiment, the GLP-2 gene is located on a plasmid in the bacterial cell. In another embodiment, the GLP-2 gene is located in the chromosome of the bacterial cell. In yet another embodiment, a native copy of the GLP-2 gene is located in the chromosome of the bacterial cell.

The GLP-2 gene may be expressed on a low-copy plasmid or a high-copy plasmid. In some embodiments, the high-copy plasmid may be useful for increasing expression of GLP-2.

In any of these embodiments, the recombinant bacteria may comprise gene sequence(s) which encode two separate effector polypeptides covalently linked together through a peptide bond or peptide linker. In one embodiment, the recombinant bacteria may comprise gene sequence(s) which encode GLP-2 covalently linked to IL-22. In one embodiment, the recombinant bacteria may comprise gene sequence(s) which encode GLP-2 joined by a peptide bond or a peptide linker to a first and a second IL-10 monomer, which are joined together by a second peptide bond or peptide linker.

IL-19, IL-20, and/or IL-24

In some embodiments, the recombinant bacteria are capable of producing IL-19, IL-20, and/or IL-24. In some embodiments, the recombinant bacteria are capable of producing IL-19, IL-20, and/or IL-24 under inducing conditions, e.g., under a condition(s) associated with inflammation. In some embodiments, the recombinant bacteria are capable of producing IL-19, IL-20 and/or IL-24 in low-oxygen conditions.

In some embodiments, the recombinant bacteria produce 0% to 2% to 4%, 4% to 6%, 6% to 8%, 8% to 10%, 10% to 12%, 12% to 14%, 14% to 16%, 16% to 18%, 18% to 20%, 20% to 25%, 25% to 30%, 30% to 35%, 35% to 40%, 40% to 45% 45% to 50%, 50% to 55%, 55% to 60%, 60% to 65%, 65% to 70% to 80%, 80% to 90%, or 90% to 100% more IL-2 than unmodified bacteria of the same bacterial subtype under the same conditions. In yet another embodiment, the recombinant bacteria produce 1.0-1.2-fold, 1.2-1.4-fold, 1.4-1.6-fold, 1.6-1.8-fold, 1.8-2-fold, or two-fold more IL-10 IL-2, IL-22, SOD, GLP-2, IL-19, IL-20 and/or IL-24 than unmodified bacteria of the same bacterial subtype under the same conditions. In yet another embodiment, the recombinant bacteria produce three-fold, four-fold, five-fold, six-fold, seven-fold, eight-fold, nine-fold, ten-fold, fifteen-fold, twenty-fold, thirty-fold, forty-fold, or fifty-fold, more IL-10 IL-2, IL-22, SOD, GLP-2, IL-19, IL-20, and/or IL-24 than unmodified bacteria of the same bacterial subtype under the same conditions.

CD47-SIRPα Pathway

CD47 is a cell surface molecule implicated in cell migration and T cell and dendritic cell activation. In addition, CD47 functions as an inhibitor of phagocytosis through ligation of signal-regulatory protein alpha (SIRPα) expressed on phagocytes, leading to tyrosine phosphatase activation and inhibition of myosin accumulation at the submembrane assembly site of the phagocytic synapse. Loss of CD47 leads to homeostatic phagocytosis of aged or damaged cells.

Elevated levels of CD47 expression are observed on multiple human tumor types, allowing tumors to escape the innate immune system through evasion of phagocytosis. This process occurs through binding of CD47 on tumor cells to SIRPα on phagocytes, thus promoting inhibition of phagocytosis and tumor survival.

Anti-CD47 antibodies have demonstrated pre-clinical activity against many different human cancers both in vitro and in mouse xenotransplantation models (Chao et al., Curr Opin Immunol. 2012; 24(2): 225-232). In addition to CD47, SIRPα can also be targeted as a therapeutic strategy; for example, anti-SIRPα antibodies administered in vitro caused phagocytosis of tumor cells by macrophages.

CD47-targeted therapies have been developed using the single 14 kDa CD47 binding domain of human SIRPα (a soluble form without the transmembrane portion) as a competitive antagonist to human CD47 (as described in Weiskopf et al., Science. 2013; 341(6141): the contents of which is herein incorporated by reference in its entirety). Because the wild type SIRPα showed relatively low affinity to CD47, mutated SIRPα were generated through in vitro evolution via yeast surface display, which were shown to act as strong binders and antagonists of CD47. These variant include CV1 (consensus variant 1) and high-affinity variant FD6, and Fc fusion proteins of these variants. The amino acid changes leading to the increased affinity are located in the dl domain of human SIRPα. Non-limiting examples of SIRPalpha variants are also described in WO/2013/109752, the contents of which is herein incorporated by reference in its entirety.

In certain embodiments, the recombinant bacteria produce one or more anti-cancer molecules that inhibit CD47 and/or inhibit SIRPα and/or inhibit or prevent the interaction between CD47 and SIRPα expressed on macrophages. For example, the genetically engineered microorganism may encode an antibody directed against CD47 and/or an antibody directed against SIRPα, e.g. a single-chain antibody against CD47 and/or a single-chain antibody against SIRPα. In another non-limiting example, the genetically engineered microorganism may encode a competitive antagonist polypeptide comprising the SIRPα CD47 binding domain Such a competitive antagonist polypeptide can function through competitive binding of CD47, preventing the interaction of CD47 with SIRPα expressed on macrophages. In some embodiments, the competitive antagonist polypeptide is soluble, e.g., is secreted from the microorganism. In some embodiments, the competitive antagonist polypeptide is displayed on the surface of the microorganism. In some embodiments, the genetically engineered microorganism encoding the competitive antagonist polypeptide encodes a wild type form of the SIRPα CD47 binding domain. In some embodiments, the genetically engineered microorganism encoding the competitive antagonist polypeptide encodes a mutated or variant form of the SIRPα CD47 binding domain. In some embodiments, the variant form is the CV1 SIRPα variant. In some embodiments, the variant form is the FD6 variant. In some embodiments, the SIRPα variant is a variant described in Weiskopf et al., and/or International Patent Publication WO/2013/109752. In some embodiments, the genetically engineered microorganism encoding the competitive antagonist polypeptide encodes a SIRPα CD47 binding domain or variant thereof fused to a stabilizing polypeptide. In some embodiments, the genetically engineered microorganism encoding the competitive antagonist polypeptide encodes a wild type form of the SIRPα CD47 binding domain fused to a stabilizing polypeptide. In a non-limiting example, the stabilizing polypeptide fused to the wild type SIRPα CD47 binding domain polypeptide is a Fc portion. In some embodiments, the stabilizing polypeptide fused to the wild type SIRPα CD47 binding domain polypeptide is the IgG Fc portion. In some embodiments, the stabilizing polypeptide fused to the wild type SIRPα CD47 binding domain polypeptide is the IgG4 Fc portion. In some embodiments, the genetically engineered microorganism encoding the competitive antagonist polypeptide encodes a mutated or variant form of the SIRPα CD47 binding domain fused to a stabilizing polypeptide. In some embodiments, the variant form fused to the stabilizing polypeptide is the CV1 SIRPα variant. In some embodiments, the variant form fused to the stabilizing polypeptide is the F6 variant. In some embodiments, the SIRPα variant fused to the stabilizing polypeptide is a variant described in Weiskopf et al., and/or International Patent Publication WO/2013/109752. In a non-limiting example, the stabilizing polypeptide fused to the variant SIRPα CD47 binding domain polypeptide is a Fc portion. In some embodiments, the stabilizing polypeptide fused to the variant SIRPα CD47 binding domain polypeptide is the IgG Fc portion. In some embodiments, the stabilizing polypeptide fused to the variant SIRPα CD47 binding domain polypeptide is an IgG4 Fc portion.

In some embodiments, the recombinant bacterium is a tumor-targeting bacterium that expresses an anti-CD47 antibody and/or anti-SIRPα antibody, e.g., a single chain antibody. In some embodiments, the recombinant bacterium is a tumor-targeting bacterium that expresses competitive antagonist SIRPα CD47 binding domain (WT or mutated to improve CD47 affinity). In some embodiments, the recombinant bacterium is a tumor-targeting bacterium that expresses an anti-CD47 antibody and/or anti-SIRPα antibody, e.g., a single chain antibody, under the control of a promoter that is activated by low-oxygen conditions. In some embodiments, the recombinant bacterium expresses a competitive antagonist SIRPα CD47 binding domain (WT or mutated variant with improved CD47 affinity) under the control of a promoter that is activated by low-oxygen conditions. In some embodiments, the recombinant bacterium expresses an anti-CD47 antibody and/or an anti-SIRPα, e.g., single chain antibody, under the control of a promoter that is activated by hypoxic conditions, or by inflammatory conditions, such as any of the promoters activated by said conditions and described herein. In some embodiments, the recombinant bacterium expresses a competitive antagonist SIRPα CD47 binding domain (WT or mutated variant with improved CD47 affinity) under the control of a promoter that is activated by hypoxic conditions, or by inflammatory conditions, such as any of the promoters activated by said conditions and described herein. In some embodiments, the recombinant bacteria expresses an anti-CD47 antibody and/or an anti-SIRPα antibody, e.g., single chain antibody, under the control of a cancer-specific promoter, a tissue-specific promoter, or a constitutive promoter, such as any of the promoters described herein. In some embodiments, the recombinant bacterium expresses a competitive antagonist SIRPα CD47 binding domain (WT or mutated variant with improved CD47 affinity) under the control of a cancer-specific promoter, a tissue-specific promoter, or a constitutive promoter, such as any of the promoters described herein. In any of these embodiments, the genetically engineered bacteria may also produce one or more anti-cancer molecules that are capable of stimulating Fc-mediated functions such as ADCC, and/or M-CSF and/or GM-CSF, resulting in a blockade of phagocytosis inhibition.

The recombinant bacteria may comprise any suitable anti-CD47 antibody, anti-SIRPα antibody or competitive SIRPα CD47 binding domain polypeptide (wild type or mutated variant with improved CD47 binding affinity) for the inhibition or prevention of the CD47-SIRPα interaction. In some embodiments, the antibody(ies) or competitive polypeptide(s) is modified and/or mutated, e.g., to enhance stability or increase CD47 antagonism.

In some embodiments, the recombinant bacteria are capable of expressing any one or more of the described circuits in low-oxygen conditions, and/or in the presence of cancer and/or the tumor microenvironment and/or the tumor microenvironment or tissue specific molecules or metabolites, and/or in the presence of molecules or metabolites associated with inflammation or immune suppression, and/or in the presence of metabolites that may be present in the gut or the tumor, and/or in the presence of metabolites that may or may not be present in vivo, and may be present in vitro during strain culture, expansion, production and/or manufacture, such as arabinose and others described herein. In some embodiments, the gene sequences(s) are controlled by a promoter inducible by such conditions and/or inducers. In some embodiments, the gene sequences(s) are controlled by a constitutive promoter, as described herein. In some embodiments, the gene sequences(s) are controlled by a constitutive promoter, and are expressed in in vivo conditions and/or in vitro conditions, e.g., during bacteria expansion, production and/or manufacture, as described herein.

IL-15

IL-15 displays pleiotropic functions in homeostasis of both innate and adaptive immune system and binds to IL-15 receptor, a heterotrimeric receptor composed of three subunits. The alpha subunit is specific for IL-15, while beta (CD122) and gamma (CD132) subunits are shared with the IL-2 receptor, and allow shared signaling through the JAK/STAT pathways. IL-15 is produced by several cell types, including dendritic cells, monocytes and macrophages. Co-expression of IL-15Rα and IL-15 produced in the same cell, allows intracellular binding of IL-15 to IL-15Rα, which is then shuttled to the cell surface as a complex. Once on the cell surface, then, the IL-15Rα of these cells is able to trans-present IL-15 to IL-15Rβ-γc of CD8 T cells, NK cells, and NK-T cells, which do not express IL-15, inducing the formation of the so-called immunological synapse. Murine and human IL-15Rα, exists both in membrane bound, and also in a soluble form. Soluble IL-15Rα (sIL-15Rα) is constitutively generated from the transmembrane receptor through proteolytic cleavage. IL-15 is critical for lymphoid development and peripheral maintenance of innate immune cells and immunological memory of T cells, in particular natural killer (NK) and CD8+ T cell populations. In contrast to IL-2, IL-15 does not promote the maintenance of Tregs and furthermore, IL-15 has been shown to protect effector T cells from IL-2-mediated activation-induced cell death.

Consequently, delivery of IL-15 is considered a promising strategy for long-term anti-tumor immunity. In a first-in-human clinical trial of recombinant human IL-15, a 10-fold expansion of NK cells and significantly increased the proliferation of 76T cells and CD8+ T cells was observed upon treatment. In addition, IL-15 suparagonists containing cytokine-receptor fusion complexes have been developed and are evaluated to increase the length of the response. These include the L-15 N72D superagonist/IL-15RαSushi-Fc fusion complex (IL-15SA/IL-15RαSu-Fc; ALT-803) (Kim et al., 2016) markedly enhances specific subpopulations of NK and memory CD8+ T cells, and mediates potent anti-tumor activity against murine breast and colon carcinomas). Thus, in some embodiments, the engineered bacteria is engineered to produce IL-15.

The biological activity of IL-15 is greatly improved by pre-associating IL-15 with a fusion protein IL-15Rα-Fc or by direct fusion with the sushi domain of IL-15Rα (hyper-IL-15) to mimic trans-presentation of IL-15 by cell-associated IL-15Rα. IL-15, either administrated alone or as a complex with IL-15Rα, exhibits potent antitumor activities in animal models (Cheng et al., Immunotherapy of metastatic and autochthonous liver cancer with IL-15/IL-15Rα fusion protein; Oncoimmunology. 2014; 3(11): e963409, and references therein)

In some embodiments, the engineered bacteria comprise sequence to encode IL-15. In some embodiments, the engineered bacteria are engineered to over-express IL-15, for example, operatively linked to a strong promoter and/or comprising more than one copy of the IL-15 gene sequence. In some embodiments, the engineered bacteria comprise sequence(s) encoding two or more copies of IL-15 gene, e.g., two, three, four, five, six or more copies of IL-15 gene. In some embodiments, the engineered bacteria produce one or more anti-cancer molecules that stimulate the production of IL-15. In some embodiments, the engineered bacteria comprise sequence to encode IL-15Rα. In some embodiments, the engineered bacteria comprise sequence to encode IL-15 and sequence to encode IL-15Rα. In some embodiments, the engineered bacteria comprise sequence to encode a fusion polypeptide comprising IL-15 and IL-15Rα. In some embodiments, the engineered bacteria comprise sequence(s) to encode IL-15 and sequence to encode a secretory peptide(s) for the secretion of IL-15. The recombinant bacteria is a tumor-targeting bacterium. The recombinant bacterium expresses IL-15 and/or expresses secretory peptides under the control of a promoter that is activated by low-oxygen conditions, by hypoxic conditions, or by inflammatory conditions, such as any of the promoters activated by said conditions and described herein. In some embodiments, the recombinant bacteria expresses IL-15 and/or expresses secretory peptide(s), under the control of a cancer-specific promoter, a tissue-specific promoter, or a constitutive promoter, such as any of the promoters described herein.

In some embodiments, the genetically engineered bacteria are capable of expressing any one or more of the described IL-15 circuits in low-oxygen conditions, and/or in the presence of cancer and/or in the tumor microenvironment, or tissue specific molecules or metabolites, and/or in the presence of molecules or metabolites associated with inflammation or immune suppression, and/or in the presence of metabolites that may be present in the gut, and/or in the presence of metabolites that may or may not be present in vivo, and may be present in vitro during strain culture, expansion, production and/or manufacture, such as arabinose and others described herein. In some embodiments, the gene sequences(s) encoding IL-15 are controlled by a promoter inducible by such conditions and/or inducers. In some embodiments, the gene sequences(s) encoding IL-15 are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein.

In some embodiments, any one or more of the described genes sequences encoding IL-15 are present on one or more plasmids (e.g., high copy or low copy) or are integrated into one or more sites in the microorganisms chromosome.

IL-12

Interleukin 12 (IL-12) is a cytokine, the actions of which create an interconnection between the innate and adaptive immunity. IL-12 is secreted by a number of immune cells, including activated dendritic cells, monocytes, macrophages, and neutrophils, as well as other cell types. IL-12 is a heterodimeric protein (IL-12-p70; IL-12-p35/p40) consisting of p35 and p40 subunits, and binds to a receptor composed of two subunits, IL-12R-131 and IL-12R-β2. IL-12 receptor is expressed constitutively or inducibly on a number of immune cells, including NK cells, T, and B lymphocytes. Upon binding of IL-12, the receptor is activated and downstream signaling through the JAK/STAT pathway initiated, resulting in the cellular response to IL-12. IL-12 acts by increasing the production of IFN-γ, which is the most potent mediator of IL-12 actions, from NK and T cells. In addition, IL-12 promotes growth and cytotoxicity of activated NK cells, CD8+ and CD4+ T cells, and shifts the differentiation of CD4+Th0 cells toward the Th1 phenotype. Further, IL-12 enhances of antibody-dependent cellular cytotoxicity (ADCC) against tumor cells and the induction of IgG and suppression of IgE production from B cells. In addition, IL-12 also plays a role in reprogramming of myeloid-derived suppressor cells, directs the Th1-type immune response and helps increase expression of MHC class I molecules (e.g., reviewed in Waldmann et al., Cancer Immunol Res March 2015 3; 219).

Thus, in some embodiments, the engineered bacteria are engineered to produce IL-12. In some embodiments, the engineered bacteria comprise sequence to encode IL-12. In some embodiments, the engineered bacteria are engineered to over-express IL-12, for example, operatively linked to a strong promoter and/or comprising more than one copy of the IL-12 gene sequence. In some embodiments, the engineered bacteria comprise sequence(s) encoding two or more copies of IL-12, e.g., two, three, four, five, six or more copies of IL-12 gene. In some embodiments, the engineered bacteria produce one or more anti-cancer molecules that stimulate the production of IL-12. In some embodiments, the engineered bacteria comprise sequence to encode IL-12 and sequence to encode a secretory peptide(s) for the secretion of IL-12. The recombinant bacteria may be a tumor-targeting bacterium. The recombinant bacterium expresses IL-12 and/or expresses secretory peptides under the control of a promoter that is activated by low-oxygen conditions, by hypoxic conditions, or by inflammatory conditions, such as any of the promoters activated by said conditions and described herein. In some embodiments, the recombinant bacteria expresses IL-12 and/or expresses secretory peptide(s), under the control of a cancer-specific promoter, a tissue-specific promoter, or a constitutive promoter, such as any of the promoters described herein.

### Inducible Regulatory Regions

In some embodiments, the bacterial cell comprises a stably maintained plasmid or chromosome carrying the gene(s) encoding payload (s), such that the payload(s) can be expressed in the host cell, and the host cell is capable of survival and/or growth in vitro, e.g., in medium, and/or in vivo, e.g., in the gut. In some embodiments, bacterial cell comprises two or more distinct payloads or operons, e.g., two or more payload genes. In some embodiments, bacterial cell comprises three or more distinct transporters or operons, e.g., three or more payload genes. In some embodiments, bacterial cell comprises 4, 5, 6, 7, 8, 9, 10, or more distinct payloads or operons, e.g., 4, 5, 6, 7, 8, 9, 10, or more payload genes.

Herein the terms “payload” “polypeptide of interest” or “polypeptides of interest”, “protein of interest”, “proteins of interest”, “payloads” “effector molecule”, “effector” refers to one or more effector molecules described herein and/or one or more enzyme(s) or polypeptide(s) function as enzymes for the production and secretion of such effector molecules.

Non-limiting examples of payloads include effector molecules, therapeutic polypeptides, anti-inflammation and/or gut barrier function enhancer molecule(s), including but not limited to, cytokines and growth factors, e.g., IL10, IL-2, IL-22, IL-27, IL-20, IL-24, IL-19, SOD, and GLP2. Non-limiting examples of payloads include cytokines and growth factors selected from IL-2, IL-15, IL-12, IL-7, IL-21, IL-18, TNF, and interferon gamma (IFN-gamma), GMCSF. In some embodiments, payloads include CXCL10, CXCL9, SIRPalpha soluble form. Non-limiting examples of payloads include, TLR agonists, for example, one or more TLR1 agonists, TLR2 agonists, TLR3 agonists, TLR4 agonists, TLR5 agonists, TLR6 agonists, TLR7 agonists, TLR8 agonists, TLR9 agonists, and TRL10 agonists. In some embodiments, the payloads are enzymes, which can be secreted. Non-limiting examples of such enzymes include hyaluronidase, bile salt hydrolase, kynureninase. In some embodiments, the effector molecules are antibodies, such as single chain antibodies. Non-limiting examples of such antibodies include Anti-CD47, Anti-VEGF, anti-HIF, anti-NRP1 antibody, anti-NRP2 antibody, anti-semaphorin3A antibody, anti-CXCR4, anti-CXCL12, anti-Galectin-3 antibody, an anti-Galectin-1 antibody, anti-Tie-2 antibody, anti-Ang1 antibody, anti-Ang4 antibody, anti-VEGFR-2 antibody, Anti-Phosphatidylserine. In some embodiments, the effectors are checkpoint inhibitors. Non-limiting examples of checkpoint inhibitors include antibodies directed against CTLA-4, PD-1, PD-L1, PD-L2, TIGIT, VISTA, LAG-3, TIM1, TIM3, CEACAM1, LAIR-1, HVEM, BTLA, CD160, CD200, CD200R, CD39, CD73, B7-H3, B7-H4, IDO, TDO, KIR, and A2aR. In some embodiments, the effectors are lytic peptides (natural peptides which can lyse cells, e.g. tumor cells) or cytotoxic peptides. As used herein, the term “polypeptide of interest” or “polypeptides of interest”, “protein of interest”, “proteins of interest”, “payload”, “payloads” further includes any or a plurality of any of the effector molecules. Such effectors may be anti-inflammation and/or gut barrier function enhancer molecule(s) or proinflammatory and/or anti-cancer molecules. As used herein, the term “gene of interest” or “gene sequence of interest” includes any or a plurality of any of the gene(s) an/or gene sequence(s) and or gene cassette(s) encoding one or more anti-inflammation and/or gut barrier function enhancer molecule(s) described herein.

Additional effector molecules, e.g., therapeutic polypeptides, which can be secreted are described in PCT/US2017/013072, filed Jan. 11, 2017; PCT/US2017/016609, filed Feb. 3, 2017; PCT/US2016/039444, filed Jun. 24, 2016; PCT/US2016/069052, filed Dec. 28, 2016; PCT/US2017/012946, filed Jan. 11, 2017; PCT/US2017/017552, filed Feb. 10, 2017; PCT/US2017/017563, filed Feb. 10, 2017, the contents of which is herein incorporated by reference in its entirety.

In some embodiments, the recombinant bacteria comprise multiple copies of the same payload gene(s). In some embodiments, the gene encoding the payload is present on a plasmid and operably linked to a directly or indirectly inducible promoter. In some embodiments, the gene encoding the payload is present on a plasmid and operably linked to a constitutive promoter. In some embodiments, the gene encoding the payload is present on a plasmid and operably linked to a promoter that is induced under low-oxygen or anaerobic conditions. In some embodiments, the gene encoding the payload is present on plasmid and operably linked to a promoter that is induced by exposure to tetracycline or arabinose, or another chemical or nutritional inducer described herein.

In some embodiments, the gene encoding the payload is present on a chromosome and operably linked to a directly or indirectly inducible promoter. In some embodiments, the gene encoding the payload is present on a chromosome and operably linked to a constitutive promoter. In some embodiments, the gene encoding the payload is present in the chromosome and operably linked to a promoter that is induced under low-oxygen or anaerobic conditions. In some embodiments, the gene encoding the payload is present on chromosome and operably linked to a promoter that is induced by exposure to tetracycline or arabinose, or another chemical or nutritional inducer described herein.

In some embodiments, the recombinant bacteria comprise two or more payloads, all of which are present on the chromosome. In some embodiments, the recombinant bacteria comprise two or more payloads, all of which are present on one or more same or different plasmids. In some embodiments, the recombinant bacteria comprise two or more payloads, some of which are present on the chromosome and some of which are present on one or more same or different plasmids.

In any of the nucleic acid embodiments described above, the one or more payload(s) for producing the effector molecules, or the therapeutic polypeptides, are operably linked to one or more directly or indirectly inducible promoter(s). In some embodiments, the one or more payload(s) are operably linked to a directly or indirectly inducible promoter that is induced under exogenous environmental conditions, e.g., conditions found in the gut, e.g., induced by metabolites found in the gut, or other specific conditions. In some embodiments, the one or more payload(s) are operably linked to a directly or indirectly inducible promoter that is induced under low-oxygen or anaerobic conditions or induced under inflammatory conditions (e.g., RNS, ROS), as described herein. In some embodiments, the one or more payload(s) are operably linked to a directly or indirectly inducible promoter that is induced under immunosuppressive conditions, e.g., as found in the tumor, as described herein. In some embodiments, the two or more gene sequence(s) are linked to a directly or indirectly inducible promoter that is induced by exposure a chemical or nutritional inducer, which may or may not be present under in vivo conditions and which may be present during in vitro conditions (such as strain culture, expansion, manufacture), such as tetracycline or arabinose, or others described herein. In some embodiments, the two or more payloads are all linked to a constitutive promoter, as described herein.

In some embodiments, the promoter is induced under in vivo conditions, e.g., the gut, as described herein. In some embodiments, the promoters is induced under in vitro conditions, e.g., various cell culture and/or cell manufacturing conditions, as described herein.

In some embodiments, the promoter that is operably linked to the gene encoding the payload is directly induced by exogenous environmental conditions (e.g., in vivo and/or in vitro and/or production/manufacturing conditions).

In some embodiments, the promoter is directly or indirectly induced by exogenous environmental conditions specific to the gut of a mammal, e.g., to the small intestine of a mammal. In some embodiments, the promoter is directly or indirectly induced by low-oxygen or anaerobic conditions such as the environment of the mammalian gut. In some embodiments, the promoter is directly or indirectly induced by molecules or metabolites that are specific to the tumor, a particular tissue or the gut of a mammal. In some embodiments, the promoter is directly or indirectly induced by a molecule that is co-administered with the bacterial cell.

FNR Dependent Regulation

The recombinant bacteria comprise a gene or gene cassette for producing an effector molecule, or a therapeutic polypeptide, wherein the gene or gene cassette is operably linked to a directly or indirectly inducible promoter that is controlled by exogenous environmental condition(s). In some embodiments, the inducible promoter is an oxygen level-dependent promoter and the effector molecule(s) is expressed in low-oxygen, microaerobic, or anaerobic conditions. For example, in low oxygen conditions, the oxygen level-dependent promoter is activated by a corresponding oxygen level-sensing transcription factor, thereby driving production of the effector molecule(s).

Bacteria have evolved transcription factors that are capable of sensing oxygen levels. Different signaling pathways may be triggered by different oxygen levels and occur with different kinetics. An oxygen level-dependent promoter is a nucleic acid sequence to which one or more oxygen level-sensing transcription factors is capable of binding, wherein the binding and/or activation of the corresponding transcription factor activates downstream gene expression. In one embodiment, the recombinant bacteria comprise a gene or gene cassette for producing a payload under the control of an oxygen level-dependent promoter. In a more specific aspect, the recombinant bacteria comprise a gene or gene cassette for producing a payload under the control of an oxygen level-dependent promoter that is activated under low-oxygen or anaerobic environments, such as the environment of the mammalian gut.

In certain embodiments, the bacterial cell comprises a gene encoding a payload expressed under the control of a fumarate and nitrate reductase regulator (FNR) responsive promoter. In E. coli, FNR is a major transcriptional activator that controls the switch from aerobic to anaerobic metabolism (Unden et al., 1997). In the anaerobic state, FNR dimerizes into an active DNA binding protein that activates hundreds of genes responsible for adapting to anaerobic growth. In the aerobic state, FNR is prevented from dimerizing by oxygen and is inactive. Exemplary FNR responsive promoters include, but are not limited to, SEQ ID NO: 151-167. FNR promoter sequences are known in the art, and any suitable FNR promoter sequence(s) may be used in the recombinant bacteria.

In some embodiments, the recombinant bacteria comprise one or more of: SEQ ID NOS: 151-157, nirB1 promoter (SEQ ID NO: 158), nirB2 promoter (SEQ ID NO: 159), nirB3 promoter (SEQ ID NO: 160), ydfZ promoter (SEQ ID NO: 161), nirB promoter fused to a strong ribosome binding site (SEQ ID NO: 162), ydfZ promoter fused to a strong ribosome binding site (SEQ ID NO: 163), fnrS, an anaerobically induced small RNA gene (fnrS1 promoter SEQ ID NO: 164 or fnrS2 promoter SEQ ID NO: 165), nirB promoter fused to a crp binding site (SEQ ID NO: 166), and fnrS fused to a crp binding site (SEQ ID NO: 167). In some embodiments, the FNR-responsive promoters at least about 80%, 85%, 90%, 95%, or 99% homologous to the sequence of any one of SEQ ID NO: 151-167.

In some embodiments, multiple distinct FNR nucleic acid sequences are inserted in the recombinant bacteria. In alternate embodiments, the recombinant bacteria comprise a gene encoding a payload expressed under the control of an alternate oxygen level-dependent promoter, e.g., DNR (Trunk et al., 2010) or ANR (Ray et al., 1997). In these embodiments, expression of the payload gene is particularly activated in a low-oxygen or anaerobic environment, such as in the gut, e.g., a mammalian gut, e.g., a human gut. In some embodiments, gene expression is further optimized by methods known in the art, e.g., by optimizing ribosomal binding sites and/or increasing mRNA stability.

In another embodiment, the recombinant bacteria comprise the gene or gene cassette for producing an effector molecule, or a therapeutic polypeptide, expressed under the control of anaerobic regulation of arginine deiminiase and nitrate reduction transcriptional regulator (ANR). In P. aeruginosa, ANR is “required for the expression of physiological functions which are inducible under oxygen-limiting or anaerobic conditions” (Winteler et al., 1996; Sawers 1991). P. aeruginosa ANR is homologous with E. coli FNR, and “the consensus FNR site (TTGAT----ATCAA) was recognized efficiently by ANR and FNR” (Winteler et al., 1996). Like FNR, in the anaerobic state, ANR activates numerous genes responsible for adapting to anaerobic growth. In the aerobic state, ANR is inactive. Pseudomonas fluorescens, Pseudomonas putida, Pseudomonas syringae, and Pseudomonas mendocina all have functional analogs of ANR (Zimmermann et al., 1991). Promoters that are regulated by ANR are known in the art, e.g., the promoter of the arcDABC operon (see, e.g., Hasegawa et al., 1998).

In other embodiments, the one or more gene sequence(s) for producing a payload are expressed under the control of an oxygen level-dependent promoter fused to a binding site for a transcriptional activator, e.g., CRP. CRP (cyclic AMP receptor protein or catabolite activator protein or CAP) plays a major regulatory role in bacteria by repressing genes responsible for the uptake, metabolism, and assimilation of less favorable carbon sources when rapidly metabolizable carbohydrates, such as glucose, are present (Wu et al., 2015). This preference for glucose has been termed glucose repression, as well as carbon catabolite repression (Deutscher, 2008; Görke and Stülke, 2008). In some embodiments, the gene or gene cassette for producing an effector molecule(s) is controlled by an oxygen level-dependent promoter fused to a CRP binding site. In some embodiments, the one or more gene sequence(s) for a payload are controlled by a FNR promoter fused to a CRP binding site. In these embodiments, cyclic AMP binds to CRP when no glucose is present in the environment. This binding causes a conformational change in CRP, and allows CRP to bind tightly to its binding site. CRP binding then activates transcription of the gene or gene cassette by recruiting RNA polymerase to the FNR promoter via direct protein-protein interactions. In the presence of glucose, cyclic AMP does not bind to CRP and transcription of the gene or gene cassette for producing an payload is repressed. In some embodiments, an oxygen level-dependent promoter (e.g., an FNR promoter) fused to a binding site for a transcriptional activator is used to ensure that the gene or gene cassette for producing a payload is not expressed under anaerobic conditions when sufficient amounts of glucose are present, e.g., by adding glucose to growth media in vitro.

In some embodiments, the recombinant bacteria comprise an oxygen level-dependent promoter from a different species, strain, or substrain of bacteria. In some embodiments, the recombinant bacteria comprise an oxygen level-sensing transcription factor, e.g., FNR, ANR or DNR, from a different species, strain, or substrain of bacteria. In some embodiments, the recombinant bacteria comprise an oxygen level-sensing transcription factor and corresponding promoter from a different species, strain, or substrain of bacteria. The heterologous oxygen-level dependent transcriptional regulator and/or promoter increases the transcription of genes operably linked to said promoter, e.g., one or more gene sequence(s) for producing the payload(s) in a low-oxygen or anaerobic environment, as compared to the native gene(s) and promoter in the bacteria under the same conditions. In certain embodiments, the non-native oxygen-level dependent transcriptional regulator is an FNR protein from N. gonorrhoeae (see, e.g., Isabella et al., 2011). In some embodiments, the corresponding wild-type transcriptional regulator is left intact and retains wild-type activity. In alternate embodiments, the corresponding wild-type transcriptional regulator is deleted or mutated to reduce or eliminate wild-type activity.

In some embodiments, the recombinant bacteria comprise a wild-type oxygen-level dependent transcriptional regulator, e.g., FNR, ANR, or DNR, and corresponding promoter that is mutated relative to the wild-type promoter from bacteria of the same subtype. The mutated promoter enhances binding to the wild-type transcriptional regulator and increases the transcription of genes operably linked to said promoter, e.g., the gene encoding the payload, in a low-oxygen or anaerobic environment, as compared to the wild-type promoter under the same conditions. In some embodiments, the recombinant bacteria comprise a wild-type oxygen-level dependent promoter, e.g., FNR, ANR, or DNR promoter, and corresponding transcriptional regulator that is mutated relative to the wild-type transcriptional regulator from bacteria of the same subtype. The mutated transcriptional regulator enhances binding to the wild-type promoter and increases the transcription of genes operably linked to said promoter, e.g., the gene encoding the payload, in a low-oxygen or anaerobic environment, as compared to the wild-type transcriptional regulator under the same conditions. In certain embodiments, the mutant oxygen-level dependent transcriptional regulator is an FNR protein comprising amino acid substitutions that enhance dimerization and FNR activity (see, e.g., Moore et al., (2006). In some embodiments, both the oxygen level-sensing transcriptional regulator and corresponding promoter are mutated relative to the wild-type sequences from bacteria of the same subtype in order to increase expression of the payload in low-oxygen conditions.

In some embodiments, the bacterial cells comprise multiple copies of the endogenous gene encoding the oxygen level-sensing transcriptional regulator, e.g., the FNR gene. In some embodiments, the gene encoding the oxygen level-sensing transcriptional regulator is present on a plasmid. In some embodiments, the gene encoding the oxygen level-sensing transcriptional regulator and the gene encoding the payload are present on different plasmids. In some embodiments, the gene encoding the oxygen level-sensing transcriptional regulator and the gene encoding the payload are present on the same plasmid.

In some embodiments, the gene encoding the oxygen level-sensing transcriptional regulator is present on a chromosome. In some embodiments, the gene encoding the oxygen level-sensing transcriptional regulator and the gene encoding the payload are present on different chromosomes. In some embodiments, the gene encoding the oxygen level-sensing transcriptional regulator and the gene encoding the payload are present on the same chromosome. In some instances, it may be advantageous to express the oxygen level-sensing transcriptional regulator under the control of an inducible promoter in order to enhance expression stability. In some embodiments, expression of the transcriptional regulator is controlled by a different promoter than the promoter that controls expression of the gene encoding the payload. In some embodiments, expression of the transcriptional regulator is controlled by the same promoter that controls expression of the payload. In some embodiments, the transcriptional regulator and the payload are divergently transcribed from a promoter region

RNS-Dependent Regulation

In some embodiments, the recombinant bacteria comprise a gene encoding a payload that is expressed under the control of an inducible promoter. In some embodiments, the recombinant bacterium that expresses a payload under the control of a promoter that is activated by inflammatory conditions. In one embodiment, the gene for producing the payload is expressed under the control of an inflammatory-dependent promoter that is activated in inflammatory environments, e.g., a reactive nitrogen species or RNS promoter.

As used herein, “reactive nitrogen species” and “RNS” are used interchangeably to refer to highly active molecules, ions, and/or radicals derived from molecular nitrogen. RNS can cause deleterious cellular effects such as nitrosative stress. RNS includes, but is not limited to, nitric oxide (NO.), peroxynitrite or peroxynitrite anion (ONOO—), nitrogen dioxide (·NO2), dinitrogen trioxide (N203), peroxynitrous acid (ONOOH), and nitroperoxycarbonate (ONOOCO2-) (unpaired electrons denoted by). Bacteria have evolved transcription factors that are capable of sensing RNS levels. Different RNS signaling pathways are triggered by different RNS levels and occur with different kinetics.

As used herein, “RNS-inducible regulatory region” refers to a nucleic acid sequence to which one or more RNS-sensing transcription factors is capable of binding, wherein the binding and/or activation of the corresponding transcription factor activates downstream gene expression; in the presence of RNS, the transcription factor binds to and/or activates the regulatory region. In some embodiments, the RNS-inducible regulatory region comprises a promoter sequence. In some embodiments, the transcription factor senses RNS and subsequently binds to the RNS-inducible regulatory region, thereby activating downstream gene expression. In alternate embodiments, the transcription factor is bound to the RNS-inducible regulatory region in the absence of RNS; in the presence of RNS, the transcription factor undergoes a conformational change, thereby activating downstream gene expression. The RNS-inducible regulatory region may be operatively linked to a gene or genes, e.g., a payload gene sequence(s), e.g., any of the payloads described herein. For example, in the presence of RNS, a transcription factor senses RNS and activates a corresponding RNS-inducible regulatory region, thereby driving expression of an operatively linked gene sequence. Thus, RNS induces expression of the gene or gene sequences.

Each regulatory region is capable of binding at least one corresponding RNS-sensing transcription factor. Examples of transcription factors that sense RNS and their corresponding RNS-responsive genes, promoters, and/or regulatory regions include, but are not limited to, those shown in Table 6.

TABLE 6
Examples of RNS-sensing transcription
factors and RNS-responsive genes

| | Primarily | |
|---|---|---|
| RNS-sensing | capable of | Examples of responsive genes, |
| transcription factor: | sensing: | promoters, and/or regulatory regions: |
| NsrR | NO | norB, aniA, nsrR, hmpA, ytfE, ygbA, |
| | | hcp, hcr, nrfA, aox |
| NorR | NO | norVW, norR |
| DNR | NO | norCB, nir, nor, nos |

In some embodiments, the recombinant bacteria comprise a tunable regulatory region that is directly or indirectly controlled by a transcription factor that is capable of sensing at least one reactive nitrogen species. The tunable regulatory region is operatively linked to a gene or genes capable of directly or indirectly driving the expression of a payload, thus controlling expression of the payload relative to RNS levels. For example, the tunable regulatory region is a RNS-inducible regulatory region, and the payload is a payload, such as any of the payloads provided herein; when RNS is present, e.g., in an inflamed tissue, a RNS-sensing transcription factor binds to and/or activates the regulatory region and drives expression of the payload gene or genes. Subsequently, when inflammation is ameliorated, RNS levels are reduced, and production of the payload is decreased or eliminated.

In some embodiments, the tunable regulatory region is a RNS-inducible regulatory region; in the presence of RNS, a transcription factor senses RNS and activates the RNS-inducible regulatory region, thereby driving expression of an operatively linked gene or genes. In some embodiments, the transcription factor senses RNS and subsequently binds to the RNS-inducible regulatory region, thereby activating downstream gene expression. In alternate embodiments, the transcription factor is bound to the RNS-inducible regulatory region in the absence of RNS; when the transcription factor senses RNS, it undergoes a conformational change, thereby inducing downstream gene expression.

In some embodiments, the tunable regulatory region is a RNS-inducible regulatory region, and the transcription factor that senses RNS is NorR. NorR “is an NO-responsive transcriptional activator that regulates expression of the norVW genes encoding flavorubredoxin and an associated flavoprotein, which reduce NO to nitrous oxide” (Spiro 2006). The recombinant bacteria may comprise any suitable RNS-responsive regulatory region from a gene that is activated by NorR. Genes that are capable of being activated by NorR are known in the art (see, e.g., Spiro 2006; Vine et al., 2011; Karlinsey et al., 2012). In certain embodiments, the recombinant bacteria comprise a RNS-inducible regulatory region from norVW that is operatively linked to a gene or genes, e.g., one or more payload gene sequence(s). In the presence of RNS, a NorR transcription factor senses RNS and activates to the norVW regulatory region, thereby driving expression of the operatively linked gene(s) and producing the payload(s).

In some embodiments, the tunable regulatory region is a RNS-inducible regulatory region, and the transcription factor that senses RNS is DNR. DNR (dissimilatory nitrate respiration regulator) “promotes the expression of the nir, the nor and the nos genes” in the presence of nitric oxide (Castiglione et al., 2009). The recombinant bacteria may comprise any suitable RNS-responsive regulatory region from a gene that is activated by DNR. Genes that are capable of being activated by DNR are known in the art (see, e.g., Castiglione et al., 2009; Giardina et al., 2008). In certain embodiments, the recombinant bacteria comprise a RNS-inducible regulatory region from norCB that is operatively linked to a gene or gene cassette, e.g., a butyrogenic gene cassette. In the presence of RNS, a DNR transcription factor senses RNS and activates to the norCB regulatory region, thereby driving expression of the operatively linked gene or genes and producing one or more payloads. In some embodiments, the DNR is Pseudomonas aeruginosa DNR. Any suitable transcriptional regulator that is controlled by exogenous environmental conditions and corresponding regulatory region may be used. Non-limiting examples include ArcA/B, ResD/E, NreA/B/C, and AirSR, and others are known in the art.

In some embodiments, the tunable regulatory region is a RNS-derepressible regulatory region, and binding of a corresponding transcription factor represses downstream gene expression; in the presence of RNS, the transcription factor no longer binds to the regulatory region, thereby derepressing the operatively linked gene or gene cassette.

In some embodiments, the tunable regulatory region is a RNS-derepressible regulatory region, and the transcription factor that senses RNS is NsrR. NsrR is “an Rrf2-type transcriptional repressor [that] can sense NO and control the expression of genes responsible for NO metabolism” (Isabella et al., 2009). The recombinant bacteria may comprise any suitable RNS-responsive regulatory region from a gene that is repressed by NsrR. In some embodiments, the NsrR is Neisseria gonorrhoeae NsrR. Genes that are capable of being repressed by NsrR are known in the art (see, e.g., Isabella et al., 2009; Dunn et al., 2010). In certain embodiments, the recombinant bacteria comprise a RNS-derepressible regulatory region from norB that is operatively linked to a gene or genes, e.g., a payload gene or genes. In the presence of RNS, an NsrR transcription factor senses RNS and no longer binds to the norB regulatory region, thereby derepressing the operatively linked a payload gene or genes and producing the encoding a payload(s).

In some embodiments, it is advantageous for the recombinant bacteria to express a RNS-sensing transcription factor that does not regulate the expression of a significant number of native genes in the bacteria. In some embodiments, the recombinant bacterium expresses a RNS-sensing transcription factor from a different species, strain, or substrain of bacteria, wherein the transcription factor does not bind to regulatory sequences in the recombinant bacterium. In some embodiments, the recombinant bacterium is Escherichia coli, and the RNS-sensing transcription factor is NsrR, e.g., from is Neisseria gonorrhoeae, wherein the Escherichia coli does not comprise binding sites for said NsrR. In some embodiments, the heterologous transcription factor minimizes or eliminates off-target effects on endogenous regulatory regions and genes in the recombinant bacteria.

In these embodiments, the recombinant bacteria may comprise a two-repressor activation regulatory circuit, which is used to express a payload. The two-repressor activation regulatory circuit comprises a first RNS-sensing repressor and a second repressor, which is operatively linked to a gene or gene cassette, e.g., encoding a payload. In one aspect of these embodiments, the RNS-sensing repressor inhibits transcription of the second repressor, which inhibits the transcription of the gene or gene cassette. Examples of second repressors useful in these embodiments, include, but are not limited to, TetR, C1, and LexA. In the absence of binding by the first repressor (which occurs in the absence of RNS), the second repressor is transcribed, which represses expression of the gene or genes. In the presence of binding by the first repressor (which occurs in the presence of RNS), expression of the second repressor is repressed, and the gene or genes, e.g., a payload gene or genes is expressed.

A RNS-responsive transcription factor may induce, derepress, or repress gene expression depending upon the regulatory region sequence used in the recombinant bacteria. One or more types of RNS-sensing transcription factors and corresponding regulatory region sequences may be present in recombinant bacteria. In some embodiments, the recombinant bacteria comprise one type of RNS-sensing transcription factor, e.g., NsrR, and one corresponding regulatory region sequence, e.g., from norB. In some embodiments, the recombinant bacteria comprise one type of RNS-sensing transcription factor, e.g., NsrR, and two or more different corresponding regulatory region sequences, e.g., from norB and aniA. In some embodiments, the recombinant bacteria comprise two or more types of RNS-sensing transcription factors, e.g., NsrR and NorR, and two or more corresponding regulatory region sequences, e.g., from norB and norR, respectively. One RNS-responsive regulatory region may be capable of binding more than one transcription factor. In some embodiments, the recombinant bacteria comprise two or more types of RNS-sensing transcription factors and one corresponding regulatory region sequence. Nucleic acid sequences of several RNS-regulated regulatory regions are known in the art (see, e.g., Spiro 2006; Isabella et al., 2009; Dunn et al., 2010; Vine et al., 2011; Karlinsey et al., 2012).

In some embodiments, the recombinant bacteria comprise a gene encoding a RNS-sensing transcription factor, e.g., the nsrR gene, that is controlled by its native promoter, an inducible promoter, a promoter that is stronger than the native promoter, e.g., the GlnRS promoter or the P(Bla) promoter, or a constitutive promoter. In some instances, it may be advantageous to express the RNS-sensing transcription factor under the control of an inducible promoter in order to enhance expression stability. In some embodiments, expression of the RNS-sensing transcription factor is controlled by a different promoter than the promoter that controls expression of the therapeutic molecule. In some embodiments, expression of the RNS-sensing transcription factor is controlled by the same promoter that controls expression of the therapeutic molecule. In some embodiments, the RNS-sensing transcription factor and therapeutic molecule are divergently transcribed from a promoter region.

In some embodiments, the recombinant bacteria comprise a gene for a RNS-sensing transcription factor from a different species, strain, or substrain of bacteria. In some embodiments, the recombinant bacteria comprise a RNS-responsive regulatory region from a different species, strain, or substrain of bacteria. In some embodiments, the recombinant bacteria comprise a RNS-sensing transcription factor and corresponding RNS-responsive regulatory region from a different species, strain, or substrain of bacteria. The heterologous RNS-sensing transcription factor and regulatory region may increase the transcription of genes operatively linked to said regulatory region in the presence of RNS, as compared to the native transcription factor and regulatory region from bacteria of the same subtype under the same conditions.

In some embodiments, the recombinant bacteria comprise a RNS-sensing transcription factor, NsrR, and corresponding regulatory region, nsrR, from Neisseria gonorrhoeae. In some embodiments, the native RNS-sensing transcription factor, e.g., NsrR, is left intact and retains wild-type activity. In alternate embodiments, the native RNS-sensing transcription factor, e.g., NsrR, is deleted or mutated to reduce or eliminate wild-type activity.

In some embodiments, the recombinant bacteria comprise multiple copies of the endogenous gene encoding the RNS-sensing transcription factor, e.g., the nsrR gene. In some embodiments, the gene encoding the RNS-sensing transcription factor is present on a plasmid. In some embodiments, the gene encoding the RNS-sensing transcription factor and the gene or gene cassette for producing the therapeutic molecule are present on different plasmids. In some embodiments, the gene encoding the RNS-sensing transcription factor and the gene or gene cassette for producing the therapeutic molecule are present on the same plasmid. In some embodiments, the gene encoding the RNS-sensing transcription factor is present on a chromosome. In some embodiments, the gene encoding the RNS-sensing transcription factor and the gene or gene cassette for producing the therapeutic molecule are present on different chromosomes. In some embodiments, the gene encoding the RNS-sensing transcription factor and the gene or gene cassette for producing the therapeutic molecule are present on the same chromosome.

In some embodiments, the recombinant bacteria comprise a wild-type gene encoding a RNS-sensing transcription factor, e.g., the NsrR gene, and a corresponding regulatory region, e.g., a norB regulatory region, that is mutated relative to the wild-type regulatory region from bacteria of the same subtype. The mutated regulatory region increases the expression of the payload in the presence of RNS, as compared to the wild-type regulatory region under the same conditions. In some embodiments, the recombinant bacteria comprise a wild-type RNS-responsive regulatory region, e.g., the norB regulatory region, and a corresponding transcription factor, e.g., NsrR, that is mutated relative to the wild-type transcription factor from bacteria of the same subtype. The mutant transcription factor increases the expression of the payload in the presence of RNS, as compared to the wild-type transcription factor under the same conditions. In some embodiments, both the RNS-sensing transcription factor and corresponding regulatory region are mutated relative to the wild-type sequences from bacteria of the same subtype in order to increase expression of the payload in the presence of RNS.

In some embodiments, the gene or gene cassette for producing the e molecule(s) is present on a plasmid and operably linked to a promoter that is induced by RNS. In some embodiments, expression is further optimized by methods known in the art, e.g., by optimizing ribosomal binding sites, manipulating transcriptional regulators, and/or increasing mRNA stability.

In some embodiments, any of the gene(s) of the present disclosure may be integrated into the bacterial chromosome at one or more integration sites. For example, one or more copies of one or more encoding a payload gene(s) may be integrated into the bacterial chromosome. Having multiple copies of the gene or gen(s) integrated into the chromosome allows for greater production of the payload(s) and also permits fine-tuning of the level of expression. Alternatively, different circuits described herein, such as any of the secretion or exporter circuits, in addition to the therapeutic gene(s) or gene cassette(s) could be integrated into the bacterial chromosome at one or more different integration sites to perform multiple different functions.

In some embodiments, the recombinant bacteria produce at least one payload in the presence of RNS to reduce local gut inflammation by at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold as compared to unmodified bacteria of the same subtype under the same conditions. Inflammation may be measured by methods known in the art, e.g., counting disease lesions using endoscopy; detecting T regulatory cell differentiation in peripheral blood, e.g., by fluorescence activated sorting; measuring T regulatory cell levels; measuring cytokine levels; measuring areas of mucosal damage; assaying inflammatory biomarkers, e.g., by qPCR; PCR arrays; transcription factor phosphorylation assays; immunoassays; and/or cytokine assay kits (Mesoscale, Cayman Chemical, Qiagen).

In some embodiments, the recombinant bacteria produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more of payload in the presence of RNS than unmodified bacteria of the same subtype under the same conditions. Certain unmodified bacteria will not have detectable levels of the payload. In embodiments using genetically modified forms of these bacteria, payload will be detectable in the presence of RNS.

ROS-Dependent Regulation

In some embodiments, the recombinant bacteria comprise a gene for producing a payload that is expressed under the control of an inducible promoter. In some embodiments, the recombinant bacterium that expresses a payload under the control of a promoter that is activated by conditions of cellular damage. In one embodiment, the gene for producing the payload is expressed under the control of an cellular damaged-dependent promoter that is activated in environments in which there is cellular or tissue damage, e.g., a reactive oxygen species or ROS promoter.

As used herein, “reactive oxygen species” and “ROS” are used interchangeably to refer to highly active molecules, ions, and/or radicals derived from molecular oxygen. ROS can be produced as byproducts of aerobic respiration or metal-catalyzed oxidation and may cause deleterious cellular effects such as oxidative damage. ROS includes, but is not limited to, hydrogen peroxide (H₂O₂), organic peroxide (ROOH), hydroxyl ion (OH—), hydroxyl radical (·OH), superoxide or superoxide anion (·O2-), singlet oxygen (1O2), ozone (O3), carbonate radical, peroxide or peroxyl radical (·O2-2), hypochlorous acid (HOCl), hypochlorite ion (OCl—), sodium hypochlorite (NaOCl), nitric oxide (NO·), and peroxynitrite or peroxynitrite anion (ONOO—) (unpaired electrons denoted by ·). Bacteria have evolved transcription factors that are capable of sensing ROS levels. Different ROS signaling pathways are triggered by different ROS levels and occur with different kinetics (Marinho et al., 2014).

As used herein, “ROS-inducible regulatory region” refers to a nucleic acid sequence to which one or more ROS-sensing transcription factors is capable of binding, wherein the binding and/or activation of the corresponding transcription factor activates downstream gene expression; in the presence of ROS, the transcription factor binds to and/or activates the regulatory region. In some embodiments, the ROS-inducible regulatory region comprises a promoter sequence. In some embodiments, the transcription factor senses ROS and subsequently binds to the ROS-inducible regulatory region, thereby activating downstream gene expression. In alternate embodiments, the transcription factor is bound to the ROS-inducible regulatory region in the absence of ROS; in the presence of ROS, the transcription factor undergoes a conformational change, thereby activating downstream gene expression. The ROS-inducible regulatory region may be operatively linked to a gene sequence or gene sequence, e.g., a sequence or sequences encoding one or more payload(s). For example, in the presence of ROS, a transcription factor, e.g., OxyR, senses ROS and activates a corresponding ROS-inducible regulatory region, thereby driving expression of an operatively linked gene sequence or gene sequences. Thus, ROS induces expression of the gene or genes. Alternatively, in the presence of ROS, a transcription factor, e.g., PerR, senses ROS and binds to a corresponding ROS-repressible regulatory region, thereby blocking expression of an operatively linked gene sequence or gene sequences. Thus, ROS represses expression of the gene or genes.

Each regulatory region is capable of binding at least one corresponding ROS-sensing transcription factor. Examples of transcription factors that sense ROS and their corresponding ROS-responsive genes, promoters, and/or regulatory regions include, but are not limited to, those shown in Table 7.

TABLE 7
Examples of ROS-sensing transcription
factors and ROS-responsive genes

| ROS-sensing | Primarily | |
|---|---|---|
| transcription | capable of | Examples of responsive genes, |
| factor: | sensing: | promoters, and/or regulatory regions: |
| OxyR | H₂O₂ | ahpC; ahpF; dps; dsbG; fhuF; flu; fur; |
| | | gor; grxA; hemH; katG; oxyS; sufA; sufB; |
| | | sufC; sufD; sufE; sufS; trxC; uxuA; yaaA; |
| | | yaeH; yaiA; ybjM; ydcH; ydeN; ygaQ; |
| | | yljA; ytfK |
| PerR | H₂O₂ | katA; ahpCF; mrgA; zoaA; fur; |
| | | hemAXCDBL; srfA |
| OhrR | Organic | ohrA |
| | peroxides | |
| | NaOCl | |
| SoxR | •O₂⁻ | soxS |
| | NO• | |
| | (also capable | |
| | of sensing | |
| | H₂O₂) | |
| RosR | H₂O₂ | rbtT; tnp16a; rluC1; tnp5a; mscL; tnp2d; |
| | | phoD; tnp15b; pstA; tnp5b; xylC; gabD1; |
| | | rluC2; cgtS9; azlC; narKGHJI; rosR |

In some embodiments, the recombinant bacteria comprise a tunable regulatory region that is directly or indirectly controlled by a transcription factor that is capable of sensing at least one reactive oxygen species. The tunable regulatory region is operatively linked to a gene or gene cassette capable of directly or indirectly driving the expression of a payload, thus controlling expression of the payload relative to ROS levels. For example, the tunable regulatory region is a ROS-inducible regulatory region, and the molecule is a payload; when ROS is present, e.g., in an inflamed tissue, a ROS-sensing transcription factor binds to and/or activates the regulatory region and drives expression of the gene sequence for the payload, thereby producing the payload. Subsequently, when inflammation is ameliorated, ROS levels are reduced, and production of the payload is decreased or eliminated.

In some embodiments, the tunable regulatory region is a ROS-inducible regulatory region; in the presence of ROS, a transcription factor senses ROS and activates the ROS-inducible regulatory region, thereby driving expression of an operatively linked gene or gene cassette. In some embodiments, the transcription factor senses ROS and subsequently binds to the ROS-inducible regulatory region, thereby activating downstream gene expression. In alternate embodiments, the transcription factor is bound to the ROS-inducible regulatory region in the absence of ROS; when the transcription factor senses ROS, it undergoes a conformational change, thereby inducing downstream gene expression.

In some embodiments, the tunable regulatory region is a ROS-inducible regulatory region, and the transcription factor that senses ROS is OxyR. OxyR “functions primarily as a global regulator of the peroxide stress response” and is capable of regulating dozens of genes, e.g., “genes involved in H₂O₂ detoxification (katE, ahpCF), heme biosynthesis (hemH), reductant supply (grxA, gor, trxC), thiol-disulfide isomerization (dsbG), Fe—S center repair (sufA-E, sufS), iron binding (yaaA), repression of iron import systems (fur)” and “OxyS, a small regulatory RNA” (Dubbs et al., 2012). The recombinant bacteria may comprise any suitable ROS-responsive regulatory region from a gene that is activated by OxyR. Genes that are capable of being activated by OxyR are known in the art (see, e.g., Zheng et al., 2001; Dubbs et al., 2012). In certain embodiments, the recombinant bacteria comprise a ROS-inducible regulatory region from oxyS that is operatively linked to a gene, e.g., a payload gene. In the presence of ROS, e.g., H2O2, an OxyR transcription factor senses ROS and activates to the oxyS regulatory region, thereby driving expression of the operatively linked payload gene and producing the payload. In some embodiments, OxyR is encoded by an E. coli oxyR gene. In some embodiments, the oxyS regulatory region is an E. coli oxyS regulatory region. In some embodiments, the ROS-inducible regulatory region is selected from the regulatory region of katG, dps, and ahpC.

In alternate embodiments, the tunable regulatory region is a ROS-inducible regulatory region, and the corresponding transcription factor that senses ROS is SoxR. When SoxR is “activated by oxidation of its [2Fe-2S] cluster, it increases the synthesis of SoxS, which then activates its target gene expression” (Koo et al., 2003). “SoxR is known to respond primarily to superoxide and nitric oxide” (Koo et al., 2003), and is also capable of responding to H2O2. The recombinant bacteria may comprise any suitable ROS-responsive regulatory region from a gene that is activated by SoxR. Genes that are capable of being activated by SoxR are known in the art (see, e.g., Koo et al., 2003). In certain embodiments, the recombinant bacteria comprise a ROS-inducible regulatory region from soxS that is operatively linked to a gene, e.g., a payload. In the presence of ROS, the SoxR transcription factor senses ROS and activates the soxS regulatory region, thereby driving expression of the operatively linked a payload gene and producing the a payload.

In some embodiments, the tunable regulatory region is a ROS-derepressible regulatory region, and binding of a corresponding transcription factor represses downstream gene expression; in the presence of ROS, the transcription factor no longer binds to the regulatory region, thereby derepressing the operatively linked gene or gene cassette.

In some embodiments, the tunable regulatory region is a ROS-derepressible regulatory region, and the transcription factor that senses ROS is OhrR. OhrR “binds to a pair of inverted repeat DNA sequences overlapping the ohrA promoter site and thereby represses the transcription event,” but oxidized OhrR is “unable to bind its DNA target” (Duarte et al., 2010). OhrR is a “transcriptional repressor [that] . . . senses both organic peroxides and NaOCl” (Dubbs et al., 2012) and is “weakly activated by H₂O₂ but it shows much higher reactivity for organic hydroperoxides” (Duarte et al., 2010). The recombinant bacteria may comprise any suitable ROS-responsive regulatory region from a gene that is repressed by OhrR. Genes that are capable of being repressed by OhrR are known in the art (see, e.g., Dubbs et al., 2012). In certain embodiments, the recombinant bacteria comprise a ROS-derepressible regulatory region from ohrA that is operatively linked to a gene or gene cassette, e.g., a payload gene. In the presence of ROS, e.g., NaOCl, an OhrR transcription factor senses ROS and no longer binds to the ohrA regulatory region, thereby derepressing the operatively linked payload gene and producing the a payload.

OhrR is a member of the MarR family of ROS-responsive regulators. “Most members of the MarR family are transcriptional repressors and often bind to the -10 or -35 region in the promoter causing a steric inhibition of RNA polymerase binding” (Bussmann et al., 2010). Other members of this family are known in the art and include, but are not limited to, OspR, MgrA, RosR, and SarZ. In some embodiments, the transcription factor that senses ROS is OspR, MgRA, RosR, and/or SarZ, and the recombinant bacteria comprises one or more corresponding regulatory region sequences from a gene that is repressed by OspR, MgRA, RosR, and/or SarZ. Genes that are capable of being repressed by OspR, MgRA, RosR, and/or SarZ are known in the art (see, e.g., Dubbs et al., 2012).

In some embodiments, the tunable regulatory region is a ROS-derepressible regulatory region, and the corresponding transcription factor that senses ROS is RosR. RosR is “a MarR-type transcriptional regulator” that binds to an “18-bp inverted repeat with the consensus sequence TTGTTGAYRYRTCAACWA (SEQ ID NO: 1085)” and is “reversibly inhibited by the oxidant H2O2” (Bussmann et al., 2010). RosR is capable of repressing numerous genes and putative genes, including but not limited to “a putative polyisoprenoid-binding protein (cg1322, gene upstream of and divergent from rosR), a sensory histidine kinase (cgtS9), a putative transcriptional regulator of the Crp/FNR family (cg3291), a protein of the glutathione S-transferase family (cg1426), two putative FMN reductases (cg1150 and cg1850), and four putative monooxygenases (cg0823, cg1848, cg2329, and cg3084)” (Bussmann et al., 2010). The recombinant bacteria may comprise any suitable ROS-responsive regulatory region from a gene that is repressed by RosR. Genes that are capable of being repressed by RosR are known in the art (see, e.g., Bussmann et al., 2010). In certain embodiments, the recombinant bacteria comprise a ROS-derepressible regulatory region from cgtS9 that is operatively linked to a gene or gene cassette, e.g., a payload. In the presence of ROS, e.g., H2O2, a RosR transcription factor senses ROS and no longer binds to the cgtS9 regulatory region, thereby derepressing the operatively linked payload gene and producing the payload.

In some embodiments, it is advantageous for the recombinant bacteria to express a ROS-sensing transcription factor that does not regulate the expression of a significant number of native genes in the bacteria. In some embodiments, the recombinant bacterium expresses a ROS-sensing transcription factor from a different species, strain, or substrain of bacteria, wherein the transcription factor does not bind to regulatory sequences in the recombinant bacterium. In some embodiments, the recombinant bacterium is Escherichia coli, and the ROS-sensing transcription factor is RosR, e.g., from Corynebacterium glutamicum, wherein the Escherichia coli does not comprise binding sites for said RosR. In some embodiments, the heterologous transcription factor minimizes or eliminates off-target effects on endogenous regulatory regions and genes in the recombinant bacteria.

In some embodiments, the tunable regulatory region is a ROS-repressible regulatory region, and the transcription factor that senses ROS is PerR. In Bacillus subtilis, PerR “when bound to DNA, represses the genes coding for proteins involved in the oxidative stress response (katA, ahpC, and mrgA), metal homeostasis (hemAXCDBL, fur, and zoaA) and its own synthesis (perR)” (Marinho et al., 2014). PerR is a “global regulator that responds primarily to H2O2” (Dubbs et al., 2012) and “interacts with DNA at the per box, a specific palindromic consensus sequence (TTATAATNATTATAA (SEQ ID NO: 1086)) residing within and near the promoter sequences of PerR-controlled genes” (Marinho et al., 2014). PerR is capable of binding a regulatory region that “overlaps part of the promoter or is immediately downstream from it” (Dubbs et al., 2012). The recombinant bacteria may comprise any suitable ROS-responsive regulatory region from a gene that is repressed by PerR. Genes that are capable of being repressed by PerR are known in the art (see, e.g., Dubbs et al., 2012).

In these embodiments, the recombinant bacteria may comprise a two repressor activation regulatory circuit, which is used to express a payload. The two repressor activation regulatory circuit comprises a first ROS-sensing repressor, e.g., PerR, and a second repressor, e.g., TetR, which is operatively linked to a gene or gene cassette, e.g., a payload. In one aspect of these embodiments, the ROS-sensing repressor inhibits transcription of the second repressor, which inhibits the transcription of the gene or gene cassette. Examples of second repressors useful in these embodiments include, but are not limited to, TetR, Cl, and LexA. In some embodiments, the ROS-sensing repressor is PerR. In some embodiments, the second repressor is TetR. In this embodiment, a PerR-repressible regulatory region drives expression of TetR, and a TetR-repressible regulatory region drives expression of the gene or gene cassette, e.g., a payload. In the absence of PerR binding (which occurs in the absence of ROS), tetR is transcribed, and TetR represses expression of the gene or gene cassette, e.g., a payload. In the presence of PerR binding (which occurs in the presence of ROS), tetR expression is repressed, and the gene or gene cassette, e.g., a payload, is expressed.

A ROS-responsive transcription factor may induce, derepress, or repress gene expression depending upon the regulatory region sequence used in the recombinant bacteria. For example, although “OxyR is primarily thought of as a transcriptional activator under oxidizing conditions. OxyR can function as either a repressor or activator under both oxidizing and reducing conditions” (Dubbs et al., 2012), and OxyR “has been shown to be a repressor of its own expression as well as that of fhuF (encoding a ferric ion reductase) and flu (encoding the antigen 43 outer membrane protein)” (Zheng et al., 2001). The recombinant bacteria may comprise any suitable ROS-responsive regulatory region from a gene that is repressed by OxyR. In some embodiments, OxyR is used in a two repressor activation regulatory circuit, as described above. Genes that are capable of being repressed by OxyR are known in the art (see, e.g., Zheng et al., 2001). Or, for example, although RosR is capable of repressing a number of genes, it is also capable of activating certain genes, e.g., the narKGHJI operon. In some embodiments, the recombinant bacteria comprise any suitable ROS-responsive regulatory region from a gene that is activated by RosR. In some embodiments, the recombinant bacteria comprise any suitable ROS-responsive regulatory region from a gene that is activated by PerR.

One or more types of ROS-sensing transcription factors and corresponding regulatory region sequences may be present in recombinant bacteria. For example, “OhrR is found in both Gram-positive and Gram-negative bacteria and can coreside with either OxyR or PerR or both” (Dubbs et al., 2012). In some embodiments, the recombinant bacteria comprise one type of ROS-sensing transcription factor, e.g., OxyR, and one corresponding regulatory region sequence, e.g., from oxyS. In some embodiments, the recombinant bacteria comprise one type of ROS-sensing transcription factor, e.g., OxyR, and two or more different corresponding regulatory region sequences, e.g., from oxyS and katG. In some embodiments, the recombinant bacteria comprise two or more types of ROS-sensing transcription factors, e.g., OxyR and PerR, and two or more corresponding regulatory region sequences, e.g., from oxyS and katA, respectively. One ROS-responsive regulatory region may be capable of binding more than one transcription factor. In some embodiments, the recombinant bacteria comprise two or more types of ROS-sensing transcription factors and one corresponding regulatory region sequence.

In some embodiments, recombinant bacteria comprise nucleic acid sequences comprising OxyR binding sites. In some embodiments, recombinant bacteria comprise a nucleic acid sequence that is at least about 80%, 85%, 90%, 95%, or 99% homologous to the DNA sequence of SEQ ID NO: 168, SEQ ID NO: 169, or SEQ ID NO: 170, or SEQ ID NO: 171, or a functional fragment thereof. In some embodiments, the regulatory region sequence is at least about 80%, 85%, 90%, 95%, or 99% homologous to the sequence of SEQ ID NO: 168, SEQ ID NO: 169, SEQ ID NO: 170, and/or SEQ ID NO: 171.

In some embodiments, the recombinant bacteria comprise a gene encoding a ROS-sensing transcription factor, e.g., the oxyR gene, that is controlled by its native promoter, an inducible promoter, a promoter that is stronger than the native promoter, e.g., the GlnRS promoter or the P(Bla) promoter, or a constitutive promoter. In some instances, it may be advantageous to express the ROS-sensing transcription factor under the control of an inducible promoter in order to enhance expression stability. In some embodiments, expression of the ROS-sensing transcription factor is controlled by a different promoter than the promoter that controls expression of the therapeutic molecule. In some embodiments, expression of the ROS-sensing transcription factor is controlled by the same promoter that controls expression of the therapeutic molecule. In some embodiments, the ROS-sensing transcription factor and therapeutic molecule are divergently transcribed from a promoter region.

In some embodiments, the recombinant bacteria comprise a gene for a ROS-sensing transcription factor from a different species, strain, or substrain of bacteria. In some embodiments, the recombinant bacteria comprise a ROS-responsive regulatory region from a different species, strain, or substrain of bacteria. In some embodiments, the recombinant bacteria comprise a ROS-sensing transcription factor and corresponding ROS-responsive regulatory region from a different species, strain, or substrain of bacteria. The heterologous ROS-sensing transcription factor and regulatory region may increase the transcription of genes operatively linked to said regulatory region in the presence of ROS, as compared to the native transcription factor and regulatory region from bacteria of the same subtype under the same conditions.

In some embodiments, the recombinant bacteria comprise a ROS-sensing transcription factor, OxyR, and corresponding regulatory region, oxyS, from Escherichia coli. In some embodiments, the native ROS-sensing transcription factor, e.g., OxyR, is left intact and retains wild-type activity. In alternate embodiments, the native ROS-sensing transcription factor, e.g., OxyR, is deleted or mutated to reduce or eliminate wild-type activity.

In some embodiments, the recombinant bacteria comprise multiple copies of the endogenous gene encoding the ROS-sensing transcription factor, e.g., the oxyR gene. In some embodiments, the gene encoding the ROS-sensing transcription factor is present on a plasmid. In some embodiments, the gene encoding the ROS-sensing transcription factor and the gene or gene cassette for producing the therapeutic molecule are present on different plasmids. In some embodiments, the gene encoding the ROS-sensing transcription factor and the gene or gene cassette for producing the therapeutic molecule are present on the same. In some embodiments, the gene encoding the ROS-sensing transcription factor is present on a chromosome. In some embodiments, the gene encoding the ROS-sensing transcription factor and the gene or gene cassette for producing the therapeutic molecule are present on different chromosomes. In some embodiments, the gene encoding the ROS-sensing transcription factor and the gene or gene cassette for producing the therapeutic molecule are present on the same chromosome.

In some embodiments, the recombinant bacteria comprise a wild-type gene encoding a ROS-sensing transcription factor, e.g., the soxR gene, and a corresponding regulatory region, e.g., a soxS regulatory region, that is mutated relative to the wild-type regulatory region from bacteria of the same subtype. The mutated regulatory region increases the expression of the payload in the presence of ROS, as compared to the wild-type regulatory region under the same conditions. In some embodiments, the recombinant bacteria comprise a wild-type ROS-responsive regulatory region, e.g., the oxyS regulatory region, and a corresponding transcription factor, e.g., OxyR, that is mutated relative to the wild-type transcription factor from bacteria of the same subtype. The mutant transcription factor increases the expression of the payload in the presence of ROS, as compared to the wild-type transcription factor under the same conditions. In some embodiments, both the ROS-sensing transcription factor and corresponding regulatory region are mutated relative to the wild-type sequences from bacteria of the same subtype in order to increase expression of the payload in the presence of ROS.

In some embodiments, the gene or gene cassette for producing the payload is present on a plasmid and operably linked to a promoter that is induced by ROS. In some embodiments, the gene or gene cassette for producing the payload is present in the chromosome and operably linked to a promoter that is induced by ROS. In some embodiments, the gene or gene cassette for producing the payload is present on a chromosome and operably linked to a promoter that is induced by exposure to tetracycline. In some embodiments, the gene or gene cassette for producing the payload is present on a plasmid and operably linked to a promoter that is induced by exposure to tetracycline. In some embodiments, expression is further optimized by methods known in the art, e.g., by optimizing ribosomal binding sites, manipulating transcriptional regulators, and/or increasing mRNA stability.

In some embodiments, the recombinant bacteria may comprise multiple copies of the gene(s) capable of producing a payload(s). In some embodiments, the gene(s) capable of producing a payload(s) is present on a plasmid and operatively linked to a ROS-responsive regulatory region. In some embodiments, the gene(s) capable of producing a payload is present in a chromosome and operatively linked to a ROS-responsive regulatory region.

Thus, in some embodiments, the recombinant bacteria produce one or more payloads under the control of an oxygen level-dependent promoter, a reactive oxygen species (ROS)-dependent promoter, or a reactive nitrogen species (RNS)-dependent promoter, and a corresponding transcription factor.

In some embodiments, the recombinant bacteria comprise a stably maintained plasmid or chromosome carrying a gene for producing a payload, such that the payload can be expressed in the host cell, and the host cell is capable of survival and/or growth in vitro, e.g., in medium, and/or in vivo. In some embodiments, a bacterium may comprise multiple copies of the gene encoding the payload. In some embodiments, the gene encoding the payload is expressed on a low-copy plasmid. In some embodiments, the low-copy plasmid may be useful for increasing stability of expression. In some embodiments, the low-copy plasmid may be useful for decreasing leaky expression under non-inducing conditions. In some embodiments, the gene encoding the payload is expressed on a high-copy plasmid. In some embodiments, the high-copy plasmid may be useful for increasing expression of the payload. In some embodiments, the gene encoding the payload is expressed on a chromosome.

Propionate and Other Promoters

In some embodiments, the recombinant bacteria comprise the gene or gene cassette for producing an effector molecule, or a therapeutic polypeptides, expressed under the control of an inducible promoter that is responsive to specific molecules or metabolites in the environment, e.g., the tumor microenvironment, a specific tissue, or the mammalian gut. For example, the short-chain fatty acid propionate is a major microbial fermentation metabolite localized to the gut (Hosseini et al., 2011). In one embodiment, the gene or gene cassette for producing an effector molecule(s) is under the control of a propionate-inducible promoter. In a more specific embodiment, the gene or gene cassette for producing the effector molecule(s) is under the control of a propionate-inducible promoter that is activated by the presence of propionate in the mammalian gut. Any molecule or metabolite found in the mammalian gut, in a healthy and/or disease state, may be used to induce payload expression. Non-limiting examples of inducers include propionate, bilirubin, aspartate aminotransferase, alanine aminotransferase, blood coagulation factors II, VII, IX, and X, alkaline phosphatase, gamma glutamyl transferase, hepatitis antigens and antibodies, alpha fetoprotein, anti-mitochondrial, smooth muscle, and anti-nuclear antibodies, iron, transferrin, ferritin, copper, ceruloplasmin, ammonia, and manganese. In alternate embodiments, the gene or gene cassette for producing an effector molecule(s) is under the control of a pBAD promoter, which is activated in the presence of the sugar arabinose.

In some embodiments, the gene or gene cassette for producing the effector molecule(s) is present on a plasmid and operably linked to a promoter that is induced under low-oxygen or anaerobic conditions. In some embodiments, the gene or gene cassette for producing the effector molecule(s) is present in the chromosome and operably linked to a promoter that is induced under low-oxygen or anaerobic conditions. In some embodiments, the gene or gene cassette for producing the effector molecule(s) is present on a plasmid and operably linked to a promoter that is induced by molecules or metabolites that are specific to the mammalian gut. In some embodiments, the gene or gene cassette for producing the effector molecule(s) is present on a chromosome and operably linked to a promoter that is induced by molecules or metabolites that are specific to the tumor and/or the mammalian gut. In some embodiments, the gene or gene cassette for producing the effector molecule(s) is present on a chromosome and operably linked to a promoter that is induced by exposure to tetracycline. In some embodiments, the gene or gene cassette for producing the effector molecule(s) is present on a plasmid and operably linked to a promoter that is induced by exposure to tetracycline. In some embodiments, expression is further optimized by methods known in the art, e.g., by optimizing ribosomal binding sites, manipulating transcriptional regulators, and/or increasing mRNA stability.

In some embodiments, the recombinant bacteria comprise a stably maintained plasmid or chromosome carrying the gene or gene cassette for producing the effector molecule(s), such that the gene or gene cassette can be expressed in the host cell, and the host cell is capable of survival and/or growth in vitro, e.g., in medium, and/or in vivo, e.g., in the gut. In some embodiments, a bacterium may comprise multiple copies of the gene or gene cassette for producing the effector molecule(s). In some embodiments, gene or gene cassette for producing the payload is expressed on a low-copy plasmid. In some embodiments, the low-copy plasmid may be useful for increasing stability of expression. In some embodiments, the low-copy plasmid may be useful for decreasing leaky expression under non-inducing conditions. In some embodiments, gene or gene cassette for producing the effector molecule(s) is expressed on a high-copy plasmid. In some embodiments, the high-copy plasmid may be useful for increasing gene or gene cassette expression. In some embodiments, gene or gene cassette for producing the effector molecule(s) is expressed on a chromosome.

In some embodiments, the recombinant bacteria comprise a regulatory region comprising a propionate promoter, which is induced in the mammalian gut. In some embodiments, the propionate promoter sequence is at least about 80%, 85%, 90%, 95%, or 99% homologous to the sequence of SEQ ID NO: 172.

Other Inducible Promoters

In some embodiments, the gene encoding the effector molecule(s) is present on a plasmid and operably linked to a promoter that is induced by one or more nutritional and/or chemical inducer(s) and/or metabolite(s). In some embodiments, the gene encoding the effector molecule(s) is present in the chromosome and operably linked to a promoter that is induced by one or more nutritional and/or chemical inducer(s) and/or metabolite(s).

In some embodiments, the bacterial cell comprises a stably maintained plasmid or chromosome carrying the one or more gene sequences(s), inducible by one or more nutritional and/or chemical inducer(s) and/or metabolite(s), encoding the effector molecule(s), such that the effector molecule(s) can be expressed in the host cell, and the host cell is capable of survival and/or growth in vitro, e.g., in medium, and/or in vivo, e.g., in the gut. In some embodiments, bacterial cell comprises two or more distinct copies of the one or more gene sequences(s) encoding the effector molecule(s), which is controlled by a promoter inducible one or more nutritional and/or chemical inducer(s) and/or metabolite(s). In some embodiments, the recombinant bacteria comprise multiple copies of the same one or more gene sequences(s) encoding the effector molecule(s), which is controlled by a promoter inducible one or more nutritional and/or chemical inducer(s) and/or metabolite(s). In some embodiments, the one or more gene sequences(s) encoding the effector molecule(s), is present on a plasmid and operably linked to a directly or indirectly inducible promoter inducible by one or more nutritional and/or chemical inducer(s) and/or metabolite(s). In some embodiments, the one or more gene sequences(s) encoding the effector molecule(s), is present on a chromosome and operably linked to a directly or indirectly inducible by one or more nutritional and/or chemical inducer(s) and/or metabolite(s).

In some embodiments, expression of one or more effector molecule(s) and/or other polypeptide(s) of interest, is driven directly or indirectly by one or more promoter(s) induced by a chemical and/or nutritional inducer and/or metabolite during in vitro growth, preparation, or manufacturing of the strain prior to in vivo administration. In some embodiments, the promoter(s) induced by a chemical and/or nutritional inducer and/or metabolite are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule that is added to in the bacterial culture to induce expression and pre-load the bacterium with the effector molecule(s) and/or other polypeptide(s) of interest prior to administration. In some embodiments, the cultures, which are induced by a chemical and/or nutritional inducer and/or metabolite, are grown aerobically. In some embodiments, the cultures, which are induced by a chemical and/or nutritional inducer and/or metabolite, are grown anaerobically.

In some embodiments, expression of one or more effector molecule(s) and/or other polypeptide(s) of interest is driven directly or indirectly by one or more arabinose inducible promoter(s) in vivo. In some embodiments, the promoter is directly or indirectly induced by a chemical and/or nutritional inducer and/or metabolite which is co-administered with the recombinant bacteria, e.g., arabinose.

The genes of arabinose metabolism are organized in one operon, AraBAD, which is controlled by the PAraBAD promoter. The PAraBAD (or Para) promoter suitably fulfills the criteria of inducible expression systems. PAraBAD displays tighter control of payload gene expression than many other systems, likely due to the dual regulatory role of AraC, which functions both as an inducer and as a repressor. Additionally, the level of ParaBAD-based expression can be modulated over a wide range of L-arabinose concentrations to fine-tune levels of expression of the payload. However, the cell population exposed to sub-saturating L-arabinose concentrations is divided into two subpopulations of induced and uninduced cells, which is determined by the differences between individual cells in the availability of L-arabinose transporter (Zhang et al., Development and Application of an Arabinose-Inducible Expression System by Facilitating Inducer Uptake in Corynebacterium glutamicum; Appl. Environ. Microbiol. August 2012 vol. 78 no. 16 5831-5838). Alternatively, inducible expression from the ParaBad can be controlled or fine-tuned through the optimization of the ribosome binding site (RBS), as described herein.

In one embodiment, the arabinose inducible promoter drives the expression of a construct comprising one or more protein(s) of interest, jointly with a second promoter, e.g., a second constitutive or inducible promoter. In some embodiments, two promoters are positioned proximally to the construct and drive its expression, wherein the arabinose inducible promoter drives expression under a first set of exogenous conditions, and the second promoter drives the expression under a second set of exogenous conditions. In a non-limiting example, the first and second conditions may be two sequential culture conditions (i.e., during preparation of the culture in a flask, fermenter or other appropriate culture vessel, e.g., arabinose and IPTG). In another non-limiting example, the first inducing conditions may be culture conditions, e.g., including arabinose presence, and the second inducing conditions may be in vivo conditions. Such in vivo conditions include low-oxygen, microaerobic, or anaerobic conditions, presence of gut metabolites, and/or metabolites administered in combination with the bacterial strain. In some embodiments, the one or more arabinose promoters drive expression of one or more protein(s) of interest, in combination with the FNR promoter driving the expression of the same gene sequence(s).

In some embodiments, one or more protein(s) of interest are knocked into the arabinose operon and are driven by the native arabinose inducible promoter

In some embodiments, the recombinant bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 174. In some embodiments, the arabinose inducible construct further comprises a gene encoding AraC, which is divergently transcribed from the same promoter as the one or more one or more protein(s) of interest. In some embodiments, the recombinant bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 174. In some embodiments, the recombinant bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 175.

In some embodiments, the recombinant bacteria comprise one or more gene sequence(s) which are inducible through a rhamnose inducible system. The genes rhaBAD are organized in one operon which is controlled by the rhaP BAD promoter. The rhaP BAD promoter is regulated by two activators, RhaS and RhaR, and the corresponding genes belong to one transcription unit which divergently transcribed in the opposite direction of rhaBAD. In the presence of L-rhamnose, RhaR binds to the rhaP RS promoter and activates the production of RhaR and RhaS. RhaS together with L-rhamnose then bind to the rhaP BAD and the rhaP T promoter and activate the transcription of the structural genes. In contrast to the arabinose system, in which AraC is provided and divergently transcribed in the gene sequence(s), it is not necessary to express the regulatory proteins in larger quantities in the rhamnose expression system because the amounts expressed from the chromosome are sufficient to activate transcription even on multi-copy plasmids. Therefore, only the rhaP BAD promoter is cloned upstream of the gene that is to be expressed. Full induction of rhaBAD transcription also requires binding of the CRP-cAMP complex, which is a key regulator of catabolite repression. Alternatively, inducible expression from the rhaBAD can be controlled or fine-tuned through the optimization of the ribosome binding site (RBS), as described herein.

In one embodiment, expression of one or more protein(s) of interest is driven directly or indirectly by one or more rhamnose inducible promoter(s). In one embodiment, expression of the payload is driven directly or indirectly by a rhamnose inducible promoter.

In some embodiments, the rhamnose inducible promoter is useful for or induced during in vivo expression of the one or more protein(s) of interest. In some embodiments, expression of one or more protein(s) of interest is driven directly or indirectly by one or more rhamnose inducible promoter(s) in vivo. In some embodiments, the promoter is directly or indirectly induced by a molecule that is co-administered with the recombinant bacteria, e.g., rhamnose

In one embodiment, the rhamnose inducible promoter drives the expression of a construct comprising one or more protein(s) of interest jointly with a second promoter, e.g., a second constitutive or inducible promoter. In some embodiments, two promoters are positioned proximally to the construct and drive its expression, wherein the rhamnose inducible promoter drives expression under a first set of exogenous conditions, and the second promoter drives the expression under a second set of exogenous conditions.

In some embodiments, the recombinant bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 176.

In some embodiments, the recombinant bacteria comprise one or more gene sequence(s) which are inducible through an Isopropyl β-D-1-thiogalactopyranoside (IPTG) inducible system or other compound which induced transcription from the Lac Promoter. IPTG is a molecular mimic of allolactose, a lactose metabolite that activates transcription of the lac operon. In contrast to allolactose, the sulfur atom in IPTG creates a non-hydrolyzable chemical blond, which prevents the degradation of IPTG, allowing the concentration to remain constant. IPTG binds to the lac repressor and releases the tetrameric repressor (lad) from the lac operator in an allosteric manner, thereby allowing the transcription of genes in the lac operon. Since IPTG is not metabolized by E. coli, its concentration stays constant and the rate of expression of Lac promoter-controlled is tightly controlled, both in vivo and in vitro. IPTG intake is independent on the action of lactose permease, since other transport pathways are also involved. Inducible expression from the PLac can be controlled or fine-tuned through the optimization of the ribosome binding site (RBS), as described herein. Other compounds which inactivate LacI, can be used instead of IPTG in a similar manner.

In one embodiment, expression of one or more protein(s) of interest is driven directly or indirectly by one or more IPTG inducible promoter(s). In some embodiments, the IPTG inducible promoter is useful for or induced during in vivo expression of the one or more protein(s) of interest. In some embodiments, expression of one or more protein(s) of interest is driven directly or indirectly by one or more IPTG inducible promoter(s) in vivo. In some embodiments, the promoter is directly or indirectly induced by a molecule that is co-administered with the recombinant bacteria, e.g., IPTG.

In one embodiment, the IPTG inducible promoter drives the expression of a construct comprising one or more protein(s) of interest jointly with a second promoter, e.g., a second constitutive or inducible promoter. In some embodiments, two promoters are positioned proximally to the construct and drive its expression, wherein the IPTG inducible promoter drives expression under a first set of exogenous conditions, and the second promoter drives the expression under a second set of exogenous conditions.

In some embodiments, the recombinant bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 177. In some embodiments, the IPTG inducible construct further comprises a gene encoding lad, which is divergently transcribed from the same promoter as the one or more one or more protein(s) of interest. In some embodiments, the recombinant bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 177. In some embodiments, the recombinant bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 180.

In some embodiments, the recombinant bacteria comprise one or more gene sequence(s) which are inducible through a tetracycline inducible system. The initial system Gossen and Bujard (Tight control of gene expression in mammalian cells by tetracycline-responsive promoters. Gossen M & Bujard H. PNAS, 1992 Jun. 15; 89(12):5547-51) developed is known as tetracycline off: in the presence of tetracycline, expression from a tet-inducible promoter is reduced. Tetracycline-controlled transactivator (tTA) was created by fusing tetR with the C-terminal domain of VP16 (virion protein 16) from herpes simplex virus. In the absence of tetracycline, the tetR portion of tTA will bind tetO sequences in the tet promoter, and the activation domain promotes expression. In the presence of tetracycline, tetracycline binds to tetR, precluding tTA from binding to the tetO sequences. Next, a reverse Tet repressor (rTetR), was developed which created a reliance on the presence of tetracycline for induction, rather than repression. The new transactivator rtTA (reverse tetracycline-controlled trans activator) was created by fusing rTetR with VP16. The tetracycline on system is also known as the rtTA-dependent system.

In one embodiment, expression of one or more protein(s) of interest is driven directly or indirectly by one or more tetracycline inducible promoter(s). In some embodiments, the tetracycline inducible promoter is useful for or induced during in vivo expression of the one or more protein(s) of interest. In some embodiments, expression of one or more protein(s) of interest and/or transcriptional regulator(s), e.g., FNRS24Y, is driven directly or indirectly by one or more tetracycline inducible promoter(s) in vivo. In some embodiments, the promoter is directly or indirectly induced by a molecule that is co-administered with the recombinant bacteria, e.g., tetracycline

In one embodiment, the tetracycline inducible promoter drives the expression of a construct comprising one or more protein(s) of interest jointly with a second promoter, e.g., a second constitutive or inducible promoter. In some embodiments, two promoters are positioned proximally to the construct and drive its expression, wherein the tetracycline inducible promoter drives expression under a first set of exogenous conditions, and the second promoter drives the expression under a second set of exogenous conditions.

In some embodiments, the recombinant bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the bolded sequences of SEQ ID NO: 181 (tet promoter is in bold). In some embodiments, the tetracycline inducible construct further comprises a gene encoding AraC, which is divergently transcribed from the same promoter as the one or more one or more protein(s) of interest. In some embodiments, the recombinant bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 182 in italics (Tet repressor is in italics). In some embodiments, the recombinant bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 182 in italics (Tet repressor is in italics).

In some embodiments, the recombinant bacteria comprise one or more gene sequence(s) whose expression is controlled by a temperature sensitive mechanism. Thermoregulators are advantageous because of strong transcriptional control without the use of external chemicals or specialized media (see, e.g., Nemani et al., Magnetic nanoparticle hyperthermia induced cytosine deaminase expression in microencapsulated E. coli for enzyme-prodrug therapy; J Biotechnol. 2015 Jun. 10; 203: 32-40, and references therein). Thermoregulated protein expression using the mutant cI857 repressor and the pL and/or pR phage λ promoters have been used to engineer recombinant bacterial strains. The gene of interest cloned downstream of the λ promoters can then be efficiently regulated by the mutant thermolabile cI857 repressor of bacteriophage λ. At temperatures below 37° C., cI857 binds to the oL or oR regions of the pR promoter and blocks transcription by RNA polymerase. At higher temperatures, the functional cI857 dimer is destabilized, binding to the oL or oR DNA sequences is abrogated, and mRNA transcription is initiated. An exemplary construct is depicted in in the figures and examples. Inducible expression from the ParaBad can be controlled or further fine-tuned through the optimization of the ribosome binding site (RBS), as described herein.

In one embodiment, expression of one or more protein(s) of interest is driven directly or indirectly by one or more thermoregulated promoter(s). In some embodiments, the thermoregulated promoter is useful for or induced during in vivo expression of the one or more protein(s) of interest. In some embodiments, expression of one or more protein(s) of interest is driven directly or indirectly by one or more thermoregulated promoter(s) in vitro. In some embodiments, the promoter is directly or indirectly induced by a molecule that is co-administered with the recombinant bacteria, e.g., temperature.

In some embodiments, expression of one or more protein(s) of interest is driven directly or indirectly by one or more thermoregulated promoter(s) during in vitro growth, preparation, or manufacturing of the strain prior to in vivo administration. In some embodiments, it may be advantageous to shut off production of the one or more protein(s) of interest. This can be done in a thermoregulated system by growing the strain at lower temperatures, e.g., 30° C. Expression can then be induced by elevating the temperature to 37° C. and/or 42° C. In some embodiments, the thermoregulated promoter(s) are induced in culture, e.g., grown in a flask, fermenter or other appropriate culture vessel, e.g., used during cell growth, cell expansion, fermentation, recovery, purification, formulation, and/or manufacture. In some embodiments, the cultures, which are induced by temperatures between 37° C. and 42° C., are grown aerobically. In some embodiments, the cultures, which are induced by induced by temperatures between 37° C. and 42° C., are grown anaerobically.

In one embodiment, the thermoregulated promoter drives the expression of a construct comprising one or more protein(s) of interest jointly with a second promoter, e.g., a second constitutive or inducible promoter. In some embodiments, two promoters are positioned proximally to the construct and drive its expression, wherein the thermoregulated promoter drives expression under a first set of exogenous conditions, and the second promoter drives the expression under a second set of exogenous conditions.

In some embodiments, the recombinant bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 183. In some embodiments, the thermoregulated construct further comprises a gene encoding mutant cI857 repressor, which is divergently transcribed from the same promoter as the one or more one or more protein(s) of interest. In some embodiments, the recombinant bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 184. In some embodiments, the recombinant bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 185.

In some embodiments, the recombinant bacteria comprise one or more gene sequence(s) which are indirectly inducible through a system driven by the PssB promoter. The Pssb promoter is active under aerobic conditions, and shuts off under anaerobic conditions.

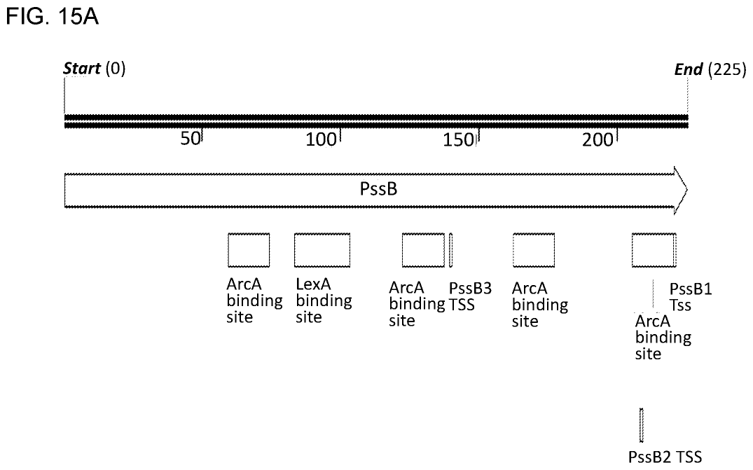

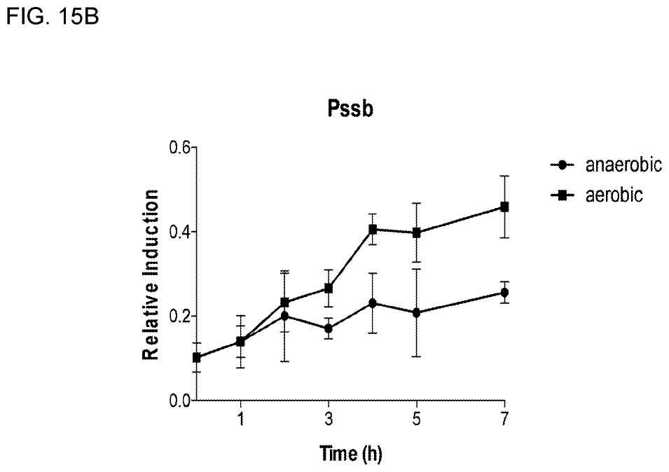

This promoter can be used to express a gene of interest under aerobic conditions. This promoter can also be used to tightly control the expression of a gene product such that it is only expressed under anaerobic conditions. In this case, the oxygen induced PssB promoter induces the expression of a repressor, which represses the expression of a gene of interest. As a result, the gene of interest is only expressed in the absence of the repressor, i.e., under anaerobic conditions. This strategy has the advantage of an additional level of control for improved fine-tuning and tighter control. FIG. 15A depicts a schematic of the gene organization of a PssB promoter.

In one embodiment, expression of one or more protein(s) of interest is indirectly regulated by a repressor expressed under the control of one or more PssB promoter(s). In some embodiments, induction of the RssB promoter(s) indirectly drives the in vivo expression of one or more protein(s) of interest. In some embodiments, induction of the RssB promoter(s) indirectly drives the expression of one or more protein(s) of interest during in vitro growth, preparation, or manufacturing of the strain prior to in vivo administration.

In a non-limiting example, this strategy can be used to control expression of thyA and/or dapA, e.g., to make a conditional auxotroph. The chromosomal copy of dapA or ThyA is knocked out. Under anaerobic conditions, dapA or thyA—as the case may be—are expressed, and the strain can grow in the absence of dap or thymidine. Under aerobic conditions, dapA or thyA expression is shut off, and the strain cannot grow in the absence of dap or thymidine. Such a strategy can, for example be employed to allow survival of bacteria under anaerobic conditions, e.g., the gut, but prevent survival under aerobic conditions (biosafety switch). In some embodiments, the recombinant bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 188. In some embodiments, the inducible promoters, as described above, drive the expression of one or more protein(s) of interest from a low-copy plasmid or a high copy plasmid or a biosafety system plasmid described herein. In some embodiments, the inducible promoters drive the expression of one or more protein(s) of interest from a construct which is integrated into the bacterial chromosome.

Constitutive Promoters

In some embodiments, the gene encoding the payload is present on a plasmid and operably linked to a constitutive promoter. In some embodiments, the gene encoding the payload is present on a chromosome and operably linked to a constitutive promoter.

In some embodiments, the constitutive promoter is active under in vivo conditions, e.g., the gut, as described herein. In some embodiments, the promoters is active under in vitro conditions, e.g., various cell culture and/or cell manufacturing conditions, as described herein. In some embodiments, the constitutive promoter is active under in vivo conditions, e.g., the gut, as described herein, and under in vitro conditions, e.g., various cell culture and/or cell production and/or manufacturing conditions, as described herein.

In some embodiments, the constitutive promoter that is operably linked to the gene encoding the payload is active in various exogenous environmental conditions (e.g., in vivo and/or in vitro and/or production/manufacturing conditions).

In some embodiments, the constitutive promoter is active in exogenous environmental conditions specific to the gut of a mammal. In some embodiments, the constitutive promoter is active in exogenous environmental conditions specific to the small intestine of a mammal. In some embodiments, the constitutive promoter is active in low-oxygen or anaerobic conditions such as the environment of the mammalian gut. In some embodiments, the constitutive promoter is active in the presence of molecules or metabolites that are specific to the gut of a mammal. In some embodiments, the constitutive promoter is directly or indirectly induced by a molecule that is co-administered with the bacterial cell. In some embodiments, the constitutive promoter is active in the presence of molecules or metabolites or other conditions, that are present during in vitro culture, cell production and/or manufacturing conditions.

Bacterial constitutive promoters are known in the art. Exemplary constitutive promoters include E. coli σ70, such as BBa_I14034 (SEQ ID NO: 189), BBa_J732021 (SEQ ID NO: 190), BBa_J742126 (SEQ ID NO: 191), BBa_J01006 (SEQ ID NO: 192), BBa_J23100 (SEQ ID NO: 193), BBa_J23101 (SEQ ID NO: 194), BBa_J23102 (SEQ ID NO: 195), BBa_J23103 (SEQ ID NO: 196), BBa_J23104 (SEQ ID NO: 197), BBa_J23105 (SEQ ID NO: 198), BBa_J23106 (SEQ ID NO: 199), BBa_J23107 (SEQ ID NO: 200), BBa_J23108 (SEQ ID NO: 201), BBa_J23109 (SEQ ID NO: 202), BBa_J23110 (SEQ ID NO: 203), BBa_J23111 (SEQ ID NO: 204), BBa_J23112 (SEQ ID NO: 205), BBa_J23113 (SEQ ID NO: 206), BBa_J23114 (SEQ ID NO: 207), BBa_J23115 (SEQ ID NO: 208), BBa_J23116 (SEQ ID NO: 209), BBa_J23117 (SEQ ID NO: 210), BBa_J23118 (SEQ ID NO: 211), BBa_J23119 (SEQ ID NO: 212), BBa_J23150 (SEQ ID NO: 213), BBa_J23151 (SEQ ID NO: 214), BBa_J44002 (SEQ ID NO: 215), BBa_J48104 (SEQ ID NO: 216), BBa_J54200 (SEQ ID NO: 217), BBa_J56015 (SEQ ID NO: 218), BBa_J64951 (SEQ ID NO: 219), BBa_K088007 (SEQ ID NO: 220), BBa_K119000 (SEQ ID NO: 221), BBa_K119001 (SEQ ID NO: 222), BBa_K1330002 (SEQ ID NO: 223), BBa_K137029 (SEQ ID NO: 224), BBa_K137030 (SEQ ID NO: 225), BBa_K137031 (SEQ ID NO: 226), BBa_K137032 (SEQ ID NO: 227), BBa_K137085 (SEQ ID NO: 228), BBa_K137086 (SEQ ID NO: 229), BBa_K137087 (SEQ ID NO: 230), BBa_K137088 (SEQ ID NO: 231), BBa_K137089 (SEQ ID NO: 232), BBa_K137090 (SEQ ID NO: 233), BBa_K137091 (SEQ ID NO: 234), BBa_K1585100 (SEQ ID NO: 235), BBa_K1585101 (SEQ ID NO: 236), BBa_K1585102 (SEQ ID NO: 237), BBa_K1585103 (SEQ ID NO: 238), BBa_K1585104 (SEQ ID NO: 239), BBa_K1585105 (SEQ ID NO: 240), BBa_K1585106 (SEQ ID NO: 241), BBa_K1585110 (SEQ ID NO: 242), BBa_K1585113 (SEQ ID NO: 243), BBa_K1585115 (SEQ ID NO: 244), BBa_K1585116 (SEQ ID NO: 245), BBa_K1585117 (SEQ ID NO: 246), BBa_K1585118 (SEQ ID NO: 247), BBa_K1585119 (SEQ ID NO: 248), BBa_K1824896 (SEQ ID NO: 249), BBa_K256002 (SEQ ID NO: 250), BBa_K256018 (SEQ ID NO: 251), BBa_K256020 (SEQ ID NO: 252), BBa_K256033 (SEQ ID NO: 253), BBa_K292000 (SEQ ID NO: 254), BBa_K292001 (SEQ ID NO: 255), BBa_K418000 (SEQ ID NO: 256), BBa_K418002 (SEQ ID NO: 257), BBa_K418003 (SEQ ID NO: 258), BBa_K823004 (SEQ ID NO: 259), BBa_K823005 (SEQ ID NO: 260), BBa_K823006 (SEQ ID NO: 261), BBa_K823007 (SEQ ID NO: 262), BBa_K823008 (SEQ ID NO: 263), BBa_K823010 (SEQ ID NO: 264), BBa_K823011 (SEQ ID NO: 265), BBa_K823013 (SEQ ID NO: 266), BBa_K823014 (SEQ ID NO: 267), BBa_M13101 (SEQ ID NO: 268), BBa_M13102 (SEQ ID NO: 269), BBa_M13103 (SEQ ID NO: 270), BBa_M13104 (SEQ ID NO: 271), BBa_M13105 (SEQ ID NO: 272), BBa_M13106 (SEQ ID NO: 273), BBa_M13108 (SEQ ID NO: 274), BBa_M13110 (SEQ ID NO: 275), BBa_M31519 (SEQ ID NO: 276), BBa_R1074 (SEQ ID NO: 277), BBa_R1075 (SEQ ID NO: 278), BBa_503331 (SEQ ID NO: 279), BBa_I14018 (SEQ ID NO: 280), and BBa_I14033 (SEQ ID NO: 281). Exemplary constitutive promoters include E. coli σS promoters, e.g., BBa_J45992 (SEQ ID NO: 282), and BBa_J45993 (SEQ ID NO: 283). Exemplary constitutive promoters further include constitutive E. coli σ32 promoters, e.g, BBa_J45504 (SEQ ID NO: 284), BBa_K1895002 (SEQ ID NO: 285), and BBa_K1895003 (SEQ ID NO: 286). Exemplary constitutive promoters further include constitutive B. subtilis σA promoters, e.g., BBa_K780003 (SEQ ID NO: 287), BBa_K823000 (SEQ ID NO: 288), BBa_K823002 (SEQ ID NO: 289), and BBa_K823003 (SEQ ID NO: 290), BBa_K14301 (SEQ ID NO: 291), BBa_K143013 (SEQ ID NO: 292). Exemplary constitutive promoters further include constitutive B. subtilis σB promoters, e.g., BBa_K143010 (SEQ ID NO: 291), BBa_K143011 (SEQ ID NO: 292), BBa_K143013 (SEQ ID NO: 293). Exemplary constitutive promoters further include BBa_K112706 (SEQ ID NO: 294) and BBa_K112707 (SEQ ID NO: 295) promoters.

Exemplary promoters from Bacteriophage T7 or SP6 or various prokaryotes include BBa_K143010 (SEQ ID NO: 293), BBa_K143011 (SEQ ID NO: 294), BBa_K143013 (SEQ ID NO: 295), BBa_J712074 (SEQ ID NO: 296), BBa_J719005 (SEQ ID NO: 297), BBa_J34814 (SEQ ID NO: 298), BBa_J64997 (SEQ ID NO: 299), BBa_K113010 (SEQ ID NO: 300), BBa_K113011 (SEQ ID NO: 301), BBa_K113012 (SEQ ID NO: 302), BBa_K1614000 (SEQ ID NO: 303), BBa_R0085 (SEQ ID NO: 304), BBa_R0180 (SEQ ID NO: 305), BBa_R0181 (SEQ ID NO: 306), BBa_R0182 (SEQ ID NO: 307), BBa_R0183 (SEQ ID NO: 308), BBa_Z0251 (SEQ ID NO: 309), BBa_Z0252 (SEQ ID NO: 310), BBa_Z0253 (SEQ ID NO: 311), BBa_J64998 (SEQ ID NO: 312), BBa_K112706 (SEQ ID NO: 313), BBa_K112707 (SEQ ID NO: 314). Exemplary promoters from yeast and various eukaryotes include BBa_I766557 (SEQ ID NO: 315), BBa_J63005 (SEQ ID NO: 316), BBa_K105027 (SEQ ID NO: 317), BBa_K105028 (SEQ ID NO: 318), BBa_K105029 (SEQ ID NO: 319), BBa_K105030 (SEQ ID NO: 320), BBa_K105031 (SEQ ID NO: 321), BBa_K122000), SEQ ID NO: 322), BBa_K124000 (SEQ ID NO: 323), BBa_K124002 (SEQ ID NO: 324), BBa_K319005 (SEQ ID NO: 325), BBa_M31201 (SEQ ID NO: 326), BBa_J766555 (SEQ ID NO: 327), BBa_I766556 (SEQ ID NO: 328), BBa_J712004 (SEQ ID NO: 329), and BBa_K076017 (SEQ ID NO: 330).

Additional exemplary promoters are listed in Table 8.

TABLE 8
Exemplary constitutive Promoters

| Name | Description |
|---|---|
| Plpp | The Plpp promoter is a natural promoter taken from the Nissle genome. |
| SEQ ID NO: 331 | In situ it is used to drive production of lpp, which is known to be the |
| | most abundant protein in the cell. Also, in some previous RNAseq |
| | experiments I was able to confirm that the lpp mRNA is one of the |
| | most abundant mRNA in Nissle during exponential growth. |
| PapFAB46 | See, e.g., Kosuri, S., Goodman, D. B. & Cambray, G. Composability of |
| SEQ ID NO: 332 | regulatory sequences controlling transcription and translation in |
| | Escherichia coli. in 1-20 (2013). doi: 10.1073/pnas. |
| PJ23101 + UP | UP element helps recruit RNA polymerase |
| element | (ggaaaatttttttaaaaaaaaaac (SEQ ID NO: 1087)) |
| SEQ ID NO: 333 | |
| PJ23107 + UP | UP element helps recruit RNA polymerase |
| element | (ggaaaatttttttaaaaaaaaaac (SEQ ID NO: 1087)) |
| SEQ ID NO: 334 | |
| PSYN23119 | UP element at 5′ end; consensus -10 region is TATAAT; the consensus |
| SEQ ID NO: 335 | -35 is TTGACA; the extended -10 region is generally TGNTATAAT |
| | (TGGTATAAT in this sequence) |

Bacterial constitutive promoters are known in the art. In some embodiments, the constitutive promoter is at least about 880%, 85%, 90%, 95%, or 99% homologous to the sequence of any one of SEQ ID NOs: 187-343.

Ribosome Binding Sites

In some embodiments, ribosome binding sites are added, switched out or replaced. By testing a few ribosome binding sites, expression levels can be fine-tuned to the desired level. Various RBS are suitable for prokaryotic expression and can be used to achieve the desired expression levels (See, e.g., Registry of standard biological parts). Exemplary ribosome binding sites include those derived from Master sequence SEQ ID NO: 336. Non limiting examples of such ribosome binding sites include BBa_J61100, BBa_J61101, BBa_J61102, BBa_J61103, BBa_J61104, BBa_J61105, BBa_J61106, BBa_J61107, BBa_J61108, BBa_J61109, BBa_J61110, BBa_J61111, BBa_J61112, BBa_J61113, BBa_J61114, BBa_J61115, BBa_J61116, BBa_J61117, BBa_J61118, BBa_J61119, BBa_J61120, BBa_J61121, BBa_J61122, BBa_J61123, BBa_J61124, BBa_J61125, BBa_J61126, BBa_J61127, BBa_J61128, BBa_J6112, BBa_J61130, BBa_J61131, BBa_J61132, BBa_J61133, BBa_J61134, BBa_J61135, BBa_J61136, BBa_J61137, BBa_J61138, BBa_J61139, BBa_B0029, BBa_B0030, BBa_B0031, BBa_B0032, BBa_B0033, BBa_B0034, BBa_B0035, and BBa_B0064 (SEQ ID NO: 336-384).

### Nucleic Acids

In some embodiments, the disclosure provides novel nucleic acids for producing and secreting GLP-2. In some embodiments, the nucleic acid encodes one or more GLP-2 or GLP-2 fusion protein polypeptides. Thus, in some embodiments, the nucleic acid comprises gene sequence(s) encoding one or more GLP-2 or GLP-2 fusion protein polypeptides. In some embodiments, the one or more GLP-2 or GLP-2 fusion protein polypeptide(s) is selected from any of SEQ ID NOs: 499-514 and 537-546. In some embodiments, the nucleic acid comprises one or more GLP-2 or GLP-2 fusion protein cassettes. In some embodiments, the nucleic acid comprises gene sequence comprising one or more GLP-2 or GLP-2 fusion protein genes selected from SEQ ID NO: 515-536 and 551-562.

In some embodiments, the nucleic acid comprises gene sequence encoding a human GLP-2 polypeptide. In certain embodiments, the human GLP-2 polypeptide has at least about 80%, 85%, 90%, 95%, or 99% identity with SEQ ID NO: 148. In some embodiments, the human GLP-2 polypeptide has at least about 85%, 86%, 87%, 88%, 89%, 90%, 91%, 92%, 93%, 94%, 95%, 96%, 97%, 98%, or 99% identity with SEQ ID NO: 148. In some specific embodiments, the human GLP-2 polypeptide comprises SEQ ID NO: 148. In other specific embodiments, the human GLP-2 polypeptide consists of SEQ ID NO: 148. In some embodiments, the nucleic acid comprises a gene sequence. In certain embodiments, the nucleic acid comprising the human GLP-2 gene sequence has at least about 80%, 85%, 90%, 95%, or 99% identity with SEQ ID NO: 523. In some embodiments, the nucleic acid comprising the human GLP-2 gene sequence has at least about 85%, 86%, 87%, 88%, 89%, 90%, 91%, 92%, 93%, 94%, 95%, 96%, 97%, 98%, or 99% identity with SEQ ID NO: 523. In some specific embodiments, the nucleic acid comprising the human GLP-2 gene sequence comprises SEQ ID NO: 523. In other specific embodiments the nucleic acid comprising the human GLP-2 gene sequence consists of SEQ ID NO: 523.

In some embodiments, the nucleic acid comprises gene sequence encoding a Fc (IgA) polypeptide. In certain embodiments, the Fc (IgA) polypeptide has at least about 80%, 85%, 90%, 95%, or 99% identity with SEQ ID NO: 499. In some embodiments, the Fc (IgA) polypeptide has at least about 85%, 86%, 87%, 88%, 89%, 90%, 91%, 92%, 93%, 94%, 95%, 96%, 97%, 98%, or 99% identity with SEQ ID NO: 499. In some specific embodiments, the Fc (IgA) polypeptide comprises SEQ ID NO: 499. In other specific embodiments, the Fc (IgA) polypeptide consists of SEQ ID NO: 499. In some embodiments, the nucleic acid comprises a gene sequence. In certain embodiments, the nucleic acid comprising the Fc (IgA) gene sequence has at least about 80%, 85%, 90%, 95%, or 99% identity with SEQ ID NO: 527. In some embodiments, the nucleic acid comprising the Fc (IgA) gene sequence has at least about 85%, 86%, 87%, 88%, 89%, 90%, 91%, 92%, 93%, 94%, 95%, 96%, 97%, 98%, or 99% identity with SEQ ID NO: 527. In some specific embodiments, the nucleic acid comprising the Fc (IgA) gene sequence comprises SEQ ID NO: 527. In other specific embodiments the nucleic acid comprising the Fc (IgA) gene sequence consists of SEQ ID NO: 527.

In some embodiments, the nucleic acid comprises a gene sequence. In certain embodiments, the nucleic acid comprising the Fc (IgA) gene sequence has at least about 80%, 85%, 90%, 95%, or 99% identity with SEQ ID NO: 528. In some embodiments, the nucleic acid comprising the Fc (IgA) gene sequence has at least about 85%, 86%, 87%, 88%, 89%, 90%, 91%, 92%, 93%, 94%, 95%, 96%, 97%, 98%, or 99% identity with SEQ ID NO: 528. In some specific embodiments, the nucleic acid comprising the Fc (IgA) gene sequence comprises SEQ ID NO: 528. In other specific embodiments the nucleic acid comprising the Fc (IgA) gene sequence consists of SEQ ID NO: 528.

In some embodiments, the nucleic acid comprises gene sequence encoding a Linker polypeptide. In certain embodiments, the Linker polypeptide has at least about 80%, 85%, 90%, 95%, or 99% identity with SEQ ID NO: 509. In some embodiments, the Linker polypeptide has at least about 85%, 86%, 87%, 88%, 89%, 90%, 91%, 92%, 93%, 94%, 95%, 96%, 97%, 98%, or 99% identity with SEQ ID NO: 509. In some specific embodiments, the Linker polypeptide comprises SEQ ID NO: 509. In other specific embodiments, the Linker polypeptide consists of SEQ ID NO: 509. In some embodiments, the nucleic acid comprises a gene sequence. In certain embodiments, the nucleic acid comprising the LINKER gene sequence has at least about 80%, 85%, 90%, 95%, or 99% identity with SEQ ID NO: 524. In some embodiments, the nucleic acid comprising the Linker gene sequence has at least about 85%, 86%, 87%, 88%, 89%, 90%, 91%, 92%, 93%, 94%, 95%, 96%, 97%, 98%, or 99% identity with SEQ ID NO: 524. In some specific embodiments, the nucleic acid comprising the Linker gene sequence comprises SEQ ID NO: 524. In other specific embodiments the nucleic acid comprising the linker gene sequence consists of SEQ ID NO: 524.

In some embodiments, the nucleic acid comprises gene sequence encoding a glp2-linker Fc (IgA) polypeptide. In certain embodiments, the glp2-linker Fc (IgA) polypeptide has at least about 80%, 85%, 90%, 95%, or 99% identity with SEQ ID NO: 537. In some embodiments, the glp2-linker Fc (IgA) polypeptide has at least about 85%, 86%, 87%, 88%, 89%, 90%, 91%, 92%, 93%, 94%, 95%, 96%, 97%, 98%, or 99% identity with SEQ ID NO: 537. In some specific embodiments, the glp2-linker Fc (IgA) polypeptide comprises SEQ ID NO: 537. In other specific embodiments, the glp2-linker Fc (IgA) polypeptide consists of SEQ ID NO: 537. In some embodiments, the nucleic acid comprises a gene sequence. In certain embodiments, the nucleic acid comprising the glp2-linker Fc (IgA) gene sequence has at least about 80%, 85%, 90%, 95%, or 99% identity with SEQ ID NO: 547. In some embodiments, the nucleic acid comprising the glp2-linker Fc (IgA) gene sequence has at least about 85%, 86%, 87%, 88%, 89%, 90%, 91%, 92%, 93%, 94%, 95%, 96%, 97%, 98%, or 99% identity with SEQ ID NO: 547. In some specific embodiments, the nucleic acid comprising the glp2-linker Fc (IgA) gene sequence comprises SEQ ID NO: 547. In other specific embodiments the nucleic acid comprising the glp2-linker Fc (IgA) gene sequence consists of SEQ ID NO: 547. In certain embodiments, the nucleic acid comprising the glp2-linker Fc (IgA) gene sequence has at least about 80%, 85%, 90%, 95%, or 99% identity with SEQ ID NO: 548. In some embodiments, the nucleic acid comprising the glp2-linker Fc (IgA) gene sequence has at least about 85%, 86%, 87%, 88%, 89%, 90%, 91%, 92%, 93%, 94%, 95%, 96%, 97%, 98%, or 99% identity with SEQ ID NO: 548. In some specific embodiments, the nucleic acid comprising the glp2-linker Fc (IgA) gene sequence comprises SEQ ID NO: 548. In other specific embodiments the nucleic acid comprising the glp2-linker Fc (IgA) gene sequence consists of SEQ ID NO: 548.

In some embodiments, the nucleic acid comprises gene sequence encoding a PhoA-GLP-2 polypeptide. In certain embodiments, the PhoA-GLP-2 polypeptide has at least about 80%, 85%, 90%, 95%, or 99% identity with SEQ ID NO: 538. In some embodiments, the PhoA-GLP-2 polypeptide has at least about 85%, 86%, 87%, 88%, 89%, 90%, 91%, 92%, 93%, 94%, 95%, 96%, 97%, 98%, or 99% identity with SEQ ID NO: 538. In some specific embodiments, the PhoA-GLP-2 polypeptide comprises SEQ ID NO: 538. In other specific embodiments, the PhoA-GLP-2 polypeptide consists of SEQ ID NO: 538. In some embodiments, the nucleic acid comprises a gene sequence. In certain embodiments, the nucleic acid comprising the PhoA-GLP-2 gene sequence has at least about 80%, 85%, 90%, 95%, or 99% identity with SEQ ID NO: 549. In some embodiments, the nucleic acid comprising the PhoA-GLP-2 gene sequence has at least about 85%, 86%, 87%, 88%, 89%, 90%, 91%, 92%, 93%, 94%, 95%, 96%, 97%, 98%, or 99% identity with SEQ ID NO: 549.

In some embodiments, the nucleic acid comprises gene sequence encoding a PhoA-GLP-2-Fc (hIgA) polypeptide. In certain embodiments, the PhoA-GLP-2-Fc (hIgA) polypeptide has at least about 80%, 85%, 90%, 95%, or 99% identity with SEQ ID NO: 511. In some embodiments, the PhoA-GLP-2-Fc (hIgA) polypeptide has at least about 85%, 86%, 87%, 88%, 89%, 90%, 91%, 92%, 93%, 94%, 95%, 96%, 97%, 98%, or 99% identity with SEQ ID NO: 511. In some specific embodiments, the PhoA-GLP-2-Fc (hIgA) polypeptide comprises SEQ ID NO: 511. In other specific embodiments, the PhoA-GLP-2-Fc (hIgA) polypeptide consists of SEQ ID NO: 511. In some embodiments, the nucleic acid comprises gene sequence encoding a PhoA-GLP-2-Fc (hIgA) polypeptide. In certain embodiments, the PhoA-GLP-2-Fc (hIgA) polypeptide has at least about 80%, 85%, 90%, 95%, or 99% identity with SEQ ID NO: 512. In some embodiments, the PhoA-GLP-2-Fc (hIgA) polypeptide has at least about 85%, 86%, 87%, 88%, 89%, 90%, 91%, 92%, 93%, 94%, 95%, 96%, 97%, 98%, or 99% identity with SEQ ID NO: 512. In some specific embodiments, the PhoA-GLP-2-Fc (hIgA) polypeptide comprises SEQ ID NO: 512. In other specific embodiments, the PhoA-GLP-2-Fc (hIgA) polypeptide consists of SEQ ID NO: 512. In some embodiments, the nucleic acid comprises a gene sequence. In certain embodiments, the nucleic acid comprising the PhoA-GLP-2-Fc (hIgA) gene sequence has at least about 80%, 85%, 90%, 95%, or 99% identity with SEQ ID NO: 528. In some embodiments, the nucleic acid comprising the PhoA-GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 528. In some specific embodiments, the nucleic acid comprising the PhoA-GLP-2-Fc (hIgA) gene sequence comprises SEQ ID NO: 528. In other specific embodiments the nucleic acid comprising the PhoA-GLP-2-Fc (hIgA) gene sequence consists of SEQ ID NO: 528.

In certain embodiments, the nucleic acid comprising the PhoA-GLP-2-Fc (hIgA) gene sequence has at least about 80%, 85%, 90%, 95%, or 99% identity with SEQ ID NO: 529. In some embodiments, the nucleic acid comprising the PhoA-GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 529. In some specific embodiments, the nucleic acid comprising the PhoA-GLP-2-Fc (hIgA) gene sequence comprises SEQ ID NO: 529. In other specific embodiments the nucleic acid comprising the PhoA-GLP-2-Fc (hIgA) gene sequence consists of SEQ ID NO: 529.

In some embodiments, the nucleic acid comprises a gene sequence. In certain embodiments, the nucleic acid comprising the PhoA-GLP-2-Fc (hIgA) gene sequence has at least about 80%, 85%, 90%, 95%, or 99% identity with SEQ ID NO: 530. In some embodiments, the nucleic acid comprising the PhoA-GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 530. In some specific embodiments, the nucleic acid comprising the PhoA-GLP-2-Fc (hIgA) gene sequence comprises SEQ ID NO: 530. In other specific embodiments the nucleic acid comprising the PhoA-GLP-2-Fc (hIgA) gene sequence consists of SEQ ID NO: 530.

In some embodiments, the nucleic acid comprises a gene sequence. In certain embodiments, the nucleic acid comprising the PhoA-GLP-2-Fc (hIgA) gene sequence has at least about 80%, 85%, 90%, 95%, or 99% identity with SEQ ID NO: 531. In some embodiments, the nucleic acid comprising the PhoA-GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 531. In some specific embodiments, the nucleic acid comprising the PhoA-GLP-2-Fc (hIgA) gene sequence comprises SEQ ID NO: 531. In other specific embodiments the nucleic acid comprising the PhoA-GLP-2-Fc (hIgA) gene sequence consists of SEQ ID NO: 531.

In some embodiments, the nucleic acid comprises gene sequence encoding a ECOLIN 19410-GLP-2 polypeptide. In certain embodiments, the ECOLIN 19410-GLP-2 polypeptide has at least about 80%, 85%, 90%, 95%, or 99% identity with SEQ ID NO: 539. In some embodiments, the ECOLIN 19410-GLP-2 polypeptide has at least about 85%, 86%, 87%, 88%, 89%, 90%, 91%, 92%, 93%, 94%, 95%, 96%, 97%, 98%, or 99% identity with SEQ ID NO: 539. In some specific embodiments, the ECOLIN 19410-GLP-2 polypeptide comprises SEQ ID NO: 539. In other specific embodiments, the ECOLIN 19410-GLP-2 polypeptide consists of SEQ ID NO: 539. In some embodiments, the nucleic acid comprises a gene sequence. In certain embodiments, the nucleic acid comprising the ECOLIN 19410-GLP-2 gene sequence has at least about 80%, 85%, 90%, 95%, or 99% identity with SEQ ID NO: 550. In some embodiments, the nucleic acid comprising the ECOLIN 19410-GLP-2 gene sequence has at least about 85%, 86%, 87%, 88%, 89%, 90%, 91%, 92%, 93%, 94%, 95%, 96%, 97%, 98%, or 99% identity with SEQ ID NO: 550. In some specific embodiments, the nucleic acid comprising the ECOLIN 19410-GLP-2 gene sequence comprises SEQ ID NO: 550. In other specific embodiments the nucleic acid comprising the ECOLIN 19410-GLP-2 gene sequence consists of SEQ ID NO: 550.

In some embodiments, the nucleic acid comprises gene sequence encoding a ECOLIN 19410-GLP-2-Fc (hIgA) polypeptide. In certain embodiments, the ECOLIN 19410-GLP-2-Fc (hIgA) polypeptide has at least about 80%, 85%, 90%, 95%, or 99% identity with SEQ ID NO: 513. In some embodiments, the ECOLIN 19410-GLP-2-Fc (hIgA) polypeptide has at least about 85%, 86%, 87%, 88%, 89%, 90%, 91%, 92%, 93%, 94%, 95%, 96%, 97%, 98%, or 99% identity with SEQ ID NO: 513. In some specific embodiments, the ECOLIN 19410-GLP-2-Fc (hIgA) polypeptide comprises SEQ ID NO: 513. In other specific embodiments, the ECOLIN 19410-GLP-2-Fc (hIgA) polypeptide consists of SEQ ID NO: 513. In some embodiments, the nucleic acid comprises gene sequence encoding a ECOLIN 19410-GLP-2-Fc (hIgA) polypeptide. In certain embodiments, the ECOLIN 19410-GLP-2-Fc (hIgA) polypeptide has at least about 80%, 85%, 90%, 95%, or 99% identity with SEQ ID NO: 514. In some embodiments, the ECOLIN 19410-GLP-2-Fc (hIgA) polypeptide has at least about 85%, 86%, 87%, 88%, 89%, 90%, 91%, 92%, 93%, 94%, 95%, 96%, 97%, 98%, or 99% identity with SEQ ID NO: 514. In some specific embodiments, the ECOLIN 19410-GLP-2-Fc (hIgA) polypeptide comprises SEQ ID NO: 514. In other specific embodiments, the ECOLIN 19410-GLP-2-Fc (hIgA) polypeptide consists of SEQ ID NO: 514.

In some embodiments, the nucleic acid comprises a gene sequence. In certain embodiments, the nucleic acid comprising the ECOLIN 19410-GLP-2-Fc (hIgA) gene sequence has at least about 80%, 85%, 90%, 95%, or 99% identity with SEQ ID NO: 533. In some embodiments, the nucleic acid comprising the ECOLIN 19410-GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 533. In some specific embodiments, the nucleic acid comprising the ECOLIN 19410-GLP-2-Fc (hIgA) gene sequence comprises SEQ ID NO: 533. In other specific embodiments the nucleic acid comprising the ECOLIN 19410-GLP-2-Fc (hIgA) gene sequence consists of SEQ ID NO: 533.

In some embodiments, the nucleic acid comprises a gene sequence. In certain embodiments, the nucleic acid comprising the ECOLIN 19410-GLP-2-Fc (hIgA) gene sequence has at least about 80%, 85%, 90%, 95%, or 99% identity with SEQ ID NO: 534. In some embodiments, the nucleic acid comprising the ECOLIN 19410-GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 534. In some specific embodiments, the nucleic acid comprising the ECOLIN 19410-GLP-2-Fc (hIgA) gene sequence comprises SEQ ID NO: 534. In other specific embodiments the nucleic acid comprising the ECOLIN 19410-GLP-2-Fc (hIgA) gene sequence consists of SEQ ID NO: 534.

In some embodiments, the nucleic acid comprises a gene sequence. In certain embodiments, the nucleic acid comprising the ECOLIN 19410-GLP-2-Fc (hIgA) gene sequence has at least about 80%, 85%, 90%, 95%, or 99% identity with SEQ ID NO: 535. In some embodiments, the nucleic acid comprising the ECOLIN 19410-GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 535. In some specific embodiments, the nucleic acid comprising the ECOLIN 19410-GLP-2-Fc (hIgA) gene sequence comprises SEQ ID NO: 535. In other specific embodiments the nucleic acid comprising the ECOLIN 19410-GLP-2-Fc (hIgA) gene sequence consists of SEQ ID NO: 535.

In some embodiments, the nucleic acid comprises a gene sequence. In certain embodiments, the nucleic acid comprising the ECOLIN 19410-GLP-2-Fc (hIgA) gene sequence has at least about 80%, 85%, 90%, 95%, or 99% identity with SEQ ID NO: 536. In some embodiments, the nucleic acid comprising the ECOLIN 19410-GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 536. In some specific embodiments, the nucleic acid comprising the ECOLIN 19410-GLP-2-Fc (hIgA) gene sequence comprises SEQ ID NO: 536. In other specific embodiments the nucleic acid comprising the ECOLIN 19410-GLP-2-Fc (hIgA) gene sequence consists of SEQ ID NO: 536.

In some embodiments, the nucleic acid comprises gene sequence encoding a mutated GLP-2 polypeptide. In certain embodiments, the mutated GLP-2 polypeptide has at least about 80%, 85%, 90%, 95%, or 99% identity with SEQ ID NO: 149. In some embodiments, the mutated GLP-2 polypeptide has at least about 85%, 86%, 87%, 88%, 89%, 90%, 91%, 92%, 93%, 94%, 95%, 96%, 97%, 98%, or 99% identity with SEQ ID NO: 149. In some specific embodiments, the mutated GLP-2 polypeptide comprises SEQ ID NO: 149. In other specific embodiments, the mutated GLP-2 polypeptide consists of SEQ ID NO: 149. In some embodiments, the nucleic acid comprises a gene sequence. In certain embodiments, the nucleic acid comprising the mutated GLP-2 gene sequence has at least about 80%, 85%, 90%, 95%, or 99% identity with SEQ ID NO: 526. In some embodiments, the nucleic acid comprising the mutated GLP-2 gene sequence has at least about 85%, 86%, 87%, 88%, 89%, 90%, 91%, 92%, 93%, 94%, 95%, 96%, 97%, 98%, or 99% identity with SEQ ID NO: 526. In some specific embodiments, the nucleic acid comprising the mutated GLP-2 gene sequence comprises SEQ ID NO: 526. In other specific embodiments the nucleic acid comprising the mutated GLP-2 gene sequence consists of SEQ ID NO: 526.

In some embodiments, the nucleic acid comprises gene sequence encoding a mutated glp2-linker Fc (IgA) polypeptide. In certain embodiments, the mutated glp2-linker Fc (IgA) polypeptide has at least about 80%, 85%, 90%, 95%, or 99% identity with SEQ ID NO: 540. In some embodiments, the mutated glp2-linker Fc (IgA) polypeptide has at least about 85%, 86%, 87%, 88%, 89%, 90%, 91%, 92%, 93%, 94%, 95%, 96%, 97%, 98%, or 99% identity with SEQ ID NO: 540. In some specific embodiments, the mutated glp2-linker Fc (IgA) polypeptide comprises SEQ ID NO: 540. In other specific embodiments, the mutated glp2-linker Fc (IgA) polypeptide consists of SEQ ID NO: 540. In some embodiments, the nucleic acid comprises a gene sequence. In certain embodiments, the nucleic acid comprising the mutated glp2-linker Fc (IgA) gene sequence has at least about 80%, 85%, 90%, 95%, or 99% identity with SEQ ID NO: 559. In some embodiments, the nucleic acid comprising the mutated glp2-linker Fc (IgA) gene sequence has at least about 85%, 86%, 87%, 88%, 89%, 90%, 91%, 92%, 93%, 94%, 95%, 96%, 97%, 98%, or 99% identity with SEQ ID NO: 559. In some specific embodiments, the nucleic acid comprising the mutated glp2-linker Fc (IgA) gene sequence comprises SEQ ID NO: 559. In other specific embodiments the nucleic acid comprising the mutated glp2-linker Fc (IgA) gene sequence consists of SEQ ID NO: 559. In certain embodiments, the nucleic acid comprising the mutated glp2-linker Fc (IgA) gene sequence has at least about 80%, 85%, 90%, 95%, or 99% identity with SEQ ID NO: 560. In some embodiments, the nucleic acid comprising the mutated glp2-linker Fc (IgA) gene sequence has at least about 85%, 86%, 87%, 88%, 89%, 90%, 91%, 92%, 93%, 94%, 95%, 96%, 97%, 98%, or 99% identity with SEQ ID NO: 560. In some specific embodiments, the nucleic acid comprising the mutated glp2-linker Fc (IgA) gene sequence comprises SEQ ID NO: 560. In other specific embodiments the nucleic acid comprising the mutated glp2-linker Fc (IgA) gene sequence consists of SEQ ID NO: 560.

In some embodiments, the nucleic acid comprises gene sequence encoding a PhoA-mutated GLP-2 polypeptide. In certain embodiments, the PhoA-mutated GLP-2 polypeptide has at least about 80%, 85%, 90%, 95%, or 99% identity with SEQ ID NO: 541. In some embodiments, the PhoA-mutated GLP-2 polypeptide has at least about 85%, 86%, 87%, 88%, 89%, 90%, 91%, 92%, 93%, 94%, 95%, 96%, 97%, 98%, or 99% identity with SEQ ID NO: 541. In some specific embodiments, the PhoA-mutated GLP-2 polypeptide comprises SEQ ID NO: 541. In other specific embodiments, the PhoA-mutated GLP-2 polypeptide consists of SEQ ID NO: 541. In some embodiments, the nucleic acid comprises a gene sequence. In certain embodiments, the nucleic acid comprising the PhoA-mutated GLP-2 gene sequence has at least about 80%, 85%, 90%, 95%, or 99% identity with SEQ ID NO: 561. In some embodiments, the nucleic acid comprising the PhoA-mutated GLP-2 gene sequence has at least about 85%, 86%, 87%, 88%, 89%, 90%, 91%, 92%, 93%, 94%, 95%, 96%, 97%, 98%, or 99% identity with SEQ ID NO: 561. In some specific embodiments, the nucleic acid comprising the PhoA-mutated GLP-2 gene sequence comprises SEQ ID NO: 561. In other specific embodiments the nucleic acid comprising the PhoA-mutated GLP-2 gene sequence consists of SEQ ID NO: 561.

In some embodiments, the nucleic acid comprises gene sequence encoding a PhoA-mutated GLP-2-Fc (hIgA) polypeptide. In certain embodiments, the PhoA-mutated GLP-2-Fc (hIgA) polypeptide has at least about 80%, 85%, 90%, 95%, or 99% identity with SEQ ID NO: 543. In some embodiments, the PhoA-mutated GLP-2-Fc (hIgA) polypeptide has at least about 85%, 86%, 87%, 88%, 89%, 90%, 91%, 92%, 93%, 94%, 95%, 96%, 97%, 98%, or 99% identity with SEQ ID NO: 543. In some specific embodiments, the PhoA-mutated GLP-2-Fc (hIgA) polypeptide comprises SEQ ID NO: 543. In other specific embodiments, the PhoA-mutated GLP-2-Fc (hIgA) polypeptide consists of SEQ ID NO: 543. In some embodiments, the nucleic acid comprises gene sequence encoding a PhoA-mutated GLP-2-Fc (hIgA) polypeptide. In certain embodiments, the PhoA-mutated GLP-2-Fc (hIgA) polypeptide has at least about 80%, 85%, 90%, 95%, or 99% identity with SEQ ID NO: 544. In some embodiments, the PhoA-mutated GLP-2-Fc (hIgA) polypeptide has at least about 85%, 86%, 87%, 88%, 89%, 90%, 91%, 92%, 93%, 94%, 95%, 96%, 97%, 98%, or 99% identity with SEQ ID NO: 544. In some specific embodiments, the PhoA-mutated GLP-2-Fc (hIgA) polypeptide comprises SEQ ID NO: 544. In other specific embodiments, the PhoA-mutated GLP-2-Fc (hIgA) polypeptide consists of SEQ ID NO: 544. In some embodiments, the nucleic acid comprises a gene sequence. In certain embodiments, the nucleic acid comprising the PhoA-mutated GLP-2-Fc (hIgA) gene sequence has at least about 80%, 85%, 90%, 95%, or 99% identity with SEQ ID NO: 551. In some embodiments, the nucleic acid comprising the PhoA-mutated GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 551. In some specific embodiments, the nucleic acid comprising the PhoA-mutated GLP-2-Fc (hIgA) gene sequence comprises SEQ ID NO: 551. In other specific embodiments the nucleic acid comprising the PhoA-mutated GLP-2-Fc (hIgA) gene sequence consists of SEQ ID NO: 551. In some embodiments, the nucleic acid comprises a gene sequence. In certain embodiments, the nucleic acid comprising the PhoA-mutated GLP-2-Fc (hIgA) gene sequence has at least about 80%, 85%, 90%, 95%, or 99% identity with SEQ ID NO: 552. In some embodiments, the nucleic acid comprising the PhoA-mutated GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 552. In some specific embodiments, the nucleic acid comprising the PhoA-mutated GLP-2-Fc (hIgA) gene sequence comprises SEQ ID NO: 552. In other specific embodiments the nucleic acid comprising the PhoA-mutated GLP-2-Fc (hIgA) gene sequence consists of SEQ ID NO: 552. In some embodiments, the nucleic acid comprises a gene sequence. In certain embodiments, the nucleic acid comprising the PhoA-mutated GLP-2-Fc (hIgA) gene sequence has at least about 80%, 85%, 90%, 95%, or 99% identity with SEQ ID NO: 553. In some embodiments, the nucleic acid comprising the PhoA-mutated GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 553. In some specific embodiments, the nucleic acid comprising the PhoA-mutated GLP-2-Fc (hIgA) gene sequence comprises SEQ ID NO: 553. In other specific embodiments the nucleic acid comprising the PhoA-mutated GLP-2-Fc (hIgA) gene sequence consists of SEQ ID NO: 553. In some embodiments, the nucleic acid comprises a gene sequence. In certain embodiments, the nucleic acid comprising the PhoA-mutated GLP-2-Fc (hIgA) gene sequence has at least about 80%, 85%, 90%, 95%, or 99% identity with SEQ ID NO: 554. In some embodiments, the nucleic acid comprising the PhoA-mutated GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 554. In some specific embodiments, the nucleic acid comprising the PhoA-mutated GLP-2-Fc (hIgA) gene sequence comprises SEQ ID NO: 554. In other specific embodiments the nucleic acid comprising the PhoA-mutated GLP-2-Fc (hIgA) gene sequence consists of SEQ ID NO: 554.

In some embodiments, the nucleic acid comprises gene sequence encoding a the ECOLIN 19410-mutated GLP-2 polypeptide. In certain embodiments, the ECOLIN 19410-mutated GLP-2 polypeptide has at least about 80%, 85%, 90%, 95%, or 99% identity with SEQ ID NO: 542. In some embodiments, the ECOLIN 19410-mutated GLP-2 polypeptide has at least about 85%, 86%, 87%, 88%, 89%, 90%, 91%, 92%, 93%, 94%, 95%, 96%, 97%, 98%, or 99% identity with SEQ ID NO: 542. In some specific embodiments, the ECOLIN 19410-mutated GLP-2 polypeptide comprises SEQ ID NO: 542. In other specific embodiments, the ECOLIN 19410-mutated GLP-2 polypeptide consists of SEQ ID NO: 542. In some embodiments, the nucleic acid comprises a gene sequence. In certain embodiments, the nucleic acid comprising the ECOLIN 19410-mutated GLP-2 gene sequence has at least about 80%, 85%, 90%, 95%, or 99% identity with SEQ ID NO: 562. In some embodiments, the nucleic acid comprising the ECOLIN 19410-mutated GLP-2 gene sequence has at least about 85%, 86%, 87%, 88%, 89%, 90%, 91%, 92%, 93%, 94%, 95%, 96%, 97%, 98%, or 99% identity with SEQ ID NO: 562. In some specific embodiments, the nucleic acid comprising the ECOLIN 19410-mutated GLP-2 gene sequence comprises SEQ ID NO: 562. In other specific embodiments the nucleic acid comprising the ECOLIN 19410-mutated GLP-2 gene sequence consists of SEQ ID NO: 562.

In some embodiments, the nucleic acid comprises gene sequence encoding a ECOLIN 19419-mutated GLP-2-Fc (hIgA) polypeptide. In certain embodiments, the ECOLIN 19419-mutated GLP-2-Fc (hIgA) polypeptide has at least about 80%, 85%, 90%, 95%, or 99% identity with SEQ ID NO: 545. In some embodiments, the ECOLIN 19419-mutated GLP-2-Fc (hIgA) polypeptide has at least about 85%, 86%, 87%, 88%, 89%, 90%, 91%, 92%, 93%, 94%, 95%, 96%, 97%, 98%, or 99% identity with SEQ ID NO: 545. In some specific embodiments, the ECOLIN 19419-mutated GLP-2-Fc (hIgA) polypeptide comprises SEQ ID NO: 545. In other specific embodiments, the ECOLIN 19419-mutated GLP-2-Fc (hIgA) polypeptide consists of SEQ ID NO: 545.

In some embodiments, the nucleic acid comprises gene sequence encoding a ECOLIN 19419-mutated GLP-2-Fc (hIgA) polypeptide. In certain embodiments, the ECOLIN 19419-mutated GLP-2-Fc (hIgA) polypeptide has at least about 80%, 85%, 90%, 95%, or 99% identity with SEQ ID NO: 546. In some embodiments, the ECOLIN 19419-mutated GLP-2-Fc (hIgA) polypeptide has at least about 85%, 86%, 87%, 88%, 89%, 90%, 91%, 92%, 93%, 94%, 95%, 96%, 97%, 98%, or 99% identity with SEQ ID NO: 546. In some specific embodiments, the ECOLIN 19419-mutated GLP-2-Fc (hIgA) polypeptide comprises SEQ ID NO: 546. In other specific embodiments, the ECOLIN 19419-mutated GLP-2-Fc (hIgA) polypeptide consists of SEQ ID NO: 546.

In some embodiments, the nucleic acid comprises a gene sequence. In certain embodiments, the nucleic acid comprising the ECOLIN 19419-mutated GLP-2-Fc (hIgA) gene sequence has at least about 80%, 85%, 90%, 95%, or 99% identity with SEQ ID NO: 555. In some embodiments, the nucleic acid comprising the ECOLIN 19419-mutated GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 555. In some specific embodiments, the nucleic acid comprising the ECOLIN 19419-mutated GLP-2-Fc (hIgA) gene sequence comprises SEQ ID NO: 555. In other specific embodiments the nucleic acid comprising the ECOLIN 19419-mutated GLP-2-Fc (hIgA) gene sequence consists of SEQ ID NO: 555.

In some embodiments, the nucleic acid comprises a gene sequence. In certain embodiments, the nucleic acid comprising the ECOLIN 19419-mutated GLP-2-Fc (hIgA) gene sequence has at least about 80%, 85%, 90%, 95%, or 99% identity with SEQ ID NO: 556. In some embodiments, the nucleic acid comprising the ECOLIN 19419-mutated GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 556. In some specific embodiments, the nucleic acid comprising the ECOLIN 19419-mutated GLP-2-Fc (hIgA) gene sequence comprises SEQ ID NO: 556. In other specific embodiments the nucleic acid comprising the ECOLIN 19419-mutated GLP-2-Fc (hIgA) gene sequence consists of SEQ ID NO: 556.

In some embodiments, the nucleic acid comprises a gene sequence. In certain embodiments, the nucleic acid comprising the ECOLIN 19419-mutated GLP-2-Fc (hIgA) gene sequence has at least about 80%, 85%, 90%, 95%, or 99% identity with SEQ ID NO: 557. In some embodiments, the nucleic acid comprising the ECOLIN 19419-mutated GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 557. In some specific embodiments, the nucleic acid comprising the ECOLIN 19419-mutated GLP-2-Fc (hIgA) gene sequence comprises SEQ ID NO: 557. In other specific embodiments the nucleic acid comprising the ECOLIN 19419-mutated GLP-2-Fc (hIgA) gene sequence consists of SEQ ID NO: 557.

In some embodiments, the nucleic acid comprises a gene sequence. In certain embodiments, the nucleic acid comprising the ECOLIN 19419-mutated GLP-2-Fc (hIgA) gene sequence has at least about 80%, 85%, 90%, 95%, or 99% identity with SEQ ID NO: 558. In some embodiments, the nucleic acid comprising the ECOLIN 19419-mutated GLP-2-Fc (hIgA) gene sequence has at least about 85%, 86%, 87%, 88%, 89%, 90%, 91%, 92%, 93%, 94%, 95%, 96%, 97%, 98%, or 99% identity with SEQ ID NO: 558. In some specific embodiments, the nucleic acid comprising the ECOLIN 19419-mutated GLP-2-Fc (hIgA) gene sequence comprises SEQ ID NO: 558. In other specific embodiments the nucleic acid comprising the ECOLIN 19419-mutated GLP-2-Fc (hIgA) gene sequence consists of SEQ ID NO: 558.

In some embodiments, the disclosure provides novel nucleic acids for producing and secreting IL-10. In some embodiments, the nucleic acid encodes one or more IL-10 or IL-10 fusion protein polypeptides. Thus, in some embodiments, the nucleic acid comprises gene sequence(s) encoding one or more IL-10 or IL-10 fusion protein polypeptides. In some embodiments, the one or more IL-10 or IL-10 fusion protein polypeptide(s) is selected from any of SEQ ID NOs: 495-498 and 563-564. In some embodiments, the nucleic acid comprises one or more IL-10 or IL-10 fusion protein cassettes. In some embodiments, the nucleic acid comprises gene sequence comprising one or more IL-10 or IL-10 fusion protein genes selected from SEQ ID NO: 488-494 and 565-566.

In some embodiments, the nucleic acid comprises gene sequence encoding a IL-10 monomer polypeptide. In certain embodiments, the IL-10 monomer polypeptide has at least about 80%, 85%, 90%, 95%, or 99% identity with SEQ ID NO: 497. In some embodiments, the IL-10 monomer polypeptide has at least about 85%, 86%, 87%, 88%, 89%, 90%, 91%, 92%, 93%, 94%, 95%, 96%, 97%, 98%, or 99% identity with SEQ ID NO: 497. In some specific embodiments, the IL-10 monomer polypeptide comprises SEQ ID NO: 497. In other specific embodiments, the IL-10 monomer polypeptide consists of SEQ ID NO: 497. In some embodiments, the nucleic acid comprises a gene sequence. In certain embodiments, the nucleic acid comprising the IL-10 monomer gene sequence has at least about 80%, 85%, 90%, 95%, or 99% identity with SEQ ID NO: 493. In some embodiments, the nucleic acid comprising the IL-10 monomer gene sequence has at least about 85%, 86%, 87%, 88%, 89%, 90%, 91%, 92%, 93%, 94%, 95%, 96%, 97%, 98%, or 99% identity with SEQ ID NO: 493. In some specific embodiments, the nucleic acid comprising the IL-10 monomer gene sequence comprises SEQ ID NO: 493. In other specific embodiments the nucleic acid comprising the IL-10 monomer gene sequence consists of SEQ ID NO: 493.

In some embodiments, the nucleic acid comprises gene sequence encoding a IL-10-linker-IL-10 polypeptide. In certain embodiments, the IL-10-linker-IL-10 polypeptide has at least about 80%, 85%, 90%, 95%, or 99% identity with SEQ ID NO: 143. In some embodiments, the IL-10-linker-IL-10 polypeptide has at least about 85%, 86%, 87%, 88%, 89%, 90%, 91%, 92%, 93%, 94%, 95%, 96%, 97%, 98%, or 99% identity with SEQ ID NO: 143. In some specific embodiments, the IL-10-linker-IL-10 polypeptide comprises SEQ ID NO: 143. In other specific embodiments, the IL-10-linker-IL-10 polypeptide consists of SEQ ID NO: 143. In some embodiments, the nucleic acid comprises a gene sequence. In certain embodiments, the nucleic acid comprising the IL-10-linker-IL-10 gene sequence has at least about 80%, 85%, 90%, 95%, or 99% identity with SEQ ID NO: 492. In some embodiments, the nucleic acid comprising the IL-10-linker-IL-10 gene sequence has at least about 85%, 86%, 87%, 88%, 89%, 90%, 91%, 92%, 93%, 94%, 95%, 96%, 97%, 98%, or 99% identity with SEQ ID NO: 492. In some specific embodiments, the nucleic acid comprising the IL-10-linker-IL-10 gene sequence comprises SEQ ID NO: 492. In other specific embodiments the nucleic acid comprising the IL-10-linker-IL-10 gene sequence consists of SEQ ID NO: 492.

In some embodiments, the nucleic acid comprises gene sequence encoding a Linker polypeptide. In certain embodiments, the Linker polypeptide has at least about 80%, 85%, 90%, 95%, or 99% identity with SEQ ID NO: 498. In some embodiments, the Linker polypeptide has at least about 85%, 86%, 87%, 88%, 89%, 90%, 91%, 92%, 93%, 94%, 95%, 96%, 97%, 98%, or 99% identity with SEQ ID NO: 498. In some specific embodiments, the Linker polypeptide comprises SEQ ID NO: 498. In other specific embodiments, the Linker polypeptide consists of SEQ ID NO: 498. In some embodiments, the nucleic acid comprises a gene sequence. In certain embodiments, the nucleic acid comprising the Linker gene sequence has at least about 80%, 85%, 90%, 95%, or 99% identity with SEQ ID NO: 494. In some embodiments, the nucleic acid comprising the Linker gene sequence has at least about 85%, 86%, 87%, 88%, 89%, 90%, 91%, 92%, 93%, 94%, 95%, 96%, 97%, 98%, or 99% identity with SEQ ID NO: 494. In some specific embodiments, the nucleic acid comprising the Linker gene sequence comprises SEQ ID NO: 494. In other specific embodiments the nucleic acid comprising the Linker gene sequence consists of SEQ ID NO: 494.

In some embodiments, the nucleic acid comprises gene sequence encoding a PhoA-IL-10 polypeptide. In certain embodiments, the PhoA-IL-10 polypeptide has at least about 80%, 85%, 90%, 95%, or 99% identity with SEQ ID NO: 563. In some embodiments, the PhoA-IL-10 polypeptide has at least about 85%, 86%, 87%, 88%, 89%, 90%, 91%, 92%, 93%, 94%, 95%, 96%, 97%, 98%, or 99% identity with SEQ ID NO: 563. In some specific embodiments, the PhoA-IL-10 polypeptide comprises SEQ ID NO: 563. In other specific embodiments, the PhoA-IL-10 polypeptide consists of SEQ ID NO: 563. In some embodiments, the nucleic acid comprises a gene sequence. In certain embodiments, the nucleic acid comprising the PhoA-IL-10 gene sequence has at least about 80%, 85%, 90%, 95%, or 99% identity with SEQ ID NO: 566. In some embodiments, the nucleic acid comprising the PhoA-IL-10 gene sequence has at least about 85%, 86%, 87%, 88%, 89%, 90%, 91%, 92%, 93%, 94%, 95%, 96%, 97%, 98%, or 99% identity with SEQ ID NO: 566. In some specific embodiments, the nucleic acid comprising the PhoA-IL-10 gene sequence comprises SEQ ID NO: 566. In other specific embodiments the nucleic acid comprising the PhoA-IL-10 gene sequence consists of SEQ ID NO: 566.

In some embodiments, the nucleic acid comprises gene sequence encoding a PhoA-IL-10-linker-IL-10 polypeptide. In certain embodiments, the PhoA-IL-10-linker-IL-10 polypeptide has at least about 80%, 85%, 90%, 95%, or 99% identity with SEQ ID NO: 496. In some embodiments, the PhoA-IL-10-linker-IL-10 polypeptide has at least about 85%, 86%, 87%, 88%, 89%, 90%, 91%, 92%, 93%, 94%, 95%, 96%, 97%, 98%, or 99% identity with SEQ ID NO: 496. In some specific embodiments, the PhoA-IL-10-linker-IL-10 polypeptide comprises SEQ ID NO: 496. In other specific embodiments, the PhoA-IL-10-linker-IL-10 polypeptide consists of SEQ ID NO: 496. In some embodiments, the nucleic acid comprises a gene sequence. In certain embodiments, the nucleic acid comprising the PhoA-IL-10-linker-IL-10 gene sequence has at least about 80%, 85%, 90%, 95%, or 99% identity with SEQ ID NO: 489. In some embodiments, the nucleic acid comprising the PhoA-IL-10-linker-IL-10 gene sequence has at least about 85%, 86%, 87%, 88%, 89%, 90%, 91%, 92%, 93%, 94%, 95%, 96%, 97%, 98%, or 99% identity with SEQ ID NO: 489. In some specific embodiments, the nucleic acid comprising the PhoA-IL-10-linker-IL-10 gene sequence comprises SEQ ID NO: 489. In other specific embodiments the nucleic acid comprising the PhoA-IL-10-linker-IL-10 gene sequence consists of SEQ ID NO: 489.

In some embodiments, the nucleic acid comprises gene sequence encoding a ECOLIN 19410-IL-10 polypeptide. In certain embodiments, the ECOLIN 19410-IL-10 polypeptide has at least about 80%, 85%, 90%, 95%, or 99% identity with SEQ ID NO: 564. In some embodiments, the ECOLIN 19410-IL-10 polypeptide has at least about 85%, 86%, 87%, 88%, 89%, 90%, 91%, 92%, 93%, 94%, 95%, 96%, 97%, 98%, or 99% identity with SEQ ID NO: 564. In some specific embodiments, the ECOLIN 19410-IL-10 polypeptide comprises SEQ ID NO: 564. In other specific embodiments, the ECOLIN 19410-IL-10 polypeptide consists of SEQ ID NO: 564. In some embodiments, the nucleic acid comprises a gene sequence. In certain embodiments, the nucleic acid comprising the ECOLIN 19410-IL-10 gene sequence has at least about 80%, 85%, 90%, 95%, or 99% identity with SEQ ID NO: 566. In some embodiments, the nucleic acid comprising the ECOLIN 19410-IL-10 gene sequence has at least about 85%, 86%, 87%, 88%, 89%, 90%, 91%, 92%, 93%, 94%, 95%, 96%, 97%, 98%, or 99% identity with SEQ ID NO: 566. In some specific embodiments, the nucleic acid comprising the ECOLIN 19410-IL-10 gene sequence comprises SEQ ID NO: 566. In other specific embodiments the nucleic acid comprising the ECOLIN 19410-IL-10 gene sequence consists of SEQ ID NO: 566.

In some embodiments, the nucleic acid comprises gene sequence encoding a ECOLIN 19410-IL-10-linker-IL-10 polypeptide. In certain embodiments, the ECOLIN 19410-IL-10-linker-IL-10 polypeptide has at least about 80%, 85%, 90%, 95%, or 99% identity with SEQ ID NO: 495. In some embodiments, the ECOLIN 19410-IL-10-linker-IL-10 polypeptide has at least about 85%, 86%, 87%, 88%, 89%, 90%, 91%, 92%, 93%, 94%, 95%, 96%, 97%, 98%, or 99% identity with SEQ ID NO: 495. In some specific embodiments, the ECOLIN 19410-IL-10-linker-IL-10 polypeptide comprises SEQ ID NO: 495. In other specific embodiments, the ECOLIN 19410-IL-10-linker-IL-10 polypeptide consists of SEQ ID NO: 495. In some embodiments, the nucleic acid comprises a gene sequence. In certain embodiments, the nucleic acid comprising the ECOLIN 19410-IL-10-linker-IL-10 gene sequence has at least about 80%, 85%, 90%, 95%, or 99% identity with SEQ ID NO: 488. In some embodiments, the nucleic acid comprising the ECOLIN 19410-IL-10-linker-IL-10 gene sequence has at least about 85%, 86%, 87%, 88%, 89%, 90%, 91%, 92%, 93%, 94%, 95%, 96%, 97%, 98%, or 99% identity with SEQ ID NO: 488. In some specific embodiments, the nucleic acid comprising the ECOLIN 19410-IL-10-linker-IL-10 gene sequence comprises SEQ ID NO: 488. In other specific embodiments, the nucleic acid comprising the ECOLIN 19410-IL-10-linker-IL-10 gene sequence consists of SEQ ID NO: 488.

In any of the above embodiments, the nucleic acid may further comprise one or more of the following sequences: (1) promoter, (2) enhancer, (3) regulatory sequence, (4) ribosome binding site—nonlimiting examples of RBS are provided herein and include SEQ ID NO: 336-384, (5) secretion tag, non-limiting examples of secretion tags are provided herein and include SEQ ID NO: 385-394 (6) leader sequence, (7) auxotrophy, (8) antibiotic resistance.

In any of these embodiments, the nucleic acid may be functionally replaced, modified, and/or mutated in order to enhance stability and/or increase polypeptide production or secretion. In some embodiments, the nucleic acid is expressed and secreted in low-oxygen conditions, in the presence of certain molecules or metabolites, in the presence of molecules or metabolites associated with inflammation or an inflammatory response, or in the presence of some other metabolite that may or may not be present in the gut, such as arabinose. Exemplary chemical inducers are described herein. In some embodiments, the nucleic acid is directly operably linked to a first promoter. In some embodiments, the nucleic acid is indirectly operably linked to a first promoter. In one embodiment, the promoter is not operably linked with the nucleic acid in nature.

In some embodiments, nucleic acid is expressed under the control of a constitutive promoter. Non-limiting examples constitutive promoters are provided herein and include SEQ ID NO: 189-335.

In another embodiment, the nucleic acid is expressed under the control of an inducible promoter. In some embodiments, the nucleic acid is expressed under the control of a promoter that is directly or indirectly induced by exogenous environmental conditions. In one embodiment, the nucleic acid is expressed under the control of a promoter that is directly or indirectly induced by low-oxygen or anaerobic conditions, wherein expression of the nucleic acid is activated under low-oxygen or anaerobic environments, such as the environment of the mammalian gut. Inducible promoters are described in more detail infra. Non-limiting examples of low oxygen inducible promoters are provided herein and include SEQ ID NO: 151-167. Non-limiting examples of OxyR inducible promoters are provided herein and include SEQ ID NO: 168-171. Non-limiting examples of promoters regulated by chemical inducers are provided herein and include SEQ ID NO: 173-188.

The nucleic acid may be present on a plasmid or chromosome in the bacterial cell. In one embodiment, the nucleic acid is located on a plasmid in the bacterial cell. In another embodiment, the nucleic acid is located in the chromosome of the bacterial cell. In yet another embodiment, a native copy of the nucleic acid is located in the chromosome of the bacterial cell.

### Multiple Mechanisms of Action

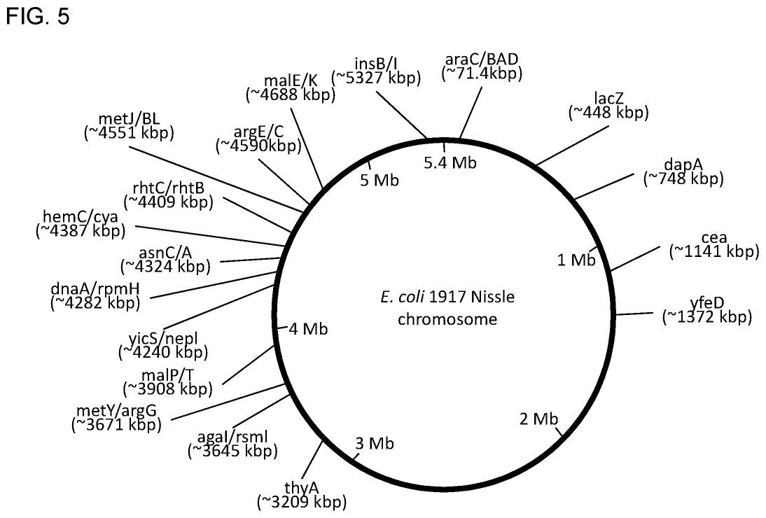

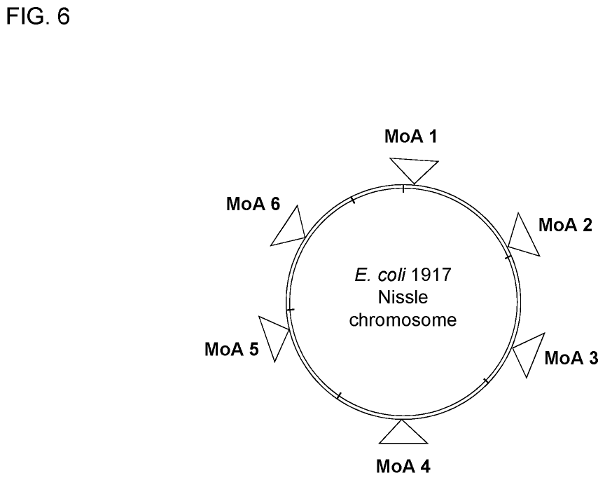

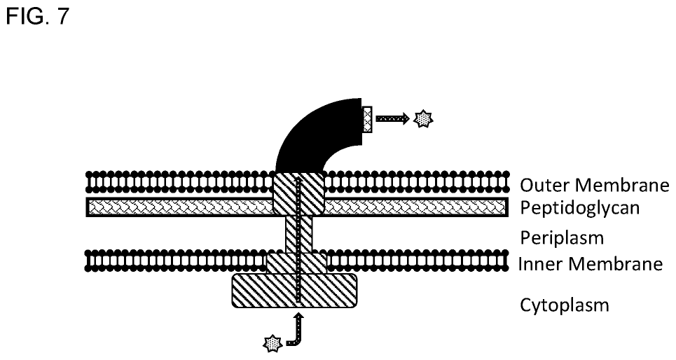

In some embodiments, the bacteria are genetically engineered to include multiple mechanisms of action (MOAs), e.g., circuits producing multiple copies of the same product (e.g., to enhance copy number) or circuits performing multiple different functions. Examples of insertion sites include, but are not limited to, malE/K, insB/I, araC/BAD, lacZ, dapA, cea, and other shown in FIG. 5. For example, the recombinant bacteria may include four copies of GLP-2 inserted at four different insertion sites, e.g., malE/K, insB/I, araC/BAD, and lacZ. Alternatively, the recombinant bacteria may include three copies of GLP-2 inserted at three different insertion sites, e.g., malE/K, insB/I, and lacZ, and three copies of a butyrogenic gene cassette inserted at three different insertion sites, e.g., dapA, cea, and araC/BA

In some embodiments, the bacteria are genetically engineered to include multiple mechanisms of action (MOAs), e.g., circuits producing multiple copies of the same product (e.g., to enhance copy number) or circuits performing multiple different functions. For example, the recombinant bacteria may include four copies of the gene, gene(s), or gene cassettes for producing the payload(s) inserted at four different insertion sites. Alternatively, the recombinant bacteria may include three copies of the gene, gene(s), or gene cassettes for producing the payload(s) inserted at three different insertion sites and three copies of the gene, gene(s), or gene cassettes for producing the payload(s) inserted at three different insertion sites.

In some embodiments, the recombinant bacteria comprise one or more of (1) one or more gene(s) or gene cassette(s) for the production of propionate as described herein, (2) one or more gene(s) or gene cassette(s) for the production of butyrate as described herein, (3) one or more gene(s) or gene cassette(s) for the production of acetate as described herein, (4) one or more gene(s) or gene cassette(s) for the production of tryptophan and/or its metabolites (including but not limited to kynurenine, indole, indole-3-acetic acid, indole-3 aldehyde, and IPA) as described herein, (5) one or more gene(s) or gene cassette(s) for the production of one or more of GLP-2 and GLP-2 analogs as described herein, (6) one or more gene(s) or gene cassette(s) for the production of human or viral or monommerized IL-10 as described herein, (7) one or more gene(s) or gene cassette(s) for the production of human IL-22 as described herein, (8) one or more gene(s) or gene cassette(s) for the production of IL-2, and/or SOD, and/or IL-27 and other interleukins as described herein, (9) one or more gene(s) or gene cassette(s) for the production of one or more transporters, e.g. for the import of tryptophan and/or metabolites as described herein, (10) one or more polypeptides for secretion, including but not limited to GLP-2 and its analogs, IL-10, and/or IL-22, SCFA and/or tryptophan synthesis and/or catabolic enzymes in wild type or in mutated form (for increased stability or metabolic activity), (11) one or more components of secretion machinery as described herein, (12) one or more auxotrophies, e.g., deltaThyA (13) one more antibiotic resistances, including but not limited to, kanamycin or chloramphenicol resistance, (14) one or more mutations/deletions to increase the flux through a metabolic pathway encoded by one or more genes or gene cassette(s), e.g mutations/deletions in genes in NADH consuming pathways, genes involved in feedback inhibition of a metabolic pathway encoded by the gene(s) or gene cassette(s) genes as described herein, (15) one or more mutations/deletions in one or more genes of the endogenous metabolic pathways, e.g., tryptophan synthesis pathway.

In some embodiments, the recombinant bacteria promote one or more of the following effector functions: (1) neutralizes TNF-α, IFN-γ, IL-1β, IL-6, IL-8, IL-17, and/or chemokines, e.g., CXCL-8 and CCL2 (2) activates include AHR (e.g., which result in IL-22 production) and (3) activates PXR, (4) inhibits HDACs, (5) activates GPR41 and/or GPR43 and/or GPR109A, (6) inhibits NF-kappaB signaling, (7) modulators of PPARgamma, (8) activates of AMPK signaling, (9) modulates GLP-1 secretion and/or (10). scavenges hydroxyl radicals and functions as antioxidants.

In some embodiments, under conditions where the gene, gene(s), or gene cassettes for producing the payload(s) is expressed, the recombinant bacteria of the disclosure produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more of the payload(s) as compared to unmodified bacteria of the same subtype under the same conditions.

In some embodiments, the recombinant bacteria produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more of a payload under inducing conditions than unmodified bacteria of the same subtype under the same conditions. Certain unmodified bacteria will not have detectable levels of the payload. In embodiments using genetically modified forms of these bacteria, the payload will be detectable under inducing conditions.

In some embodiments, quantitative PCR (qPCR) is used to amplify, detect, and/or quantify mRNA expression levels of the gene, gene(s), or gene cassettes for producing the payload(s). Primers may be designed and used to detect mRNA in a sample according to methods known in the art. In some embodiments, a fluorophore is added to a sample reaction mixture that may contain payload RNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the payload(s).

In some embodiments, the recombinant bacteria comprise gene sequence(s) encoding one or more therapeutic peptides for secretion, e.g., IL-10, IL-2, IL-22, IL-27, SOD, GLP-2 and kynurenine, as described herein.

In some embodiments, the recombinant bacteria comprise a butyrate gene cassette and are capable of producing butyrate and comprise a gene sequence encoding IL-10. In some embodiments, the recombinant bacteria comprise a butyrate gene cassette and are capable of producing butyrate and comprise a gene sequence encoding IL-2. In some embodiments, the recombinant bacteria comprise a butyrate gene cassette and are capable of producing butyrate and comprise a gene sequence encoding IL-22. In some embodiments, the recombinant bacteria comprise a butyrate gene cassette and are capable of producing butyrate and comprise a gene sequence encoding IL-27. In some embodiments, the recombinant bacteria comprise a butyrate gene cassette and are capable of producing butyrate and comprise a gene sequence encoding SOD. In some embodiments, the recombinant bacteria comprise a butyrate gene cassette and are capable of producing butyrate and comprise a gene sequence encoding GLP-2. In some embodiments, the recombinant bacteria comprise a butyrate gene cassette and are capable of producing butyrate and are capable of producing kyurenine.

In some embodiments, the recombinant bacteria comprise a butyrate gene cassette and are capable of producing butyrate and comprise a gene sequence encoding IL-10 and one or more gene sequences encoding IL-2, IL-22, IL-27, GLP-2, and SOD.

In some embodiments, the recombinant bacteria comprise a butyrate gene cassette and are capable of producing butyrate and comprise a gene sequence encoding IL-2 and one or more gene sequences encoding IL-10, IL-22, IL-27, GLP-2, and SOD.

In any of these embodiments, the bacteria can produce kynurenine. In some embodiments, the recombinant bacteria comprise a butyrate gene cassette and are capable of producing butyrate and comprise a gene sequence encoding IL-22 and one or more gene sequences encoding IL-2, IL-10, IL-27, GLP-2, and SOD.

In some embodiments, the recombinant bacteria comprise a butyrate gene cassette and are capable of producing butyrate and comprise a gene sequence encoding SOD and one or more gene sequences encoding IL-2, IL-22, IL-27, GLP-2, and IL-10.

In some embodiments, the recombinant bacteria comprise a gene sequence(s) encoding IL-27 and a gene sequence encoding one or more molecules selected from IL-2, IL-22, IL-10, GLP-2, and SOD.

In some embodiments, the recombinant bacteria comprise a gene sequence encoding GLP-2 and a gene sequence(s) encoding one or more molecules selected from IL-2, IL-22, IL-27, IL-10, and SOD.

In any of these embodiments the bacteria comprise a propionate gene cassette and can produce propionate. In any of these embodiments, the recombinant bacteria can produce kynurenine. In any of these embodiments, the genetically engineered bacteria are capable of producing butyrate. In any of these embodiments, the recombinant bacteria are capable of producing acetate.

In any of these combination embodiments, the GLP-2 polypeptide may be a GLP-2 fusion protein. In some embodiments, the GLP-2 fusion protein is a GLP-2-IgA fusion protein as described herein. In any of these combination embodiments, the IL-10 polypeptide may be a fusion protein. In some embodiments, the IL-10 fusion protein is a IL-10-Linker-IL-10 fusion protein as described herein.

In any of these combination embodiments, the recombinant bacteria may comprise gene sequence(s) which encode two separate effector polypeptides covalently linked together through a peptide bond or peptide linker. In one embodiment, the recombinant bacteria may comprise gene sequence(s) which encode GLP-2 covalently linked to IL-22. In one embodiment, the recombinant bacteria may comprise gene sequence(s) which encode GLP-2 joined by a peptide bond or a peptide linker to a first and a second IL-10 monomer, which are joined together by a second peptide bond or peptide linker.

The gene sequence(s) encoding therapeutic peptides for secretion may be expressed under the control of a constitutive promoter or an inducible promoter. The gene sequence(s) encoding the one or more therapeutic peptides for secretion are expressed under the control of a promoter that is directly or indirectly induced by exogenous environmental conditions, e.g., low-oxygen or anaerobic conditions, wherein expression of the gene sequence(s) encoding the one or more therapeutic peptides for secretion are activated under low-oxygen or anaerobic environments, such as the environment of the mammalian gut. Alternatively, the gene sequence(s) encoding the one or more therapeutic peptides for secretion are expressed under the control of a promoter that is directly or indirectly induced by inflammatory conditions. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, and a P_{TetR} promoter, each of which are described in more detail herein. Inducible promoters are described in more detail infra.

The at least one gene encoding the therapeutic peptides for secretion may be present on a plasmid or chromosome in the bacterial cell. In one embodiment, a native copy of the gene sequence(s) encoding the therapeutic peptides for secretion are located in the chromosome of the bacterial cell, and at least one gene encoding the therapeutic peptides for secretion from a different species of bacteria are located on a plasmid in the bacterial cell. In yet another embodiment, a native copy of the gene sequence(s) encoding the therapeutic peptides for secretion are located on a plasmid in the bacterial cell, and at least one gene encoding the therapeutic peptides for secretion from a different species of bacteria are located on a plasmid in the bacterial cell. In yet another embodiment, a native copy of the gene sequence(s) encoding the therapeutic peptides for secretion are located in the chromosome of the bacterial cell, and at least one gene encoding the therapeutic peptides for secretion from a different species of bacteria are located in the chromosome of the bacterial cell.

In some embodiments, the gene sequence(s) encoding the one or more therapeutic peptides for secretion are expressed on a low-copy plasmid. In some embodiments, the gene sequence(s) encoding the one or more therapeutic peptides for secretion are expressed on a high-copy plasmid. In some embodiments, the high-copy plasmid may be useful for increasing expression of the therapeutic peptides for secretion.

In some embodiments, a recombinant bacterial cell comprising at least one gene encoding the therapeutic peptides for secretion are expressed on a high-copy plasmid do not increase tryptophan catabolism as compared to a recombinant bacterial cell comprising the same gene expressed on a low-copy plasmid in the absence of a heterologous importer of tryptophan and/or its metabolites and additional copies of a native importer of tryptophan and/or its metabolites. In alternate embodiments, the importer of tryptophan and/or its metabolites is used in conjunction with a high-copy plasmid.

In some embodiments, the recombinant bacteria described above further comprise one or more of the modifications, mutations, and/or deletions in endogenous genes described herein.

In some embodiments, the genetically engineered microorganism further comprises a mutation and/or deletion in ldhA. In some embodiments, the genetically engineered microorganism further comprises a mutation and/or deletion in frdA. In some embodiments, the genetically engineered microorganism further comprises a mutation and/or deletion in adhE. In some embodiments, the genetically engineered microorganism further comprises a mutation and/or deletion in one or more of ldhA, frdA, and adhE.

In some embodiments, surface display could be used to display a protein of interest on the surface of the genetically modified bacterium. In some embodiments, the recombinant bacteria and/or microorganisms encode one or more gene(s) and/or gene cassette(s) encoding a protein of interest, e.g., an effector molecule, which is anchored or displayed on the surface of the bacteria and/or microorganisms.

### Induction of Payloads During Strain Culture

In some embodiments, it is desirable to pre-induce payload or protein of interest expression and/or payload activity prior to administration. Such payload or protein of interest may be an effector intended for secretion or may be an enzyme which catalyzes a metabolic reaction to produce an effector. In other embodiments, the protein of interest is an enzyme which catabolizes a harmful metabolite. In such situations, the strains are pre-loaded with active payload or protein of interest. In such instances, the recombinant bacteria express one or more protein(s) of interest, under conditions provided in bacterial culture during cell growth, expansion, purification, fermentation, and/or manufacture prior to administration in vivo. Such culture conditions can be provided in a flask, fermenter or other appropriate culture vessel, e.g., used during cell growth, cell expansion, fermentation, recovery, purification, formulation, and/or manufacture. As used herein, the term “bacterial culture” or bacterial cell culture” or “culture” refers to bacterial cells or microorganisms, which are maintained or grown in vitro during several production processes, including cell growth, cell expansion, recovery, purification, fermentation, and/or manufacture. As used herein, the term “fermentation” refers to the growth, expansion, and maintenance of bacteria under defined conditions. Fermentation may occur under a number of cell culture conditions, including anaerobic or low oxygen or oxygenated conditions, in the presence of inducers, nutrients, at defined temperatures, and the like.

Culture conditions are selected to achieve optimal activity and viability of the cells, while maintaining a high cell density (high biomass) yield. A number of cell culture conditions and operating parameters are monitored and adjusted to achieve optimal activity, high yield and high viability, including oxygen levels (e.g., low oxygen, microaerobic, aerobic), temperature of the medium, and nutrients and/or different growth media, chemical and/or nutritional inducers and other components provided in the medium.

In some embodiments, the one or more protein(s) of interest and are directly or indirectly induced, while the strains is grown up for in vivo administration. Without wishing to be bound by theory, pre-induction may boost in vivo activity. This is particularly important in proximal regions of the gut which are reached first by the bacteria, e.g., the small intestine. If the bacterial residence time in this compartment is relatively short, the bacteria may pass through the small intestine without reaching full in vivo induction capacity. In contrast, if a strain is pre-induced and preloaded, the strains are already fully active, allowing for greater activity more quickly as the bacteria reach the intestine. Ergo, no transit time is “wasted”, in which the strain is not optimally active. As the bacteria continue to move through the intestine, in vivo induction occurs under environmental conditions of the gut (e.g., low oxygen, or in the presence of gut metabolites).

In one embodiment, expression of one or more payload(s), is induced during cell growth, cell expansion, fermentation, recovery, purification, formulation, and/or manufacture. In one embodiment, expression of several different proteins of interest is induced during cell growth, cell expansion, fermentation, recovery, purification, formulation, and/or manufacture. In one embodiment, expression of one or more payload(s), is driven from the same promoter as a multicistronic message. In one embodiment, expression of one or more payload(s) is driven from the same promoter as two or more separate messages. In one embodiment, expression of one or more payload(s) is driven from the one or more different promoters.

In some embodiments, the strains are administered without any pre-induction protocols during strain growth prior to in vivo administration.

Anaerobic Induction

In some embodiments, cells are induced under anaerobic or low oxygen conditions in culture. In such instances, cells are grown (e.g., for 1.5 to 3 hours) until they have reached a certain OD, e.g., ODs within the range of 0.1 to 10, indicating a certain density e.g., ranging from 1×10\hat{ }8 to 1×10\hat{ }11, and exponential growth and are then switched to anaerobic or low oxygen conditions for approximately 3 to 5 hours. In some embodiments, strains are induced under anaerobic or low oxygen conditions, e.g. to induce FNR promoter activity and drive expression of one or more payload(s) under the control of one or more FNR promoters.

In one embodiment, expression of one or more payload(s), is under the control of one or more anaerobic or low oxygen inducible promoter(s), e.g., FNR promoter(s), and is induced during cell growth, cell expansion, fermentation, recovery, purification, formulation, and/or manufacture under anaerobic or low oxygen conditions. In one embodiment, expression of several different proteins of interest is under the control of one or more anaerobic or low oxygen inducible promoter(s), e.g., FNR promoter(s) and is induced during cell growth, cell expansion, fermentation, recovery, purification, formulation, and/or manufacture under anaerobic or low oxygen conditions.

In one embodiment, expression of two or more payload(s), is under the control of one or more anaerobic or low oxygen inducible promoter(s), e.g., FNR promoter(s), and is driven from the same promoter in the form of a multicistronic message under anaerobic or low oxygen conditions. In one embodiment, expression of one or more payload(s), is under the control of one or more anaerobic or low oxygen inducible promoter(s), e.g., FNR promoter(s), and is driven from the same promoter as two or more separate messages under anaerobic or low oxygen conditions. In one embodiment, expression of one or more payload(s) under the control of one or more anaerobic or low oxygen inducible promoter(s), e.g., FNR promoter(s), and is driven from the one or more different promoters under anaerobic or low oxygen conditions.

Without wishing to be bound by theory, strains that comprise one or more payload(s) under the control of an FNR promoter, may allow expression of payload(s) from these promoters in vitro, under anaerobic or low oxygen culture conditions, and in vivo, under the low oxygen conditions found in the gut.

In some embodiments, promoters inducible by arabinose, IPTG, rhamnose, tetracycline, and/or other chemical and/or nutritional inducers can be induced under anaerobic or low oxygen conditions in the presence of the chemical and/or nutritional inducer. In some embodiments, strains may comprise a combination of gene sequence(s), some of which are under control of FNR promoters and others which are under control of promoters induced by chemical and/or nutritional inducers. In some embodiments, strains may comprise one or more payload gene sequence(s) under the control of one or more FNR promoter(s) and one or more payload gene sequence(s) which are induced by a one or more chemical and/or nutritional inducer(s), including, but not limited to, arabinose, IPTG, rhamnose, tetracycline, and/or other chemical and/or nutritional inducers described herein or known in the art. In some embodiments, strains may comprise one or more payload gene sequence(s) and/or under the control of one or more FNR promoter(s), and one or more payload gene sequence(s) under the control of a one or more constitutive promoter(s) described herein. In some embodiments, strains may comprise one or more payload gene sequence(s) under the control of an FNR promoter and one or more payload gene sequence(s) under the control of a one or more thermoregulated promoter(s) described herein.

In one embodiment, expression of one or more payload gene sequence(s) is under the control of one or more promoter(s) regulated by chemical and/or nutritional inducers and is induced during cell growth, cell expansion, fermentation, recovery, purification, formulation, and/or manufacture under anaerobic and/or low oxygen conditions. In one embodiment, the chemical and/or nutritional inducer is arabinose and the promoter is inducible by arabinose. In one embodiment, the chemical and/or nutritional inducer is IPTG and the promoter is inducible by IPTG. In one embodiment, the chemical and/or nutritional inducer is rhamnose and the promoter is inducible by rhamnose. In one embodiment, the chemical and/or nutritional inducer is tetracycline and the promoter is inducible by tetracycline.

In one embodiment, expression of one or more payload(s), is under the control of one or more promoter(s) regulated by chemical and/or nutritional inducers and is driven from the same promoter in the form of a multicistronic message under anaerobic and/or low oxygen conditions. In one embodiment, expression of one or more payload(s), is under the control of one or more promoter(s) regulated by chemical and/or nutritional inducers and is driven from the same promoter as two or more separate messages under anaerobic and/or low oxygen conditions. In one embodiment, expression of one or more payload(s), is under the control of one or more promoter(s) regulated by chemical and/or nutritional inducers and is driven from the one or more different promoters under anaerobic and/or low oxygen conditions.

In one embodiment, strains may comprise a combination of gene sequence(s), some of which are under control of a first inducible promoter and others which are under control of a second inducible promoter, both induced by chemical and/or nutritional inducers, under anaerobic or low oxygen conditions. In one embodiment, strains may comprise a combination of gene sequence(s), some of which are under control of a first inducible promoter and others which are under control of a second inducible promoter, both induced by chemical and/or nutritional inducers. In some embodiments, the strains comprise gene sequence(s) under the control of a third inducible promoter, e.g., an anaerobic/low oxygen promoter, e.g., FNR promoter. In one embodiment, strains may comprise a combination of gene sequence(s), some of which are under control of a first inducible promoter, e.g., a chemically induced promoter or a low oxygen promoter and others which are under control of a second inducible promoter, e.g. a temperature sensitive promoter. In one embodiment, strains may comprise a combination of gene sequence(s), some of which are under control of a first inducible promoter, e.g., a FNR promoter and others which are under control of a second inducible promoter, e.g. a temperature sensitive promoter. In one embodiment, strains may comprise a combination of gene sequence(s), some of which are under control of a first inducible promoter, e.g., a chemically induced and others which are under control of a second inducible promoter, e.g. a temperature sensitive promoter. In some embodiments, strains may comprise one or more payload gene sequence(s) under the control of an FNR promoter and one or more payload gene sequence(s) under the control of a one or more promoter(s) which are induced by a one or more chemical and/or nutritional inducer(s), including, but not limited to, by arabinose, IPTG, rhamnose, tetracycline, and/or other chemical and/or nutritional inducers described herein or known in the art. Additionally the strains may comprise a construct which is under thermoregulatory control. In some embodiments, the bacteria strains further comprise payload sequence(s) under the control of one or more constitutive promoter(s) active under low oxygen conditions.

Aerobic Induction

In some embodiments, it is desirable to prepare, pre-load and pre-induce the strains under aerobic conditions. This allows more efficient growth and viability, and, in some cases, reduces the build-up of toxic metabolites. In such instances, cells are grown (e.g., for 1.5 to 3 hours) until they have reached a certain OD, e.g., ODs within the range of 0.1 to 10, indicating a certain density e.g., ranging from 1×10\hat{ }8 to 1×10\hat{ }11, and exponential growth and are then induced through the addition of the inducer or through other means, such as shift to a permissive temperature, for approximately 3 to 5 hours.

In some embodiments, promoters inducible by arabinose, IPTG, rhamnose, tetracycline, and/or other chemical and/or nutritional inducers described herein or known in the art can be induced under aerobic conditions in the presence of the chemical and/or nutritional inducer during cell growth, cell expansion, fermentation, recovery, purification, formulation, and/or manufacture. In one embodiment, expression of one or more payload(s) is under the control of one or more promoter(s) regulated by chemical and/or nutritional inducers and is induced during cell growth, cell expansion, fermentation, recovery, purification, formulation, and/or manufacture under aerobic conditions.

In one embodiment, expression of one or more payload(s), is under the control of one or more promoter(s) regulated by chemical and/or nutritional inducers and is driven from the same promoter in the form of a multicistronic message under aerobic conditions. In one embodiment, expression of one or more payload(s), is under the control of one or more promoter(s) regulated by chemical and/or nutritional inducers and is driven from the same promoter as two or more separate messages under aerobic conditions. In one embodiment, expression of one or more payload(s), is under the control of one or more promoter(s) regulated by chemical and/or nutritional inducers and is driven from the one or more different promoters under aerobic conditions.

In one embodiment, the chemical and/or nutritional inducer is arabinose and the promoter is inducible by arabinose. In one embodiment, the chemical and/or nutritional inducer is IPTG and the promoter is inducible by IPTG. In one embodiment, the chemical and/or nutritional inducer is rhamnose and the promoter is inducible by rhamnose. In one embodiment, the chemical and/or nutritional inducer is tetracycline and the promoter is inducible by tetracycline.

In some embodiments, promoters regulated by temperature are induced during cell growth, cell expansion, fermentation, recovery, purification, formulation, and/or manufacture. In one embodiment, expression of one or more payload(s) is driven directly or indirectly by one or more thermoregulated promoter(s) and is induced during cell growth, cell expansion, fermentation, recovery, purification, formulation, and/or manufacture under aerobic conditions.

In one embodiment, expression of one or more payload(s) is driven directly or indirectly by one or more thermoregulated promoter(s) and is driven from the same promoter in the form of a multicistronic message under aerobic conditions. In one embodiment, expression of one or more payload(s) is driven directly or indirectly by one or more thermoregulated promoter(s) and is driven from the same promoter as two or more separate messages under aerobic conditions. In one embodiment, expression of one or more payload(s) is driven directly or indirectly by one or more thermoregulated promoter(s) and is driven from the one or more different promoters under aerobic conditions.

In one embodiment, strains may comprise a combination of gene sequence(s), some of which are under control of a first inducible promoter and others which are under control of a second inducible promoter, both induced under aerobic conditions. In some embodiments, a strain comprises three or more different promoters which are induced under aerobic culture conditions.

In one embodiment, strains may comprise a combination of gene sequence(s), some of which are under control of a first inducible promoter and others which are under control of a second inducible promoter, both induced by chemical and/or nutritional inducers. In one embodiment, strains may comprise a combination of gene sequence(s), some of which are under control of a first inducible promoter, e.g. a chemically inducible promoter, and others which are under control of a second inducible promoter, e.g. a temperature sensitive promoter under aerobic culture conditions. In some embodiments two or more chemically induced promoter gene sequence(s) are combined with a thermoregulated construct described herein. In one embodiment, the chemical and/or nutritional inducer is arabinose and the promoter is inducible by arabinose. In one embodiment, the chemical and/or nutritional inducer is IPTG and the promoter is inducible by IPTG. In one embodiment, the chemical and/or nutritional inducer is rhamnose and the promoter is inducible by rhamnose. In one embodiment, the chemical and/or nutritional inducer is tetracycline and the promoter is inducible by tetracycline.

In one embodiment, strains may comprise a combination of gene sequence(s), some of which are under control of a first inducible promoter, e.g., a FNR promoter and others which are under control of a second inducible promoter, e.g. a temperature sensitive promoter. In one embodiment, strains may comprise a combination of gene sequence(s), some of which are under control of a first inducible promoter, e.g., a chemically induced and others which are under control of a second inducible promoter, e.g. a temperature sensitive promoter. In some embodiments, strains may comprise one or more payload gene sequence(s) under the control of an FNR promoter and one or more payload gene sequence(s) under the control of a one or more promoter(s) which are induced by a one or more chemical and/or nutritional inducer(s), including, but not limited to, by arabinose, IPTG, rhamnose, tetracycline, and/or other chemical and/or nutritional inducers described herein or known in the art. Additionally the strains may comprise a construct which is under thermoregulatory control. In some embodiments, the bacteria strains further comprise payload sequences under the control of one or more constitutive promoter(s) active under aerobic conditions.

In some embodiments, genetically engineered strains comprise gene sequence(s) which are induced under aerobic culture conditions. In some embodiments, these strains further comprise FNR inducible gene sequence(s) for in vivo activation in the gut. In some embodiments, these strains do not further comprise FNR inducible gene sequence(s) for in vivo activation in the gut.

In some embodiments, genetically engineered strains comprise gene sequence(s), which are arabinose inducible under aerobic culture conditions. In some embodiments, these strains do not further comprise FNR inducible gene sequence(s) for in vivo activation in the gut.

In some embodiments, genetically engineered strains comprise gene sequence(s), which are IPTG inducible under aerobic culture conditions. In some embodiments, these strains further comprise FNR inducible gene sequence(s) for in vivo activation in the gut. In some embodiments, these strains do not further comprise FNR inducible gene sequence(s) for in vivo activation in the gut.

In some embodiments, genetically engineered strains comprise gene sequence(s) which are arabinose inducible under aerobic culture conditions. In some embodiments, such a strain further comprises sequence(s) which are IPTG inducible under aerobic culture conditions. In some embodiments, these strains further comprise FNR inducible gene payload sequence(s) for in vivo activation in the gut. In some embodiments, these strains do not further comprise FNR inducible gene sequence(s) for in vivo activation in the gut.

As evident from the above non-limiting examples, genetically engineered strains comprise inducible gene sequence(s) which can be induced numerous combinations. For example, rhamnose or tetracycline can be used as an inducer with the appropriate promoters in addition or in lieu of arabinose and/or IPTG or with thermoregulation. Additionally, such bacterial strains can also be induced with the chemical and/or nutritional inducers under anaerobic conditions.

Microaerobic Induction

In some embodiments, viability, growth, and activity are optimized by pre-inducing the bacterial strain under microaerobic conditions. In some embodiments, microaerobic conditions are best suited to “strike a balance” between optimal growth, activity and viability conditions and optimal conditions for induction; in particular, if the expression of the one or more payload(s) are driven by an anaerobic and/or low oxygen promoter, e.g., a FNR promoter. In such instances, cells are grown (e.g., for 1.5 to 3 hours) until they have reached a certain OD, e.g., ODs within the range of 0.1 to 10, indicating a certain density e.g., ranging from 1×10\hat{ }8 to 1×10 \hat{ }11, and exponential growth and are then induced through the addition of the inducer or through other means, such as shift to at a permissive temperature, for approximately 3 to 5 hours.

In one embodiment, expression of one or more payload(s) is under the control of one or more FNR promoter(s) and is induced during cell growth, cell expansion, fermentation, recovery, purification, formulation, and/or manufacture under microaerobic conditions.

In one embodiment, expression of one or more payload(s), is under the control of one or more FNR promoter(s) and is driven from the same promoter in the form of a multicistronic message under microaerobic conditions. In one embodiment, expression of one or more payload(s), is under the control of one or more FNR promoter(s) and is driven from the same promoter as two or more separate messages under microaerobic conditions. In one embodiment, expression of one or more payload(s), is under the control of one or more FNR promoter(s) and is driven from the one or more different promoters under microaerobic conditions.

Without wishing to be bound by theory, strains that comprise one or more payload(s) under the control of an FNR promoter, may allow expression of payload(s) from these promoters in vitro, under microaerobic culture conditions, and in vivo, under the low oxygen conditions found in the gut.

In some embodiments, promoters inducible by arabinose, IPTG, rhamnose, tetracycline, and/or other chemical and/or nutritional inducers can be induced under microaerobic conditions in the presence of the chemical and/or nutritional inducer. In particular, strains may comprise a combination of gene sequence(s), some of which are under control of FNR promoters and others which are under control of promoters induced by chemical and/or nutritional inducers. In some embodiments, strains may comprise one or more payload gene sequence(s) sequence(s) under the control of one or more FNR promoter(s) and one or more payload gene sequence(s) under the control of a one or more promoter(s) which are induced by a one or more chemical and/or nutritional inducer(s), including, but not limited to, arabinose, IPTG, rhamnose, tetracycline, and/or other chemical and/or nutritional inducers described herein or known in the art. In some embodiments, strains may comprise one or more payload gene sequence(s) under the control of one or more FNR promoter(s), and one or more payload gene sequence(s) under the control of a one or more constitutive promoter(s) described herein. In some embodiments, strains may comprise one or more payload gene sequence(s) under the control of an FNR promoter and one or more payload gene sequence(s) under the control of a one or more thermoregulated promoter(s) described herein.

In one embodiment, expression of one or more payload(s) is under the control of one or more promoter(s) regulated by chemical and/or nutritional inducers and is induced during cell growth, cell expansion, fermentation, recovery, purification, formulation, and/or manufacture under microaerobic conditions.

In one embodiment, expression of one or more payload(s), is under the control of one or more promoter(s) regulated by chemical and/or nutritional inducers and is driven from the same promoter in the form of a multicistronic message under microaerobic conditions. In one embodiment, expression of one or more payload(s), is under the control of one or more promoter(s) regulated by chemical and/or nutritional inducers and is driven from the same promoter as two or more separate messages under microaerobic conditions. In one embodiment, expression of one or more payload(s), is under the control of one or more promoter(s) regulated by chemical and/or nutritional inducers and is driven from the one or more different promoters under microaerobic conditions.

In one embodiment, strains may comprise a combination of gene sequence(s), some of which are under control of a first inducible promoter and others which are under control of a second inducible promoter, both induced by chemical and/or nutritional inducers, under microaerobic conditions. In one embodiment, strains may comprise a combination of gene sequence(s), some of which are under control of a first inducible promoter and others which are under control of a second inducible promoter, both induced by chemical and/or nutritional inducers. In some embodiments, the strains comprise gene sequence(s) under the control of a third inducible promoter, e.g., an anaerobic/low oxygen promoter or microaerobic promoter, e.g., FNR promoter. In one embodiment, strains may comprise a combination of gene sequence(s), some of which are under control of a first inducible promoter, e.g., a chemically induced promoter or a low oxygen or microaerobic promoter and others which are under control of a second inducible promoter, e.g. a temperature sensitive promoter. In one embodiment, strains may comprise a combination of gene sequence(s), some of which are under control of a first inducible promoter, e.g., a FNR promoter and others which are under control of a second inducible promoter, e.g. a temperature sensitive promoter. In one embodiment, strains may comprise a combination of gene sequence(s), some of which are under control of a first inducible promoter, e.g., a chemically induced and others which are under control of a second inducible promoter, e.g. a temperature sensitive promoter. In some embodiments, strains may comprise one or more payload gene sequence(s) under the control of an FNR promoter and one or more payload gene sequence(s) under the control of a one or more promoter(s) which are induced by a one or more chemical and/or nutritional inducer(s), including, but not limited to, by arabinose, IPTG, rhamnose, tetracycline, and/or other chemical and/or nutritional inducers described herein or known in the art. Additionally the strains may comprise a construct which is under thermoregulatory control. In some embodiments, the bacteria strains further comprise payload under the control of one or more constitutive promoter(s) active under low oxygen conditions.

Induction of Strains Using Phasing, Pulsing and/or Cycling

In some embodiments, cycling, phasing, or pulsing techniques are employed during cell growth, expansion, recovery, purification, fermentation, and/or manufacture to efficiently induce and grow the strains prior to in vivo administration. This method is used to “strike a balance” between optimal growth, activity, cell health, and viability conditions and optimal conditions for induction; in particular, if growth, cell health or viability are negatively affected under inducing conditions. In such instances, cells are grown (e.g., for 1.5 to 3 hours) in a first phase or cycle until they have reached a certain OD, e.g., ODs within the range of 0.1 to 10, indicating a certain density e.g., ranging from 1×10 \hat{ }8 to 1×10\hat{ }11, and are then induced through the addition of the inducer or through other means, such as shift to a permissive temperature (if a promoter is thermoregulated), or change in oxygen levels (e.g., reduction of oxygen level in the case of induction of an FNR promoter driven construct) for approximately 3 to 5 hours. In a second phase or cycle, conditions are brought back to the original conditions which support optimal growth, cell health and viability. Alternatively, if a chemical and/or nutritional inducer is used, then the culture can be spiked with a second dose of the inducer in the second phase or cycle.

In some embodiments, two cycles of optimal conditions and inducing conditions are employed (i.e, growth, induction, recovery and growth, induction). In some embodiments, three cycles of optimal conditions and inducing conditions are employed. In some embodiments, four or more cycles of optimal conditions and inducing conditions are employed. In a non-liming example, such cycling and/or phasing is used for induction under anaerobic and/or low oxygen conditions (e.g., induction of FNR promoters). In one embodiment, cells are grown to the optimal density and then induced under anaerobic and/or low oxygen conditions. Before growth and/or viability are negatively impacted due to stressful induction conditions, cells are returned to oxygenated conditions to recover, after which they are then returned to inducing anaerobic and/or low oxygen conditions for a second time. In some embodiments, these cycles are repeated as needed.

In some embodiments, growing cultures are spiked once with the chemical and/or nutritional inducer. In some embodiments, growing cultures are spiked twice with the chemical and/or nutritional inducer. In some embodiments, growing cultures are spiked three or more times with the chemical and/or nutritional inducer. In a non-limiting example, cells are first grown under optimal growth conditions up to a certain density, e.g., for 1.5 to 3 hour) to reached an of 0.1 to 10, until the cells are at a density ranging from 1×10 \hat{ }8 to 1×10\hat{ }11. Then the chemical inducer, e.g., arabinose or IPTG, is added to the culture. After 3 to 5 hours, an additional dose of the inducer is added to re-initiate the induction. Spiking can be repeated as needed.

In some embodiments, phasing or cycling changes in temperature in the culture. In another embodiment, adjustment of temperature may be used to improve the activity of a payload. For example, lowering the temperature during culture may improve the proper folding of the payload. In such instances, cells are first grown at a temperature optimal for growth (e.g., 37 C). In some embodiments, the cells are then induced, e.g., by a chemical inducer, to express the payload. Concurrently or after a set amount of induction time, the temperature in the media is lowered, e.g., between 25 and 35 C, to allow improved folding of the expressed payload.

In some embodiments, payload(s) are under the control of different inducible promoters, for example two different chemical inducers. In other embodiments, the payload is induced under low oxygen conditions or microaerobic conditions and a second payload is induced by a chemical inducer.

In one embodiment, expression of one or more payload(s) is under the control of one or more FNR promoter(s) and is induced during cell growth, cell expansion, fermentation, recovery, purification, formulation, and/or manufacture by using phasing or cycling or pulsing or spiking techniques.

In one embodiment, expression of one or more payload(s), is under the control of one or more FNR promoter(s) and is driven from the same promoter in the form of a multicistronic message through the employment of phasing or cycling or pulsing or spiking techniques. In one embodiment, expression of one or more payload(s), is under the control of one or more FNR promoter(s) and is driven from the same promoter as two or more separate messages through the employment of phasing or cycling or pulsing or spiking techniques. In one embodiment, expression of one or more payload(s), is under the control of one or more FNR promoter(s) and is driven from the one or more different promoters through the employment of phasing or cycling or pulsing or spiking techniques.

In some embodiments, promoters inducible by arabinose, IPTG, rhamnose, tetracycline, and/or other chemical and/or nutritional inducers can be induced through the employment of phasing or cycling or pulsing or spiking techniques in the presence of the chemical and/or nutritional inducer. In particular, strains may comprise a combination of gene sequence(s), some of which are under control of FNR promoters and others which are under control of promoters induced by chemical and/or nutritional inducers. In some embodiments, strains may comprise one or more payload gene sequence(s) under the control of one or more FNR promoter(s) and one or more payload gene sequence(s) under the control of a one or more promoter(s) which are induced by a one or more chemical and/or nutritional inducer(s), including, but not limited to, arabinose, IPTG, rhamnose, tetracycline, and/or other chemical and/or nutritional inducers described herein or known in the art. In some embodiments, strains may comprise one or more payload gene sequence(s) under the control of one or more FNR promoter(s), and one or more payload gene sequence(s) under the control of a one or more constitutive promoter(s) described herein and are induced through the employment of phasing or cycling or pulsing or spiking techniques. In some embodiments, strains may comprise one or more payload gene sequence(s) under the control of an FNR promoter and one or more payload gene sequence(s) under the control of a one or more thermoregulated promoter(s) described herein, and are induced through the employment of phasing or cycling or pulsing or spiking techniques.

Any of the strains described herein can be grown through the employment of phasing or cycling or pulsing or spiking techniques. In one embodiment, expression of one or more payload(s) is under the control of one or more promoter(s) regulated by chemical and/or nutritional inducers and is induced during cell growth, cell expansion, fermentation, recovery, purification, formulation, and/or manufacture under anaerobic and/or low oxygen conditions.

In one embodiment, expression of one or more payload(s), is under the control of one or more promoter(s) regulated by chemical and/or nutritional inducers and is driven from the same promoter in the form of a multicistronic message and which are induced through the employment of phasing or cycling or pulsing or spiking techniques. In one embodiment, expression of one or more payload(s), is under the control of one or more promoter(s) regulated by chemical and/or nutritional inducers and is driven from the same promoter as two or more separate messages and is grown through the employment of phasing or cycling or pulsing or spiking techniques. In one embodiment, expression of one or more payload(s), is under the control of one or more promoter(s) regulated by chemical and/or nutritional inducers and is driven from the one or more different promoters, all of which are induced through the employment of phasing or cycling or pulsing or spiking techniques.

In one embodiment, strains may comprise a combination of gene sequence(s), some of which are under control of a first inducible promoter and others which are under control of a second inducible promoter, both induced by chemical and/or nutritional inducers, through the employment of phasing or cycling or pulsing or spiking techniques. In one embodiment, strains may comprise a combination of gene sequence(s), some of which are under control of a first inducible promoter and others which are under control of a second inducible promoter, both induced by chemical and/or nutritional inducers through the employment of phasing or cycling or pulsing or spiking techniques. In some embodiments, the strains comprise gene sequence(s) under the control of a third inducible promoter, e.g., an anaerobic/low oxygen promoter, e.g., FNR promoter. In one embodiment, strains may comprise a combination of gene sequence(s), some of which are under control of a first inducible promoter, e.g., a chemically induced promoter or a low oxygen promoter and others which are under control of a second inducible promoter, e.g. a temperature sensitive promoter. In one embodiment, strains may comprise a combination of gene sequence(s), some of which are under control of a first inducible promoter, e.g., a FNR promoter and others which are under control of a second inducible promoter, e.g. a temperature sensitive promoter. In one embodiment, strains may comprise a combination of gene sequence(s), some of which are under control of a first inducible promoter, e.g., a chemically induced and others which are under control of a second inducible promoter, e.g. a temperature sensitive promoter. In some embodiments, strains may comprise one or more payload gene sequence(s) under the control of an FNR promoter and one or more payload gene sequence(s) under the control of a one or more promoter(s) which are induced by a one or more chemical and/or nutritional inducer(s), including, but not limited to, by arabinose, IPTG, rhamnose, tetracycline, and/or other chemical and/or nutritional inducers described herein or known in the art. Additionally the strains may comprise a construct which is under thermoregulatory control. In some embodiments, the bacteria strains further comprise payload sequence(s) under the control of one or more constitutive promoter(s) active under low oxygen conditions. Any of the strains described in these embodiments may be induced through the employment of phasing or cycling or pulsing or spiking techniques.

Aerobic Induction of the FNR Promoter

FNRS24Y is a mutated form of FNR which is more resistant to inactivation by oxygen, and therefore can activate FNR promoters under aerobic conditions (see e.g., Jervis AJ The O2 sensitivity of the transcription factor FNR is controlled by Ser24 modulating the kinetics of [4Fe-4S] to [2Fe-2S] conversion, Proc Natl Acad Sci USA. 2009 Mar. 24; 106(12):4659-64, the contents of which is herein incorporated by reference in its entirety). In some embodiments, an oxygen bypass system shown and described in figures and examples is used. In this oxygen bypass system, FNRS24Y is induced by addition of arabinose and then drives the expression of the protein of interest (e.g., one or more anti-cancer effector(s) described herein) by binding and activating the FNR promoter under aerobic conditions. Thus, strains can be grown, produced or manufactured efficiently under aerobic conditions, while being effectively pre-induced and pre-loaded, as the system takes advantage of the strong FNR promoter resulting in of high levels of expression of the protein of interest. This system does not interfere with or compromise in vivo activation, since the mutated FNRS24Y is no longer expressed in the absence of arabinose, and wild type FNR then binds to the FNR promoter and drives expression of the protein of interest, e.g., one or more gut barrier enhancer or anti-inflammatory effector(s) described herein.

In some embodiments, FNRS24Y is expressed during aerobic culture growth and induces a gene of interest. In other embodiments described herein, a second payload expression can also be induced aerobically, e.g., by arabinose. In a non-limiting example, a protein of interest and FNRS24Y can in some embodiments be induced simultaneously, e.g., from an arabinose inducible promoter. In some embodiments, FNRS24Y and the protein of interest are transcribed as a bicistronic message whose expression is driven by an arabinose promoter. In some embodiments, FNRS24Y is knocked into the arabinose operon, allowing expression to be driven from the endogenous Para promoter.

In some embodiments, a Lad promoter and IPTG induction are used in this system (in lieu of Para and arabinose induction). In some embodiments, a rhamnose inducible promoter is used in this system. In some embodiments, a temperature sensitive promoter is used to drive expression of FNRS24Y.

### Secretion

In any of the embodiments described herein, in which the genetically engineered organism, e.g., engineered bacteria, produces a protein, polypeptide, or peptide, DNA, RNA, small molecule or other molecule intended to be secreted from the microorganism, the engineered microorganism may comprise a secretion mechanism and corresponding gene sequence(s) encoding the secretion system.

In some embodiments, the recombinant bacteria further comprise a native secretion mechanism or non-native secretion mechanism that is capable of secreting the molecule from the bacterial cytoplasm in the extracellular environment. Many bacteria have evolved sophisticated secretion systems to transport substrates across the bacterial cell envelope. Substrates, such as small molecules, proteins, and DNA, may be released into the extracellular space or periplasm (such as the gut lumen or other space), injected into a target cell, or associated with the bacterial membrane.

In Gram-negative bacteria, secretion machineries may span one or both of the inner and outer membranes. In some embodiments, the recombinant bacteria further comprise a non-native double membrane-spanning secretion system. Double membrane-spanning secretion systems include, but are not limited to, the type I secretion system (T1SS), the type II secretion system (T2SS), the type III secretion system (T3SS), the type IV secretion system (T4SS), the type VI secretion system (T6SS), and the resistance-nodulation-division (RND) family of multi-drug efflux pumps (Pugsley 1993; Gerlach et al., 2007; Collinson et al., 2015; Costa et al., 2015; Reeves et al., 2015; WO2014138324A1, incorporated herein by reference). Examples of such secretion systems are shown in figures and examples. Mycobacteria, which have a Gram-negative-like cell envelope, may also encode a type VII secretion system (T7SS) (Stanley et al., 2003). With the exception of the T2SS, double membrane-spanning secretions generally transport substrates from the bacterial cytoplasm directly into the extracellular space or into the target cell. In contrast, the T2SS and secretion systems that span only the outer membrane may use a two-step mechanism, wherein substrates are first translocated to the periplasm by inner membrane-spanning transporters, and then transferred to the outer membrane or secreted into the extracellular space. Outer membrane-spanning secretion systems include, but are not limited to, the type V secretion or autotransporter system or autosecreter system (T5SS), the curli secretion system, and the chaperone-usher pathway for pili assembly (Saier, 2006; Costa et al., 2015).

In some embodiments in which the one or more proteins of interest, effector molecules or therapeutic proteins are secreted or exported from the microorganism, the engineered microorganism comprises gene sequence(s) that includes a secretion tag. In some embodiments, the one or more proteins of interest, effector molecules or therapeutic proteins include a “secretion tag” of either RNA or peptide origin to direct the one or more proteins of interest, effector molecules or therapeutic proteins to specific secretion systems. For example, a secretion tag for the Type I Hemolysin secretion system is encoded in the C-terminal 53 amino acids of the alpha hemolysin protein (HlyA).

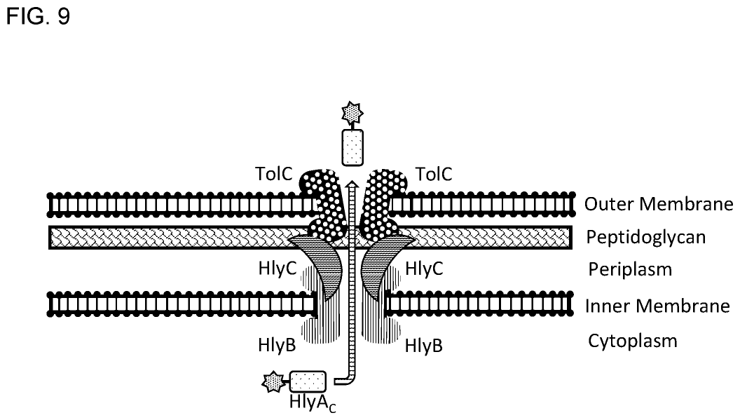

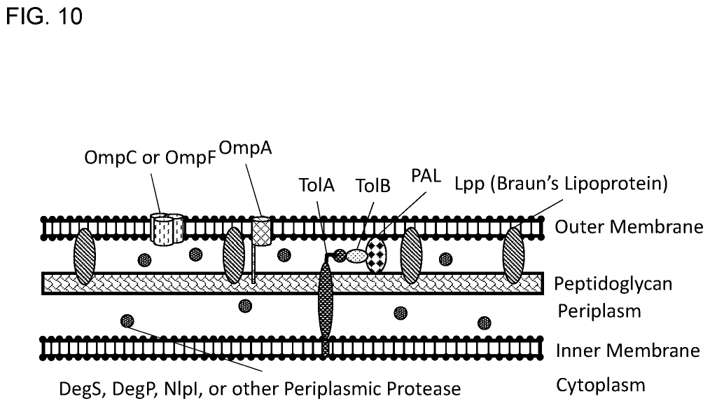

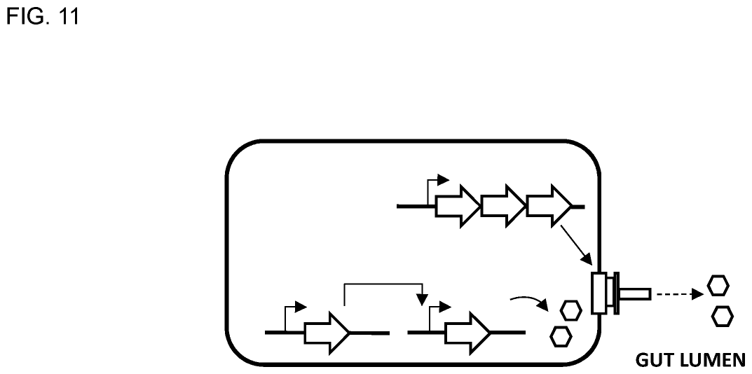

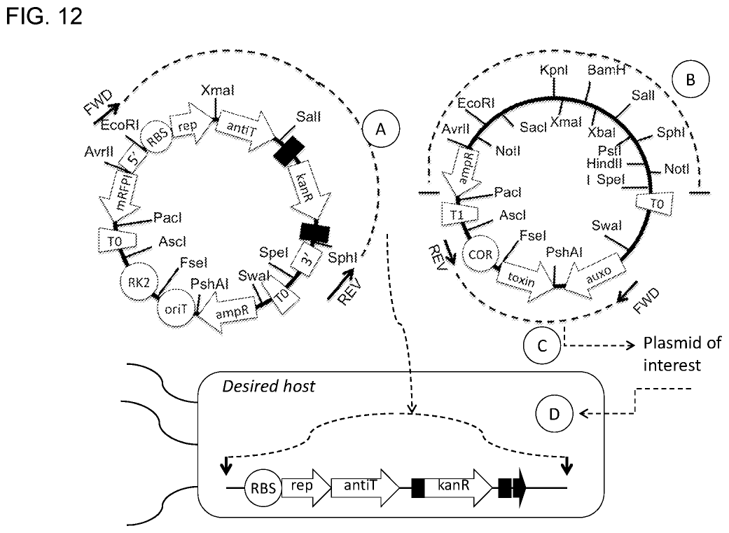

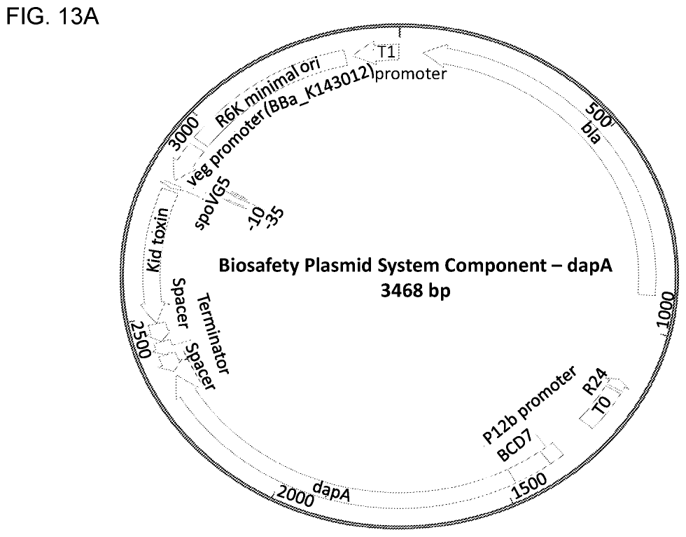

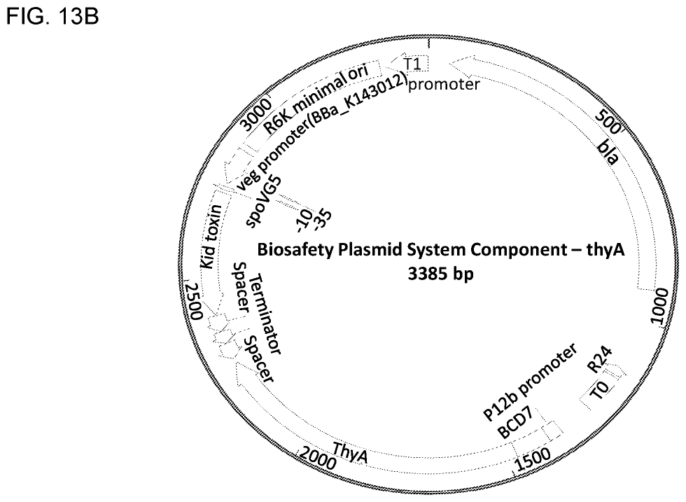

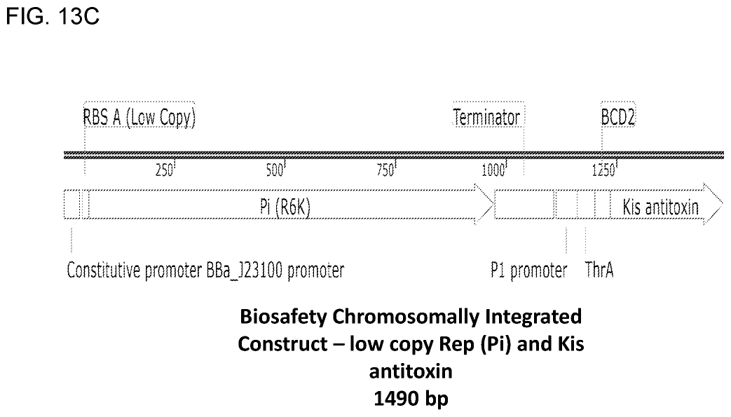

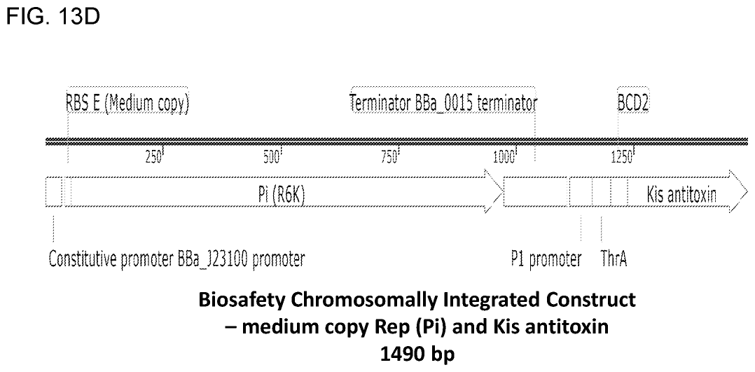

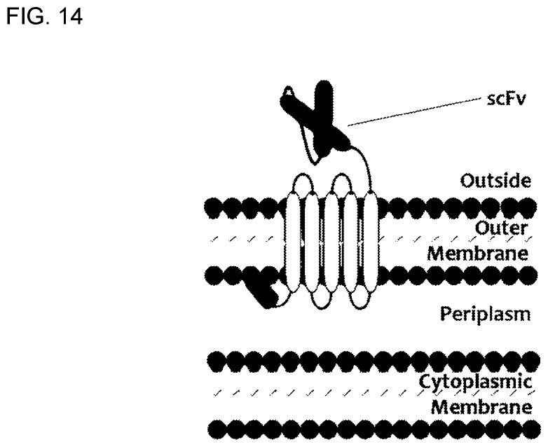

In some embodiments, a Hemolysin-based Secretion System is used to secrete the molecule of interest, e.g., therapeutic peptide. Type I Secretion systems offer the advantage of translocating their passenger peptide directly from the cytoplasm to the extracellular space, obviating the two-step process of other secretion types. FIG. 9 shows the alpha-hemolysin (HlyA) of uropathogenic Escherichia coli. This pathway uses HlyB, an ATP-binding cassette transporter; HlyD, a membrane fusion protein; and TolC, an outer membrane protein. The assembly of these three proteins forms a channel through both the inner and outer membranes. HlyB inserts into inner membrane to form a pore, HlyD aligns HlyB with TolC (outer membrane pore) thereby forming a channel through inner and outer membrane. Natively, this channel is used to secrete HlyA, however, to secrete the therapeutic peptide of the present disclosure, the secretion signal-containing C-terminal portion of HlyA is fused to the C-terminal portion of a therapeutic peptide (star) to mediate secretion of this peptide. The C-terminal secretion tag can be removed by either an autocatalytic or protease-catalyzed e.g., OmpT cleavage thereby releasing the one or more proteins of interest or therapeutic proteins into the extracellular milieu. In some embodiments the one or more proteins of interest or therapeutic proteins contain expressed as fusion protein with the 53 amino acids of the C termini of alpha-hemolysin (hlyA) of E. coli CFT073 (C terminal secretion tag).

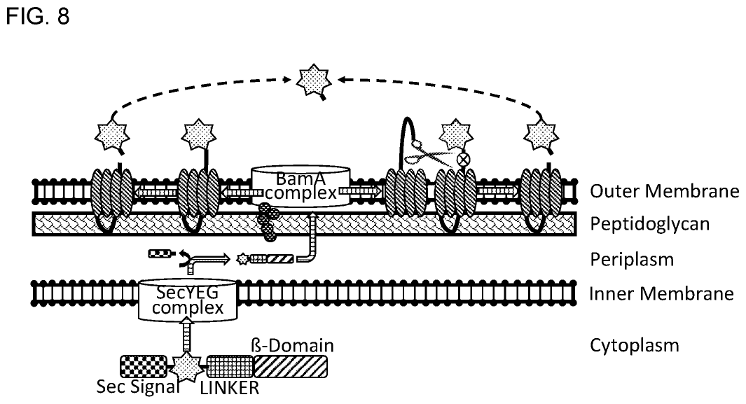

In some embodiments, a Type V Autotransporter Secretion System is used to secrete the molecule of interest, e.g., therapeutic peptide or effector molecule. The Type V Auto-secretion System utilizes an N-terminal Sec-dependent peptide tag (inner membrane) and C-terminal tag (outer-membrane). This system uses the Sec-system to get from the cytoplasm to the periplasm. The C-terminal tag then inserts into the outer membrane forming a pore through which the “passenger protein” threads through. Due to the simplicity of the machinery and capacity to handle relatively large protein fluxes, the Type V secretion system is attractive for the extracellular production of recombinant proteins. As shown in FIG. 8, a therapeutic peptide or effector molecule (star) can be fused to an N-terminal secretion signal, a linker, and the beta-domain of an autotransporter. The N-terminal, Sec-dependent signal sequence directs the protein to the SecA-YEG machinery which moves the protein across the inner membrane into the periplasm, followed by subsequent cleavage of the signal sequence. The Beta-domain is recruited to the Bam complex (‘Beta-barrel assembly machinery′) where the beta-domain is folded and inserted into the outer membrane as a beta-barrel structure. The therapeutic peptide or effector molecule is threaded through the hollow pore of the beta-barrel structure ahead of the linker sequence. Once across the outer membrane, the passenger is released from the membrane-embedded C-terminal tag by either an autocatalytic, intein-like mechanism (left side of Bam complex) or via a membrane-bound protease (black scissors; right side of Bam complex) (i.e., OmpT). For example, a membrane-associated peptidase to a complimentary protease cut site in the linker. Thus, in some embodiments, the secreted molecule, such as a heterologous protein or peptide comprises an N-terminal secretion signal, a linker, and beta-domain of an autotransporter so as to allow the molecule to be secreted from the bacteria.

The N-terminal tag is removed by the Sec system. Thus, in some embodiments, the secretion system is able to remove this tag before secreting the one or more proteins of interest, effector molecule or therapeutic proteins, from the engineered bacteria. In the Type V auto-secretion-mediated secretion the N-terminal peptide secretion tag is removed upon translocation of the “passenger” peptide from the cytoplasm into the periplasmic compartment by the native Sec system. Further, once the auto-secretor is translocated across the outer membrane the C-terminal secretion tag can be removed by either an autocatalytic or protease-catalyzed e.g., OmpT cleavage thereby releasing the molecule(s) into the extracellular milieu.

In some embodiments, the recombinant bacteria comprise a type III or a type III-like secretion system (T3SS) from Shigella, Salmonella, E. coli, Bivrio, Burkholderia, Yersinia, Chlamydia, or Pseudomonas. The traditional T3SS is capable of transporting a protein from the bacterial cytoplasm to the host cytoplasm through a needle complex. In the Type III traditional secretion system, the basal body closely resembles the flagella, however, instead of a “tail”/whip, the traditional T3SS has a syringe to inject the passenger proteins into host cells. The secretion tag is encoded by an N-terminal peptide (lengths vary and there are several different tags, see PCT/US14/020972). The N-terminal tag is not removed from the polypeptides in this secretion system.

The T3SS may be modified to secrete the molecule from the bacterial cytoplasm, but not inject the molecule into the host cytoplasm. Thus, the molecule is secreted into the gut lumen, tumor microenvironment, or other extracellular space. In some embodiments, the recombinant bacteria comprise said modified T3SS and are capable of secreting the molecule of interest from the bacterial cytoplasm. In some embodiments, the secreted molecule, such as a heterologous protein or peptide comprises a type III secretion sequence that allows the molecule of interest to be secreted from the bacteria.

In the Flagellar modified Type III Secretion, the tag is encoded in 5′-untranslated region of the mRNA and thus there is no peptide tag to cleave/remove. This modified system does not contain the “syringe” portion and instead uses the basal body of the flagella structure as the pore to translocate across both membranes and out through the forming flagella. If the fliC/fliD genes (encoding the flagella “tail”/whip) are disrupted the flagella cannot fully form and this promotes overall secretion. In some embodiments, the tail portion can be removed entirely.

In some embodiments, a flagellar type III secretion pathway is used to secrete the molecule of interest. In some embodiments, an incomplete flagellum is used to secrete a therapeutic peptide of interest by recombinantly fusing the peptide to an N-terminal flagellar secretion signal of a native flagellar component. In this manner, the intracellularly expressed chimeric peptide can be mobilized across the inner and outer membranes into the surrounding host environment.

For example, a modified flagellar type III secretion apparatus in which untranslated DNA fragment upstream of the gene fliC (encoding flagellin), e.g., a 173-bp region, is fused to the gene encoding the heterologous protein or peptide can be used to secrete polypeptides of interest (See, e.g., Majander et al., Extracellular secretion of polypeptides using a modified Escherichia coli flagellar secretion apparatus. Nat Biotechnol. 2005 April; 23(4):475-81). In some cases, the untranslated region from the fliC loci may not be sufficient to mediate translocation of the passenger peptide through the flagella. Here it may be necessary to extend the N-terminal signal into the amino acid coding sequence of FliC, for example, by using the 173 bp of untranslated region along with the first 20 amino acids of FliC (see, e.g., Duan et al., Secretion of Insulinotropic Proteins by Commensal Bacteria: Rewiring the Gut To Treat Diabetes, Appl. Environ. Microbiol. December 2008 vol. 74 no. 23 7437-7438).

In alternate embodiments, the recombinant bacteria further comprise a non-native single membrane-spanning secretion system. Single membrane-spanning transporters may act as a component of a secretion system, or may export substrates independently. Such transporters include, but are not limited to, ATP-binding cassette translocases, flagellum/virulence-related translocases, conjugation-related translocases, the general secretory system (e.g., the SecYEG complex in E. coli), the accessory secretory system in mycobacteria and several types of Gram-positive bacteria (e.g., Bacillus anthracis, Lactobacillus johnsonii, Corynebacterium glutamicum, Streptococcus gordonii, Staphylococcus aureus), and the twin-arginine translocation (TAT) system (Saier, 2006; Rigel and Braunstein, 2008; Albiniak et al., 2013). It is known that the general secretory and TAT systems can both export substrates with cleavable N-terminal signal peptides into the periplasm, and have been explored in the context of biopharmaceutical production. The TAT system may offer particular advantages, however, in that it is able to transport folded substrates, thus eliminating the potential for premature or incorrect folding. In certain embodiments, the recombinant bacteria comprise a TAT or a TAT-like system and are capable of secreting the molecule of interest from the bacterial cytoplasm. One of ordinary skill in the art would appreciate that the secretion systems disclosed herein may be modified to act in different species, strains, and subtypes of bacteria, and/or adapted to deliver different payloads.

In order to translocate a protein, e.g., therapeutic polypeptide, to the extracellular space, the polypeptide must first be translated intracellularly, mobilized across the inner membrane and finally mobilized across the outer membrane. Many effector proteins (e.g., therapeutic polypeptides)—particularly those of eukaryotic origin—contain disulphide bonds to stabilize the tertiary and quaternary structures. While these bonds are capable of correctly forming in the oxidizing periplasmic compartment with the help of periplasmic chaperones, in order to translocate the polypeptide across the outer membrane the disulphide bonds must be reduced and the protein unfolded again.

One way to secrete properly folded proteins in gram-negative bacteria—particularly those requiring disulphide bonds—is to target the reducing-environment periplasm in conjunction with a destabilizing outer membrane. In this manner the protein is mobilized into the oxidizing environment and allowed to fold properly. In contrast to orchestrated extracellular secretion systems, the protein is then able to escape the periplasmic space in a correctly folded form by membrane leakage. These “leaky” gram-negative mutants are therefore capable of secreting bioactive, properly disulphide-bonded polypeptides. In some embodiments, the recombinant bacteria have a “leaky” or de-stabilized outer membrane. Destabilizing the bacterial outer membrane to induce leakiness can be accomplished by deleting or mutagenizing genes responsible for tethering the outer membrane to the rigid peptidoglycan skeleton, including for example, lpp, ompC, ompA, ompF, tolA, tolB, pal, degS, degP, and nlpl. Lpp is the most abundant polypeptide in the bacterial cell existing at ˜500,000 copies per cell and functions as the primary ‘staple’ of the bacterial cell wall to the peptidoglycan. 1.

Silhavy, T. J., Kahne, D. & Walker, S. The bacterial cell envelope. Cold Spring Harb Perspect Biol 2, a000414 (2010). TolA-PAL and OmpA complexes function similarly to Lpp and are other deletion targets to generate a leaky phenotype. Additionally, leaky phenotypes have been observed when periplasmic proteases are inactivated. The periplasm is very densely packed with protein and therefore encode several periplasmic proteins to facilitate protein turnover. Removal of periplasmic proteases such as degS, degP or nlpl can induce leaky phenotypes by promoting an excessive build-up of periplasmic protein. Mutation of the proteases can also preserve the effector polypeptide by preventing targeted degradation by these proteases. Moreover, a combination of these mutations may synergistically enhance the leaky phenotype of the cell without major sacrifices in cell viability. Thus, in some embodiments, the engineered bacteria have one or more deleted or mutated membrane genes. In some embodiments, the engineered bacteria have a deleted or mutated lpp gene. In some embodiments, the engineered bacteria have one or more deleted or mutated gene(s), selected from ompA, ompA, and ompF genes. In some embodiments, the engineered bacteria have one or more deleted or mutated gene(s), selected from tolA, tolB, and pal genes. In some embodiments, the engineered bacteria have one or more deleted or mutated periplasmic protease genes. In some embodiments, the engineered bacteria have one or more deleted or mutated periplasmic protease genes selected from degS, degP, and nlpl. In some embodiments, the engineered bacteria have one or more deleted or mutated gene(s), selected from lpp, ompA, ompF, tolA, tolB, pal, degS, degP, and nlpl genes.

To minimize disturbances to cell viability, the leaky phenotype can be made inducible by placing one or more membrane or periplasmic protease genes, e.g., selected from lpp, ompA, ompF, tolA, tolB, pal, degS, degP, and nlpl, under the control of an inducible promoter. For example, expression of lpp or other cell wall stability protein or periplasmic protease can be repressed in conditions where the therapeutic polypeptide needs to be delivered (secreted). For instance, under inducing conditions a transcriptional repressor protein or a designed antisense RNA can be expressed which reduces transcription or translation of a target membrane or periplasmic protease gene. Conversely, overexpression of certain peptides can result in a destabilized phenotype, e.g., overexpression of colicins or the third topological domain of TolA, wherein peptide overexpression can be induced in conditions in which the therapeutic polypeptide needs to be delivered (secreted). These sorts of strategies would decouple the fragile, leaky phenotypes from biomass production. Thus, in some embodiments, the engineered bacteria have one or more membrane and/or periplasmic protease genes under the control of an inducible promoter.

Table 9 and Table 10 below lists secretion systems for Gram positive bacteria and Gram negative bacteria.

TABLE 9
Secretion systems for gram positive bacteria

| Bacterial Strain | Relevant Secretion System |
|---|---|
| C. novyi-NT (Gram+) | Sec pathway |
| | Twin- arginine (TAT) pathway |
| C. butryicum (Gram+) | Sec pathway |
| | Twin- arginine (TAT) pathway |
| Listeria monocytogenes (Gram +) | Sec pathway |
| | Twin- arginine (TAT) pathway |

TABLE 10
Secretion Systems for Gram negative bacteria
Protein secretary pathways (SP) in gram-negative bacteria and their descendants

| | | | | | | | |
|---|---|---|---|---|---|---|---|
| Type | | | | | | # | Energy |
| (Abbrev.) | Name | TC#² | Bacteria | Archaea | Eukarya | Proteins/System | Source |

IMPS - Gram-negative bacterial inner membrane channel-forming translocases

| | | | | | | | |
|---|---|---|---|---|---|---|---|
| ABC | ATP binding | 3.A.1 | + | + | + | 3-4 | ATP |
| (SIP) | cassette translocase | | | | | | |
| SEC | General secretory | 3.A.5 | + | + | + | ~12 | GTP OR |
| (IISP) | translocase | | | | | | ATP + PMF |
| Fla/Path | Flagellum/virulence- | 3.A.6 | + | − | − | >10 | ATP |
| (IIISP) | related translocase | | | | | | |
| Conj | Conjugation-related | 3.A.7 | + | − | − | >10 | ATP |
| (IVSP) | translocase | | | | | | |
| Tat (IISP) | Twin-arginine | 2.A.64 | + | + | + | 2-4 | PMF |
| | targeting translocase | | | | (chloroplasts) | | |
| Oxal | Cytochrome oxidase | 2.A.9 | + | + | + | 1 | None or |
| (YidC) | biogenesis family | | | | (mitochondria | | PMF |
| | | | | | chloroplasts) | | |
| MscL | Large conductance | 1.A.22 | + | + | + | 1 | None |
| | mechanosensitive | | | | | | |
| | channel family | | | | | | |
| Holins | Holin functional | 1.E.1•21 | + | − | − | 1 | None |
| | superfamily | | | | | | |

Eukaryotic Organelles

| | | | | | | | |
|---|---|---|---|---|---|---|---|
| MPT | Mitochondrial | 3.A.B | − | − | + | >20 | ATP |
| | protein translocase | | | | (mitochondrial) | | |
| CEPT | Chloroplast envelope | 3.A.9 | (+) | − | + | ≥3 | GTP |
| | protein translocase | | | | (chloroplasts) | | |
| Bcl-2 | Eukaryotic Bcl-2 family | 1.A.21 | − | − | + | 1? | None |
| | (programmed cell death) | | | | | | |

Gram-negative bacterial outer membrane channel-forming translocases

| | | | | | | | |
|---|---|---|---|---|---|---|---|
| MTB | Main terminal branch of | 3.A.15 | +^{b} | − | − | ~14 | ATP; |
| (IISP) | the general secretory | | | | | | PMF |
| | translocase | | | | | | |
| FUP AT-1 | Fimbrial usher protein | 1.B.11 | +^{b} | − | − | 1 | None |
| | Autotransporter-1 | 1.B.12 | +^{b} | | − | 1 | None |
| AT-2 | Autotransporter-2 | 1.B.40 | +^{b} | − | − | 1 | None |
| OMF | | 1.B.17 | +^{b} | | +(?) | 1 | None |
| (ISP) | | | | | | | |
| TPS | | .B.20 | + | − | + | 1 | None |
| Secretin | | .B.22 | +^{b} | | − | 1 | None |
| (IISP and IISP) | | | | | | | |
| OmpIP | Outer membrane | 1.B.33 | + | − | + | ≥4 | None |
| | insertion porin | | | | (mitochondria; | | |
| | | | | | chloroplasts) | | |

The above tables for gram positive and gram negative bacteria list secretion systems that can be used to secrete polypeptides and other molecules from the engineered bacteria (Milton H. Saier, Jr. Microbe/Volume 1, Number 9, 2006 “Protein Secretion Systems in Gram-Negative Bacteria Gram-negative bacteria possess many protein secretion-membrane insertion systems that apparently evolved independently”, the contents of which is herein incorporated by reference in its entirety).

In some embodiments, the recombinant bacterial comprise a native or non-native secretion system described herein for the secretion of a molecule, e.g., a cytokine, antibody (e.g., scFv), metabolic enzyme, e.g., kynureninase, an others described herein.

Polypeptide Sequences of exemplary secretion tags include PhoA (SEQ ID NO: 385), PhoA (SEQ ID NO: 386), OmpF (SEQ ID NO: 387), cvaC (SEQ ID NO: 388), TorA (SEQ ID NO: 389), fdnG (SEQ ID NO: 390), dmsA (SEQ ID NO: 391), PelB (SEQ ID NO: 392), HlyA secretion signal (SEQ ID NO: 393), and HlyA secretion signal (SEQ ID NO: 394). In some embodiments, secretion tags of endogenous secreted proteins from E. coli can be used to secrete the protein of interest. Exemplary secretion tags from secreted E coli Nissle include ECOLIN_05715 Secretion signal (SEQ ID NO: 395), ECOLIN_16495 Secretion signal (SEQ ID NO: 396), ECOLIN_19410 Secretion signal (SEQ ID NO:397), and ECOLIN_19880 Secretion signal (SEQ ID NO:398). Additional secretion tags include adhesion (SEQ ID NOS: 1091 and 1099), DsbA (SEQ ID NOS: 1092 and 1100), GltI (SEQ ID NOS: 1093 and 1101), GspD (SEQ ID NOS: 1089 and 1102), HdeB (SEQ ID NOS: 1090 and 1103), MalE (SEQ ID NOS: 1094 and 1104), OppA (SEQ ID NOS: 1095 and 1105), PelB (SEQ ID NOS: 1096 and 1106), PhoA (SEQ ID NOS: 1097 and 1107) and PpiA (SEQ ID NOS: 1098 and 1108).

In some embodiments, recombinant bacteria comprise a nucleic acid sequence that encodes a polypeptide which is at least about 80%, 85%, 90%, 95%, or 99% homologous to one or more of the sequences of SEQ ID NOS: 385-398 and 1089-1108, or a nucleic acid sequence which is at least about 80%, 85%, 90%, 95%, or 99% homologous to one or more of the sequences of SEQ ID NOS: 385-398 and 1089-1108. Any secretion tag or secretion system can be combined with any cytokine described herein, and can be used to generate a construct (plasmid based or integrated) which is driven by an directly or indirectly inducible or constitutive promoter described herein. In some embodiments, the secretion system is used in combination with one or more genomic mutations, which leads to the leaky or diffusible outer membrane phenotype (DOM), including but not limited to, lpp, nlP, tolA, PAL.

In some embodiments, the secretion system is selected from the type III flagellar, modified type III flagellar, type I (e.g., hemolysin system), type II, type IV, type V, type VI, and type VII secretion systems, resistance-nodulation-division (RND) multi-drug efflux pumps, a single membrane secretion system, Sec and, TAT secretion systems.

Any of the secretion systems described herein may according to the disclosure be employed to secrete the polypeptides of interest. In some embodiments, the therapeutic proteins secreted by the recombinant bacteria are modified to increase resistance to proteases, e.g. intestinal proteases.

In some embodiments, the genetically engineered microorganisms are capable of expressing any one or more of the described circuits in low-oxygen conditions, and/or in the gut, or tissue specific molecules or metabolites, and/or in the presence of molecules or metabolites associated with inflammation or immune suppression, and/or in the presence of metabolites that may be present in the gut, and/or in the presence of metabolites that may or may not be present in vivo, and may be present in vitro during strain culture, expansion, production and/or manufacture, such as arabinose and others described herein. In some embodiments, the gene sequences(s) are controlled by a promoter inducible by such conditions and/or inducers. In some embodiments, the gene sequences(s) are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein.

In some embodiments, any one or more of the described circuits are present on one or more plasmids (e.g., high copy or low copy) or are integrated into one or more sites in the microorganisms chromosome. Also, in some embodiments, the genetically engineered microorganisms are further capable of expressing any one or more of the described circuits and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art and (7) one or more circuits for the production or degradation of one or more metabolites described herein (8) combinations of one or more of such additional circuits.

Non-limiting examples of proteins of interest are described herein. These polypeptides may be mutated to increase stability, resistance to protease digestion, and/or activity.

TABLE 11
Comparison of Secretion systems for secretion of polypeptide from engineered bacteria

| Secretion System | Tag | Cleavage | Advantages | Other features |
|---|---|---|---|---|
| Modified Type III | mRNA (or | No cleavage | No peptide | May not be as suited for |
| (flagellar) | N-terminal) | necessary | tag | larger proteins |
| | | | Endogenous | Deletion of flagellar genes |
| Type V | N- and C- | Yes | Large proteins | 2-step secretion |
| autotransport | terminal | | Endogenous | |
| | | | Cleavable | |
| Type I | C-terminal | No | | Tag; Exogenous Machinery |
| Diffusible Outer | N-terminal | Yes | Disulfide bond | May affect cell |
| Membrane (DOM) | | | formation | fragility/survivability/ |
| | | | | growth/yield |

In some embodiments, the therapeutic polypeptides of interest are secreted using components of the flagellar type III secretion system. In a non-limiting example, such a therapeutic polypeptide of interest are secreted via Type I Hemolysin Secretion, is assembled behind a fliC-5′UTR (e.g., 173-bp untranslated region from the fliC loci), and is driven by the native promoter. In other embodiments, the expression of the therapeutic peptide of interested secreted using components of the flagellar type III secretion system is driven by a tet-inducible promoter. In alternate embodiments, an inducible promoter such as oxygen level-dependent promoters (e.g., FNR-inducible promoter), promoters induced by IBD specific molecules or promoters induced by inflammation or an inflammatory response (RNS, ROS promoters), and promoters induced by a metabolite that may or may not be naturally present (e.g., can be exogenously added) in the gut, e.g., arabinose is used. In some embodiments, the therapeutic polypeptide of interest is expressed from a plasmid (e.g., a medium copy plasmid). In some embodiments, the therapeutic polypeptide of interest is expressed from a construct which is integrated into fliC locus (thereby deleting fliC), where it is driven by the native FliC promoter. In some embodiments, an N terminal part of FliC (e.g., the first 20 amino acids of FliC) is included in the construct, to further increase secretion efficiency.

In some embodiments, the therapeutic polypeptides of interest are secreted via Type I Hemolysin Secretion, are secreted using via a diffusible outer membrane (DOM) system. In some embodiments, the therapeutic polypeptide of interest is fused to a N-terminal Sec-dependent secretion signal. Non-limiting examples of such N-terminal Sec-dependent secretion signals include PhoA, OmpF, OmpA, and cvaC. In alternate embodiments, the therapeutic polypeptide of interest is fused to a Tat-dependent secretion signal. Exemplary Tat-dependent tags include TorA, FdnG, and DmsA.

In certain embodiments, the recombinant bacteria comprise deletions or mutations in one or more of the outer membrane and/or periplasmic proteins. Non-limiting examples of such proteins, one or more of which may be deleted or mutated, include lpp, pal, tolA, and/or nlpl. In some embodiments, lpp is deleted or mutated. In some embodiments, pal is deleted or mutated. In some embodiments, tolA is deleted or mutated. In other embodiments, nlpl is deleted or mutated. In yet other embodiments, certain periplasmic proteases are deleted or mutated, e.g., to increase stability of the polypeptide in the periplasm. Non-limiting examples of such proteases include degP and ompT. In some embodiments, degP is deleted or mutated. In some embodiments, ompT is deleted or mutated. In some embodiments, degP and ompT are deleted or mutated.

In some embodiments, the therapeutic polypeptides of interest are secreted via Type I Hemolysin Secretion, are secreted via a Type V Auto-secretor (pic Protein) Secretion. In some embodiments, the therapeutic protein of interest is expressed as a fusion protein with the native Nissle auto-secreter E. coli_01635 (where the original passenger protein is replaced with the therapeutic polypeptides of interest.

In some embodiments, the therapeutic polypeptides of interest are secreted via Type I Hemolysin Secretion, are secreted via Type I Hemolysin Secretion. In one embodiment, therapeutic polypeptide of interest is expressed as fusion protein with the 53 amino acids of the C terminus of alpha-hemolysin (hlyA) of E. coli CFT073.

### Surface Display

In some embodiments, the recombinant bacteria and/or microorganisms encode one or more gene(s) and/or gene cassette(s) encoding an anti-cancer molecule which is anchored or displayed on the surface of the bacteria and/or microorganisms. Examples of the anti-cancer molecules which are displayed or anchored to the bacteria and/or microorganism, are any of the anti-cancer molecules described herein, and include but are not limited to antibodies, e.g., scFv fragments, and tumor-specific antigens or neoantigens. In a non-limiting example, the antibodies or scFv fragments which are anchored or displayed on the bacterial cell surface are directed against checkpoint inhibitors described herein, including, but not limited to, CLTLA4, PD-1, PD-L1.

In some embodiments, the recombinant bacteria comprise one or more gene sequence(s) encoding therapeutic polypeptide or effector molecule, e.g., a ScFv, which is anchored or displayed on the surface of the bacteria, and which remains anchored while exerting its effector function. In other embodiments, the recombinant bacteria encoding the surface-displayed therapeutic polypeptide, e.g., the antibodies or scFv fragments, lyse before, during or after exerting their effector function. In some embodiments, the recombinant bacteria encode a therapeutic peptide that is temporarily attached to the cell surface and which dissociates from the bacterium before, during, or after exerting its function.

In some embodiments, shorter peptides or polypeptides, e.g. peptides or polypeptides of less than 60 amino acids of length, are displayed on the cell surface of the recombinant bacteria. In some embodiments, such shorter peptides or polypeptides comprise a immune modulatory effector molecule. Non-limiting examples of such therapeutic polypeptides are described herein.

Several strategies for the display of shorter peptides or polypeptides on the surface of gram negative bacteria are known in the art, and are for example described in Georgiou et al., Display of heterologous proteins on the surface of microorganisms: from the screening of combinatorial libraries to live recombinant vaccines: Nat Biotechnol. 1997 January; 15(1):29-34, the contents of which is herein incorporated by reference in its entirety. These systems all share a common theme, targeting recombinant proteins to the cell surface by the construction of gene fusions using sequences from membrane-anchoring domains of surface proteins.

Non-limiting examples of such strategies are described in Tables 12-14.

TABLE 12
Exemplary Cell Surface Display Strategies

| | Exemplary | | Localization of |
|---|---|---|---|
| | carrier | | heterologous |
| Carrier protein | organism | Type of fusion | polypeptide |
| LamB | E. coli | Sandwich fusion | Cell surface |
| PhoE | E. coli | Sandwich fusion | Cell surface |
| OprF | Pseudomonas | Sandwich fusion | Cell surface |
| Gram negative | E. coli | C-terminal or | Periplasmic side or |
| lipoproteins | | sandwich fusion | outer membrane/ |
| | | | Cell surface |
| Lpp-OmpA | E. coli | C-terminal fusion | Cell surface |
| VirG | Shigella | N-terminal | Cell surface |
| | | fusion | |
| IgA | Neisseria | N-terminal | Cell surface |
| | | fusion | |
| Flagellin (FliC) | E. coli | Sandwich fusion | Cell surface |
| Flagellin (FliC) | E. coli | Sandwich fusion | Cell surface |
| FimH (type I | E. coli | Sandwich fusion | Cell surface |
| pili) | | | |
| PapA (Pap pili) | E. coli | Sandwich fusion | Cell surface |
| PulA | Klebsiella | C-terminal fusion | Cell surface/ |
| | | | extracellular fluid |

TABLE 13
Exemplary Cell Surface Display Strategies

| | |
|---|---|
| Carrier | Passenger size |

Outer membrane Proteins

| | | |
|---|---|---|
| OmpA | 15-514 | aa |
| OmprF | 17-43 | aa |
| LamB | 11-232 | aa |
| OmpS | 38-115 | aa |
| OmpC | 162 | aa |
| PhoE | 8-32 | aa |
| Invasin | 18 | aa |
| LppOmpA | < or =40 | kDa |

Lipoproteins

| TraT | 11-98 | aa |
|---|---|---|
| PAL | Approx.. 250 aa | |
| OprI | 16 | aa |

| | |
|---|---|
| Inp | Less than or equal 47 kDa |

Autotransporters

| Igabeta | 12 | kDa |
|---|---|---|
| VirGbeta | Approx.. 50 kDa | |
| AIDA-1 | 12-40 | kDa |

Secreted

Pullulanase

Subunits of Surface Appendages

| | | |
|---|---|---|
| Flagellae | 11-115 | aa |
| Fimbriae | 7-52 | aa |

S-layer proteins

| | | |
|---|---|---|
| RsaA | 12 | aa |

TABLE 14
Exemplary Cell Surface Strategies

| | | |
|---|---|---|
| | | Passenger |
| Outer membrane protein | Type of fusion | size (kDa) |

Outer membrane protein

| | | |
|---|---|---|
| eCPX derived from OmpX | Biterminal | 0.8-1.6 |
| FhuA | Insertional | 1.1-3.3 |
| LamB | Insertional | 1.2-25.5 |
| Omp1 | C-terminal | 56 |
| OmpA | Insertional | 1-50 |
| OmpC | Insertional, | 18-52 |
| | C-terminal | |
| OmpT | | 35 |
| OprF | C-terminal | 50 |
| PgsA | C-terminal | 34-77 |
| Wza-omp orf1/OmpU/Omp26La | C-terminal | 27-50 |

Surface Appendages

| | | |
|---|---|---|
| F Pillin | Insertional | 1.6 |
| Fimbria (FimH and FimA) | Insertional | 1-4 |
| Flagellin (FliC and FliD) | Insertional | 1.2-33 |

Lipoproteins

| | | |
|---|---|---|
| INP | C-terminal | 7-119 |
| Lpp = OmpA | C-terminal | 27-74 |
| PAL | N-terminal | 29 |
| Tat-dependent lipoprotein | C-terminal | 27 |
| TraT | Insertional, C-terminal | 1.2-11 |

Virulence Factors

| | | |
|---|---|---|
| AIDA-1 | N-terminal | 12-65 |
| EaeA | C-terminal | 3.9-31.6 |
| EspP | N-terminal | 20 |
| EstA | N-terminal | 38-60 |
| Invasin | C-terminal | 1.1 |
| MSP1a | N-terminal | 4.6 |

In some embodiments, the recombinant bacteria comprise one or more gene sequence(s) encoding one or more short therapeutic peptides or polypeptides fused into surface exposed loops of outer membrane proteins (OMPs), e.g., from enteric bacteria. In a non-limiting example, the short therapeutic peptides or polypeptides expressed by the recombinant bacteria are inserted into the outer membrane protein LamB, e.g., from E. coli, and displayed on the bacterial cell surface. Extracellular display of peptides through Insertion of peptides into surface exposed loops of LamB is for example described in Hofnung et al., Methods Cell Biol. 34:77-105, and Charbit, A. et al., 1987. J. Immunol. 139:1658-1664.

In another non-limiting example, the short therapeutic peptides or polypeptides encoded by one or more gene sequence(s) comprised in the recombinant bacteria are inserted into the outer membrane protein PhoE, e.g., from E. coli, and displayed on the bacterial cell surface. The PhoE protein is another abundant outer membrane protein of E. coli K-12, which has a trimeric structure and functions as a pore for small molecules. Analysis of the primary structure of PhoE revealed 16 beta sheets which traverse through the membranes, and eight hypervariable regions exposed at the surface of the cell. One or more of these cell surface exposed regions of PhoE protein can be used to insert heterologous peptides. For example, antigenic determinants of pathogenic organisms have been presented in one or more cell surface exposed regions of PhoE protein (e.g., as described in Aterberg et al., 1990; Vaccine. 1990 February; 8(1):85-91).

In some embodiments, the recombinant bacteria comprise one or more gene sequence(s) encoding one or more short therapeutic peptides or polypeptides fused to protein components of extracellular appendages. Several systems have been described, in which extracellular appendages, such as pili and flagella are used to display peptides of interest at the bacterial cell surface. Examples of flagellar and pilar proteins used include FliC, a major structural component of the E. coli flagellum, and PapA, the major subunit of the Pap pilus. In one embodiment, the recombinant bacteria comprise one or more gene sequence(s) encoding one or more components of a FLITRX system. The FLITRX system is an E. coli display system based on the use of fusion protein of FliC and thioredoxin, a small redox protein which represents a highly versatile scaffold that allows peptide inserts to assume a confirmation compatible with binding to other proteins. In the FLITRX system, thioredoxin is fused into a dispensable region of FliC. Then, heterologous peptides can be inserted within the thioredoxin domain in the FliC fusion, and are surface exposed. Other scaffolding proteins are known in the art, some of which may replace thioredoxin as a scaffolding protein in this system.

In some embodiments, the recombinant bacteria comprise a FimH fusion protein, in which the therapeutic peptide of interest is fused to FimH, an adhesin of type 1 fimbriae, e.g., from E. coli. FimH adhesin chimeras containing as many as 56 foreign amino acids in certain positions are transported to the bacterial surface as components of the fimbrial organelles (Pallesen et al., Microbiology 141: 2839-2848).

In some embodiments, the recombinant bacteria comprise one or more gene sequence(s) encoding a fusion protein in which the therapeutic peptide of interest is fused to the major subunit of F11 fimbriae, e.g., from E. coli. Hypervariable regions of the major subunit of F11 fimbriae can be used for insertion of heterologous peptides, e.g., antigenic epitopes (Van Die et al., Mol. Gen. Genet. 222: 297-303).

In one embodiment, the recombinant bacteria comprise one or more gene sequence(s) encoding a papA fusion protein, in which the therapeutic peptide of interest is fused to papA. In some embodiments, peptides of interest are inserted following either codon 7 or 68 of the coding sequence for the mature portion of PapA, as peptides in the area of amino acids 7 and 68 of PapA are localized at the external side of the pilus (Steidler et al., J Bacteriol. 1993 December; 175(23):7639-43).

In some embodiments, the recombinant bacteria comprise one or more gene sequence(s), which encode polypeptides larger than 60 amino acids, e g, immune modulatory effector, and which are displayed on the bacterial cell surface. In some embodiments, the recombinant bacteria comprise one or more gene sequence(s), which encode a fusion protein, in which a therapeutic peptide of interest, e.g., a polypeptide greater than 60 amino acids in length, is fused to a lipoprotein from a gram negative bacterium, or one or more fragments thereof.

In one embodiment, the recombinant bacteria comprise one or more gene sequence(s), which encode a fusion protein, in which a therapeutic protein of interest is fused to peptidoglycan associated lipoprotein (PAL) or a fragment thereof. The fusion protein in located in the periplasm and can be displayed externally upon permeablization of the outer membrane. For example, a PAL-scFv fusion protein was shown to bind its antigen and to be tightly bound to the murein layer of the cell envelope (Fuchs et al., Biotechnology (N Y). 1991 December; 9(12):1369-72). The PAL-scFv fusion was located in the periplasm and bound to the murein layer, and after permeabilization of the outer membrane, the scFv became accessible to externally added antigen. In some embodiments, the recombinant bacteria comprising a fusion protein for surface display further have a permeable outer membrane. Mutations and/or deletions resulting in a leaky outer membrane are described elsewhere herein.

In one embodiment, the recombinant bacteria encode a fusion protein, in which a therapeutic protein of interest, e.g., a immune modulatory effector, is fused to residues of the major lipoprotein of a gram negative bacterium, e.g., E. coli. In one embodiment, the recombinant bacteria encode a fusion protein, in which a therapeutic protein of interest, is fused to the signal peptide and the nine N-terminal amino acid residues of the major lipoprotein of a gram negative bacterium, e.g., E. coli. These residues of the E. coli major lipoprotein function as a hydrophobic membrane anchor. For example, a fusion construct of these residues with a therapeutic polypeptide, in this case a scFv fragment, resulted in specific accumulation of an immunoreactive and cell-bound polypeptide in E. coli (Laukkanen et al., Mol. Microbiol. 4:1259-1268).

In one embodiment, the recombinant bacteria encode a fusion protein, in which a therapeutic protein of interest, is inserted into the TraT protein of a gram negative bacterium, e.g., E. coli, e.g. at position 180. The TraT protein is a surface-exposed lipoprotein, specified by plasmids of the IncF group, that mediates serum resistance and surface exclusion. Taylor et al. showed that insertion of the C3 epitope of polio virus, e.g., at position 180, allowed exposure of the antigen to the cell surface, while the oligomeric conformation of the wild-type protein was maintained (Taylor et al., Mol Microbiol. 1990 August; 4(8):1259-68).

In one embodiment, the recombinant bacteria comprise one or more genes and/or gene cassettes encoding a fusion protein comprising a Lpp-OmpA display vehicle comprising the N terminal outer membrane signal from the major lipoprotein (Lpp) fused to a domain from the outer membrane protein OmpA, fused to the therapeutic polypeptide of interest. In this system, the Lpp signal peptide mediates localization, and OmpA provides the framework for the display of the therapeutic protein of interest. Lpp-OmpA fusions have been used to display several proteins between 20 and 54 kDa in size on the surface of E. coli (see, e.g., Staphopoulos et al.). For example, Fransco et al fused beta-lactamase to the N-terminal targeting sequence of Lpp and an OmpA fragment containing 5 of the 8 membrane spanning loops of the native protein. This fusion protein was assembled on the cell surface and the beta-lactamase domain was stably anchored in the cell wall (Fransisco et al., PNAS Vol 89, pp. 2713-2717, 1992).

In one embodiment, the Type II secretion pathway or a variation thereof is used to for transient or longer duration display of therapeutic proteins of interest on the bacterial cell surface, e.g., the IgA protease secretion pathway of Neisseria or the VirG protein pathway of Shigella. In one embodiment, the IgA protease secretion pathway is used to export and display therapeutic peptides of interest on the cell surface of gram negative bacteria. The IgA proteases of Neisseria gonorrhoeae and Hemophilus influenza use a variation of the most common, Type II secretion pathway, to achieve extracellular export independent of any other gene products. The IgA genes of Neisseria species encode extracellular proteins that cleave human IgA1 antibody. The iga gene alone is sufficient to direct selected extracellular secretion of IgA protease in Neisseria, Salmonella, and E. coli species (Klauser et al., 1993, EMBO J 9:1991-1999, and references therein). The mature IgA protease is processed in several steps from a large precursor by signal peptidase and autoproteolytic cleavage. The precursor consists of four domains: (1) an aminoterminal signal peptide which mediates inner membrane transport; (2) the protease domain (3) the alpha domain, a basic alpha helical region which is secreted with the protease and (4) the autotransporter beta domain which harbors the essential function for outer membrane transport. Essentially, the C-terminal beta autotransporter domain of the IgA protease forms a channel in the outer membrane that mediates the export of the N terminal domain across the membrane, which in turn becomes transiently displayed on the external surface of the bacteria. The alpha domain and protease domain are then released through proteolytic cleavage. Klauser et al. (1993), showed that replacement of the native N-terminal domains of IgA protease of N. gonorrhoeae with the cholera toxin B resulted in the surface presentation of the passenger polypeptide in S. typhymurium. In another study, the signal sequence and the C-terminal beta autotransporter domain of the IgA protease of Neisseria gonorrhoeae was used to translocate and display a scFv directed against a porcine epidemic diarrhea virus epitope on the bacterial cell surface of E. coli (Pyo et al., Vaccine (27) (2009) 2030-2036).

Thus, in one embodiment, the recombinant bacteria encode an IgA protease fragment in which the alpha domain is substituted with a therapeutic protein of interest, and fused to a functional IgA protease beta-domain, which mediates export through the outer membrane. Without wishing to be bound by theory, IgA protease activity is eliminated in such a fusion protein, and therefore the autoproteoulytic release of the fusion protein into the medium does not occur, resulting in the display of the therapeutic protein of interest on the cell surface of the gram-negative host bacterium.

The secretion of VirG protein from Shigella is similar to the export system utilized by the IgA protease of Neisseria (Suzuki et al., 1995; J Biol. Chem 270:30874-30880, and references therein). Thus, in some embodiments, the recombinant bacteria encode a fusion protein comprising a therapeutic protein of interest fused to the membrane spanning region of VirG, resulting in surface display of the therapeutic protein of interest. The VirG gene on the large plasmid of Shigella has been shown to be responsible for the localized deposition of filamentous actin (F-actin) trailing from one pole of invading bacterial cells and extending in a filament through the host epithelial cytoplasm. VirG is a surface-exposed outer membrane protein consisting of three distinctive domains, the N-terminal signal sequence (amino acids 1-52), the id α-domain (amino acids 53-758), and the dC-terminal β-core (amino acids 759-1102). Suzuki et al. (1995) showed that the fusion of a foreign protein such as MalE or PhoA protein to the N terminus 37-kDa VirG portion resulted in the transport of the passenger polypeptides from the periplasm to the external side of the outer membrane, indicating that the C-terminal 37-kDa VirG portion embedded in the outer membrane is involved in the translocation of the preceding VirG portion or the heterologous or passenger polypeptide from the periplasmic space to the external side of the outer membrane, in a manner homologous to the IgA protease beta-domain. In some embodiments, the recombinant bacteria comprise one or more gene(s) or gene cassette(s) encoding a fusion protein, in which a C-terminal 37-kDa VirG protein fragment is fused to a therapeutic protein of interest.

In some embodiments, the recombinant bacteria comprise one or more gene(s) or gene cassette(s) encoding a fusion protein, in which a therapeutic protein of interest is fused to pullulanase for temporary surface display. Pullulanase is specifically released into the medium by Klebsiella pneumonieae, and exists as a fully exposed, cell surface-bound intermediate before it is released into the medium from early stationary growth phase onwards. Cell-surface anchoring is accomplished by an N-terminal fatty acyl modification whose chemical composition is identical to that of other bacterial protein.

Unlike the IgA protease, the lipoprotein pullulanase (PulA) of Klebsiella pneumoniae, which is also exported via a type II secretion mechanism, requires 14 genes for its translocation across the outer membrane. For example, Pugsley and coworkers have shown that the lipoprotein pullulanase (PulA) can facilitate translocation of the periplasmic enzyme beta-lactamase across the outer membrane. In particular, in E. coli strains expressing all pullulanase secretion genes, pullulanase-beta-lactamase hybrid protein molecules containing an N-terminal 834-amino-acid pullulanase segment were efficiently transported to the cell surface. Of note, pullulanase hybrids remain only temporarily attached to the bacterial surface and are subsequently released into the medium (Kornacker and Pugsley, Mol. Microbiol. 4:1101-1109, and references therein). Accordingly, in some embodiments, the recombinant bacteria comprise one or more gene sequence(s) comprising a complete set of pullulanase genes required for secretion and fusion protein comprising a therapeutic protein of interest fused to a N-terminal pullulanase polypeptide fragment, e.g., as described by Kornacker and Pugsley. In some embodiments, the fusion proteins comprising N-terminal pullulanase polypeptide fused to the therapeutic protein of interest, are transiently displayed on the surface of the bacterial cell, and subsequently released into the media or extracellular space.

In one embodiment, the recombinant bacteria comprise one or more gene(s) or gene cassette(s) encoding a fusion protein in which the ice nucleation protein (INP) from Pseudomonas syringae anchors a therapeutic protein of interest in the cell wall. INP is a secretory protein that catalyzes extracellular ice formation as the ice nuclei. INP has been found in a number of Gram-negative species, including P. syringae, Erwinia herbicola, Xanthomonas campestris, and Pseudomonas fluorescens. Four genes in P. syringae strains, inaK, inaV, and inaZ, and inaQ exhibit high similarities in sequences and in primary organization (Li et al., Int J Biol Sci. 2012; 8(8): 1097-1108). All INPs (1200 aa to 1500 aa) comprise of three distinct structural domains: (1) the N-terminal domain (approximately 15% of the total sequence), which is relatively hydrophobic and which is are potentially capable of being coupled to the mannan-phosphatidylinositol group in the outer membrane through N-glycan (Asp) or O-glycan (Ser, Thr) linkages; (2) the C-terminal domain (approximately 4%), which is a relatively hydrophilic terminus; and (3) the central repeating domain (CRD) (approximately 81%), which constitutes contiguous repeats given by 16-residue (or 48-residue) periodicities with a consensus octapeptide (Ala-Gly-Tyr-Gly-Ser-Thr-Leu-Thr (SEQ ID NO: 1130)). INPs have been employed in various bacterial cell-surface display systems including E. coli, Zymomonas mobilis, Salmonellas sp., Vibrio anguillarum, Pseudomonas putida, and cyanobacteria, in all of which INPs were able to target a heterologous protein onto the surface of the host cell. Moreover, the N-terminal region alone was shown to direct translocation of foreign proteins to the cell surface and can be employed as a potential cell surface display motif (Li et al., Biotechnol Bioeng. 2004; 85(2):214-21). Accordingly, in some embodiments, the recombinant bacteria comprise IMP fusions for surface display of a therapeutic peptide of interest. In some embodiments, the N-terminal region of the INP protein is fused to the polypeptide of interest for surface display.

INP proteins further have modifiable internal repeating units, i.e., CRD length is adjustable, which is allows flexibility in protein fusion length (Jung et al., 1998), and also can accommodate larger polypeptides. For example, the INP-based display systems were used to successfully express a 90 kDA protein on the cell surface of E. coli (Wu et al., 2006; FEMS Microbiology Letters, Volume 256, Issue 1; Pages 119-125).

It is understood by those skilled in the art that translocation of such fusion or hybrid proteins described herein requires a “translocation-competent” conformation, e.g., the formation of disulfide bonds, e.g., in the periplasmic space, may be undesirable and inhibit translocation through the outer membrane (see, e.g., Klauser et al., 1990), or alternatively may be required for, (or at least not impede) translocation through the outer membrane (see, e.g., Puggsley, 1992; Proc Natl Acad Sci USA.; 89(24): 12058-12062). In some embodiments, the recombinant bacteria comprise one or more gene sequence(s) encoding for a fusion protein in which disulfide bonds are prevented from forming prior to the translocation to the cell surface. In some embodiments, the recombinant bacteria comprise one or more gene(s) or gene cassette(s) encoding for a fusion protein in which disulfide bonds are formed prior to translocation to the cell surface.

Expression systems for the display of proteins in Gram-positive bacteria have also been developed. Consequently, in some embodiments, gram positive bacteria are engineered to display therapeutic proteins of interest on their cell surface. Uhlen et al. used fusions to the cell-wall bound, X-domain of protein A, for the display of foreign peptides up to 88 amino acids long to the surface of Staphylococcus strains. For example one study describes an expression system to allow targeting of heterologous proteins to the cell surface of Staphylococcus xylosus, a coagulase-negative gram-positive bacterium (Hansson et al., J Bacteriol. 1992 July; 174(13):4239-45).

The expression of recombinant gene fragments, fused between gene fragments encoding the signal peptide and the cell surface-binding regions of staphylococcal protein A, targets the resulting fusion proteins to the outer bacterial cell surface via the membrane-anchoring region and the highly charged cell wall-spanning region of staphylococcal protein A. Accordingly, in some embodiments, the recombinant bacteria comprise one or more gene sequences encoding a therapeutic polypeptide fused between gene fragments encoding the signal peptide and the cell surface-binding regions of staphylococcal protein A.

E. coli-staphylococcus shuttle vectors have been constructed by taking advantage of the promoter, signal sequence, and propeptide region from the lipase gene construct derived from S. hyicus and the cell surface attachment part of staphylococcal protein A. This system has been investigated for the surface display of heterologous polypeptides on S. carnosus (Samuelson et al., J Bacteriol. 1995; 177(6):1470-6). In some embodiments, the recombinant bacteria comprise one or more gene sequence(s) encoding a therapeutic polypeptide fusion protein comprising promoter, signal sequence, and propeptide region from the lipase gene construct derived from S. hyicus and the cell surface attachment part of staphylococcal protein A.

In other studies, the fibrillary M6 proteins of Streptococcus pyrogenes was employed as a carrier for antigen delivery in Streptococcus cells. (Pozzi et al., 1992; Infect. Immunm. 60: 1902-1907). In some embodiments, the recombinant bacteria comprise one or more gene sequence(s) comprising therapeutic polypeptide fusion proteins comprising the fibrillary M6 proteins of Streptococcus pyrogenes for cell surface display of the therapeutic polypeptide.

In some embodiments, the recombinant bacteria comprise one or more gene(s) or gene cassette(s) encoding a polypeptide of interest which is displayed on the cell surface through a fusion with an intimin or invasin. Intimins and invasins belong to a family of bacterial adhesins which specifically interact with various eukaryotic cell surface receptors, thereby mediating bacterial adherence and invasion. Both intimins and invasins provide a structural scaffold ideally suited to the cell surface display.

In some embodiments, the recombinant bacteria comprise one or more gene(s) or gene cassette(s) encoding a polypeptide of interest which is displayed on the cell surface through a fusion with an intimin, e.g., with the Enterohemorragic E. coli Intimin EaeA protein or a carboxy-terminal truncation thereof (e.g., as described in Wentzel et al, Intimin EaeA J Bacteriol. 2001; 183(24): 7273-7284). For example, N-terminal 489 amino acids of invasin are sufficient to promote the localization of a fusion protein to the cell surface.

In some embodiments, the recombinant bacteria comprise one or more gene(s) or gene cassette(s) encoding a polypeptide of interest which is displayed on the cell surface through a fusion with an invasin, e.g. Enterohemorrhagic E. coli invasion, or a carboxyterminal truncation thereof. For example, N-terminal 539 amino acids of intimin were sufficient to promote outer membrane localization of a fusion protein (Liu et al., Mol Microbiol. 1999; 34(1):67-81).

In some embodiments, the recombinant bacteria comprise one or more gene(s) or gene cassette(s) encoding a polypeptide of interest which is displayed on the cell surface through a fusion with Bacillus anthracis exosporal protein (BclA) as an anchoring motif. The BclA is an exosporium protein, a hair-like protein surrounding the B. anthracis spore. In a nonlimiting example, a polypeptide of interest is linked to the C-terminus of N-terminal domain (21 amino acids) of BclA, e.g., as described in Park et al..

Various other anchoring motifs have been developed including OprF, OmpC, and OmpX, In some embodiments, the recombinant bacteria comprise one or more gene(s) or gene cassette(s) encoding a polypeptide of interest which is displayed on the cell surface through a fusion with OprF, OmpC, and OmpX.

In some embodiments, the therapeutic polypeptides of interest are permanently displayed on the cell surface of the recombinant bacterium. In some embodiments, the therapeutic polypeptides of interest are transiently displayed on the cell surface of the recombinant bacterium.

In some embodiments, the therapeutic polypeptides are displayed in strains, e.g., described herein which display a leaky phenotype. Such strains have deactivating mutations in one or more of genes encoding a protein that tethers the outer membrane to the peptidoglycan skeleton, e.g., lpp, ompC, ompA, ompF, tolA, tolB, pal, and/or one or more genes encoding a periplasmic protease, e.g., degS, degP, nlpl.

In some embodiments, one or more ScFvs are displayed on the bacterial cell surface, alone or in combination with other therapeutic polypeptides of interest.

In some embodiments, a cell surface display strategy or circuit is combined with a secretion strategy or circuit in one bacterium. In some embodiments, the same polypeptide is both displayed and secreted. In some embodiments, a first polypeptide is displayed and a second is secreted. In some embodiments, a display strategy or circuit strategy is combined with a circuit for the intracellular production of an enzyme and consequentially intracellular catabolism of its substrate. In some embodiments, a display strategy or display circuit is combined with a circuit for the intracellular production of a secreted effector molecule described herein.

In some embodiments, the expression of the surface displayed polypeptide or fusion protein is driven by an inducible promoter. In some embodiments, the inducible promoter is an oxygen level-dependent promoter (e.g., FNR-inducible promoter). In some embodiments, the inducible promoter is induced by gut-specific and/or tumor-specific or promoters induced by inflammation or an inflammatory response (RNS, ROS promoters), or promoters induced by a metabolite that may or may not be naturally present (e.g., can be exogenously added) in the gut, e.g., arabinose. In alternate embodiments, expression of the surface displayed polypeptides or polypeptide fusion proteins is driven by a constitutive promoter.

In some embodiments, the expression of the surface displayed polypeptide or fusion protein is plasmid based. In some embodiments, the gene sequence(s) encoding the antibodies or scFv fragments for surface display is chromosomally inserted.

Essential Genes and Auxotrophs

As used herein, the term “essential gene” refers to a gene which is necessary to for cell growth and/or survival. Bacterial essential genes are well known to one of ordinary skill in the art, and can be identified by directed deletion of genes and/or random mutagenesis and screening (see, e.g., Zhang, 2009, Nucl. Acids Res., 37:D455-D458 and Gerdes et al., Curr. Opin. Biotechnol., 17(5):448-456, the entire contents of each reference are incorporated herein by reference).

An “essential gene” may be dependent on the circumstances and environment in which an organism lives. For example, a mutation of, modification of, or excision of an essential gene may result in the recombinant bacteria of the disclosure becoming an auxotroph. An auxotrophic modification is intended to cause bacteria to die in the absence of an exogenously added nutrient essential for survival or growth because they lack the gene(s) necessary to produce that essential nutrient.

An auxotrophic modification is intended to cause bacteria to die in the absence of an exogenously added nutrient essential for survival or growth because they lack the gene(s) necessary to produce that essential nutrient. In some embodiments, any of the recombinant bacteria described herein also comprise a deletion or mutation in a gene required for cell survival and/or growth. In one embodiment, the essential gene is a DNA synthesis gene, for example, thyA. In another embodiment, the essential gene is a cell wall synthesis gene, for example, dapA. In yet another embodiment, the essential gene is an amino acid gene, for example, serA or MetA. Any gene required for cell survival and/or growth may be targeted, including but not limited to, cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thi1, as long as the corresponding wild-type gene product is not produced in the bacteria.

Table 15 lists exemplary bacterial genes which may be disrupted or deleted to produce an auxotrophic strain. These include, but are not limited to, genes required for oligonucleotide synthesis, amino acid synthesis, and cell wall synthesis.

TABLE 15
Non-limiting Examples of Bacterial Genes
Useful for Generation of an Auxotroph

| Amino Acid | Oligonucleotide | Cell Wall |
|---|---|---|
| cysE | thyA | dapA |
| glnA | uraA | dapB |
| ilvD | | dapD |
| leuB | | dapE |
| lysA | | dapF |
| serA | | |
| metA | | |
| glyA | | |
| hisB | | |
| ilvA | | |
| pheA | | |
| proA | | |
| thrC | | |
| trpC | | |
| tyrA | | |

Table 16 shows the survival of various amino acid auxotrophs in the mouse gut, as detected 24 hrs and 48 hrs post-gavage. These auxotrophs were generated using BW25113, a non-Nissle strain of E. coli.

TABLE 16
Survival of amino acid auxotrophs in the mouse gut

| Gene | AA Auxotroph | Pre-Gavage | 24 hours | 48 hours |
|---|---|---|---|---|
| argA | Arginine | Present | Present | Absent |
| cysE | Cysteine | Present | Present | Absent |
| glnA | Glutamine | Present | Present | Absent |
| glyA | Glycine | Present | Present | Absent |
| hisB | Histidine | Present | Present | Present |
| ilvA | Isoleucine | Present | Present | Absent |
| leuB | Leucine | Present | Present | Absent |
| lysA | Lysine | Present | Present | Absent |
| metA | Methionine | Present | Present | Present |
| pheA | Phenylalanine | Present | Present | Present |
| proA | Proline | Present | Present | Absent |
| serA | Serine | Present | Present | Present |
| thrC | Threonine | Present | Present | Present |
| trpC | Tryptophan | Present | Present | Present |
| tyrA | Tyrosine | Present | Present | Present |
| ilvD | Valine/Isoleucine/ | Present | Present | Absent |
| | Leucine | | | |
| thyA | Thiamine | Present | Absent | Absent |
| uraA | Uracil | Present | Absent | Absent |
| flhD | FlhD | Present | Present | Present |

For example, thymine is a nucleic acid that is required for bacterial cell growth; in its absence, bacteria undergo cell death. The thyA gene encodes thimidylate synthetase, an enzyme that catalyzes the first step in thymine synthesis by converting dUMP to dTMP (Sat et al., 2003). In some embodiments, the bacterial cell of the disclosure is a thyA auxotroph in which the thyA gene is deleted and/or replaced with an unrelated gene. A thyA auxotroph can grow only when sufficient amounts of thymine are present, e.g., by adding thymine to growth media in vitro, or in the presence of high thymine levels found naturally in the human gut in vivo. In some embodiments, the bacterial cell of the disclosure is auxotrophic in a gene that is complemented when the bacterium is present in the mammalian gut. Without sufficient amounts of thymine, the thyA auxotroph dies. In some embodiments, the auxotrophic modification is used to ensure that the bacterial cell does not survive in the absence of the auxotrophic gene product (e.g., outside of the gut).

Diaminopimelic acid (DAP) is an amino acid synthetized within the lysine biosynthetic pathway and is required for bacterial cell wall growth (Meadow et al., 1959; Clarkson et al., 1971). In some embodiments, any of the recombinant bacteria described herein is a dapD auxotroph in which dapD is deleted and/or replaced with an unrelated gene. A dapD auxotroph can grow only when sufficient amounts of DAP are present, e.g., by adding DAP to growth media in vitro. Without sufficient amounts of DAP, the dapD auxotroph dies. In some embodiments, the auxotrophic modification is used to ensure that the bacterial cell does not survive in the absence of the auxotrophic gene product (e.g., outside of the gut).

In other embodiments, the recombinant bacterium of the present disclosure is a uraA auxotroph in which uraA is deleted and/or replaced with an unrelated gene. The uraA gene codes for UraA, a membrane-bound transporter that facilitates the uptake and subsequent metabolism of the pyrimidine uracil (Andersen et al., 1995). A uraA auxotroph can grow only when sufficient amounts of uracil are present, e.g., by adding uracil to growth media in vitro. Without sufficient amounts of uracil, the uraA auxotroph dies. In some embodiments, auxotrophic modifications are used to ensure that the bacteria do not survive in the absence of the auxotrophic gene product (e.g., outside of the gut).

In complex communities, it is possible for bacteria to share DNA. In very rare circumstances, an auxotrophic bacterial strain may receive DNA from a non-auxotrophic strain, which repairs the genomic deletion and permanently rescues the auxotroph. Therefore, engineering a bacterial strain with more than one auxotroph may greatly decrease the probability that DNA transfer will occur enough times to rescue the auxotrophy. In some embodiments, the recombinant bacteria comprise a deletion or mutation in two or more genes required for cell survival and/or growth.

Other examples of essential genes include, but are not limited to yhbV, yagG, hemB, secD, secF, ribD, ribE, thiL, dxs, ispA, dnaX, adk, hemH, lpxH, cysS, fold, rplT, infC, thrS, nadE, gapA, yeaZ, aspS, argS, pgsA, yefM, metG, folE, yejM, gyrA, nrdA, nrdB, folC, accD, fabB, gltX, ligA, zipA, dapE, dapA, der, hisS, ispG, suhB, tadA, acpS, era, mc, ftsB, eno, pyrG, chpR, lgt, fbaA, pgk, yqgD, metK, yqgF, plsC, ygiT, pare, ribB, cca, ygjD, tdcF, yraL, yihA, ftsN, murl, murB, birA, secE, nusG, rplJ, rplL, rpoB, rpoC, ubiA, plsB, lexA, dnaB, ssb, alsK, groS, psd, orn, yjeE, rpsR, chpS, ppa, valS, yjgP, yjgQ, dnaC, ribF, lspA, ispH, dapB, folA, imp, yabQ, ftsL, ftsI, murE, murF, mraY, murD, ftsW, murG, murC, ftsQ, ftsA, ftsZ, lpxC, secM, secA, can, folK, hemL, yadR, dapD, map, rpsB, infB, nusA, ftsH, obgE, rpmA, rplU, ispB, murA, yrbB, yrbK, yhbN, rpsI, rplM, degS, mreD, mreC, mreB, accB, accC, yrdC, def, fmt, rplQ, rpoA, rpsD, rpsK, rpsM, entD, mrdB, mrdA, nadD, hlepB, rpoE, pssA, yfiO, rplS, trmD, rpsP, ffh, grpE, yfjB, csrA, ispF, ispD, rplW, rplD, rplC, rpsJ, fusA, rpsG, rpsL, trpS, yrfF, asd, rpoH, ftsX, ftsE, ftsY, frr, dxr, ispU, rfaK, kdtA, coaD, rpmB, dfp, dut, gmk, spot, gyrB, dnaN, dnaA, rpmH, rnpA, yidC, tnaB, glmS, glmU, wzyE, hemD, hemC, yigP, ubiB, ubiD, hemG, secY, rplO, rpmD, rpsE, rplR, rplF, rpsH, rpsN, rplE, rplX, rplN, rpsQ, rpmC, rplP, rpsC, rplV, rpsS, rplB, cdsA, yaeL, yaeT, lpxD, fabZ, lpxA, lpxB, dnaE, accA, tilS, proS, yafF, tsf, pyrH, olA, rlpB, leuS, lnt, glnS, fldA, cydA, infA, cydC, ftsK, lolA, serS, rpsA, msbA, lpxK, kdsB, mukF, mukE, mukB, asnS, fabA, mviN, me, yceQ, fabD, fabG, acpP, tmk, holB, lolC, lolD, lolE, purB, ymfK, minE, mind, pth, rsA, ispE, lolB, hemA, prfA, prmC, kdsA, topA, ribA, fabl, racR, dicA, ydfB, tyrS, ribC, ydiL, pheT, pheS, yhhQ, bcsB, glyQ, yibJ, and gpsA. Other essential genes are known to those of ordinary skill in the art.

In some embodiments, the recombinant bacterium of the present disclosure is a synthetic ligand-dependent essential gene (SLiDE) bacterial cell. SLiDE bacterial cells are synthetic auxotrophs with a mutation in one or more essential genes that only grow in the presence of a particular ligand (see Lopez and Anderson, ACS Synthetic Biology (2015), the entire contents of which are expressly incorporated herein by reference).

In some embodiments, the SLiDE bacterial cell comprises a mutation in an essential gene. In some embodiments, the essential gene is selected from the group consisting of pheS, dnaN, tyrS, metG, and adk. In some embodiments, the essential gene is dnaN comprising one or more of the following mutations: H191N, R240C, 1317S, F319V, L340T, V347I, and S345C. In some embodiments, the essential gene is dnaN comprising the mutations H191N, R240C, I317S, F319V, L340T, V347I, and S345C. In some embodiments, the essential gene is pheS comprising one or more of the following mutations: F125G, P183T, P184A, R186A, and I188L. In some embodiments, the essential gene is pheS comprising the mutations F125G, P183T, P184A, R186A, and I188L. In some embodiments, the essential gene is tyrS comprising one or more of the following mutations: L36V, C38A and F40G. In some embodiments, the essential gene is tyrS comprising the mutations L36V, C38A and F40G. In some embodiments, the essential gene is metG comprising one or more of the following mutations: E45Q, N47R, I49G, and A51C. In some embodiments, the essential gene is metG comprising the mutations E45Q, N47R, I49G, and A51C. In some embodiments, the essential gene is adk comprising one or more of the following mutations: I4L, L5I and L6G. In some embodiments, the essential gene is adk comprising the mutations I4L, L5I and L6G.

In some embodiments, the recombinant bacterium is complemented by a ligand. In some embodiments, the ligand is selected from the group consisting of benzothiazole, indole, 2-aminobenzothiazole, indole-3-butyric acid, indole-3-acetic acid, and L-histidine methyl ester. For example, bacterial cells comprising mutations in metG (E45Q, N47R, I49G, and A51C) are complemented by benzothiazole, indole, 2-aminobenzothiazole, indole-3-butyric acid, indole-3-acetic acid or L-histidine methyl ester. Bacterial cells comprising mutations in dnaN (H191N, R240C, I317S, F319V, L340T, V347I, and S345C) are complemented by benzothiazole, indole or 2-aminobenzothiazole. Bacterial cells comprising mutations in pheS (F125G, P183T, P184A, R186A, and I188L) are complemented by benzothiazole or 2-aminobenzothiazole. Bacterial cells comprising mutations in tyrS (L36V, C38A, and F40G) are complemented by benzothiazole or 2-aminobenzothiazole. Bacterial cells comprising mutations in adk (I4L, L5I and L6G) are complemented by benzothiazole or indole.

In some embodiments, the recombinant bacterium comprises more than one mutant essential gene that renders it auxotrophic to a ligand. In some embodiments, the bacterial cell comprises mutations in two essential genes. For example, in some embodiments, the bacterial cell comprises mutations in tyrS (L36V, C38A, and F40G) and metG (E45Q, N47R, I49G, and A51C). In other embodiments, the bacterial cell comprises mutations in three essential genes. For example, in some embodiments, the bacterial cell comprises mutations in tyrS (L36V, C38A, and F40G), metG (E45Q, N47R, I49G, and A51C), and pheS (F125G, P183T, P184A, R186A, and I188L).

In some embodiments, the recombinant bacterium is a conditional auxotroph, wherein the expression of a heterologous gene is activated by an exogenous environmental signal. In some embodiments, the recombinant bacterium is a conditional auxotroph whose essential gene(s) is replaced using an arabinose system. In the absence of arabinose, the AraC transcription factor adopts a conformation that represses transcription of the essential gene under the control of the araBAD promoter and the bacterial cell cannot survive. In the presence of arabinose, the AraC transcription factor undergoes a conformational change that allows it to bind to and activate the araBAD promoter, which induces expression of the essential gene and maintains viability of the bacterial cell.

In some embodiments, the recombinant bacterium of the disclosure is an auxotroph and also comprises kill-switch circuitry, such as any of the kill-switch components and systems described herein. For example, the recombinant bacteria may comprise a deletion or mutation in an essential gene required for cell survival and/or growth, for example, in a DNA synthesis gene, for example, thyA, cell wall synthesis gene, for example, dapA and/or an amino acid gene, for example, serA or MetA and may also comprise a toxin gene that is regulated by one or more transcriptional activators that are expressed in response to an environmental condition(s) and/or signal(s) (such as the described arabinose system) or regulated by one or more recombinases that are expressed upon sensing an exogenous environmental condition(s) and/or signal(s) (such as the recombinase systems described herein). Other embodiments are described in Wright et al., ACS Synthetic Biology (2015) 4: 307-16, the entire contents of which are expressly incorporated herein by reference. In some embodiments, the recombinant bacterium of the disclosure is an auxotroph and also comprises kill-switch circuitry, such as any of the kill-switch components and systems described herein, as well as another biosecurity system, such a conditional origin of replication (Wright et al., 2015). In other embodiments, auxotrophic modifications may also be used to screen for mutant bacteria that produce the effector molecule.

Antibiotic Resistance

In some embodiments, the recombinant bacteria comprise one or more genes providing resistance to antibiotics. As used herein, “Antibiotics” are substances that kill bacteria (bactericidal), or inhibit bacterial growth (bacteriostatic). Antibiotics may be natural products, many common antibiotics used in labs today are semi-synthetic or fully synthetic compounds. An antibiotic resistance gene can be added to a bacterium of interest, either on a plasmid or integrated into the chromosome in conjunction with the gene of interest, allowing the simple detection of bacteria containing the gene of interest by growing the bacteria on selective media containing the antibiotic.

In some embodiments, the recombinant bacteria comprise one or more genes providing resistance to aminoglycosides. In some embodiments, the recombinant bacteria comprise one or more genes providing resistance to a beta-lactam. In some embodiments, the recombinant bacteria comprise one or more genes providing resistance to glycopeptides. In some embodiments, the recombinant bacteria comprise one or more genes providing resistance to macrolides. In some embodiments, the recombinant bacteria comprise one or more genes providing resistance to polypeptide antibiotics. In some embodiments, the recombinant bacteria comprise one or more genes providing resistance to a tetracyclin.

In some embodiments, the recombinant bacteria comprise one or more genes providing resistance to Kanamycin. In some embodiments, the recombinant bacteria comprise one or more genes providing resistance to Spectinomycin. In some embodiments, the recombinant bacteria comprise one or more genes providing resistance to Streptomycin. In some embodiments, the recombinant bacteria comprise one or more genes providing resistance to Ampicillin. In some embodiments, the recombinant bacteria comprise one or more genes providing resistance to Carbenicillin. In some embodiments, the recombinant bacteria comprise one or more genes providing resistance to Bleomycin. In some embodiments, the recombinant bacteria comprise one or more genes providing resistance to Erythromycin. In some embodiments, the recombinant bacteria comprise one or more genes providing resistance to Polymyxin B. In some embodiments, the recombinant bacteria comprise one or more genes providing resistance to Tetracycline. In some embodiments, the recombinant bacteria comprise one or more genes providing resistance to Chloramphenicol.

Genetic Regulatory Circuits

In some embodiments, the recombinant bacteria comprise multi-layered genetic regulatory circuits for expressing the constructs described herein (see, e.g., U.S. Provisional Application No. 62/184,811 and PCT/US2016/39434, both of which are incorporated herein by reference in their entireties). The genetic regulatory circuits are useful to screen for mutant bacteria that produce an effector molecule or rescue an auxotroph. In certain embodiments, the invention provides methods for selecting recombinant bacteria that produce one or more genes of interest.

In some embodiments, the invention provides recombinant bacteria comprising a gene or gene cassette for producing a therapeutic molecule (e.g., butyrate) and a T7 polymerase-regulated genetic regulatory circuit. For example, the recombinant bacteria comprise a first gene encoding a T7 polymerase, wherein the first gene is operably linked to a FNR-responsive promoter; a second gene or gene cassette for producing a therapeutic molecule (e.g., butyrate), wherein the second gene or gene cassette is operably linked to a T7 promoter that is induced by the T7 polymerase; and a third gene encoding an inhibitory factor, lysY, that is capable of inhibiting the T7 polymerase. In the presence of oxygen, FNR does not bind the FNR-responsive promoter, and the therapeutic molecule (e.g., butyrate) is not expressed. LysY is expressed constitutively (P-lac constitutive) and further inhibits T7 polymerase. In the absence of oxygen, FNR dimerizes and binds to the FNR-responsive promoter, T7 polymerase is expressed at a level sufficient to overcome lysY inhibition, and the therapeutic molecule (e.g., butyrate) is expressed. In some embodiments, the lysY gene is operably linked to an additional FNR binding site. In the absence of oxygen, FNR dimerizes to activate T7 polymerase expression as described above, and also inhibits lysY expression.

In some embodiments, the invention provides recombinant bacteria comprising a gene or gene cassette for producing a therapeutic molecule (e.g., butyrate) and a protease-regulated genetic regulatory circuit. For example, the recombinant bacteria comprise a first gene encoding an mf-lon protease, wherein the first gene is operably linked to a FNR-responsive promoter; a second gene or gene cassette for producing a therapeutic molecule operably linked to a Tet regulatory region (TetO); and a third gene encoding an mf-lon degradation signal linked to a Tet repressor (TetR), wherein the TetR is capable of binding to the Tet regulatory region and repressing expression of the second gene or gene cassette. The mf-lon protease is capable of recognizing the mf-lon degradation signal and degrading the TetR. In the presence of oxygen, FNR does not bind the FNR-responsive promoter, the repressor is not degraded, and the therapeutic molecule is not expressed. In the absence of oxygen, FNR dimerizes and binds the FNR-responsive promoter, thereby inducing expression of the mf-lon protease. The mf-lon protease recognizes the mf-lon degradation signal and degrades the TetR, and the therapeutic molecule is expressed.

In some embodiments, the invention provides recombinant bacteria comprising a gene or gene cassette for producing a therapeutic molecule and a repressor-regulated genetic regulatory circuit. For example, the recombinant bacteria comprise a first gene encoding a first repressor, wherein the first gene is operably linked to a FNR-responsive promoter; a second gene or gene cassette for producing a therapeutic molecule operably linked to a first regulatory region comprising a constitutive promoter; and a third gene encoding a second repressor, wherein the second repressor is capable of binding to the first regulatory region and repressing expression of the second gene or gene cassette. The third gene is operably linked to a second regulatory region comprising a constitutive promoter, wherein the first repressor is capable of binding to the second regulatory region and inhibiting expression of the second repressor. In the presence of oxygen, FNR does not bind the FNR-responsive promoter, the first repressor is not expressed, the second repressor is expressed, and the therapeutic molecule is not expressed. In the absence of oxygen, FNR dimerizes and binds the FNR-responsive promoter, the first repressor is expressed, the second repressor is not expressed, and the therapeutic molecule is expressed.

Examples of repressors useful in these embodiments include, but are not limited to, ArgR, TetR, ArsR, AscG, Lad, CscR, DeoR, DgoR, FruR, GalR, GatR, CI, LexA, RafR, QacR, and PtxS (US20030166191).

In some embodiments, the invention provides recombinant bacteria comprising a gene or gene cassette for producing a therapeutic molecule and a regulatory RNA-regulated genetic regulatory circuit. For example, the recombinant bacteria comprise a first gene encoding a regulatory RNA, wherein the first gene is operably linked to a FNR-responsive promoter, and a second gene or gene cassette for producing a therapeutic molecule. The second gene or gene cassette is operably linked to a constitutive promoter and further linked to a nucleotide sequence capable of producing an mRNA hairpin that inhibits translation of the therapeutic molecule. The regulatory RNA is capable of eliminating the mRNA hairpin and inducing translation via the ribosomal binding site. In the presence of oxygen, FNR does not bind the FNR-responsive promoter, the regulatory RNA is not expressed, and the mRNA hairpin prevents the therapeutic molecule from being translated. In the absence of oxygen, FNR dimerizes and binds the FNR-responsive promoter, the regulatory RNA is expressed, the mRNA hairpin is eliminated, and the therapeutic molecule is expressed.

In some embodiments, the invention provides recombinant bacteria comprising a gene or gene cassette for producing a therapeutic molecule and a CRISPR-regulated genetic regulatory circuit. For example, the recombinant bacteria comprise a Cas9 protein; a first gene encoding a CRISPR guide RNA, wherein the first gene is operably linked to a FNR-responsive promoter; a second gene or gene cassette for producing a therapeutic molecule, wherein the second gene or gene cassette is operably linked to a regulatory region comprising a constitutive promoter; and a third gene encoding a repressor operably linked to a constitutive promoter, wherein the repressor is capable of binding to the regulatory region and repressing expression of the second gene or gene cassette. The third gene is further linked to a CRISPR target sequence that is capable of binding to the CRISPR guide RNA, wherein said binding to the CRISPR guide RNA induces cleavage by the Cas9 protein and inhibits expression of the repressor. In the presence of oxygen, FNR does not bind the FNR-responsive promoter, the guide RNA is not expressed, the repressor is expressed, and the therapeutic molecule is not expressed. In the absence of oxygen, FNR dimerizes and binds the FNR-responsive promoter, the guide RNA is expressed, the repressor is not expressed, and the therapeutic molecule is expressed.

In some embodiments, the invention provides recombinant bacteria comprising a gene or gene cassette for producing a therapeutic molecule and a recombinase-regulated genetic regulatory circuit. For example, the recombinant bacteria comprise a first gene encoding a recombinase, wherein the first gene is operably linked to a FNR-responsive promoter, and a second gene or gene cassette for producing a therapeutic molecule operably linked to a constitutive promoter. The second gene or gene cassette is inverted in orientation (3′ to 5′) and flanked by recombinase binding sites, and the recombinase is capable of binding to the recombinase binding sites to induce expression of the second gene or gene cassette by reverting its orientation (5′ to 3′). In the presence of oxygen, FNR does not bind the FNR-responsive promoter, the recombinase is not expressed, the gene or gene cassette remains in the 3′ to 5′ orientation, and no functional therapeutic molecule is produced. In the absence of oxygen, FNR dimerizes and binds the FNR-responsive promoter, the recombinase is expressed, the gene or gene cassette is reverted to the 5′ to 3′ orientation, and a functional therapeutic molecule is produced.

In some embodiments, the invention provides recombinant bacteria comprising a gene or gene cassette for producing a therapeutic molecule and a polymerase- and recombinase-regulated genetic regulatory circuit. For example, the recombinant bacteria comprise a first gene encoding a recombinase, wherein the first gene is operably linked to a FNR-responsive promoter; a second gene or gene cassette for producing a therapeutic molecule operably linked to a T7 promoter; a third gene encoding a T7 polymerase, wherein the T7 polymerase is capable of binding to the T7 promoter and inducing expression of the therapeutic molecule. The third gene encoding the T7 polymerase is inverted in orientation (3′ to 5′) and flanked by recombinase binding sites, and the recombinase is capable of binding to the recombinase binding sites to induce expression of the T7 polymerase gene by reverting its orientation (5′ to 3′). In the presence of oxygen, FNR does not bind the FNR-responsive promoter, the recombinase is not expressed, the T7 polymerase gene remains in the 3′ to 5′ orientation, and the therapeutic molecule is not expressed. In the absence of oxygen, FNR dimerizes and binds the FNR-responsive promoter, the recombinase is expressed, the T7 polymerase gene is reverted to the 5′ to 3′ orientation, and the therapeutic molecule is expressed.

Synthetic gene circuits expressed on plasmids may function well in the short term but lose ability and/or function in the long term (Danino et al., 2015). In some embodiments, the recombinant bacteria comprise stable circuits for expressing genes of interest over prolonged periods. In some embodiments, the recombinant bacteria are capable of producing a therapeutic molecule and further comprise a toxin-anti-toxin system that simultaneously produces a toxin (hok) and a short-lived anti-toxin (sok), wherein loss of the plasmid causes the cell to be killed by the long-lived toxin (Danino et al., 2015). In some embodiments, the recombinant bacteria further comprise alp7 from B. subtilis plasmid pL20 and produces filaments that are capable of pushing plasmids to the poles of the cells in order to ensure equal segregation during cell division (Danino et al., 2015).

Host-Plasmid Mutual Dependency

In some embodiments, the recombinant bacteria also comprise a plasmid that has been modified to create a host-plasmid mutual dependency. In certain embodiments, the mutually dependent host-plasmid platform is antibiotic independent plasmid system (AIPS) (Wright et al., 2015). In some embodiments, the AIPS comprises (i) a conditional origin of replication, in which the requisite replication initiator protein is provided in trans; (ii) an auxotrophic modification that is rescued by the host via genomic translocation and is also compatible for use in rich media; and/or (iii) a nucleic acid sequence which encodes a broad-spectrum toxin. The toxin gene may be used to select against plasmid spread by making the plasmid DNA itself disadvantageous for strains not expressing the anti-toxin (e.g., a wild-type bacterium). In some embodiments, the AIPS plasmid is stable for at least 100 generations without antibiotic selection. In some embodiments, the AIPS plasmid does not disrupt growth of the host. The AIPS plasmid is used to greatly reduce unintentional plasmid propagation in the recombinant bacteria.

The mutually dependent host-plasmid platform may be used alone or in combination with other biosafety mechanisms, such as those described herein (e.g., kill switches, auxotrophies). In some embodiments, the recombinant bacteria comprise a AIPS plasmid. In other embodiments, the recombinant bacteria comprise a AIPS plasmid and/or one or more kill switches. In other embodiments, the recombinant bacteria comprise a AIPS plasmid and/or one or more auxotrophies. In still other embodiments, the recombinant bacteria comprise a AIPS plasmid, one or more kill switches, and/or one or more auxotrophies.

Synthetic gene circuits express on plasmids may function well in the short term but lose ability and/or function in the long term (Danino et al., 2015). In some embodiments, the recombinant bacteria comprise stable circuits for expressing genes of interest over prolonged periods. In some embodiments, the recombinant bacteria are capable of producing a secreted effector molecule described herein and further comprise a toxin-anti-toxin system that simultaneously produces a toxin (hok) and a short-lived anti-toxin (sok), wherein loss of the plasmid causes the cell to be killed by the long-lived toxin (Danino et al., 2015; as shown in the figures and examples). In some embodiments, the recombinant bacteria further comprise alp7 from B. subtilis plasmid pL20 and produces filaments that are capable of pushing plasmids to the poles of the cells in order to ensure equal segregation during cell division (Danino et al., 2015).

Kill Switch

In some embodiments, the recombinant bacteria also comprise a kill switch (see, e.g., U.S. Provisional Application Nos. 62/183,935, 62/263,329, 62/277,654, and International Patent Publication WO2016/210373, the contents of each of which is incorporated herein by reference in their entireties). The kill switch is intended to actively kill recombinant bacteria in response to external stimuli. As opposed to an auxotrophic mutation where bacteria die because they lack an essential nutrient for survival, the kill switch is triggered by a particular factor in the environment that induces the production of toxic molecules within the microbe that cause cell death.

Bacteria comprising kill switches have been engineered for in vitro research purposes, e.g., to limit the spread of a biofuel-producing microorganism outside of a laboratory environment. Bacteria engineered for in vivo administration to treat a disease may also be programmed to die at a specific time after the expression and delivery of a heterologous gene or genes, for example, an effector molecule, or after the subject has experienced the therapeutic effect. For example, in some embodiments, the kill switch is activated to kill the bacteria after a period of time following expression of the effector molecule, e.g., GLP-2 or others described herein. In some embodiments, the kill switch is activated in a delayed fashion following expression of the effector molecule. Alternatively, the bacteria may be engineered to die after the bacterium has spread outside of a disease site. Specifically, it may be useful to prevent long-term colonization of subjects by the microorganism, spread of the microorganism outside the area of interest (for example, outside the gut or the tumor microenvironment) within the subject, or spread of the microorganism outside of the subject into the environment (for example, spread to the environment through the stool of the subject). Examples of such toxins that can be used in kill-switches include, but are not limited to, bacteriocins, lysins, and other molecules that cause cell death by lysing cell membranes, degrading cellular DNA, or other mechanisms. Such toxins can be used individually or in combination. The switches that control their production can be based on, for example, transcriptional activation (toggle switches; see, e.g., Gardner et al., 2000), translation (riboregulators), or DNA recombination (recombinase-based switches), and can sense environmental stimuli such as anaerobiosis or reactive oxygen species. These switches can be activated by a single environmental factor or may require several activators in AND, OR, NAND and NOR logic configurations to induce cell death. For example, an AND riboregulator switch is activated by tetracycline, isopropyl β-D-1-thiogalactopyranoside (IPTG), and arabinose to induce the expression of lysins, which permeabilize the cell membrane and kill the cell. IPTG induces the expression of the endolysin and holin mRNAs, which are then derepressed by the addition of arabinose and tetracycline. All three inducers must be present to cause cell death. Examples of kill switches are known in the art (Callura et al., 2010).

Kill-switches can be designed such that a toxin is produced in response to an environmental condition or external signal (e.g., the bacteria is killed in response to an external cue) or, alternatively designed such that a toxin is produced once an environmental condition no longer exists or an external signal is ceased.

Thus, in some embodiments, the recombinant bacteria of the disclosure are further programmed to die after sensing an exogenous environmental signal, for example, in low-oxygen conditions, in the presence of ROS, or in the presence of RNS. In some embodiments, the recombinant bacteria of the present disclosure comprise one or more genes encoding one or more recombinase(s), whose expression is induced in response to an environmental condition or signal and causes one or more recombination events that ultimately leads to the expression of a toxin which kills the cell. In some embodiments, the at least one recombination event is the flipping of an inverted heterologous gene encoding a bacterial toxin which is then constitutively expressed after it is flipped by the first recombinase. In one embodiment, constitutive expression of the bacterial toxin kills the recombinant bacterium. In these types of kill-switch systems once the engineered bacterial cell senses the exogenous environmental condition and expresses the heterologous gene of interest, the recombinant bacterial cell is no longer viable.

In another embodiment in which the recombinant bacteria of the present disclosure express one or more recombinase(s) in response to an environmental condition or signal causing at least one recombination event, the recombinant bacterium further expresses a heterologous gene encoding an anti-toxin in response to an exogenous environmental condition or signal. In one embodiment, the at least one recombination event is flipping of an inverted heterologous gene encoding a bacterial toxin by a first recombinase. In one embodiment, the inverted heterologous gene encoding the bacterial toxin is located between a first forward recombinase recognition sequence and a first reverse recombinase recognition sequence. In one embodiment, the heterologous gene encoding the bacterial toxin is constitutively expressed after it is flipped by the first recombinase. In one embodiment, the anti-toxin inhibits the activity of the toxin, thereby delaying death of the recombinant bacterium. In one embodiment, the recombinant bacterium is killed by the bacterial toxin when the heterologous gene encoding the anti-toxin is no longer expressed when the exogenous environmental condition is no longer present.

In another embodiment, the at least one recombination event is flipping of an inverted heterologous gene encoding a second recombinase by a first recombinase, followed by the flipping of an inverted heterologous gene encoding a bacterial toxin by the second recombinase. In one embodiment, the inverted heterologous gene encoding the second recombinase is located between a first forward recombinase recognition sequence and a first reverse recombinase recognition sequence. In one embodiment, the inverted heterologous gene encoding the bacterial toxin is located between a second forward recombinase recognition sequence and a second reverse recombinase recognition sequence. In one embodiment, the heterologous gene encoding the second recombinase is constitutively expressed after it is flipped by the first recombinase. In one embodiment, the heterologous gene encoding the bacterial toxin is constitutively expressed after it is flipped by the second recombinase. In one embodiment, the recombinant bacterium is killed by the bacterial toxin. In one embodiment, the recombinant bacterium further expresses a heterologous gene encoding an anti-toxin in response to the exogenous environmental condition. In one embodiment, the anti-toxin inhibits the activity of the toxin when the exogenous environmental condition is present, thereby delaying death of the recombinant bacterium. In one embodiment, the recombinant bacterium is killed by the bacterial toxin when the heterologous gene encoding the anti-toxin is no longer expressed when the exogenous environmental condition is no longer present.

In one embodiment, the at least one recombination event is flipping of an inverted heterologous gene encoding a second recombinase by a first recombinase, followed by flipping of an inverted heterologous gene encoding a third recombinase by the second recombinase, followed by flipping of an inverted heterologous gene encoding a bacterial toxin by the third recombinase.

In one embodiment, the at least one recombination event is flipping of an inverted heterologous gene encoding a first excision enzyme by a first recombinase. In one embodiment, the inverted heterologous gene encoding the first excision enzyme is located between a first forward recombinase recognition sequence and a first reverse recombinase recognition sequence. In one embodiment, the heterologous gene encoding the first excision enzyme is constitutively expressed after it is flipped by the first recombinase. In one embodiment, the first excision enzyme excises a first essential gene. In one embodiment, the programmed recombinant bacterial cell is not viable after the first essential gene is excised.

In one embodiment, the first recombinase further flips an inverted heterologous gene encoding a second excision enzyme. In one embodiment, the inverted heterologous gene encoding the second excision enzyme is located between a second forward recombinase recognition sequence and a second reverse recombinase recognition sequence. In one embodiment, the heterologous gene encoding the second excision enzyme is constitutively expressed after it is flipped by the first recombinase. In one embodiment, the recombinant bacterium dies or is no longer viable when the first essential gene and the second essential gene are both excised. In one embodiment, the recombinant bacterium dies or is no longer viable when either the first essential gene is excised or the second essential gene is excised by the first recombinase.

In one embodiment, the recombinant bacterium dies after the at least one recombination event occurs. In another embodiment, the recombinant bacterium is no longer viable after the at least one recombination event occurs.

In any of these embodiment, the recombinase can be a recombinase selected from the group consisting of: BxbI, PhiC31, TP901, BxbI, PhiC31, TP901, HK022, HP1, R4, Int1, Int2, Int3, Int4, Int5, Int6, Int7, Int8, Int9, Int10, Int11, Int12, Int13, Int14, Int15, Int16, Int17, Int18, Int19, Int20, Int21, Int22, Int23, Int24, Int25, Int26, Int27, Int28, Int29, Int30, Int31, Int32, Int33, and Int34, or a biologically active fragment thereof.

In the above-described kill-switch circuits, a toxin is produced in the presence of an environmental factor or signal. In another aspect of kill-switch circuitry, a toxin may be repressed in the presence of an environmental factor (not produced) and then produced once the environmental condition or external signal is no longer present. Such kill switches are called repression-based kill switches and represent systems in which the bacterial cells are viable only in the presence of an external factor or signal, such as arabinose or other sugar. The disclosure provides recombinant bacterial cells which express one or more heterologous gene(s) upon sensing arabinose or other sugar in the exogenous environment. In this aspect, the recombinant bacterial cells contain the araC gene, which encodes the AraC transcription factor, as well as one or more genes under the control of the araBAD promoter. In the absence of arabinose, the AraC transcription factor adopts a conformation that represses transcription of genes under the control of the araBAD promoter. In the presence of arabinose, the AraC transcription factor undergoes a conformational change that allows it to bind to and activate the araBAD promoter, which induces expression of the desired gene, for example tetR, which represses expression of a toxin gene. In this embodiment, the toxin gene is repressed in the presence of arabinose or other sugar. In an environment where arabinose is not present, the tetR gene is not activated and the toxin is expressed, thereby killing the bacteria. The arabinose system can also be used to express an essential gene, in which the essential gene is only expressed in the presence of arabinose or other sugar and is not expressed when arabinose or other sugar is absent from the environment.

Thus, in some embodiments in which one or more heterologous gene(s) are expressed upon sensing arabinose in the exogenous environment, the one or more heterologous genes are directly or indirectly under the control of the araBAD promoter (P_{araBAD}). In some embodiments, the expressed heterologous gene is selected from one or more of the following: a heterologous therapeutic gene, a heterologous gene encoding an anti-toxin, a heterologous gene encoding a repressor protein or polypeptide, for example, a TetR repressor, a heterologous gene encoding an essential protein not found in the bacterial cell, and/or a heterologous encoding a regulatory protein or polypeptide.

Arabinose inducible promoters are known in the art, including Para, P_{araB}, P_{araC}, and P_{araBAD}. In one embodiment, the arabinose inducible promoter is from E. coli. In some embodiments, the P_{araC} promoter and the P_{araBAD} promoter operate as a bidirectional promoter, with the P_{araBAD} promoter controlling expression of a heterologous gene(s) in one direction, and the P_{araC} (in close proximity to, and on the opposite strand from the P_{araBAD} promoter), controlling expression of a heterologous gene(s) in the other direction. In the presence of arabinose, transcription of both heterologous genes from both promoters is induced. However, in the absence of arabinose, transcription of both heterologous genes from both promoters is not induced.

In one exemplary embodiment of the disclosure, the recombinant bacteria of the present disclosure contains a kill-switch having at least the following sequences: a P_{araBAD} promoter operably linked to a heterologous gene encoding a Tetracycline Repressor Protein (TetR), a P_{arac} promoter operably linked to a heterologous gene encoding AraC transcription factor, and a heterologous gene encoding a bacterial toxin operably linked to a promoter which is repressed by the Tetracycline Repressor Protein (P_{TetR}). In the presence of arabinose, the AraC transcription factor activates the P_{araBAD} promoter, which activates transcription of the TetR protein which, in turn, represses transcription of the toxin. In the absence of arabinose, however, AraC suppresses transcription from the P_{araBAD} promoter and no TetR protein is expressed. In this case, expression of the heterologous toxin gene is activated, and the toxin is expressed. The toxin builds up in the recombinant bacterial cell, and the recombinant bacterial cell is killed. In one embodiment, the araC gene encoding the AraC transcription factor is under the control of a constitutive promoter and is therefore constitutively expressed.

In one embodiment of the disclosure, the recombinant bacterium further comprises an anti-toxin under the control of a constitutive promoter. In this situation, in the presence of arabinose, the toxin is not expressed due to repression by TetR protein, and the anti-toxin protein builds-up in the cell. However, in the absence of arabinose, TetR protein is not expressed, and expression of the toxin is induced. The toxin begins to build-up within the recombinant bacterial cell. The recombinant bacterial cell is no longer viable once the toxin protein is present at either equal or greater amounts than that of the anti-toxin protein in the cell, and the recombinant bacterial cell will be killed by the toxin.

In another embodiment of the disclosure, the recombinant bacterium further comprises an anti-toxin under the control of the P_{araBAD} promoter. In this situation, in the presence of arabinose, TetR and the anti-toxin are expressed, the anti-toxin builds up in the cell, and the toxin is not expressed due to repression by TetR protein. However, in the absence of arabinose, both the TetR protein and the anti-toxin are not expressed, and expression of the toxin is induced. The toxin begins to build-up within the recombinant bacterial cell. The recombinant bacterial cell is no longer viable once the toxin protein is expressed, and the recombinant bacterial cell will be killed by the toxin.

In another exemplary embodiment of the disclosure, the recombinant bacteria of the present disclosure contains a kill-switch having at least the following sequences: a_{ParaBAD} promoter operably linked to a heterologous gene encoding an essential polypeptide not found in the recombinant bacterial cell (and required for survival), and a P_{araC} promoter operably linked to a heterologous gene encoding AraC transcription factor. In the presence of arabinose, the AraC transcription factor activates the P_{araBAD} promoter, which activates transcription of the heterologous gene encoding the essential polypeptide, allowing the recombinant bacterial cell to survive. In the absence of arabinose, however, AraC suppresses transcription from the P_{araBAD} promoter and the essential protein required for survival is not expressed. In this case, the recombinant bacterial cell dies in the absence of arabinose. In some embodiments, the sequence of P_{araBAD} promoter operably linked to a heterologous gene encoding an essential polypeptide not found in the recombinant bacterial cell can be present in the bacterial cell in conjunction with the TetR/toxin kill-switch system described directly above. In some embodiments, the sequence of P_{araBAD} promoter operably linked to a heterologous gene encoding an essential polypeptide not found in the recombinant bacterial cell can be present in the bacterial cell in conjunction with the TetR/toxin/anti-toxin kill-switch system described directly above.

In yet other embodiments, the bacteria may comprise a plasmid stability system with a plasmid that produces both a short-lived anti-toxin and a long-lived toxin. In this system, the bacterial cell produces equal amounts of toxin and anti-toxin to neutralize the toxin. However, if/when the cell loses the plasmid, the short-lived anti-toxin begins to decay. When the anti-toxin decays completely the cell dies as a result of the longer-lived toxin killing it.

In some embodiments, the engineered bacteria of the present disclosure further comprise the gene(s) encoding the components of any of the above-described kill-switch circuits.

In any of the above-described embodiments, the bacterial toxin may be selected from the group consisting of a lysin, Hok, Fst, TisB, LdrD, Kid, SymE, MazF, FlmA, Ibs, XCV2162, dinJ, CcdB, MazF, ParE, YafO, Zeta, hicB, relB, yhaV, yoeB, chpBK, hipA, microcin B, microcin B17, microcin C, microcin C7-051, microcin J25, microcin ColV, microcin 24, microcin L, microcin D93, microcin L, microcin E492, microcin H47, microcin 147, microcin M, colicin A, colicin E1, colicin K, colicin N, colicin U, colicin B, colicin Ia, colicin Ib, colicin 5, colicin 10, colicin S4, colicin Y, colicin E2, colicin E7, colicin E8, colicin E9, colicin E3, colicin E4, colicin E6, colicin E5, colicin D, colicin M, and cloacin DF13, or a biologically active fragment thereof.

In any of the above-described embodiments, the anti-toxin may be selected from the group consisting of an anti-lysin, Sok, RNAII, IstR, RdlD, Kis, SymR, MazE, FlmB, Sib, ptaRNA1, yafQ, CcdA, MazE, ParD, yafN, Epsilon, HicA, relE, prlF, yefM, chpBI, hipB, MccE, MccE^{CTD}, MccF, Cai, ImmE1, Cki, Cni, Cui, Cbi, Iia, Imm, Cfi, Im10, Csi, Cyi, Im2, Im7, Im8, Im9, Im3, Im4, ImmE6, cloacin immunity protein (Cim), ImmE5, ImmD, and Cmi, or a biologically active fragment thereof.

In one embodiment, the bacterial toxin is bactericidal to the recombinant bacterium. In one embodiment, the bacterial toxin is bacteriostatic to the recombinant bacterium.

In some embodiments, the recombinant bacterium provided herein is an auxotroph. In one embodiment, the recombinant bacterium is an auxotroph selected from a cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thi1 auxotroph. In some embodiments, the engineered bacteria have more than one auxotrophy, for example, they may be a ΔthyA and ΔdapA auxotroph.

In some embodiments, the recombinant bacterium provided herein further comprises a kill-switch circuit, such as any of the kill-switch circuits provided herein. For example, in some embodiments, the recombinant bacteria further comprise one or more genes encoding one or more recombinase(s) under the control of an inducible promoter and an inverted toxin sequence. In some embodiments, the recombinant bacteria further comprise one or more genes encoding an anti-toxin. In some embodiments, the engineered bacteria further comprise one or more genes encoding one or more recombinase(s) under the control of an inducible promoter and one or more inverted excision genes, wherein the excision gene(s) encode an enzyme that deletes an essential gene. In some embodiments, the recombinant bacteria further comprise one or more genes encoding an anti-toxin. In some embodiments, the engineered bacteria further comprise one or more genes encoding a toxin under the control of a promoter having a TetR repressor binding site and a gene encoding the TetR under the control of an inducible promoter that is induced by arabinose, such as P_{araBAD}. In some embodiments, the recombinant bacteria further comprise one or more genes encoding an anti-toxin.

In some embodiments, the recombinant bacterium is an auxotroph comprising a therapeutic payload and further comprises a kill-switch circuit, such as any of the kill-switch circuits described herein.

In some embodiments of the above described recombinant bacteria, the gene or gene cassette for producing the effector molecule is present on a plasmid in the bacterium and operatively linked on the plasmid to the inducible promoter. In other embodiments, the gene or gene cassette for producing the effector molecule is present in the bacterial chromosome and is operatively linked in the chromosome to the inducible promoter.

Methods of Screening

Mutagenesis

In some embodiments, the inducible promoter is operably linked to a detectable product, e.g., GFP, and can be used to screen for mutants. In some embodiments, the inducible promoter is mutagenized, and mutants are selected based upon the level of detectable product, e.g., by flow cytometry, fluorescence-activated cell sorting (FACS) when the detectable product fluoresces. In some embodiments, one or more transcription factor binding sites is mutagenized to increase or decrease binding. In alternate embodiments, the wild-type binding sites are left intact and the remainder of the regulatory region is subjected to mutagenesis. In some embodiments, the mutant promoter is inserted into the recombinant bacteria to increase expression of the effector molecule under inducing conditions, as compared to unmutated bacteria of the same subtype under the same conditions. In some embodiments, the inducible promoter and/or corresponding transcription factor is a synthetic, non-naturally occurring sequence.

In some embodiments, the gene encoding an effector molecule is mutated to increase expression and/or stability of said molecule under inducing conditions, as compared to unmutated bacteria of the same subtype under the same conditions. In some embodiments, one or more of the genes in a gene cassette for producing an effector molecule is mutated to increase expression of said molecule under inducing conditions, as compared to unmutated bacteria of the same subtype under the same conditions. In some embodiments, the efficacy or activity of any of the importers and exporters for metabolites of interest can be improved through mutations in any of these genes. Mutations increase uptake or export of such metabolites, including but not limited to, tryptophan, e.g., under inducing conditions, as compared to unmutated bacteria of the same subtype under the same conditions. Methods for directed mutation and screening are known in the art.

Generation of Bacterial Strains with Enhance Ability to Transport Metabolites of Interest

Due to their ease of culture, short generation times, very high population densities and small genomes, microbes can be evolved to unique phenotypes in abbreviated timescales. Adaptive laboratory evolution (ALE) is the process of passaging microbes under selective pressure to evolve a strain with a preferred phenotype. Most commonly, this is applied to increase utilization of carbon/energy sources or adapting a strain to environmental stresses (e.g., temperature, pH), whereby mutant strains more capable of growth on the carbon substrate or under stress will outcompete the less adapted strains in the population and will eventually come to dominate the population.

This same process can be extended to any essential metabolite by creating an auxotroph. An auxotroph is a strain incapable of synthesizing an essential metabolite and must therefore have the metabolite provided in the media to grow. In this scenario, by making an auxotroph and passaging it on decreasing amounts of the metabolite, the resulting dominant strains should be more capable of obtaining and incorporating this essential metabolite.

For example, if the biosynthetic pathway for producing a metabolite of interest is disrupted a strain capable of high-affinity capture of the metabolite of interest can be evolved via ALE. First, the strain is grown in varying concentrations of the auxotrophic metabolite of interest, until a minimum concentration to support growth is established. The strain is then passaged at that concentration, and diluted into lowering concentrations of the metabolite of interest at regular intervals. Over time, cells that are most competitive for the metabolite of interest—at growth-limiting concentrations—will come to dominate the population. These strains will likely have mutations in their metabolite of interest-transporters resulting in increased ability to import the essential and limiting metabolite of interest.

Similarly, by using an auxotroph that cannot use an upstream metabolite to form the metabolite of interest, a strain can be evolved that not only can more efficiently import the upstream metabolite, but also convert the metabolite into the essential downstream metabolite of interest. These strains will also evolve mutations to increase import of the upstream metabolite, but may also contain mutations which increase expression or reaction kinetics of downstream enzymes, or that reduce competitive substrate utilization pathways.

A metabolite innate to the microbe can be made essential via mutational auxotrophy and selection applied with growth-limiting supplementation of the endogenous metabolite. However, phenotypes capable of consuming non-native compounds can be evolved by tying their consumption to the production of an essential compound. For example, if a gene from a different organism is isolated which can produce an essential compound or a precursor to an essential compound this gene can be recombinantly introduced and expressed in the heterologous host. This new host strain will now have the ability to synthesize an essential nutrient from a previously non-metabolizable substrate.

Hereby, a similar ALE process can be applied by creating an auxotroph incapable of converting an immediately downstream metabolite and selecting in growth-limiting amounts of the non-native compound with concurrent expression of the recombinant enzyme. This will result in mutations in the transport of the non-native substrate, expression and activity of the heterologous enzyme and expression and activity of downstream native enzymes. It should be emphasized that the key requirement in this process is the ability to tether the consumption of the non-native metabolite to the production of a metabolite essential to growth.

Once the basis of the selection mechanism is established and minimum levels of supplementation have been established, the actual ALE experimentation can proceed. Throughout this process several parameters must be vigilantly monitored. It is important that the cultures are maintained in an exponential growth phase and not allowed to reach saturation/stationary phase. This means that growth rates must be check during each passaging and subsequent dilutions adjusted accordingly. If growth rate improves to such a degree that dilutions become large, then the concentration of auxotrophic supplementation should be decreased such that growth rate is slowed, selection pressure is increased and dilutions are not so severe as to heavily bias subpopulations during passaging. In addition, at regular intervals cells should be diluted, grown on solid media and individual clones tested to confirm growth rate phenotypes observed in the ALE cultures.

Predicting when to halt the stop the ALE experiment also requires vigilance. As the success of directing evolution is tied directly to the number of mutations “screened” throughout the experiment and mutations are generally a function of errors during DNA replication, the cumulative cell divisions (CCD) acts as a proxy for total mutants which have been screened. Previous studies have shown that beneficial phenotypes for growth on different carbon sources can be isolated in about 10^{11.2} CCD¹. This rate can be accelerated by the addition of chemical mutagens to the cultures—such as N-methyl-N-nitro-N-nitrosoguanidine (NTG)—which causes increased DNA replication errors. However, when continued passaging leads to marginal or no improvement in growth rate the population has converged to some fitness maximum and the ALE experiment can be halted.

At the conclusion of the ALE experiment, the cells should be diluted, isolated on solid media and assayed for growth phenotypes matching that of the culture flask. Best performers from those selected are then prepped for genomic DNA and sent for whole genome sequencing. Sequencing with reveal mutations occurring around the genome capable of providing improved phenotypes, but will also contain silent mutations (those which provide no benefit but do not detract from desired phenotype). In cultures evolved in the presence of NTG or other chemical mutagen, there will be significantly more silent, background mutations. If satisfied with the best performing strain in its current state, the user can proceed to application with that strain. Otherwise the contributing mutations can be deconvoluted from the evolved strain by reintroducing the mutations to the parent strain by genome engineering techniques. See Lee, D.-H., Feist, A. M., Barrett, C. L. & Palsson, B. Ø. Cumulative Number of Cell Divisions as a Meaningful Timescale for Adaptive Laboratory Evolution of Escherichia coli. PLoS ONE 6, e26172 (2011).

Similar methods can be used to generate E. coli Nissle mutants that consume or import metabolites, including, but not limited to, tryptophan.

### Pharmaceutical Compositions and Formulations

Pharmaceutical compositions comprising one or more recombinant bacteria, alone or in combination with prophylactic agents, therapeutic agents, and/or pharmaceutically acceptable carriers are provided.

In certain embodiments, the pharmaceutical composition comprises one species, strain, or subtype of bacteria that are engineered to comprise the genetic modifications described herein, e.g., to produce an effector molecule. In alternate embodiments, the pharmaceutical composition comprises two or more species, strains, and/or subtypes of bacteria that are each engineered to comprise the genetic modifications described herein, e.g., to produce an effector molecule.

In certain embodiments, a combination of two or more different genetically engineered microorganisms can be administered at the same time. In some embodiments, the method comprises administering the a subject a combination of two or more genetically engineered microorganisms, a first microorganism which expresses a first payload, and at least a second microorganism which expresses a second payload. In some embodiments, the method comprises compositions comprising a combination of two or more genetically engineered microorganisms, a first microorganisms which expresses a first payload, and at least a second microorganism which expresses a second payload.

The pharmaceutical compositions described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, PA). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The genetically engineered microorganisms may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, immediate-release, pulsatile-release, delayed-release, or sustained release). Suitable dosage amounts for the recombinant bacteria may range from about 10e5 to 10e12 bacteria, e.g., approximately 10e5 bacteria, approximately 10e6 bacteria, approximately 10e7 bacteria, approximately 10e8 bacteria, approximately 10e9 bacteria, approximately 10e10 bacteria, approximately 10e11 bacteria, or approximately 10e12 bacteria. The composition may be administered once or more daily, weekly, or monthly. The composition may be administered before, during, or following a meal. In one embodiment, the pharmaceutical composition is administered before the subject eats a meal. In one embodiment, the pharmaceutical composition is administered concurrently with a meal. In one embodiment, the pharmaceutical composition is administered after the subject eats a meal.

The recombinant bacteria may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the recombinant bacteria may be formulated in a solution of sodium bicarbonate, e.g., 1 molar solution of sodium bicarbonate (to buffer an acidic cellular environment, such as the stomach, for example). The recombinant bacteria may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The genetically engineered microorganisms may be administered intravenously, e.g., by infusion or injection. The genetically engineered microorganisms of the disclosure may be administered intrathecally. In some embodiments, the genetically engineered microorganisms may be administered orally. The genetically engineered microorganisms disclosed herein may be administered topically and formulated in the form of an ointment, cream, transdermal patch, lotion, gel, shampoo, spray, aerosol, solution, emulsion, or other form well known to one of skill in the art. See, e.g., “Remington's Pharmaceutical Sciences,” Mack Publishing Co., Easton, PA. In an embodiment, for non-sprayable topical dosage forms, viscous to semi-solid or solid forms comprising a carrier or one or more excipients compatible with topical application and having a dynamic viscosity greater than water are employed. Suitable formulations include, but are not limited to, solutions, suspensions, emulsions, creams, ointments, powders, liniments, salves, etc., which may be sterilized or mixed with auxiliary agents (e.g., preservatives, stabilizers, wetting agents, buffers, or salts) for influencing various properties, e.g., osmotic pressure. Other suitable topical dosage forms include sprayable aerosol preparations wherein the active ingredient in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms. Examples of such additional ingredients are well known in the art. In one embodiment, the pharmaceutical composition comprising the recombinant bacteria may be formulated as a hygiene product. For example, the hygiene product may be an antibacterial formulation, or a fermentation product such as a fermentation broth. Hygiene products may be, for example, shampoos, conditioners, creams, pastes, lotions, and lip balms.

The genetically engineered microorganisms disclosed herein may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Tablets or capsules can be prepared by conventional means with pharmaceutically acceptable excipients such as binding agents (e.g., pregelatinised maize starch, polyvinylpyrrolidone, hydroxypropyl methylcellulose, carboxymethylcellulose, polyethylene glycol, sucrose, glucose, sorbitol, starch, gum, kaolin, and tragacanth); fillers (e.g., lactose, microcrystalline cellulose, or calcium hydrogen phosphate); lubricants (e.g., calcium, aluminum, zinc, stearic acid, polyethylene glycol, sodium lauryl sulfate, starch, sodium benzoate, L-leucine, magnesium stearate, talc, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, and common membranes include, but are not limited to, polylactide, polyglycolic acid, polyanhydride, other biodegradable polymers, alginate-polylysine-alginate (APA), alginate-polymethylene-co-guanidine-alginate (A-PMCG-A), hydroymethylacrylate-methyl methacrylate (HEMA-MMA), multilayered HEMA-MMA-MAA, polyacrylonitrilevinylchloride (PAN-PVC), acrylonitrile/sodium methallylsulfonate (AN-69), polyethylene glycol/poly pentamethylcyclopentasiloxane/polydimethylsiloxane (PEG/PD5/PDMS), poly N,N-dimethyl acrylamide (PDMAAm), siliceous encapsulates, cellulose sulphate/sodium alginate/polymethylene-co-guanidine (CS/A/PMCG), cellulose acetate phthalate, calcium alginate, k-carrageenan-locust bean gum gel beads, gellan-xanthan beads, poly(lactide-co-glycolides), carrageenan, starch poly-anhydrides, starch polymethacrylates, polyamino acids, and enteric coating polymers.

In some embodiments, the genetically engineered microorganisms are enterically coated for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of the genetically engineered microorganisms described herein.

In one embodiment, the genetically engineered microorganisms of the disclosure may be formulated in a composition suitable for administration to pediatric subjects. As is well known in the art, children differ from adults in many aspects, including different rates of gastric emptying, pH, gastrointestinal permeability, etc. (Ivanovska et al., Pediatrics, 134(2):361-372, 2014). Moreover, pediatric formulation acceptability and preferences, such as route of administration and taste attributes, are critical for achieving acceptable pediatric compliance. Thus, in one embodiment, the composition suitable for administration to pediatric subjects may include easy-to-swallow or dissolvable dosage forms, or more palatable compositions, such as compositions with added flavors, sweeteners, or taste blockers. In one embodiment, a composition suitable for administration to pediatric subjects may also be suitable for administration to adults.

In one embodiment, the composition suitable for administration to pediatric subjects may include a solution, syrup, suspension, elixir, powder for reconstitution as suspension or solution, dispersible/effervescent tablet, chewable tablet, gummy candy, lollipop, freezer pop, troche, chewing gum, oral thin strip, orally disintegrating tablet, sachet, soft gelatin capsule, sprinkle oral powder, or granules. In one embodiment, the composition is a gummy candy, which is made from a gelatin base, giving the candy elasticity, desired chewy consistency, and longer shelf-life. In some embodiments, the gummy candy may also comprise sweeteners or flavors.

In one embodiment, the composition suitable for administration to pediatric subjects may include a flavor. As used herein, “flavor” is a substance (liquid or solid) that provides a distinct taste and aroma to the formulation. Flavors also help to improve the palatability of the formulation. Flavors include, but are not limited to, strawberry, vanilla, lemon, grape, bubble gum, and cherry.

In certain embodiments, the genetically engineered microorganisms may be orally administered, for example, with an inert diluent or an assimilable edible carrier. The compound may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation.

In another embodiment, the pharmaceutical composition comprising the recombinant bacteria may be a comestible product, for example, a food product. In one embodiment, the food product is milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods (such as infant cakes), nutritional food products, animal feeds, or dietary supplements. In one embodiment, the food product is a fermented food, such as a fermented dairy product. In one embodiment, the fermented dairy product is yogurt. In another embodiment, the fermented dairy product is cheese, milk, cream, ice cream, milk shake, or kefir. In another embodiment, the recombinant bacteria are combined in a preparation containing other live bacterial cells intended to serve as probiotics. In another embodiment, the food product is a beverage. In one embodiment, the beverage is a fruit juice-based beverage or a beverage containing plant or herbal extracts. In another embodiment, the food product is a jelly or a pudding. Other food products suitable for administration of the recombinant bacteria are well known in the art. For example, see U.S. 2015/0359894 and US 2015/0238545, the entire contents of each of which are expressly incorporated herein by reference. In yet another embodiment, the pharmaceutical composition is injected into, sprayed onto, or sprinkled onto a food product, such as bread, yogurt, or cheese.

In some embodiments, the composition is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated. The pharmaceutical compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents.

The genetically engineered microorganisms described herein may be administered intranasally, formulated in an aerosol form, spray, mist, or in the form of drops, and conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). Pressurized aerosol dosage units may be determined by providing a valve to deliver a metered amount. Capsules and cartridges (e.g., of gelatin) for use in an inhaler or insufflator may be formulated containing a powder mix of the compound and a suitable powder base such as lactose or starch.

The genetically engineered microorganisms may be administered and formulated as depot preparations. Such long acting formulations may be administered by implantation or by injection, including intravenous injection, subcutaneous injection, local injection, direct injection, or infusion. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, disclosed herein are pharmaceutically acceptable compositions in single dosage forms. Single dosage forms may be in a liquid or a solid form. Single dosage forms may be administered directly to a patient without modification or may be diluted or reconstituted prior to administration. In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, e.g., by infusion.

Single dosage forms of the pharmaceutical composition may be prepared by portioning the pharmaceutical composition into smaller aliquots, single dose containers, single dose liquid forms, or single dose solid forms, such as tablets, granulates, nanoparticles, nanocapsules, microcapsules, microtablets, pellets, or powders, which may be enterically coated or uncoated. A single dose in a solid form may be reconstituted by adding liquid, typically sterile water or saline solution, prior to administration to a patient.

In other embodiments, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

Dosage regimens may be adjusted to provide a therapeutic response. Dosing can depend on several factors, including severity and responsiveness of the disease, route of administration, time course of treatment (days to months to years), and time to amelioration of the disease. For example, a single bolus may be administered at one time, several divided doses may be administered over a predetermined period of time, or the dose may be reduced or increased as indicated by the therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician. Toxicity and therapeutic efficacy of compounds provided herein can be determined by standard pharmaceutical procedures in cell culture or animal models. For example, LD50, ED50, EC50, and IC50 may be determined, and the dose ratio between toxic and therapeutic effects (LD50/ED50) may be calculated as the therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

### Methods of Treatment

Another aspect provides methods of treating autoimmune disorders, cancer, metabolic diseases, diseases relating to inborn errors of metabolism, neurological or neurodegenerative diseases, diarrheal diseases, IBD, related diseases, and other diseases that benefit from reduced gut inflammation and/or enhanced gut barrier function. In some embodiments, the invention provides for the use of at least one genetically engineered species, strain, or subtype of bacteria described herein for the manufacture of a medicament. In some embodiments, the invention provides for the use of at least one genetically engineered species, strain, or subtype of bacteria described herein for the manufacture of a medicament for treating autoimmune disorders, cancer, metabolic diseases, diseases relating to inborn errors of metabolism, neurological or neurodegenerative diseases, diarrheal diseases, IBD, related diseases, and other diseases that benefit from reduced gut inflammation and/or enhanced gut barrier function. In some embodiments, the invention provides at least one genetically engineered species, strain, or subtype of bacteria described herein for use in treating autoimmune disorders, cancer, metabolic diseases, diseases relating to inborn errors of metabolism, neurological or neurodegenerative diseases, diarrheal diseases, IBD, related diseases, and other diseases that benefit from reduced gut inflammation and/or enhanced gut barrier function.

In some embodiments, the diarrheal disease is selected from the group consisting of acute watery diarrhea, e.g., cholera, acute bloody diarrhea, e.g., dysentery, and persistent diarrhea. In some embodiments, the IBD or related disease is selected from the group consisting of Crohn's disease, ulcerative colitis, collagenous colitis, lymphocytic colitis, diversion colitis, Behcet's disease, intermediate colitis, short bowel syndrome, ulcerative proctitis, proctosigmoiditis, left-sided colitis, pancolitis, and fulminant colitis.

In some embodiments, the disease or condition is an autoimmune disorder selected from the group consisting of acute disseminated encephalomyelitis (ADEM), acute necrotizing hemorrhagic leukoencephalitis, Addison's disease, agammaglobulinemia, alopecia areata, amyloidosis, ankylosing spondylitis, anti-GBM/anti-TBM nephritis, antiphospholipid syndrome (APS), autoimmune angioedema, autoimmune aplastic anemia, autoimmune dysautonomia, autoimmune hemolytic anemia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenic purpura (ATP), autoimmune thyroid disease, autoimmune urticarial, axonal & neuronal neuropathies, Balo disease, Behcet's disease, bullous pemphigoid, cardiomyopathy, Castleman disease, celiac disease, Chagas disease, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogan's syndrome, cold agglutinin disease, congenital heart block, Coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, experimental allergic encephalomyelitis, Evans syndrome, fibrosing alveolitis, giant cell arteritis (temporal arteritis), giant cell myocarditis, glomerulonephritis, Goodpasture's syndrome, granulomatosis with polyangiitis (GPA), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, idiopathic thrombocytopenic purpura (ITP), IgA nephropathy, IgG4-related sclerosing disease, immunoregulatory lipoproteins, inclusion body myositis, interstitial cystitis, juvenile arthritis, juvenile idiopathic arthritis, juvenile myositis, Kawasaki syndrome, Lambert-Eaton syndrome, leukocytoclastic vasculitis, lichen planus, lichen sclerosus, ligneous conjunctivitis, linear IgA disease (LAD), lupus (systemic lupus erythematosus), chronic Lyme disease, Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynaud's phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, subacute bacterial endocarditis (SBE), Susac's syndrome, sympathetic ophthalmia, Takayasu's arteritis, temporal arteritis/giant cell arteritis, thrombocytopenic purpura (TTP), Tolosa-Hunt syndrome, transverse myelitis, type 1 diabetes, asthma, ulcerative colitis, undifferentiated connective tissue disease (UCTD), uveitis, vasculitis, vesiculobullous dermatosis, vitiligo, and Wegener's granulomatosis. In some embodiments, the invention provides methods for reducing, ameliorating, or eliminating one or more symptom(s) associated with these diseases, including but not limited to diarrhea, bloody stool, mouth sores, perianal disease, abdominal pain, abdominal cramping, fever, fatigue, weight loss, iron deficiency, anemia, appetite loss, weight loss, anorexia, delayed growth, delayed pubertal development, and inflammation of the skin, eyes, joints, liver, and bile ducts. In some embodiments, the invention provides methods for reducing gut inflammation and/or enhancing gut barrier function, thereby ameliorating or preventing a systemic autoimmune disorder, e.g., asthma (Arrieta et al., 2015).

The method may comprise preparing a pharmaceutical composition with at least one genetically engineered species, strain, or subtype of bacteria described herein, and administering the pharmaceutical composition to a subject in a therapeutically effective amount.

In certain embodiments, the pharmaceutical composition described herein is administered to reduce gut inflammation, enhance gut barrier function, and/or treat or prevent an autoimmune disorder in a subject. In some embodiments, the methods of the present disclosure may reduce gut inflammation in a subject by at least about 10%, 20%, 25%, 30%, 40%, 50%, 60%, 70%, 75%, 80%, 85%, 90%, 95%, or more as compared to levels in an untreated or control subject. In some embodiments, the methods of the present disclosure may enhance gut barrier function in a subject by at least about 10%, 20%, 25%, 30%, 40%, 50%, 60%, 70%, 75%, 80%, 85%, 90%, 95%, or more as compared to levels in an untreated or control subject. In some embodiments, changes in inflammation and/or gut barrier function are measured by comparing a subject before and after administration of the pharmaceutical composition. In some embodiments, the method of treating or ameliorating the autoimmune disorder and/or the disease or condition associated with gut inflammation and/or compromised gut barrier function allows one or more symptoms of the disease or condition to improve by at least about 10%, 20%, 30%, 40%, 50%, 60%, 70%, 80%, 90%, 95%, or more.

In some embodiments, reduction is measured by comparing the levels of inflammation in a subject before and after administration of the pharmaceutical composition. In one embodiment, the levels of inflammation is reduced in the gut of the subject. In one embodiment, gut barrier function is enhanced in the gut of the subject. In another embodiment, levels of inflammation is reduced in the blood of the subject. In another embodiment, the levels of inflammation is reduced in the plasma of the subject. In another embodiment, levels of inflammation is reduced in the brain of the subject.

In one embodiment, the pharmaceutical composition described herein is administered to reduce levels of inflammation in a subject to normal levels. In another embodiment, the pharmaceutical composition described herein is administered to reduce levels of inflammation in a subject below normal.

In some embodiments, the method of treating the autoimmune disorder allows one or more symptoms of the condition or disorder to improve by at least about 10%, 20%, 30%, 40%, 50%, 60%, 70%, 80%, 90%, 95%, or more. In some embodiments, the method of treating the disorder, allows one or more symptoms of the condition or disorder to improve by at least about two-fold, three-fold, four-fold, five-fold, six-fold, seven-fold, eight-fold, nine-fold, or ten-fold.

Before, during, and after the administration of the pharmaceutical composition, gut inflammation and/or barrier function in the subject may be measured in a biological sample, such as blood, serum, plasma, urine, fecal matter, peritoneal fluid, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions to enhance gut barrier function and/or to reduce gut inflammation to baseline levels, e.g., levels comparable to those of a healthy control, in a subject. In some embodiments, the methods may include administration of the compositions to reduce gut inflammation to undetectable levels in a subject, or to less than about 1%, 2%, 5%, 10%, 20%, 25%, 30%, 40%, 50%, 60%, 70%, 75%, or 80% of the subject's levels prior to treatment. In some embodiments, the methods may include administration of the compositions to enhance gut barrier function in a subject by about 1%, 2%, 5%, 10%, 20%, 25%, 30%, 40%, 50%, 60%, 70%, 75%, 80%, 90%, 100% or more of the subject's levels prior to treatment.

In certain embodiments, the recombinant bacteria are E. coli Nissle. The recombinant bacteria may be destroyed, e.g., by defense factors in the gut or blood serum (Sonnenborn et al., 2009) or by activation of a kill switch, several hours or days after administration. Thus, the pharmaceutical composition comprising the recombinant bacteria may be re-administered at a therapeutically effective dose and frequency. In alternate embodiments, the recombinant bacteria are not destroyed within hours or days after administration and may propagate and colonize the gut.

The pharmaceutical composition may be administered alone or in combination with one or more additional therapeutic agents, e.g., corticosteroids, aminosalicylates, anti-inflammatory agents. In some embodiments, the pharmaceutical composition is administered in conjunction with an anti-inflammatory drug (e.g., mesalazine, prednisolone, methylprednisolone, butesonide), an immunosuppressive drug (e.g., azathioprine, 6-mercaptopurine, methotrexate, cyclosporine, tacrolimus), an antibiotic (e.g., metronidazole, ornidazole, clarithromycin, rifaximin, ciprofloxacin, anti-TB), other probiotics, and/or biological agents (e.g., infliximab, adalimumab, certolizumab pegol) (Triantafillidis et al., 2011). An important consideration in the selection of the one or more additional therapeutic agents is that the agent(s) should be compatible with the recombinant bacteria, e.g., the agent(s) must not kill the bacteria. In one embodiments, the bacterial cells disclosed herein are administered to a subject once daily. In another embodiment, the bacterial cells disclosed herein are administered to a subject twice daily. In another embodiment, the bacterial cells disclosed herein are administered to a subject in combination with a meal, prior to a meal, or after a meal. The dosage of the pharmaceutical composition and the frequency of administration may be selected based on the severity of the symptoms and the progression of the disease. The appropriate therapeutically effective dose and/or frequency of administration can be selected by a treating clinician.

Another aspect provides methods of treating cancer. In some embodiments, the invention provides methods for reducing, ameliorating, or eliminating one or more symptom(s) associated with cancer. In some embodiments, the cancer is selected from adrenal cancer, adrenocortical carcinoma, anal cancer, appendix cancer, bile duct cancer, bladder cancer, bone cancer (e.g., Ewing sarcoma tumors, osteosarcoma, malignant fibrous histiocytoma), brain cancer (e.g., astrocytomas, brain stem glioma, craniopharyngioma, ependymoma), bronchial tumors, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, gestational trophoblastic disease, heart cancer, Kaposi sarcoma, kidney cancer, largyngeal cancer, hypopharyngeal cancer, leukemia (e.g., acute lymphoblastic leukemia, acute myeloid leukemia, chronic lymphocytic leukemia, chronic myelogenous leukemia), liver cancer, lung cancer, lymphoma (e.g., AIDS-related lymphoma, Burkitt lymphoma, cutaneous T cell lymphoma, Hodgkin lymphoma, Non-Hodgkin lymphoma, primary central nervous system lymphoma), malignant mesothelioma, multiple myeloma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma, skin cancer (e.g., basal cell carcinoma, melanoma), small intestine cancer, stomach cancer, teratoid tumor, testicular cancer, throat cancer, thymus cancer, thyroid cancer, unusual childhood cancers, urethral cancer, uterine cancer, uterine sarcoma, vaginal cancer, vulvar cancer, Waldenström macrogloblulinemia, and Wilms tumor. In some embodiments, the symptom(s) associated thereof include, but are not limited to, anemia, loss of appetite, irritation of bladder lining, bleeding and bruising (thrombocytopenia), changes in taste or smell, constipation, diarrhea, dry mouth, dysphagia, edema, fatigue, hair loss (alopecia), infection, infertility, lymphedema, mouth sores, nausea, pain, peripheral neuropathy, tooth decay, urinary tract infections, and/or problems with memory and concentration.

In certain embodiments, administering the pharmaceutical composition to the subject reduces cell proliferation, tumor growth, and/or tumor volume in a subject. In some embodiments, the methods of the present disclosure may reduce cell proliferation, tumor growth, and/or tumor volume by at least about 10%, 20%, 25%, 30%, 40%, 50%, 60%, 70%, 75%, 80%, 85%, 90%, 95%, or more as compared to levels in an untreated or control subject. In some embodiments, reduction is measured by comparing cell proliferation, tumor growth, and/or tumor volume in a subject before and after administration of the pharmaceutical composition. In some embodiments, the method of treating or ameliorating a cancer in a subject allows one or more symptoms of the cancer to improve by at least about 10%, 20%, 30%, 40%, 50%, 60%, 70%, 80%, 90%, 95%, or more.

Before, during, and after the administration of the pharmaceutical composition, cancerous cells and/or biomarkers in a subject may be measured in a biological sample, such as blood, serum, plasma, urine, peritoneal fluid, and/or a biopsy from a tissue or organ. In some embodiments, the methods may include administration of the compositions to reduce tumor volume in a subject to an undetectable size, or to less than about 1%, 2%, 5%, 10%, 20%, 25%, 30%, 40%, 50%, 60%, 70%, 75%, 80%, or 90% of the subject's tumor volume prior to treatment. In other embodiments, the methods may include administration of the compositions to reduce the cell proliferation rate or tumor growth rate in a subject to an undetectable rate, or to less than about 1%, 2%, 5%, 10%, 20%, 25%, 30%, 40%, 50%, 60%, 70%, 75%, 80%, or 90% of the rate prior to treatment.

For genetically engineered microorganisms expressing immune-based anti-cancer molecules, e.g., a single-chain CTLA-4 antibody, responses patterns may be different than for traditional cytotoxic therapies. For example, tumors treated with immune-based therapies may enlarge before they regress, and/or new lesions may appear (Agarwala et al., 2015). Increased tumor size may be due to heavy infiltration with lymphocytes and macrophages that are normally not present in tumor tissue. Additionally, response times may be slower than response times associated with standard therapies, e.g., cytotoxic therapies. In some embodiments, delivery of the anti-cancer molecule may modulate the growth of a subject's tumor and/or ameliorate the symptoms of a cancer while temporarily increasing the volume and/or size of the tumor.

The recombinant bacteria may be destroyed, e.g., by defense factors in tissues or blood serum (Sonnenborn et al., 2009), or by activation of a kill switch, several hours or days after administration. Thus, the pharmaceutical composition comprising the gene or gene cassette for producing the anti-cancer molecule may be re-administered at a therapeutically effective dose and frequency. In alternate embodiments, the recombinant bacteria are not destroyed within hours or days after administration and may propagate and colonize the tumor.

The pharmaceutical composition may be administered alone or in combination with one or more additional therapeutic agents, e.g., a chemotherapeutic drug such a methotrexate. An important consideration in selecting the one or more additional therapeutic agents is that the agent(s) should be compatible with the recombinant bacteria of the invention, e.g., the agent(s) must not kill the bacteria. In some studies, the efficacy of anticancer immunotherapy, e.g., CTLA-4 or PD-1 inhibitors, requires the presence of particular bacterial strains in the microbiome (Ilda et al., 2013; Vetizou et al., 2015; Sivan et al., 2015). In some embodiments, the pharmaceutical composition is administered with one or more commensal or probiotic bacteria, e.g., Bifidobacterium or Bacteroides.

In some embodiments, the recombinant bacteria are administered sequentially, simultaneously, or subsequently to dosing with one or more chemotherapeutic agents selected from Trabectedin®, Belotecan®, Cisplatin®, Carboplatin®, Bevacizumab®, Pazopanib®, 5-Fluorouracil, Capecitabine®, Irinotecan®, and Oxaliplatin®. In some embodiments, the recombinant bacteria are administered sequentially, simultaneously, or subsequently to dosing with gemcitabine (Gemzar). In any of these embodiments, the one or more bacteria are administered systemically or orally or intratumorally.

The immunostimulatory activity of bacterial DNA is mimicked by synthetic oligodeoxynucleotides (ODNs) expressing unmethylated CpG motifs. Bode et al., Expert Rev Vaccines. 2011 April; 10(4): 499-511. CpG DNA as a vaccine adjuvant. When used as vaccine adjuvants, CpG ODNs improve the function of professional antigen-presenting cells and boost the generation of humoral and cellular vaccine-specific immune responses. In some embodiments, CpG can be administered in combination with the genetically engineered bacteria of the invention.

In one embodiment, the genetically engineered microorganisms are administered in combination with tumor cell lysates.

The dosage of the pharmaceutical composition and the frequency of administration may be selected based on the severity of the symptoms and the progression of the cancer. The appropriate therapeutically effective dose and the frequency of administration can be selected by a treating clinician.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The Examples do not in any way limit the disclosure.

### Example 1. Generation of Constructs for Overproducing Therapeutic Molecules for Secretion

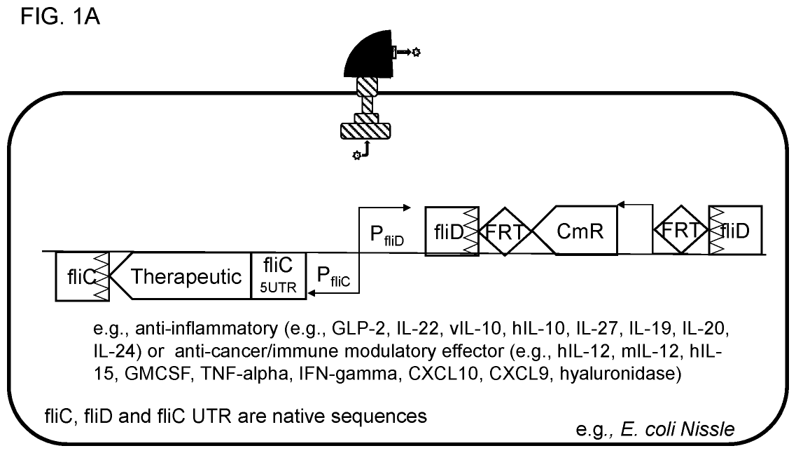

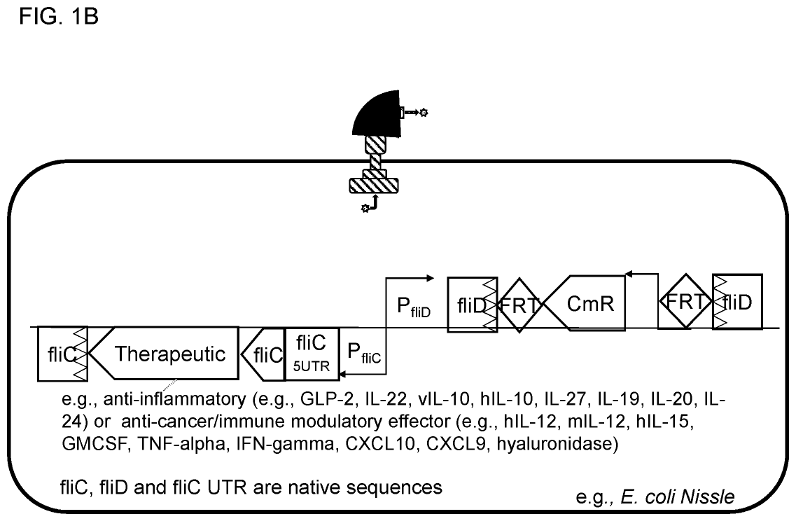

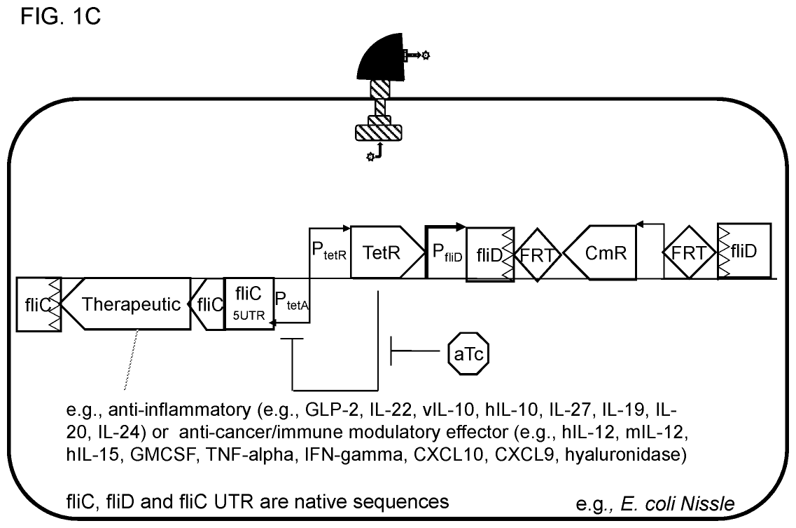

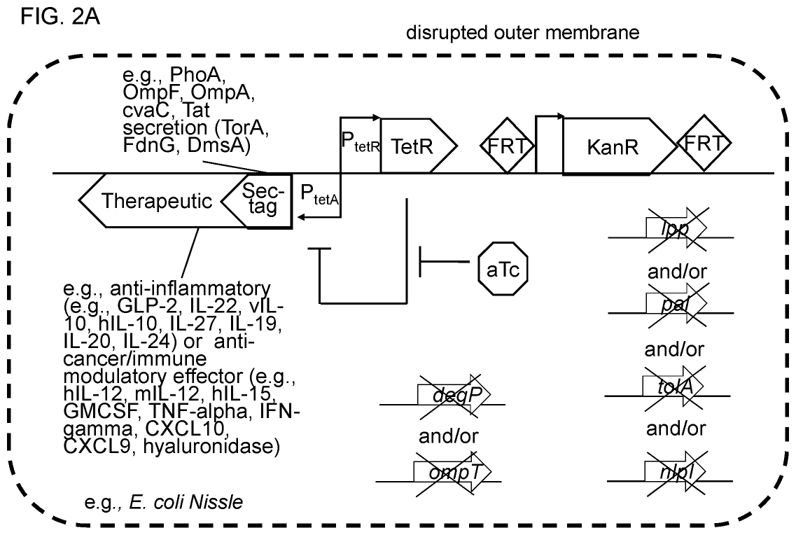

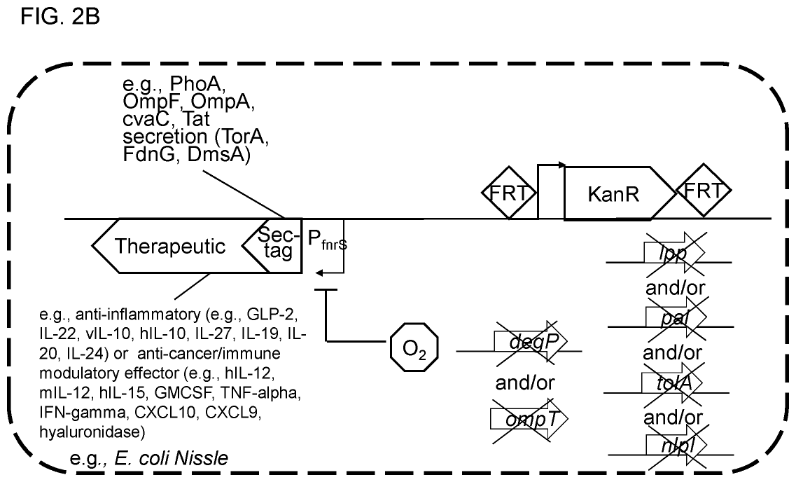

To produce strain capable of secreting anti-inflammatory or gut barrier enhancer polypeptides, e.g., GLP2, IL-22, IL-10 (viral or human), several constructs are designed employing different secretion strategies. The organization of exemplary constructs is shown in FIG. 1A, FIG. 1B, FIG. 1C, FIG. 2A and FIG. 2B. Various constructs are synthesized, and cloned into vector pBR322 for transformation of E. coli. In some embodiments, the constructs encoding the effector molecules are integrated into the genome. In some embodiments, the constructs encoding the effector molecules are on a plasmid, e.g., a medium copy plasmid.

### Example 2. GLP-2-Secreting Strains

Engineered strains comprising GLP-2 constructs were prepared. The first strain comprises a deletion in PAL and a plasmid expressing GLP-2 with an OmpF secretion tag from a tetracycline-inducible promoter. The second strain comprises the same PAL deletion and the same plasmid expressing GLP-2, further comprising a deletion in degP. Additional strains are shown in Table 17 below.

TABLE 17
GLP-2 Secreting strains
DOM mut

PAL::CmR Ptet-ompF-GLP2
PAL::CmR ompT::Kan Ptet-ompF-GLP2
PAL::CmR ompT::Kan phoA-GLP2 fusion

### Example 3. IL-22-Secreting Strains

A synthetic construct was generated in which expression of IL-22 is controlled by the tetracycline-inducible promoter (Pₜₑₜ), which is derepressed via the addition of the tetracycline analog anhydrotetracycline (aTc), and translation is driven by a strong ribosome binding site (RBS) located immediately upstream from the IL-22 coding sequence. To promote translocation to the periplasm, a 21-amino acid PhoA-secretion tag was added to the N-terminus of IL-22.

The corresponding engineered element was constructed using a synthetic DNA cassette encoding the IL-22 protein coding sequence (IDT Technologies, Coralville, Iowa) which was cloned into an initial plasmid vector, creating the plasmid Logic435. The IL-22 sequence was later amplified and cloned using Gibson assembly technology and the NEBuilder Hifi Mastermix (NEB). The final pBR322-based plasmid was sequence-verified by Sanger sequencing (Genewiz) and designated Logic522.

To create a Gram-negative bacterium capable of secreting bioactive proteins, a diffusible outer membrane (DOM) phenotype was engineered in the E. coli Nissle background. A series of DOM mutants were created by deleting different periplasmic proteins leading to a ‘leaky’ phenotype. Deletions of several different genes were tested including lpp, pal, tolA and nlpI. For example, the pal mutant (SYN3000) showed a good secretion phenotype with little-to-no deleterious effect on growth rate while supporting strong production of effectors in the extracellular medium. Logic522 was inserted into SYN3000 to create the IL-22 secretion strain, SYN3001.

Additional constructs were also prepared. For example, one strain comprises a deletion in PAL and a plasmid expressing IL-22 with an OmpF secretion tag from a tetracycline-inducible promoter and another strain comprises the same PAL deletion and the same plasmid expressing IL-22, further comprising a deletion in degP. Additional strains are shown in Table 18.

TABLE 18
IL-22-secreting strains
Genotype

Lpp (delta lpp::CmR expressing PhoA-IL22 from Ptet)
nlpI (delta nlpl::CmR expressing PhoA-IL22 from Ptet)
tolA (delta tolA::CmR expressing PhoA-IL22 from Ptet)
PAL (delta pal::CmR expressing PhoA-IL22 from Ptet)

### Example 4. IL-10-Secreting Strains

In order to generate strains which secrete human IL-10, a base strain was used which has a “leaky membrane” phenotype (SYN1557), comprising delta PAL DOM background). For plasmid-based secretion construct, a codon optimized human IL10 sequence was combined with a number of secretion signals to determine the optimal configuration. Recently the Nissle genome was mined bioinformatically for signal sequences larger than the 21 AA PhoA tag. This yielded several candidates including the signal sequences from: ECOLIN_05715 (52 AA) (SEQ ID NO: 460), ECOLIN_16495 (40 AA) (SEQ ID NO: 464), ECOLIN_19410 (33 AA) (SEQ ID NO: 468) and ECOLIN_19880 (53 AA) (SEQ ID NO: 472). Additional signal peptides dsba, tolb, tort, ompA and pelB. Monomer IL10 or dimer IL10 (two IL10 monomer linked by a peptide linker) was constructed in combination with these signal peptides. These signal sequences were codon optimized and synthesized along with optimized RBS sequences, then inserted upstream of an optimized hIL10 sequence in a high copy pUC57 backbone. All of the candidate hIL10 constructs were then transformed into the delta PAL DOM background to test for secretion. Tables 19-23 list exemplary strains/construct sequences.

TABLE 19
Non-limiting IL-10 Construct Polynucleotide Sequences

| Description | SEQ ID NO |
|---|---|
| Construct comprising secretion tag of ECOLIN | SEQ ID NO: 488 |
| 19410 - human IL-10 - Linker (7aa) - human IL-10 | |
| Construct comprising PhoA secretion tag - | SEQ ID NO: 489 |
| human IL-10 - Linker (7aa) - human IL-10 | |
| PhoA Secretion Tag | SEQ ID NO: 490 |
| ECOLIN 19410 Tag | SEQ ID NO: 491 |
| human IL-10 - Linker (7aa) - human IL-10 | SEQ ID NO: 492 |
| Human IL-10 | SEQ ID NO: 493 |
| Linker | SEQ ID NO: 494 |

TABLE 20
Non-limiting IL-10 Construct Polypeptide Sequences

| Description | SEQ ID NO |
|---|---|
| Construct comprising secretion tag of ECOLIN | SEQ ID NO: 495 |
| 19410 - human IL-10 - Linker (7aa) - human IL-10 | |
| Construct comprising PhoA secretion tag - | SEQ ID NO: 496 |
| human IL-10 - Linker (7aa) - human IL-10 | |
| PhoA Secretion Tag | SEQ ID NO: 385 |
| ECOLIN 19410 Tag | SEQ ID NO: 397 |
| human IL-10 - Linker (7aa) - human IL-10 | SEQ ID NO: 143 |
| Human IL-10 | SEQ ID NO: 497 |
| Linker | SEQ ID NO: 498 |

TABLE 21
Non-limiting IL-10 Construct Polynucleotide Sequences

| Description | SEQ ID NO |
|---|---|
| Construct comprising secretion tag of dsba - | SEQ ID NO: 1220 |
| human IL-10 - Linker (7aa) - human IL-10 | |
| Construct comprising tolb secretion tag - | SEQ ID NO: 1221 |
| human IL-10 - Linker (7aa) - human IL-10 | |
| Construct comprising tort secretion tag - | SEQ ID NO: 1222 |
| human IL-10 - Linker (7aa) - human IL-10 | |
| Construct comprising ompA secretion tag - | SEQ ID NO: 1223 |
| human IL-10 - Linker (7aa) - human IL-10 | |
| Construct comprising pelB secretion tag - | SEQ ID NO: 1224 |
| human IL-10 - Linker (7aa) - human IL-10 | |
| dsba Secretion Tag | SEQ ID NO: 1112 |
| tolb Secretion Tag | SEQ ID NO: 1113 |
| tort Secretion Tag | SEQ ID NO: 1114 |
| ompA Secretion Tag | SEQ ID NO: 1115 |
| pelB Secretion Tag | SEQ ID NO: 1116 |
| Linker | SEQ ID NO: 1227 |
| Construct comprising secretion tag of dsba - | SEQ ID NO: 1220 |
| human IL-10 | |
| Construct comprising tolb secretion tag - | SEQ ID NO: 1228 |
| human IL-10 | |
| Construct comprising tort secretion tag - | SEQ ID NO: 1229 |
| human IL-10 | |

TABLE 22
Non-limiting IL-10 Construct Polypeptide Sequences

| Description | SEQ ID NO |
|---|---|
| Construct comprising secretion tag of dsba - | SEQ ID NO: 1230 |
| human IL-10 - Linker (7aa) - human IL-10 | |
| Construct comprising tolb secretion tag - | SEQ ID NO: 1231 |
| human IL-10 - Linker (7aa) - human IL-10 | |
| Construct comprising tort secretion tag - | SEQ ID NO: 1232 |
| human IL-10 - Linker (7aa) - human IL-10 | |
| Construct comprising ompA secretion tag - | SEQ ID NO: 1233 |
| human IL-10 - Linker (7aa) - human IL-10 | |
| Construct comprising pelB secretion tag - | SEQ ID NO: 1234 |
| human IL-10 - Linker (7aa) - human IL-10 | |
| dsba Secretion Tag | SEQ ID NO: 1092 |
| tolb Secretion Tag | SEQ ID NO: 1109 |
| tort Secretion Tag | SEQ ID NO: 1110 |
| ompA Secretion Tag | SEQ ID NO: 1111 |
| pelB Secretion Tag | SEQ ID NO: 1096 |
| Linker | SEQ ID NO: 1055 |
| Construct comprising secretion tag of dsba - | SEQ ID NO: 1237 |
| human IL-10 | |
| Construct comprising tolb secretion tag - | SEQ ID NO: 1238 |
| human IL-10 | |
| Construct comprising tort secretion tag - | SEQ ID NO: 1239 |
| human IL-10 | |

TABLE 23
Exemplary IL-10-secreting strains

| STRAIN | Genotype | Circuit |
|---|---|---|
| SYN1557 | pal::Cm | None |
| SYN2898 | pal::Cm | Ptet-OmpFss-hIL10 p15a |
| SYN2890 | pal::Cm | Ptet-ECOLIN_05715ss-hIL10 pUC57 |
| SYN2892 | pal::Cm | Ptet-ECOLIN_19410ss-hIL10 pUC57 |
| SYN2893 | pal::Cm | Ptet-ECOLIN_19880ss-hIL10 pUC57 |
| SYN3204 | pal::Cm | Ptet-dsba-mono-hIL10-linker-hIL10 pUC |
| SYN3205 | pal::Cm | Ptet-tolb-mono-hIL10-linker-hIL10 pUC |
| SYN3206 | pal::Cm | Ptet-tort-mono-hIL10-linker-hIL10 pUC |
| SYN3207 | pal::Cm | Ptet-ompA-mono-hIL10-linker-hIL10 pUC |
| SYN3208 | pal::Cm | Ptet-pelB-mono-hIL10-linker-hIL10 pUC |
| SYN3209 | pal::Cm | Ptet-dsba-mono-hIL10 pUC |
| SYN3210 | pal::Cm | Ptet-tolb-mono-hIL10 pUC |
| SYN3211 | pal::Cm | Ptet-tort-mono-hIL10 pUC |

### Example 5. IL-10-Secreting Strains (Dimerized IL-10)

To generate an engineered E. coli Nissle strain capable of secreting biologically active Interleukin 10 (IL-10), a construct was generated in which two interleukin-10 monomer subunits were covalently linked by a linker. The design used several components to accomplish the production of the dimerized human IL-10 protein product. To facilitate the dimerization of recombinant human IL-10 protein produced from E. coli Nissle, a 7 amino acid linker of ‘GGGSGGG (SEQ ID NO: 1055)’ was inserted between two monomer human IL-10 proteins to produce a forced dimer human IL-10 (diIL-10) fusion protein. The DNA sequence encoding this diIL-10 fusion protein was codon-optimized using publicly available methods. To promote translocation to the periplasm, a 32-amino acid secretion tag from the acid stress chaperone HdeB in E. coli (ECOLIN_19410) and designated tag 19410 was added to the N-terminus of the diIL-10 fusion protein. This tag not only mediates secretion into the periplasm but is putatively cleaved upon secretion preserving the N-terminal identity of the mature diIL-10 fusion protein. The DNA sequence containing the elements of the inducible Ptet promoter, ribosome binding site, diIL-10 coding sequence and other necessary linkers were synthesized by IDT Technologies and subsequently cloned into a high copy number plasmid vector provided by Life Technologies.

To create an E. coli Nissle derivative capable of secreting bioactive proteins, a engineered a diffusible outer membrane (DOM) phenotype was generated by deleting the gene encoding the periplasmic protein PAL. The resulting diffusible chassis strain was designated SYN1557. The plasmid was transformed into SYN1557 to create the dimerized human IL-10 (diIL-10) secretion strain SYN3139. SEQ ID NO: 488-498 and SEQ ID NO: 143, 385, and 397 are non-limiting examples of construct sequences.

### Example 6. GLP-2-Secreting Strains

To generate an engineered E. coli Nissle strain capable of secreting biologically active glucagon-like peptide 2 (GLP-2) a GLP2-FcIgA fusion protein was constructed.

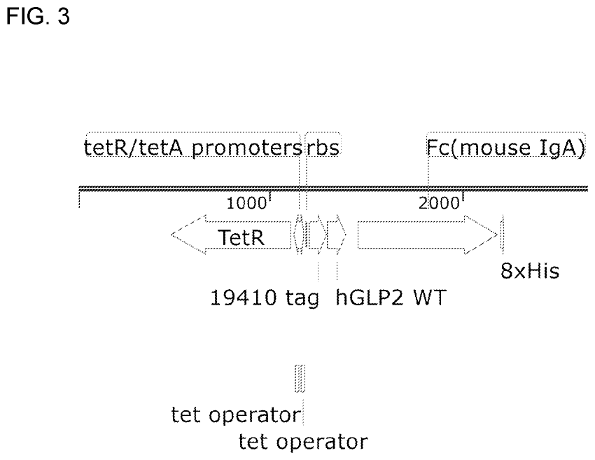

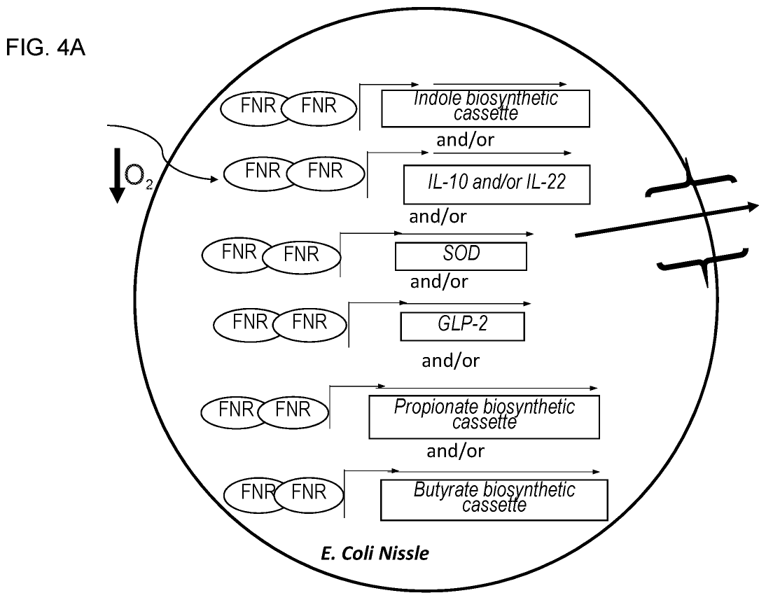

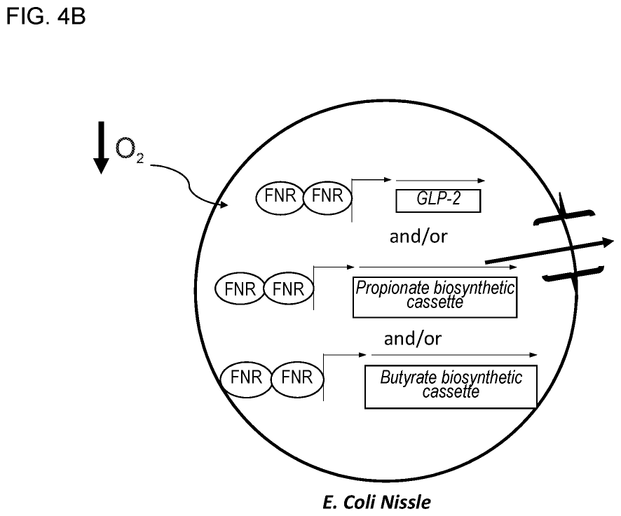

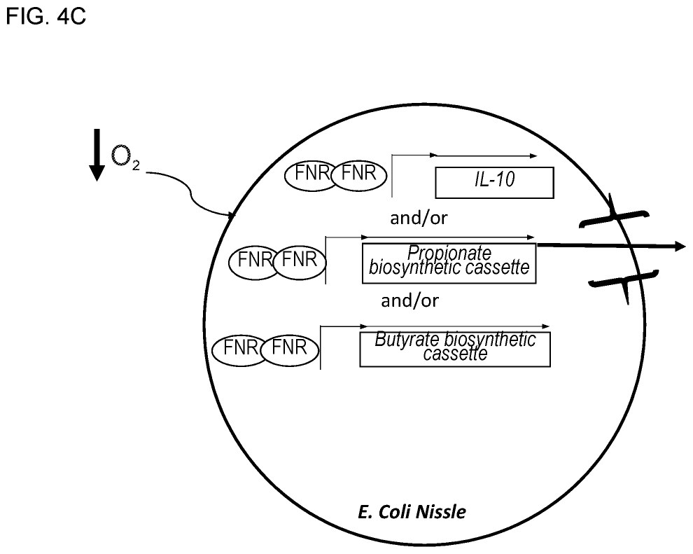

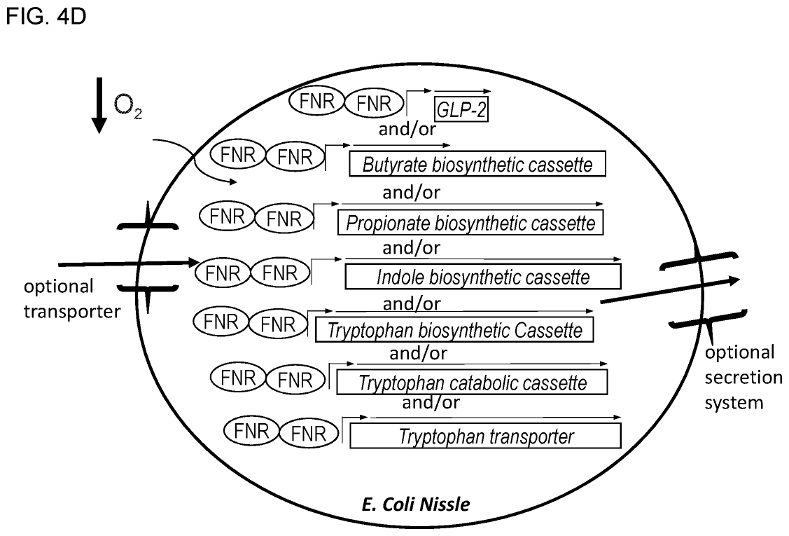

To produce the recombinant GLP2-FcIgA-HIS fusion protein, a synthetic construct was devised as illustrated in FIG. 3. The design uses several components to accomplish the production of the GLP2-FcIgA-HIS fusion protein product. The 33 amino acid human glucagon-like peptide-2 (GLP-2) with the sequence of SEQ ID NO: 149 is fused to the N-terminus of the Fc portion of mouse IgA protein linked by a 20 amino acid linker. The Fc portion of mouse IgA used herein contains the hinge, CH2 and CH3 regions. An 8×HIS tag (SEQ ID NO: 1056) is fused to the C-terminus of FcIgA connected with a linker to facilitate downstream processing and detection. Expression of the gene encoding the GLP2-FcIgA-HIS fusion protein is controlled by the tetracycline-inducible promoter (Ptet) which is derepressed via the addition of the tetracycline analog anhydrotetracycline (aTc); translation is driven by a strong ribosome binding site (RBS) located upstream of the GLP2-FcIgA-HIS coding sequence. To promote translocation to the periplasm, a 32-amino acid secretion tag originally discovered in house from the acid stress chaperone HdeB in E. coli (ECOLIN_19410) and designated tag 19410 was added to the N-terminus of GLP2-FcIgA-HIS fusion protein. This tag not only mediates secretion into the periplasm but is putatively cleaved upon secretion preserving the N-terminal identity of the mature GLP2-FcIgA-HIS fusion protein. The DNA sequence containing the Ptet promoter, RBS, coding sequence for GLP2-FcIgA-HIS and other necessary linkers were synthesized by IDT Technologies and subsequently cloned into a high copy number plasmid vector provided by IDT Technologies.

To create a Gram-negative bacterium capable of secreting bioactive proteins, a diffusible outer membrane (DOM) phenotype in the E. coli Nissle background was engineered by deleting the gene encoding the periplasmic protein PAL. This alteration results in an increased rate of diffusion of periplasmic proteins to the external environment without compromising cell growth properties. The resulting ‘leaky’ chassis strain is designated SYN1557. The plasmid was transformed into SYN1557 to create the GLP2-FcIgA-HIS secretion strain SYN3074. SEQ ID NO: 499-536, 148, 149, 385, and 397 are of non-limiting examples of construct sequences for the expression and secretion of GLP-2 and other polypeptide fusion proteins for secretion.

TABLE 24
Non-limiting GLP-2 Construct Polypeptide Sequences

| Description | Sequence |
|---|---|
| Human IgA Fc | SEQ ID |
| | NO: 499 |
| Human IgA hinge region | SEQ ID |
| | NO: 500 |
| Construct comprising secretion tag from ECOLIN 19410 - | SEQ ID |
| human GLP2 - linker - Fc (mouse IgA) - 8X His Tag | NO: 501 |
| Construct comprising secretion tag from ECOLIN 19410 - | SEQ ID |
| human GLP2 - linker - Fc (mouse IgA) | NO: 502 |
| Construct comprising PhoA secretion tag -human GLP2 - | SEQ ID |
| linker - Fc (mouse IgA) - 8X His Tag | NO: 503 |
| Construct comprising PhoA secretion tag -human GLP2 - | SEQ ID |
| linker - Fc (mouse IgA) | NO: 504 |
| Construct comprising PhoA secretion tag -mutated | SEQ ID |
| human GLP2 - linker - Fc (mouse IgA) - 8X His Tag | NO: 505 |
| Construct comprising PhoA secretion tag -mutated | SEQ ID |
| human GLP2 - linker - Fc (mouse IgA) | NO: 506 |
| Construct comprising ECOLIN 19410 secretion tag -mutated | SEQ ID |
| human GLP2 - linker - Fc (mouse IgA) - 8X His Tag | NO: 507 |
| Construct comprising ECOLIN 19410 secretion tag -mutated | SEQ ID |
| human GLP2 - linker - Fc (mouse IgA) | NO: 508 |
| ECOLIN 19410 secretion tag | SEQ ID |
| | NO: 397 |
| Human GLP2 | SEQ ID |
| | NO: 148 |
| Linker | SEQ ID |
| | NO: 509 |
| Fc (mouse IgA) | SEQ ID |
| | NO: 510 |
| PhoA | SEQ ID |
| | NO: 385 |
| mutated human GLP2 | SEQ ID |
| | NO: 149 |
| Construct comprising PhoA secretion tag -human GLP2 - | SEQ ID |
| linker - Fc (human IgA) | NO: 512 |
| Construct comprising ECOLIN 19410 secretion tag - | SEQ ID |
| human GLP2 - linker - Fc (human IgA) - 8X His Tag | NO: 513 |
| Construct comprising ECOLIN 19410 secretion tag - | SEQ ID |
| human GLP2 - linker - Fc (human IgA) | NO: 514 |
| Construct comprising PhoA secretion tag -human GLP2 - | SEQ ID |
| linker - Fc (human IgA) - 8X His Tag | NO: 511 |

TABLE 25
Non-limiting GLP-2 Construct Polynucleotide Sequences

| Description | Sequence |
|---|---|
| Construct comprising secretion tag from ECOLIN | SEQ ID |
| 19410 -human GLP2- linker-Fc (mouse IgA) - 8X His Tag | NO: 515 |
| Construct comprising secretion tag from ECOLIN | SEQ ID |
| 19410 -human GLP2- linker-Fc (mouse IgA) | NO: 516 |
| Construct comprising PhoA secretion tag -human GLP2 - | SEQ ID |
| linker - Fc (mouse IgA) - 8X His Tag | NO: 517 |
| Construct comprising PhoA secretion tag -human GLP2 - | SEQ ID |
| linker - Fc (mouse IgA) | NO: 518 |
| Construct comprising PhoA secretion tag -mutated human | SEQ ID |
| GLP2 - linker - Fc (mouse IgA) - 8X His Tag | NO: 519 |
| Construct comprising PhoA secretion tag -mutated human | SEQ ID |
| GLP2 - linker - Fc (mouse IgA) | NO: 520 |
| Construct comprising ECOLIN 19410 secretion tag -mutated | SEQ ID |
| human GLP2 - linker - Fc (mouse IgA) - 8X His Tag | NO: 521 |
| Construct comprising ECOLIN 19410 secretion tag -mutated | SEQ ID |
| human GLP2 - linker - Fc (mouse IgA) | NO: 522 |
| Human GLP2 | SEQ ID |
| | NO: 523 |
| Linker | SEQ ID |
| | NO: 524 |
| Fc (mouse IgA) | SEQ ID |
| | NO: 525 |
| PhoA tag | SEQ ID |
| | NO: 490 |
| Mutated human GLP2 | SEQ ID |
| | NO: 526 |
| Fc (human IgA) | SEQ ID |
| | NO: 527 |
| Second sequence encoding Fc (human IgA) | SEQ ID |
| | NO: 528 |
| Construct comprising PhoA secretion tag -human GLP2 - | SEQ ID |
| linker - Fc (human IgA) - 8X His Tag | NO: 529 |
| Second construct comprising PhoA secretion tag -human | SEQ ID |
| GLP2 - linker - Fc (human IgA) - 8X His Tag | NO: 530 |
| Construct comprising PhoA secretion tag -human GLP2 - | SEQ ID |
| linker - Fc (human IgA) | NO: 531 |
| Second construct comprising PhoA secretion tag -human | SEQ ID |
| GLP2 - linker - Fc (human IgA) | NO: 532 |
| Construct comprising secretion tag from ECOLIN | SEQ ID |
| 19410 -human GLP2- linker-Fc (human IgA) - - 8X His Tag | NO: 533 |
| Second construct comprising secretion tag from ECOLIN | SEQ ID |
| 19410 -human GLP2-linker-Fc (human IgA) - - 8X His Tag | NO: 534 |
| Construct comprising secretion tag from ECOLIN | SEQ ID |
| 19410 -human GLP2- linker-Fc (human IgA) | NO: 535 |
| Second construct comprising secretion tag from ECOLIN | SEQ ID |
| 19410 -human GLP2-linker-Fc (human IgA) | NO: 536 |

### Example 7. Generation of Constructs and Bacteria for Cytokine Secretion

To produce strains capable of secreting immune modulatory polypeptides, e.g., cytokines, such as hIL-12, mIL-12, hIL-15, GMCSF, TNF-alpha, IFN-gamma, CXCL9 and CXCL10, several constructs were designed employing different secretion strategies. Various cytokine constructs were synthesized, and cloned into vector pBR322 for transformation of E. coli. In some embodiments, the constructs encoding the effector molecules are integrated into the genome. In some embodiments, the constructs encoding the effector molecules are on a plasmid, e.g., a medium copy plasmid.

Polypeptide sequences of exemplary cytokines used in the constructs include hIL-12a (SEQ ID NO: 1131), hIL-1213 (SEQ ID NO: 1132); mIL-12 (SEQ ID NO: 1131); mIL-1213 (SEQ ID NO: 1133); hIL-15 (SEQ ID NO: 1134); GMCSF (SEQ ID NO: 1135); TNF-alpha (extracellular portion) (SEQ ID NO: 1136); IFN-gamma (SEQ ID NO: 1137); CXCL10 (SEQ ID NO: 1138); and CXCL9 (SEQ ID NO: 1139). In some embodiments, recombinant bacteria comprise a nucleic acid sequence that encodes a polypeptide which is at least about 80%, 85%, 90%, 95%, or 99% homologous to the DNA sequence of SEQ ID NO: 1131-1139.

Nucleotide sequences of exemplary cytokines (codon optimized for expression in E. coli) used in the constructs include hIL-12a (SEQ ID NO: 1140), hIL-1213 (SEQ ID NO: 1141); mIL-12 (SEQ ID NO: 1142); mIL-1213 (SEQ ID NO: 1143); hIL-15 (SEQ ID NO: 1144); GMCSF (SEQ ID NO: 1145); TNF-alpha (extracellular portion) (SEQ ID NO: 1146); IFN-gamma (SEQ ID NO: 1147); and CXCL10 (SEQ ID NO: 1148). In some embodiments, recombinant bacteria comprise a nucleic acid sequence that is at least about 80%, 85%, 90%, 95%, or 99% homologous to the DNA sequence of SEQ ID Nos 1140-1148.

Exemplary secretion Tags and FliC components include fliC-FliC20 (SEQ ID NO: 894); flic-RBS (SEQ ID NO: 895-897); RBS-phoA (SEQ ID NO: 898); phoA (SEQ ID NO: 889); RBS-ompF (SEQ ID NO: 900); ompF (SEQ ID NO: 901); RBS-cvaC (SEQ ID NO: 902); cvaC (SEQ ID NO: 903); RBS-phoA (Optimized) (SEQ ID NO: 904); optimized phoA (SEQ ID NO: 905); RBS-TorA (SEQ ID NO: 906); TorA (SEQ ID NO: 907); RBS-TorA alternate (SEQ ID NO: 908); TorA (alternate) (SEQ ID NO: 909); RBS-fdnG (SEQ ID NO: 910); fdnG (SEQ ID NO: 911); RBS-dmsA (SEQ ID NO: 912); dmsA (SEQ ID NO: 913) and PelB. In some embodiments, recombinant bacteria comprise a nucleic acid sequence that is at least about 80%, 85%, 90%, 95%, or 99% homologous to the DNA sequence of SEQ ID Nos 894-913.

Exemplary promoter sequences and miscellaneous construct sequences include TetR/TetA Promoter (SEQ ID NO: 914); fliC Promoter (SEQ ID NO: 915); FnrS Promoter (SEQ ID NO: 916); DOM Construct Terminator (SEQ ID NO: 917); FRT Site (SEQ ID NO: 918); and Kanamycin Resistance Cassette (for integration in between FRT sites) (SEQ ID NO: 919). In some embodiments, recombinant bacteria comprise a nucleic acid sequence that is at least about 80%, 85%, 90%, 95%, or 99% homologous to the DNA sequence of SEQ ID NOs: 914-919.

Non-limiting examples of secretion constructs include human IL-12a construct with a N terminal OmpF secretion tag (sec-dependent secretion system) (SEQ ID NO: 920); human IL-12a construct with a N terminal PhoA secretion tag (SEQ ID NO: 921); human IL-12a construct with a N terminal TorA secretion tag (sec-dependent secretion system) (SEQ ID NO: 922); human IL-12b construct with a N terminal OmpF secretion tag (sec-dependent secretion system) (SEQ ID NO: 923); human IL-12b construct with a N terminal PhoA secretion tag (SEQ ID NO: 924); human IL-12 construct with a N terminal TorA secretion tag (sec-dependent secretion system) (SEQ ID NO: 925); murine IL-12a construct with a N terminal OmpF secretion tag (sec-dependent secretion system) (SEQ ID NO: 926); murine IL-12a construct with a N terminal PhoA secretion tag (SEQ ID NO: 927); murine IL-12a construct with a N terminal TorA secretion tag (sec-dependent secretion system) (SEQ ID NO: 928); murine IL-12b construct with a N terminal OmpF secretion tag (sec-dependent secretion system) (SEQ ID NO: 929); murine IL-12b construct with a N terminal PhoA secretion tag (SEQ ID NO: 930); murine IL-12b construct with a N terminal TorA secretion tag (sec-dependent secretion system) (SEQ ID NO: 931); human GMCSF construct with a N terminal OmpF secretion tag (sec-dependent secretion system) (SEQ ID NO: 932); human GMCSF construct with a N terminal PhoA secretion tag (SEQ ID NO: 933); human GMCSF construct with a N terminal TorA secretion tag (sec-dependent secretion system) (SEQ ID NO: 934); human IL-15 construct with a N terminal OmpF secretion tag (sec-dependent secretion system) (SEQ ID NO: 935); human IL-15 construct with a N terminal PhoA secretion tag (SEQ ID NO: 936); human IL-15 construct with a N terminal TorA secretion tag (sec-dependent secretion system) (SEQ ID NO: 937); human TNFa construct with a N terminal OmpF secretion tag (sec-dependent secretion system) (SEQ ID NO: 938); human TNFa construct with a N terminal PhoA secretion tag (SEQ ID NO: 939); human TNFa construct with a N terminal TorA secretion tag (sec-dependent secretion system) (SEQ ID NO: 940); human IFNg construct with a N terminal OmpF secretion tag (sec-dependent secretion system) (SEQ ID NO: 941); human IFNg construct with a N terminal PhoA secretion tag (SEQ ID NO: 942); human IFNg construct with a N terminal TorA secretion tag (sec-dependent secretion system) (SEQ ID NO: 943); murine CXCL10 construct with a N terminal OmpF secretion tag (sec-dependent secretion system) (SEQ ID NO: 1149); murine CXCL10 construct with a N terminal PhoA secretion tag (SEQ ID NO: 1150); murine CXCL10 construct with a N terminal TorA secretion tag (sec-dependent secretion system) (SEQ ID NO: 1151); human CXCL9 construct with a N terminal OmpF secretion tag (sec-dependent secretion system) (SEQ ID NO: 1152); human CXCL9 construct with a N terminal PhoA secretion tag; (SEQ ID NO: 1153); human CXCL9 construct with a N terminal TorA secretion tag (sec-dependent secretion system) (SEQ ID NO: 1154); murine CXCL9 construct with a N terminal OmpF secretion tag (sec-dependent secretion system) (SEQ ID NO: 1155); murine CXCL9 construct with a N terminal PhoA secretion tag (SEQ ID NO: 1156); murine CXCL9 construct with a N terminal TorA secretion tag (sec-dependent secretion system) (SEQ ID NO: 1157). In some embodiments, recombinant bacteria comprise a nucleic acid sequence encoding a polypeptide that is at least about 80%, at least about 85%, at least about 90%, at least about 95%, or at least about 99% homologous to the DNA sequence of SEQ ID NOs: 920-943 and 1149-1157.

Non-limiting examples of secretion constructs include human IL-12a construct with a N terminal PhoA secretion tag (SEQ ID NO: 953); human IL-12b construct with a N terminal PhoA secretion tag (SEQ ID NO: 954); murine IL-12a construct with a N terminal PhoA secretion tag; (SEQ ID NO: 955); murine IL-12b construct with a N terminal PhoA secretion tag; (SEQ ID NO: 956); human 11-15 construct with a N terminal PhoA secretion tag (SEQ ID NO: 957); human GMCSF construct with a N terminal PhoA secretion tag (SEQ ID NO: 958); human TNFa construct with a N terminal PhoA secretion tag (SEQ ID NO: 959); human IFNg construct with a N terminal PhoA secretion tag; (SEQ ID NO: 960); human CXCL10 construct with a N terminal PhoA secretion tag; (SEQ ID NO: 1158); murine CXCL10 construct with a N terminal PhoA secretion tag (SEQ ID NO: 1159); human CXCL9 construct with a N terminal PhoA secretion tag (SEQ ID NO: 1160); murine CXCL9 construct with a N terminal PhoA secretion tag (SEQ ID NO: 1161). In some embodiments, recombinant bacteria comprise a nucleic acid sequence that is at least about 80%, at least about 85%, at least about 90%, at least about 95%, or at least about 99% homologous to the DNA sequence of SEQ ID NOs: 953-960 and 1158-1161.

Additional examples of secretion constructs include Ptet.phoA-hIL12b-phoA-hIL12a (SEQ ID NO: 965); Ptet.phoA-mIL12b-phoA-mIL12a (SEQ ID NO: 966); Ptet.phoA-IL15 (SEQ ID NO: 967); Ptet.phoA-GMCSF (SEQ ID NO: 968); Ptet-phoA-TNFa (SEQ ID NO: 969); Ptet-phoA-IFNg (SEQ ID NO: 970); Ptet-phoA-hCXCL10 (SEQ ID NO:1162); Ptet-phoA-mCXCL10 (SEQ ID NO: 1163); Ptet-phoA-hCXCL9 (SEQ ID NO: 1164); and Ptet-phoA-mCXCL9 (SEQ ID NO: 1165). In some embodiments, recombinant bacteria comprise a nucleic acid sequence that is at least about 80%, at least about 85%, at least about 90%, at least about 95%, or at least about 99% homologous to the DNA sequence of SEQ ID NOs: 965-970 and 1162-1165.

### Example 8. Functional Assay for CXCL10

To determine whether CXCL10 secreted from any of the strains described above is functional, a Chemotaxis Assay is performed, essentially as described in Mikucki et al. (Mikucki, et al., Nat Commun. 2015; 6: 7458, the contents of which is herein incorporated by reference in its entirety).

Briefly, cell trace labeled naïve (or expanded) human CD8+ T cells are transferred into 24-well transwell plate (5 uM pore) with T cells on top (5×10⁵ cells) and rCXCL10/supernatants on bottom, in the presence or absence of PTX or anti-CXCL10). Cells are incubated for 3 hrs, and migrated cells are counted by flow cytometry. Alternatively, phosphoAKT is measured by flow, western or ELISA.

### Example 9. Cytokine Secretion (mIL-12 and hIL-12)

To determine whether the mIL-12 and hIL-12 expressed by engineered bacteria is secreted, the concentration of IL-12 in the bacterial supernatant from engineered strains comprising mIL-12 or hIL-12 secretion constructs/strains was measured. The strains comprise either a deletion in Lpp (lpp::Cm), nlpI (nlpI:Cm), tolA (tolA::Cm), or PAL (PAL::Cm). All strains further comprise either a plasmid expressing hIL-12 with a PhoA secretion tag or a plasmid expressing mIL-12 with a PhoA secretion tag from a tetracycline-inducible promoter.

E. coli Nissle strains were grown overnight in LB medium. Cultures were diluted 1:200 in LB and grown shaking (200 rpm) for 2 hours. Cultures were diluted to an optical density of 0.5 at which time anhydrous tetracycline (ATC) was added to cultures at a concentration of 100 ng/mL to induce expression of hIL-12. After 12 hours of induction, cells were spun down, and supernatant was collected. To generate cell free medium, the clarified supernatant was further filtered through a 0.22 micron filter to remove any remaining bacteria and placed on ice. Additionally, to detect intracellular recombinant protein production, pelleted were bacteria washed and resuspended in BugBuster™ (Millipore) with protease inhibitors and Ready-Lyse Lysozyme Solution (Epicentre), resulting in lysate concentrated 10-fold compared to original culture conditions. After incubation at room temperature for 10 minutes insoluble debris is spun down at 20 min at 12,000 rcf at 4° C. then placed on ice until further processing.

The concentration of mIL-12 or hIL-12 in the cell-free medium and in the bacterial cell extract was measured by mIL-12 ELISA (RnD Systems, Minneapolis, MN) or hIL-12 ELISA (RnD Systems, Minneapolis, MN), according to manufacturer's instructions. All samples were run in triplicate, and a standard curve was used to calculate secreted levels of mIL-12 or hIL-12. Standard curves were generated using recombinant mIL-12 or hIL-12. Wild type Nissle was included in the ELISA as a negative control, and no signal was observed. Table 26 and Table 27 summarize levels of mIL-12 and hIL-12 measured in the respective supernatants. The data show that both mIL-12 and h-IL-12 are secreted at various levels from the different bacterial strains.

TABLE 26
Concentration of mIL-12 secreted into the media

| | | | [mIL-12] |
|---|---|---|---|
| | | | (ng/ml) in |
| ID | Genotype | Construct | the medium |
| SYN1825 | Lpp (lpp::Cm) | pBR322.Ptet.phoA-mIL12 | 0.2 |
| SYN1826 | nlpI (nlpI::Cm) | pBR322.Ptet.phoA-mIL12 | 0.1 |
| SYN1827 | tolA | pBR322.Ptet.phoA-mIL12 | 0.1 |
| | (tolA::Cm) | | |
| SYN1828 | PAL (PAL::Cm) | pBR322.Ptet.phoA-mIL12 | 0.3 |

TABLE 27
Concentration of Secreted hIL-12

| | | | [hIL-12] |
|---|---|---|---|
| | | | (ng/ml) |
| ID | Genotype | Construct | in the medium |
| SYN1821 | Lpp (lpp::Cm) | pBR322.Ptet.phoA-hIL12 | 0.9 |
| SYN1822 | nlpl (nlpI::Cm) | pBR322.Ptet.phoA-hIL12 | 0.5 |
| SYN1823 | tolA | pBR322.Ptet.phoA-hIL12 | 0.5 |
| | (tolA::Cm) | | |
| SYN1824 | PAL | pBR322.Ptet.phoA-hIL12 | 0.3 |
| | (PAL::Cm) | | |

### Example 10. Cytokine Secretion (IL-15)

To determine whether the hIL-15 expressed by engineered bacteria is secreted, the concentration of hIL-15 in the bacterial supernatant from engineered strains comprising hIL-15 secretion constructs/strains was measured. The strains comprise either a deletion in Lpp (lpp::Cm), nlpI (nlpI:Cm), tolA (tolA::Cm), or PAL (PAL::Cm). All strains further comprise a plasmid expressing hIL-15 with a PhoA secretion tag.

E. coli Nissle strains were grown, induced and processed as described in the previous example for hIL12 and hIL-12.

The concentration of hIL-15 in the cell-free medium and in the bacterial cell extract was measured by hIL-15 ELISA (RnD Systems, Minneapolis, MN), according to manufacturer's instructions. All samples were run in triplicate, and a standard curve was used to calculate secreted levels of hIL-15. Standard curves were generated using recombinant hIL-15. Wild type Nissle was included in the ELISA as a negative control, and no signal was observed. Table 28 summarizes levels of hIL-15 measured in the respective supernatants. The data show that hIL-15 is secreted at various levels from the different bacterial strains.

TABLE 28
Concentration of Secreted hIL-15

| | | | [IL-15] |
|---|---|---|---|
| | | | (ng/ml) |
| ID | Genotype | Construct | in the medium |
| SYN1817 | Lpp (lpp::Cm) | pBR322.Ptet.phoA-IL15 | 27.9 |
| SYN1818 | nlpI (nlpI::Cm) | pBR322.Ptet.phoA-IL15 | 30.4 |
| SYN1819 | tolA (tolA::Cm) | pBR322.Ptet.phoA-IL15 | 33.8 |
| SYN1820 | PAL (PAL::Cm) | pBR322.Ptet.phoA-IL15 | 38.0 |

### Example 11. Functional Testing of Secreted IL-15

To test whether the IL-15 secreted from any of the strains described above is functional, a CTLL-2 proliferation assay is conducted as described in Soman et al. (Soman, G. et al., J Immunol Methods. 2009; 348(1-2): 83-94, the entire contents is incorporated by reference).

Briefly, CTLL-2 are grown in 200U/mL IL-2 and rinsed and equilibrated in media lacking IL-2. Cells are then seeded into 96-well plate (5×10⁴ cells/well) and exposed to 2× titration of rhIL-15 or supernatants starting at 2 ng/mL (or 20 IU/mL) (w or w/o anti-IL-15). Cells are incubated for 48 hrs and the cell titer is measured using a plate reader.

### Example 12. Cytokine Secretion (GMCSF)

To determine whether hGMCSF expressed by engineered bacteria is secreted, the concentration of hGMCSF in the bacterial supernatant from engineered strains comprising hGMCSF secretion constructs/strains was measured. The strains comprise either a deletion in Lpp (lpp::Cm), nlpI (nlpI:Cm), tolA (tolA::Cm), or PAL (PAL::Cm). All strains further comprise a plasmid expressing hGMCSF with a PhoA secretion tag.

E. coli Nissle strains were grown, induced and processed as described in the previous example for hIL12 and hIL-12.

The concentration of hGMCSF in the cell-free medium and in the bacterial cell extract was measured by hGMCSF ELISA (RnD Systems, Minneapolis, MN), according to manufacturer's instructions. All samples were run in triplicate, and a standard curve was used to calculate secreted levels of hGMCSF. Standard curves were generated using recombinant hGMCSF. Wild type Nissle was included in the ELISA as a negative control, and no signal was observed. Table 29 summarizes levels of hGMCSF measured in the respective supernatants. The data show that hGMCSF is secreted at various levels from the different bacterial strains.

TABLE 29
Concentration of Secreted GMCSF

| | | | | [GMCSF] | [GMCSF] |
|---|---|---|---|---|---|
| | | | | (ng/ml) | (ng/ml) |
| | | | | in the | in the |
| | | | | medium | medium |
| | | High copy | | High copy | Low copy |
| ID | Genotype | construct | Low copy construct | plasmid | plasmid |
| SYN094 | WT | None | None | 0.0 | 0.0 |
| SYN2036/ | lpp | PUC.Ptet.phoA- | PUN UNSX-TetR-Ptet- | 45.8 | 44.7 |
| SYN2093 | | GMCSF | phoA-GMCSF-UNS9 | | |
| SYN2038/ | PAL | PUC.Ptet.phoA- | PUN UNSX-TetR-Ptet- | 114.3 | 98.8 |
| SYN2103 | | GMCSF | phoA-GMCSF-UNS9 | | |
| SYN2037/ | nlpI | PUC.Ptet.phoA- | pUN UNSX-TetR-Ptet- | 39.9 | 44.0 |
| SYN2095 | | GMCSF | phoA-GMCSF-UNS9 | | |

### Example 13. Cytokine Secretion (TNFa)

To determine whether hTNFa expressed by engineered bacteria is secreted, the concentration of hTNFa in the bacterial supernatant from engineered strains comprising hTNFa secretion constructs/strains was measured. The strains comprise either a deletion in Lpp (lpp::Cm), nlpI (nlpI:Cm), tolA (tolA::Cm), or PAL (PAL::Cm). All strains further comprise a plasmid expressing hTNFa with a PhoA secretion tag.

E. coli Nissle strains were grown, induced and processed as described in the previous example for hIL12 and hIL-12.

The concentration of hTNFa in the cell-free medium and in the bacterial cell extract was measured by hTNFa ELISA (RnD Systems, Minneapolis, MN), according to manufacturer's instructions. All samples were run in triplicate, and a standard curve was used to calculate secreted levels of hTNFa. Standard curves were generated using recombinant hTNFa. Wild type Nissle was included in the ELISA as a negative control, and no signal was observed. Table 30 summarizes levels of hTNFa measured in the respective supernatants. The data show that hTNFa is secreted at various levels from the different bacterial strains.

TABLE 30
Concentration of Secreted TNFa

| | | | Secreted [TNFa] |
|---|---|---|---|
| Strain | Genotype | Construct | ng/ml |
| SYN094 | WT | None | 0 |
| SYN2541 | lpp::Cm | Nissle Ptet-phoA-TNFa | 129.6 |
| SYN2542 | nlpI::Cm | Nissle Ptet-phoA-TNFa | 345.3 |
| SYN2543 | PAL::Cm | Nissle Ptet-phoA-TNFa | >400 |
| SYN2544 | TrpE | HA3/4::Plpp-pKYNase | >400 |
| | PAL::Cm | Ptet-phoA-TNFa | |
| SYN2545 | TrpE | HA3/4::PSyn-pKYNase | >400 |
| | PAL::Cm | Ptet-phoA-TNFa | |

### Example 14. Cytokine Secretion (hIFNg)

To determine whether hIFNg expressed by engineered bacteria is secreted, the concentration of hIFNg in the bacterial supernatant from engineered strains comprising hTNFa secretion constructs/strains was measured. The strains comprise either a deletion in Lpp (lpp::Cm), nlpI (nlpI:Cm), tolA (tolA::Cm), or PAL (PAL::Cm). All strains further comprise a plasmid expressing hIFNg with a PhoA secretion tag.

E. coli Nissle strains were grown, induced and processed as described in the previous example for hIL12 and hIL-12.

The concentration of hIFNg in the cell-free medium and in the bacterial cell extract was measured by hIFNg ELISA (RnD Systems, Minneapolis, MN), according to manufacturer's instructions. All samples were run in triplicate, and a standard curve was used to calculate secreted levels of hIFNg. Standard curves were generated using recombinant hIFNg. Wild type Nissle was included in the ELISA as a negative control, and no signal was observed. Table 31 summarizes levels of hIFNg measured in the respective supernatants. The data show that hIFNg is secreted at various levels from the different bacterial strains.

TABLE 31
Concentration of Secreted IFNg

| | | | Secreted |
|---|---|---|---|
| Strain | Genotype | Construct | [IFNg] ng/ml |
| SYN094 | WT | None | 0 |
| SYN2546 | lpp::Cm | Nissle Ptet-phoA-IFNg | 44.9 |
| SYN2547 | nlpI::Cm | Nissle Ptet-phoA-IFNg | 51.5 |
| SYN2548 | PAL::Cm | Nissle Ptet-phoA-IFNg | 85.9 |
| SYN2549 | TrpE | HA3/4::Plpp-pKYNase | 39.1 |
| | PAL::Cm | Ptet-phoA-IFNg | |
| SYN2550 | TrpE | HA3/4::PSyn-pKYNase | 87.6 |
| | PAL::Cm | Ptet-phoA-IFNg | |

Table 32 provides a summary of the levels of secretion obtained for each cytokine, and lists some structural features of the cytokine which may explain some of the differences in secretion levels observed.

TABLE 32
Summary of Secretion Results

| | | | | | | Secretion |
|---|---|---|---|---|---|---|
| | Size | | O-linked | N-linked | Disulphide | level |
| Therapeutic | (Dal) | Stoichiometry | Glycosylation | Glycosylation | Bonds | (ng/mL) |
| hIL-12 | 57238 | Heterodimer | 1 | 4 | 7 | 0.9 |
| mIL-12 | 57496 | Heterodimer | 0 | 5 | 4 | 0.2 |
| hIL-15 | 14715 | Monomer | 0 | 1 | 2 | 38.0 |
| GMCSF | 14477 | Monomer | 4 | 2 | 2 | 114.0 |
| TNF-alpha | 17353 | Monomer | 1 | 0 | 1 | >400 |
| IFN-gamma | 16177 | Homodimer | 0 | 2 | 0 | 87.6 |

### Example 15. α-PD1-scFv Expression in E. coli

To determine whether a functional scFv can be expressed in E coli, an anti-PD1-scFv fragment was generated based on J43 monoclonal antibody, which reacts with mouse PD-1.

Mouse monoclonal antibody J43 sequence was obtained from patent EP 1445264 A1. Next, the single-chain variable fragment (scFv) was designed. A fragment containing tet promoter, a ribosome binding site, the designed J43-scFv, a C terminal V5 tag and a C terminal hexa-histidine tag (SEQ ID NO: 1166) was synthesized by IDTDNA. The construct was cloned into the pCR™-Blunt II-TOPOO Vector (Invitrogen) and transformed into E. coli DH5a as described herein to generate the plasmid pUC-ptet-J43scFv-V5-HIS (SEQ ID NO: 975). Additional sequences include nucleotide sequence for J43-Anti-PD1-scFv-V5-HIS (SEQ ID NO: 976); scFv Heavy chain (SEQ ID NO: 977); scFv light chain (SEQ ID NO: 978); scFv Linker (SEQ ID NO: 979). Polypeptide sequence of J43-Anti-PD1-scFV is included in SEQ ID NO: 980.

In some embodiments, the PD1-scFv is at least about 80%, 85%, 90%, 95%, or 99% homologous to the sequence of SEQ ID NOs: 975-980.

E. coli comprising either tet-inducible J43-Anti-PD1-scFv-V5 or wild type controls were grown overnight in LB medium. Cultures were diluted 1:40 in LB and grown shaking (250 rpm) to an optical density of 0.8 at which time anhydrous tetracycline (ATC) was added to cultures at a concentration of 100 ng/mL to induce expression of J43-Anti-PD1-scFv-V5. Same amount of tetracycline was added to wild type control cultures. After 4 hrs of induction, bacteria were pelleted, washed in PBS, and harvested, resuspended in 2 mL sonication buffer (PBS), and lysed by sonication on ice. Insoluble debris was spun down twice for 15 min at 12,000 rpm at 4° C.

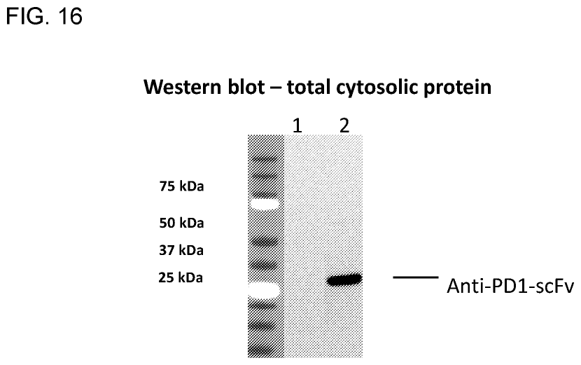

Protein concentration was determined by BCA protein assay, and isolated extracts from wild type and strains comprising the Ptet-J43-Anti-PD1-scFV-V5 were analyzed by Western blot. Proteins were transferred onto PVDF membranes and J43-Anti-PD1-scFv was detected with an HRP-conjugated anti-V5 antibody (Biolegend). Results are shown in FIG. 16. A single band was detected at 27 kDa in lane 2 (extract from J43-Anti-PD1-scFv-V5 strain). No bands were detected in lane 1 (wild type extract).

To determine whether the single-chain antibody purified from E. coli DH5a functionally binds to the target protein, PD1, an ELISA assay was performed. Plates were absorbed overnight at 4° C. with 100 itL of 2 itg/mL per well of PD1 (Rndsystems). Wells were blocked with 2% BSA in PBS/0.1% Tween-20 for 2 hours at room temperature. After three washes, wells were incubated with bacterial extracts (J43-scFv-V5 or wild type-neg-ctrl) for 1 hour at room temperature. Wells were washed 4 times with PBST (PBS/0.1% Tween-20) and incubated with a HRP-conjugated anti-V5 antibody (Biolegend) in blocking solution for 40 min. Following incubation, wells were washed 4 times with PBST and then stained using a 3,3′,5,5′-tetramethylbenzidine (TMB). Signal intensities were measured using an ELISA reader at 450 nm. Results are shown in Table 33 and indicate that the antibody expressed by the recombinant bacteria can bind to PD1 specifically.

TABLE 33
ELISA Binding Assay

| | PBS | mPD1 | IgG | 2′ |
|---|---|---|---|---|
| 1′ antibody | coating | coating | coating | antibody |
| Wild type-neg-ctrl | 0.11 | 0.13 | 0.12 | α-V5-HRP |
| J43-scFv-V5 | 0.11 | 1.41 | 0.13 | α-V5-HRP |
| Wild type-neg-ctrl (1/2) | 0.10 | 0.09 | 0.10 | α-V5-HRP |
| J43-scFv-V5 (1/2) | 0.10 | 10.90 | 0.11 | α-V5-HRP |

Next, recombinant J43-Anti-scFv-V5 was expression using pET22b vector harvesting a C-terminal poly-histidine tag and purified using immobilized metal ion affinity chromatography. Protein concentration was determined by absorption at 280 nm and purity was confirmed by Coomassie gel (data not shown).

To determine whether anti-PD1-scFv expressed in E. coli binds to surface PD1 on mouse EL4 cells, flow cytometric analysis was performed using EL4 cells. EL4 are a mouse lymphoma cell line which expresses PD1 on its cell surface.

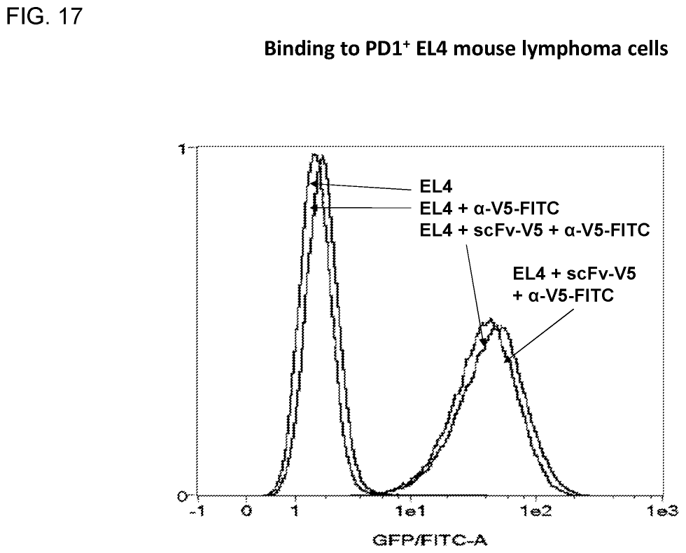

EL4 cells were grown in Dulbecco's Modified Eagle's Medium (DMEM) with 10% FBS. Cells were spun down, supernatant was aspirated, pellet was resuspended in 1 ml D-PBS, transferred into chilled assay tubes (1×106 cells), and washed 2-3 times in D-PBS with 0.5% BSA. Cells were resuspended in PBS with 0.5% BSA, to which the purified scFv-V5 and anti-V5-FITC antibody were added and incubated for 1 hour at room temperature. Negative control left out scFv-V5. Cells were resuspended in 0.5 ml PBS and analyzed on a flow cytometer. Results are shown in FIG. 17. A population shift is observed only when the purified anti-PD1-scFv-V5 and anti-V5-FITC were both present (two different batches were shown), relative to samples with EL4 alone and EL4 plus secondary antibody only.

### Example 16. Secretion of Anti-mPD1-scFv

To generate recombinant bacteria which are capable of secreting anti-mPD1-scFv, constructs were generated according to methods described herein as shown in Table 34.

TABLE 34
Strains for secretion of anti-mPD1-scFv

| Strain Number | Genotype | Construct |
|---|---|---|
| SYN2790 | Nissle delta nlpI::CmR | pUC-ptet-OmpF-FLAG-J43scFv-V5-HIS |
| SYN2767 | Nissle delta tolA::CmR | pUC-ptet-OmpF-FLAG-J43scFv-V5-HIS |
| SYN2768 | Nissle delta PAL::CmR | pUC-ptet-OmpF-FLAG-J43scFv-V5-HIS |
| SYN2769 | Nissle delta lpp::CmR | pUC-ptet-OmpF-FLAG-J43scFv-V5-HIS |
| SYN2770 | Nissle delta nlpI::CmR | pUC-ptet-PhoA-FLAG-J43scFv-V5-HIS |
| SYN2771 | Nissle delta tolA::CmR | pUC-ptet-PhoA-FLAG-J43scFv-V5-HIS |
| SYN2772 | Nissle delta PAL::CmR | pUC-ptet-PhoA-FLAG-J43scFv-V5-HIS |
| SYN2773 | Nissle delta lpp::CmR | pUC-ptet-PhoA-FLAG-J43scFv-V5-HIS |
| SYN2774 | Nissle delta nlpI::CmR | pUC-ptet-PelB-FLAG-J43scFv-V5-HIS |
| SYN2775 | Nissle delta tolA::CmR | pUC-ptet-PelB-FLAG-J43scFv-V5-HIS |
| SYN2776 | Nissle delta PAL::CmR | pUC-ptet-PelB-FLAG-J43scFv-V5-HIS |
| SYN2777 | Nissle delta lpp::CmR | pUC-ptet-PelB-FLAG-J43scFv-V5-HIS |

Exemplary secretion construct sequences include Ptet-phoA-FLAG-J43-scFv-V5-HIS (SEQ ID NO: 981); phoA-FLAG-J43-scFv-V5-HIS (SEQ ID NO: 982); Ptet-ompF-FLAG-J43-scFv-V5-HIS (SEQ ID NO: 983); ompF-FLAG-J43-scFv-V5-HIS (SEQ ID NO: 984); Ptet-PelB-FLAG-J43-scFv-V5-HIS (SEQ ID NO: 985); and PelB-FLAG-J43-scFv-V5-HIS (SEQ ID NO: 986). In some embodiments, the scFv Secretion Construct is at least about 80%, 85%, 90%, 95%, or 99% homologous to the sequence of SEQ ID NOs: 981-986.

E. coli Nissle comprising plasmid based construct comprising tet-inducible J43-Anti-scFv-V5 with PhoA, OmpF or PelB secretion tags or wild type control were grown overnight in LB medium. Cultures were diluted 1:100 in LB and grown shaking (200 rpm) to an optical density of 0.8 at which time cultures were cooled down to room temperature and anhydrous tetracycline (ATC) was added to cultures at a concentration of 100 ng/mL to induce expression of PhoA-, OmpF- or PelB-J43-Anti-scFv-V5. No tetracycline was added to wild type Nissle cultures. After 18 hrs of induction at room temperature, bacteria were pelleted, and the supernatant was collected and placed on ice.

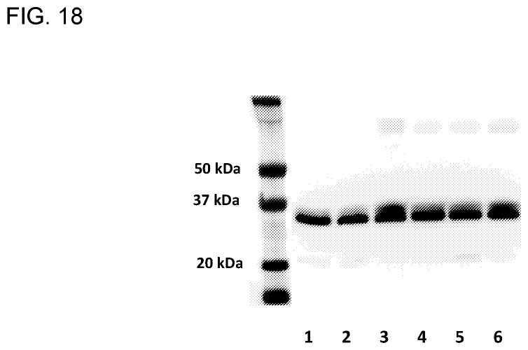

Protein concentration in the medium and the cell lysates was determined by BCA protein assay, and isolated extracts and media from wild type and strains comprising the Ptet-J43-anti-scFv-V5 were analyzed by Western blot. Proteins were transferred onto PVDF membranes and J43-anti-scFv detected with an HRP-conjugated anti-V5 antibody (Biolegend). Results are shown in FIG. 18. A single band was detected around 34 kDa in lane 1-6 corresponding to extracts from SYN2767, SYN2769, SYN2771, SYN2773, SYN2775 and SYN2777 respectively.

To determine whether the secreted J43-anti-scFv in E coli Nissle binds to PD1 on mouse cells, flow cytometric analysis was performed using EL4 cells. EL4 are a mouse lymphoma cell line which expresses PD1 on its cell surface.

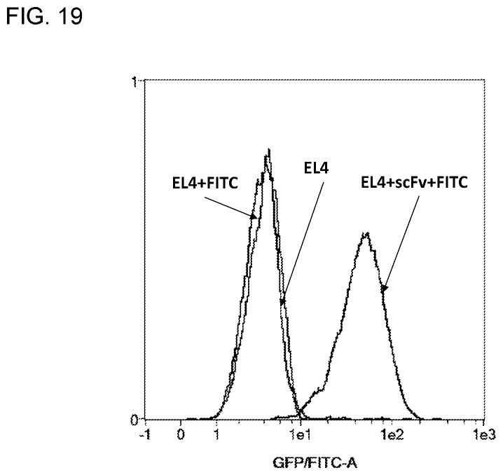

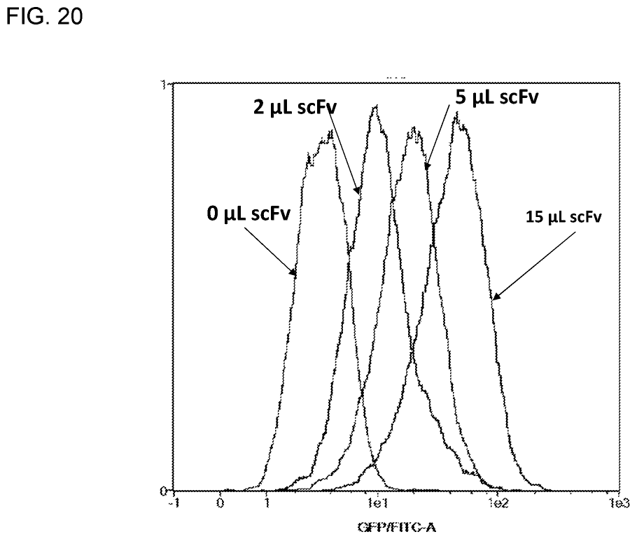

EL4 cells were grown in Dulbecco's Modified Eagle's Medium (DMEM) with 10% FBS and 1% Penicillin-Streptomycin Cells were spun down, supernatant was aspirated, pellet was resuspended in 1 ml D-PBS, transferred into chilled assay tubes (1×1⁰1\6 cells), and washed 3 times in D-PBS. Cells were resuspended in D-PBS with 0.5% BSA, to which the purified scFv-V5 and anti-V5-FITC antibody were added and incubated for 1 hour at room temperature. Negative control left out secreted J43-scFv-V5. Cells were then resuspended in 0.5 ml PBS and analyzed on a flow cytometer. Results are shown in (FIG. 19). A population shift is observed only when the secreted anti-PD1-scFv-V5 (1′ antibody) and anti-V5-FITC (2′ antibody) were both present, relative to samples with EL4 alone and EL4 plus secondary antibody only. A similar study was conducted with different amounts of the secreted scFv (0, 2, 5, and 15 jut), and a dose-dependent staining of the EL4 cells was observed (FIG. 20).

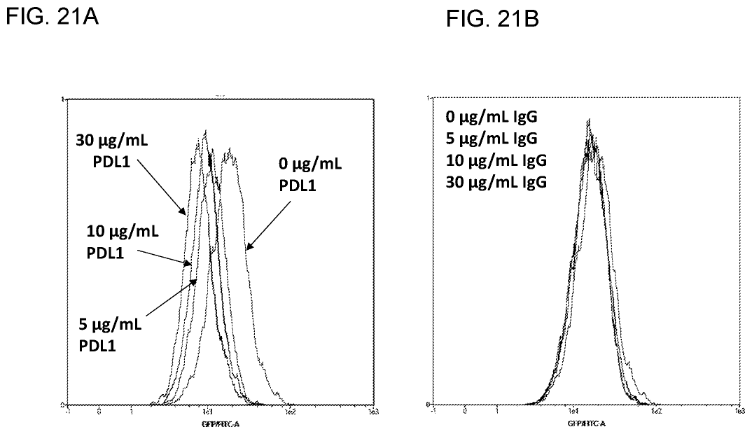

Next, a competition assay was conducted to determine whether PDL1 could inhibit the binding of the anti-PD1-scFv secreted by the recombinant bacteria from binding to murine PD1. EL4 cells were grown and flow cytometry protocol was conducted essentially as described above except that PDL1 was added at various concentrations (0, 5, 10, and 30 μg/mL) during the incubation of the secreted anti-PD1-scFv-V5. Rat-IgG was used as a negative control of secreted scFv. Results are shown in FIG. 21A and FIG. 21B. PDL1 competed in a dose dependent manner against the binding of secreted anti-mPD1-scFv to mPD1 on the surface of EL4 cells. Negative control of Rat-IgG protein did not show similar dose dependent binding competition.

### Example 17. Display of Anti-mPD1-scFv on E coli Nissle Cell Surface

To generate recombinant bacteria which are capable of displaying anti-mPD1-scFv on the Nissle cell surface, constructs were generated according to methods described herein as shown in Table 35. Selected display anchors include invasion display tag (SEQ ID NO: 990), LppOmpA display tag (SEQ ID NO: 991) and IntiminN display tag (SEQ ID NO: 992).

TABLE 35
Strains for display of anti-mPD1-scFv

| Strain | Host Strain | |
|---|---|---|
| Number | Genotype | Construct |
| SYN2797 | wt Nissle | p15A-Kan-ptet-Invasin-FLAG-J43scFv-V5-HIS |
| | | (SEQ ID NO: 987) |
| SYN2798 | wt Nissle | p15A-Kan-ptet-LppOmpA-FLAG-J43scFv-V5-HIS |
| | | (SEQ ID NO: 988) |
| SYN2799 | wt Nissle | p15A-Kan-ptet-IntiminN-FLAG-J43scFv-V5-HIS |
| | | (SEQ ID NO: 989) |

In some embodiments, the scFv Display Construct Sequence is at least about 80%, 85%, 90%, 95%, or 99% homologous to the sequence of SEQ ID NO: 987, SEQ ID NO: 988, and/or SEQ ID NO: 989.

In some embodiments, the display anchor is at least about 80%, 85%, 90%, 95%, or 99% homologous to the sequence of SEQ ID NO: 990, SEQ ID NO: 991, and/or SEQ ID NO: 992.

E. coli Nissle comprising a plasmid based construct comprising tet-inducible ptet-LppOmpA-anti-PD1-scFv was grown overnight in LB medium. Cultures were diluted 1:100 in LB and grown shaking (200 rpm) to an optical density of 0.8 at which time culture was cooled down to room temperature and anhydrous tetracycline (ATC) was added to cultures at a concentration of 100 ng/mL to induce expression of ptet-LppOmpA-J43-scFv for 18 hours.

To determine whether the single-chain antibody was displayed on the surface of the genetically engineered E. coli Nissle and functionally binds to PD1 a whole cell ELISA assay was performed. 1⁰1\9 cells were blocked using PBS with 2% BSA for 1 h at room temperature and biotinylated-mPD1 was added and incubated for 1 h at room temperature. Afterwards, cells were washed 3 times with PBST (PBS/0.1% Tween-20) and incubated with a streptavidin conjugated HRP in blocking solution for 40 min. Following incubation, wells were washed 3 times with PBST and resuspended in PBS, then stained using a 3,3′,5,5′-tetramethylbenzidine (TMB) substrate kit per the manufacturer's instructions (Thermofisher). Biotinylated IgG and plain PBS were used instead of mPD1 as negative controls. Cells were removed by centrifugation and supernatants were collected. Signal intensities of supernatant were measured using an ELISA reader at 450 nm. Results are shown in Table 36 and indicate that the J43-scFv (anti-mPD1) is displayed on the surface of the recombinant bacteria and can bind to mPD1.

TABLE 36
Nissle Surface Display ELISA Assay

| | | Primary | Secondary |
|---|---|---|---|
| Strain | OD450 | antibody | antibody |
| SYN2798 (p15A-ptet-LppOmpA-anti-PD1-scFv) | 0.125 | PBS only | Strp-HRP |
| SYN2798 (p15A-ptet-LppOmpA-anti-PD1-scFv) | 0.133 | mIgG-strp | Strp-HRP |
| SYN2798 (p15A-ptet-LppOmpA-anti-PD1-scFv) | 0.421 | mPD1-strp | Strp-HRP |

### Example 18. Anti-CD47 scFv Expression in E. coli

To determine whether a functional anti-CD47-scFv can be expressed in E. coli, an anti-CD47-scFv fragment was generated based on B6H12 and 5F9 monoclonal antibodies, which reacts with human CD47.

Monoclonal antibody B6H12 and 5F9 (anti human CD47) sequences were obtained from published patent (US 20130142786 A1). Next, the single-chain variable fragment (scFv) targeting human CD47 was designed. A fragment containing tet promoter, a ribosome binding site, the designed antihCD47-scFv, a C terminal V5 tag and a C terminal hexa-histidine tag (SEQ ID NO: 1166) was synthesized by IDTDNA. The construct was cloned into the pCR™-Blunt II-TOPOO Vector (Invitrogen) and transformed into E. coli DH5a as described herein to generate the plasmid pUC-ptet-B6H12antihCD47scFv-V5-HIS (SEQ ID NO: 993) and pUC-ptet-5F9antihCD47scFv-V5-HIS (SEQ ID NO: 995). B6H12-anti-CD47-scFv polypeptide sequence refers to SEQ ID NO: 994 and 5F9-anti-CD47-scFv polypeptide sequence refers to SEQ ID NO: 996.

In some embodiments, the Anti-CD47 scFv sequences is at least about 80%, 85%, 90%, 95%, or 99% homologous to the sequence of SEQ ID NO: 993, SEQ ID NO: 994, SEQ ID NO: 995, and/or SEQ ID NO: 996.

E. coli DH5a comprising pUC-ptet-B6H12antihCD47scFv-V5-HIS or pUC-ptet-5F9antihCD47scFv-V5-HIS were grown overnight in LB medium. Cultures were diluted 1:100 in LB and grown shaking (200 rpm) to an optical density of 0.8 at which time cultures were cooled down to room temperature and anhydrous tetracycline (ATC) was added to cultures at a concentration of 100 ng/mL to induce expression of ptet-scFv for 18 hours and then bacteria were pelleted, washed in PBS, and harvested, resuspended in 2 mL PBS buffer and lysed by sonication on ice. Insoluble debris is spun down twice for 15 min at 12,000 rpm at 4° C.

To determine whether the anti CD47 single-chain antibody expressed in E. coli DH5a functionally binds to the target protein, an ELISA assay was performed. Plates were absorbed overnight at 4° C. with 100 μL of 2 μg/mL per well of target proteins (humanCD47, mouseCD47, IgG and PBS, from Rndsystems). Wells were blocked with 2% BSA in PBS/0.1% Tween-20 for 2 hours at room temperature. After three washes, wells were incubated with bacterial extracts for 1 hour at room temperature. Wells were washed 4times with PBST (PBS/0.1% Tween-20) and incubated with a HRP-conjugated anti-V5 antibody(Biolegend) in blocking solution for 40 min. Following incubation, wells were washed 4 times with PBST and then stained using a 3,3′,5,5′-tetramethylbenzidine (TMB). Signal intensities were measured using an ELISA reader at 450 nm. Results are shown in Table 37 and indicate that the antiCD47-scFv expressed by the recombinant bacteria can bind to humanCD47 specifically.

TABLE 37
ELISA Binding Assay

| | | Primary | Secondary | |
|---|---|---|---|---|
| Strain | Coating | antibody | antibody | OD450 |
| SYN2936 (pUC-Ptet- | PBS | B6H12-scFv | Anti-V5-HRP | 0.047 |
| B6H12scFv-V5-HIS) | | extracts | | |
| SYN2936 (pUC-Ptet- | IgG | B6H12-scFv | Anti-V5-HRP | 0.064 |
| B6H12scFv-V5-HIS) | | extracts | | |
| SYN2936 (pUC-Ptet- | hCD47 | B6H12-scFv | Anti-V5-HRP | 1.587 |
| B6H12scFv-V5-HIS) | | extracts | | |
| SYN2936 (pUC-Ptet- | mCD47 | B6H12-scFv | Anti-V5-HRP | 0.053 |
| B6H12scFv-V5-HIS) | | extracts | | |
| SYN2937 (pUC-Ptet- | PBS | 5F9-scFv | Anti-V5-HRP | 0.048 |
| 5F9scFv-V5-HIS) | | extracts | | |
| SYN2937 (pUC-Ptet- | IgG | 5F9-scFv | Anti-V5-HRP | 0.057 |
| 5F9scFv-V5-HIS) | | extracts | | |
| SYN2937 (pUC-Ptet- | hCD47 | 5F9-scFv | Anti-V5-HRP | 1.838 |
| 5F9scFv-V5-HIS) | | extracts | | |
| SYN2937 (pUC-Ptet- | mCD47 | 5F9-scFv | Anti-V5-HRP | 0.053 |
| 5F9scFv-V5-HIS) | | extracts | | |

### Example 19. Tumor Pharmacokinetics for E. coli Nissle over a 7 Day Period

Tumor pharmacokinetics of streptomycin resistant Nissle were determined using a CT26 tumor model. CT26 cells obtained from ATCC were cultured according to guidelines provided. Approximately −1e6cells/mouse in PBS were implanted subcutaneously into the right flank of each animal (BalbC/J (female, 8 weeks)), and tumor growth was monitored for approximately 10 days. When the tumors reached about −100-150 mm3, animals were randomized into groups for dosing.

For intratumoral injection, bacterial strain (Streptomycin resistant Nissle (SYN094) was grown in LB broth until reaching an absorbance at 600 nm (A600 nm) of 0.4 (corresponding to 2×108 colony-forming units (CFU)/mL) and washed twice in PBS. The suspension was diluted in PBS or saline so that 100 microL can be injected at the appropriate doses intratumorally into tumor-bearing mice.

Approximately 10 days after CT 26 implantation, bacteria were suspended in 0.1 ml of PBS and mice were injected (1e7 and 1e8 cells/dose) with 100 ul intratumorally as follows: Group 1-SYN94 IT 10e7, 1.5 h (n=3); Group 2-SYN94 IT 10e8, 1.5 hh (n=3); Group 3-SYN94 IT 10e7, 4 h (n=3); Group 4-SYN94 IT 10e8, 4 h (n=3); Group 5-SYN94 IT 10e7, 24 h (n=3); Group 6-SYN94 IT 10e8, 24 h (n=3); Group 7-SYN94 IT 10e7, 72 h (n=3); Group 8-SYN94 IT 10e8, 72 h (n=3); Group 9-SYN94 IT 10e7, 7d (n=3); Group 10-SYN94 IT 10e8, 7d (n=3).

On day 1, animals were dosed intratumorally (IT) with 100u1 SYN94 at the two doses. At 1.5 and 4 h post dose, tumor, liver, lung and DLN tissue and blood was harvested. On day 2 (24 h post dose), on day 4 (72 hours post dose), and on day 7 (7d post dose) tissues and blood was harvested in the same manner as on Day 1.

In order to determine the CFU of bacteria in each sample, the blood samples were serially diluted, and the tissue samples were homogenized in PBS and serially diluted. Dilutions were plated onto LB plates containing streptomycin. The plates were incubated at 37° C. overnight, and colonies were counted. Bacterial counts in the tumor tissue were similar at both doses.

### Example 20. Tumor Pharmacokinetics for E coli Nissle and E coli Nissle DOM mutants

Tumor pharmacokinetics of streptomycin resistant Nissle and a Nissle DOM mutant (Nissle delta PAL::CmR) were compared in a CT26 tumor model.

CT26 cells obtained from ATCC were cultured according to guidelines provided.

Approximately −1e6cells/mouse in PBS were implanted subcutaneously into the right flank of each animal (BalbC/J (female, 8 weeks)), and tumor growth was monitored for approximately 10 days. When the tumors reached about −100-150 m^{m}3, animals were randomized into groups for dosing.

For intratumoral injection, bacterial strains (Streptomycin resistant Nissle (SYN094) and SYN1557 (Nissle delta PAL::CmR) were grown in LB broth until reaching an absorbance at 600 nm (A600 nm) of 0.4 (corresponding to 2×10⁸ colony-forming units (CFU)/mL) and washed twice in PBS. The suspension was diluted in PBS or saline so that 100 microL can be injected at the appropriate doses intratumorally into tumor-bearing mice.

Approximately 10 days after CT 26 implantation, bacteria were suspended in 0.1 ml of PBS and mice were injected (1e7 cells/dose) with 100 ul intratumorally as follows: Group 1-SYN1557, 1.5 h (n=3); Group 2-SYN94, 1.5 h (n=3); Group 3-SYN1557, 4 h (n=3); Group 4-SYN94, 4 h (n=3); Group 5-SYN1557, 24 h (n=3); Group 6-SYN94, 24 h (n=3); Group 7-SYN1557, 72 h (n=3); Group 8-SYN94, 72 h (n=3); Group 9-SYN1557, 7d (n=3); Group 10-SYN94, 7d (n=3).

On day 1, animals were dosed intratumorally (IT) with 100u1 SYN94 or SYN1557 (1e7 cells/dose). At 1.5 and 4 h post dose, tumor tissue and blood was harvested. For blood collection 20u1 was used for bacterial plating-process, and the rest of sample was used for serum. On day 2 (24 h post dose), on day 4 (72 hours post dose), and on day 7 (7d post dose) tumor tissue and blood was harvested the same as on Day 1.

In order to determine the CFU of bacteria in each sample, the blood samples were serially diluted, and the tumor sample was homogenized in PBS and serially diluted. Dilutions were plated onto LB plates containing streptomycin or chloramphenicol. The plates were incubated at 37° C. overnight, and colonies were counted. Bacterial counts in the tumor tissue were similar in both strains, and no bacteria were detected in the blood. These results indicate that both the wild type and the DOM mutant Nissle can survive in the tumor environment.

### Example 21. Secretion of murine CD40L

To generate recombinant bacteria which are capable of secreting CD40L, mCD40L1(47-260) and mCD40L2(122-260) constructs as shown in Table 38 were generated according to methods described herein. mCD40L1(47-260) and mCD40L2(122-260) correspond to extracellular portion of the full length mCD40L and soluble form of mCD4L, respectively. Nucleotide and polypeptide sequences for phoA-mCD40L1 (47-260) comprising a FLAG tag, V5 Tag, and His Tag are shown in SEQ ID NO: 1167 and 1169, respectively. Nucleotide and polypeptide sequences for phoA-mCD40L1 (112-260) comprising a FLAG tag, V5 Tag, and His Tag are shown in SEQ ID NO: 1168 and 1170, respectively. Polypeptide sequence for extracellular portion of human CD40L (47-261) is shown in SEQ ID NO: 1171.

TABLE 38
Strains for secretion of murine CD40L

| Strain Number | Genotype | Construct |
|---|---|---|
| SYN1557 | Nissle delta PAL::CmR | |
| (parental) | | |
| SYN3366 | Nissle delta PAL::CmR | pUC-ptet-phoA-mCD40L1 |
| | | (47-260) -V5-HIS |
| SYN3367 | Nissle delta PAL::CmR | pUC- ptet-phoA-mCD40L2 |
| | | (112-260) -V5-HIS |

E. coli Nissle comprising plasmid-based tet-inducible constructs comprising ptet-PhoA-CD40L1 (47-260) (SYN3366) and tet-PhoA-CD40L2 (112-260) (SYN3367) and the parental control strain SYN1557 were grown overnight in LB medium. Cultures were diluted 1:100 in LB and grown shaking (200 rpm) to an optical density of 0.8, at which time the cultures were cooled down to room temperature and anhydrous tetracycline (ATC) was added to cultures at a concentration of 100 ng/mL to induce expression of mCD40L1 and mCD40L2.

After 18 hours of induction, cells were spun down, and supernatant was collected. To generate cell free medium, the clarified supernatant was further filtered through a 0.22 micron filter to remove any remaining bacteria and placed on ice.

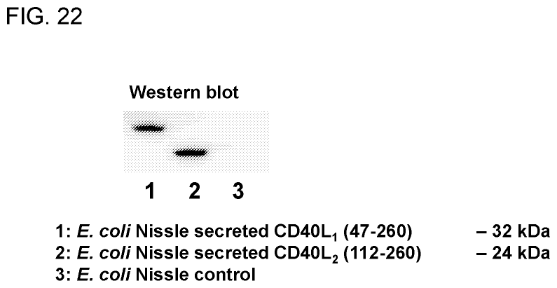

Supernatants were then analyzed by western blot. Proteins from 25 μL supernatant were transferred onto PVDF membranes and mCD40-L1 and mCD40L-2 were detected with an HRP-conjugated anti-V5 antibody (Biolegend). Results are shown in FIG. 22. A single band was detected around 32 kDa and 24 kDa for mCD40L1 and mCD40L2, respectively.

To determine whether the mCD40L secreted by the genetically engineered E. coli Nissle captured in the clarified supernatant can functionally bind to mCD40 and/or is detected by an anti-mCD40L antibody, an ELISA assay was performed by coating the plate with mCD40 or anti-mCD40L antibody. Results are shown in Table 39 and indicate that mCD40L1 (47-260) and mCD40L2 (112-260) secreted by the recombinant bacteria and can bind to mCD40.

TABLE 39
CD40 ELISA Binding Assay

Coating Materials

| Samples | mCD40 | Anti-mCD40L | IgG | PBS |
|---|---|---|---|---|
| PBS | 0.051 | 0.063 | 0.049 | 0.047 |
| Control | 0.054 | 0.065 | 0.052 | 0.054 |
| Secreted | 0.231 | 0.394 | 0.052 | 0.049 |
| CD40-L1 | | | | |
| Secreted | 0.639 | 0.825 | 0.052 | 0.05 |
| CD40-L2 | | | | |

### Example 22. Secretion of SIRPα and Variants and anti-CD47 scFv

To generate recombinant bacteria which are capable of secreting anti-SIRPA, constructs shown in Table 40 were generated according to methods described herein. Sequences for use in the secretion constructs include PhoA-mSIRPα(32-373)-tagged with FLAG, V5 and HIS tag (SEQ ID NO: 1172); PhoA-mCV1SIRPα tagged with FLAG, V5 and HIS tags (SEQ ID NO: 1173); mCV1SIRPα (SEQ ID NO: 1174); PhoA-mFD62XSIRPα tagged with FLAG, V5 and HIS tags (SEQ ID NO: 1175); PhoA-mCV1SIRPα-hIgG4 with V5 and HIS tags (SEQ ID NO: 1176); PhoA-mCV1SIRPα-hIgG4 (SEQ ID NO: 1177); mCV1SIRPα-hIgG4 (SEQ ID NO: 1177); PhoA-mFD6SIRPα-hIgG4 tagged with FLAG, V5 and HIS (SEQ ID NO: 1178); mFD6SIRPα-hIgG4 (SEQ ID NO: 1179); hIgG4 (SEQ ID NO: 1180); and PhoA-Anti-mouse CD47 scFv tagged with FLAG, V5 and HIS tags (SEQ ID NO: 1181).

Sequences of the corresponding polypeptides include PhoA-mSIRPα(32-373)-tagged with FLAG, V5 and HIS tag (SEQ ID NO: 1182); PhoA-mCV1SIRPα tagged with FLAG, V5 and HIS tags (SEQ ID NO: 1183); mCV1SIRPα (SEQ ID NO: 1184); PhoA-mFD62xSIRPα tagged with FLAG, V5 and HIS (SEQ ID NO: 1185); mFD6SIRPα (SEQ ID NO: 1186); PhoA-mCV1SIRPα-hIgG4 tagged with V5 and HIS (SEQ ID NO: 1187); PhoA-mCV1SIRPα-hIgG4 (SEQ ID NO: 1188); mCV1SIRPα-hIgG4 (SEQ ID NO: 1189); PhoA-mFD6SIRPα-hIgG4 tagged with FLAG, V5 and HIS (SEQ ID NO: 1190); PhoA-mFD6SIRPα-hIgG4 (SEQ ID NO: 1191); mFD6SIRPα-hIgG4 (SEQ ID NO: 1192); mFD6SIRPα (SEQ ID NO: 1193); hIgG4 (SEQ ID NO: 1194); PhoA-Anti-mouse CD47 scFv tagged with V5 and HIS tags (SEQ ID NO: 1195); hSIRPα (31-373) extracellular (SEQ ID NO: 1196); hSIRPα (374-394) helical (SEQ ID NO: 1197); and hSIRPα (395-504) cytoplasmic (SEQ ID NO: 1198)

TABLE 40
Strains for secretion of SIRPα, SIRPα variants and mCD47 scFv Ligand

| Strain Number | Genotype | Construct |
|---|---|---|
| SYN1557 (parental) | Nissle delta PAL::CmR | |
| SYN2996 | Nissle delta PAL::CmR | p15A-ptet-PhoA-FLAG-mSIRPa(32-373)-V5-HIS |
| SYN3159 | Nissle delta PAL::CmR | pUC-ptet-PhoA-FLAG-CV1sirpα-V5-HIS |
| SYN3160 | Nissle delta PAL::CmR | pUC-ptet-PhoA-FLAG-FD6x2sirpα-V5-HIS |
| SYN3021 | Nissle delta PAL::CmR | pUC-ptet-PhoA-SIRPαCV1hIgG4-V5-HIS |
| SYN3020 | Nissle delta PAL::CmR | pUC-ptet-PhoA-FD6sirpαhIgG4-V5-HIS |
| SYN3161 | Nissle delta PAL::CmR | pUC-tet-PhoA-αmCD47scFv-V5-HIS |

E. coli Nissle strains SYN1557, SYN2996, SYN3159, SYN3160, SYN3021, SYN3020, and SYN3161, were grown overnight in LB medium. Cultures were diluted 1:100 in LB and grown shaking (200 rpm) to an optical density of 0.8 at which time culture was cooled down to room temperature and anhydrous tetracycline (ATC) was added to cultures at a concentration of 100 ng/mL to induce expression of SIRPα or SIRPα variants, or CD47 scFvs.

After 18 hours of induction, cells were spun down, and supernatants were collected. To generate cell free medium, the clarified supernatants were further filtered through a 0.22 micron filter to remove any remaining bacteria and placed on ice.

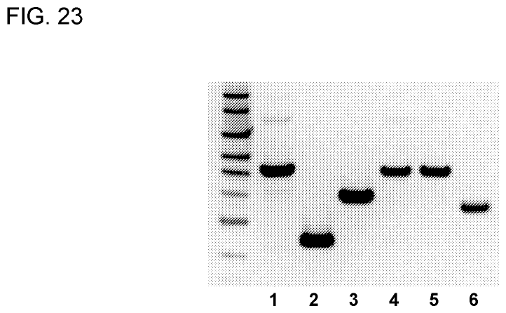

Supernatant was then analyzed by western blot. Proteins in 25 μL supernatant were transferred onto PVDF membranes and SIRPα and SIRPα variants and anti-CD47 scFv were detected using anti-V5-HRP antibody (Biolegend). Results are shown in FIG. 23. A single band was detected around 46 kDa for WT mSIRPα, 20 kDa for CV1SIRPα, 33 kDa for FD6x2 SIRPα, 42 kDa for FD6SIRPα-IgG4, 42 kDa for CV1SIRPα-IgG4, and 30 kDa for anti-CD47 scFv, respectively.

To determine whether the wild type SIRPα, SIRPα variants, and anti-CD47-scFvs secreted by the genetically engineered E. coli Nissle can functionally bind to CD47 and/or are detected by an anti-SIRPα antibody, an ELISA assay was performed by coating the plate with corresponding antibodies or ligands. Results are shown in Table 41 and indicate that both the secreted mSIRPα and anti-mCD47scFv by the recombinant bacteria can bind to mCD47.

TABLE 41
SIRPα/CD47 ELISA Binding Assay

Coating Materials

| Samples | mCD47 | Anti-mSIRPa | IgG | PBS |
|---|---|---|---|---|
| PBS | 0.051 | 0.054 | 0.054 | 0.051 |
| Control | 0.056 | 0.052 | 0.053 | 0.054 |
| Secreted WT | 1.069 | 0.829 | 0.056 | 0.059 |
| mSIRPα | | | | |

| | Coating Materials | | |
|---|---|---|---|
| Samples | mCD47 | IgG | PBS |
| PBS | 0.046 | 0.053 | 0.048 |
| Control | 0.049 | 0.051 | 0.048 |
| Secreted | 0.527 | 0.067 | 0.053 |
| anti-mCD47 scFv | | | |

To determine whether the SIRPα, SIRPα variants, and anti-CD47 scFv secreted from in E coli Nissle bind to CD47 on mouse cells, flow cytometric analysis was performed using CT26 cells. CT26 is a mouse colon carcinoma cell line which expresses CD47 on its cell surface.

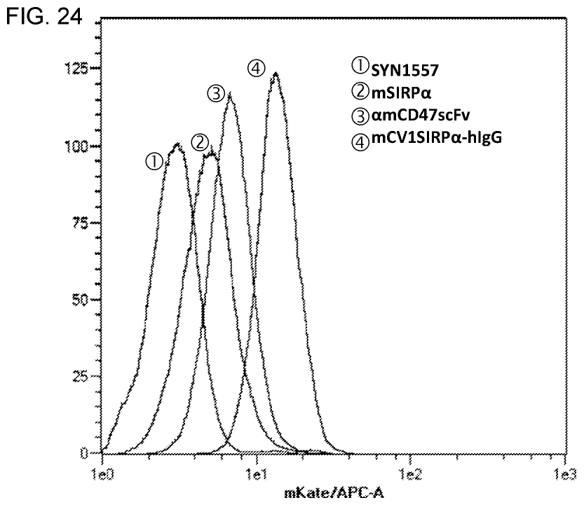

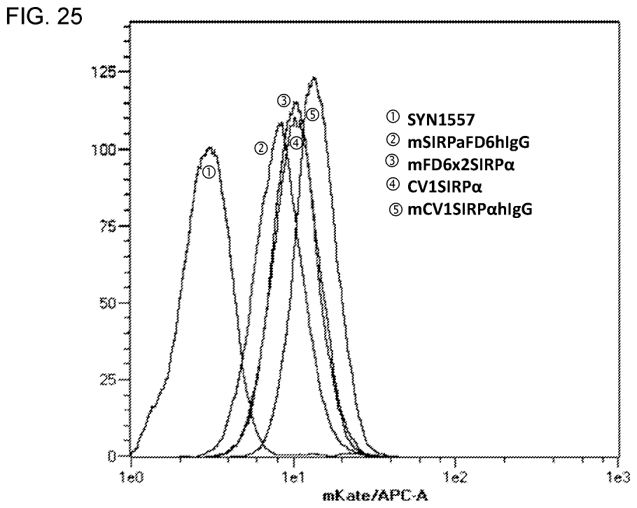

CT26 cells were grown in Dulbecco's Modified Eagle's Medium (DMEM) with 10% FBS and 1% Penicillin-Streptomycin. Cells were spun down, supernatant was aspirated, pellet was resuspended in 1 ml D-PBS, transferred into chilled assay tubes (1×10\hat{ }6 cells), and washed 3 times in D-PBS. Cells were resuspended in D-PBS with 0.5% BSA, to which the 10 jut of the supernatant (10× concentrated) were added and incubated for 1 hour at 4 C. Supernatant from SYN1557 was used as a baseline negative control. Cells were then resuspended in 0.5 ml PBS, diluted to proper concentration and analyzed on a flow cytometer. Results are shown in (FIG. 24 and FIG. 25). A population shift relative to baseline is observed for the samples containing the secreted SIRPα, SIRPα variants, and anti-CD47 scFv, with the greatest shift observed with SYN3021, expressing CV1SIRPα-IgG4.

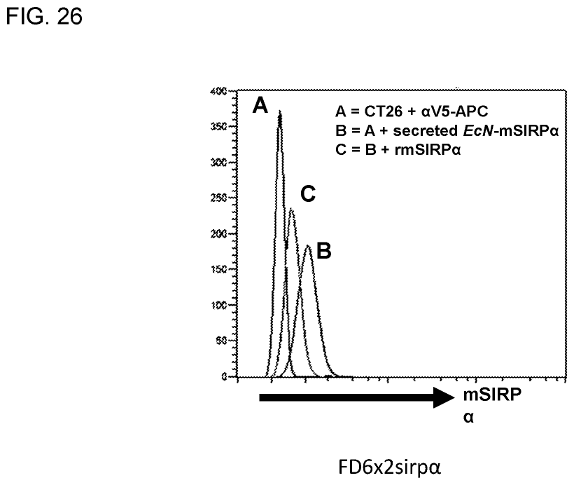

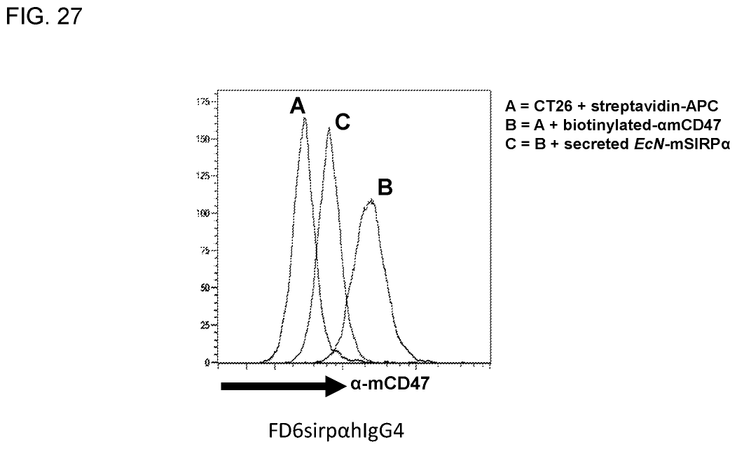

Next, a competition assay was conducted to determine whether the murine SIRPα secreted by the recombinant bacteria could compete with the binding of a recombinant mSIRPα or an anti-CD47 antibody to murine CD47 on CT26 cells. CT26 cells were grown and flow cytometry protocol was conducted essentially as described above except that recombinant SIRPα (Rndsystems) or an anti-CD47 antibody (Biolegend) was added during the incubation of the secreted FD6x2sirpα or FD6sirpαhIgG4. FIG. 26 and FIG. 27 show the results of the competition with recombinant SIRPalpha and anti-CD47 antibody, respectively. Both recombinant SIRPalpha and anti-CD47 antibody were able to compete with the secreted SIRPalpha for the binding to CD47 on the CT26 cells.

### Example 23. Secretion of Hyaluronidase

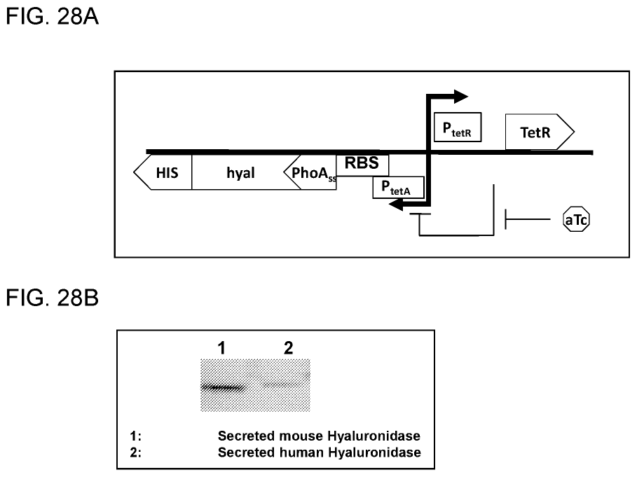

To generate recombinant bacteria which are capable of secreting hyaluronidase, constructs were generated according to methods described herein as shown in FIG. 28A (SYN2998: Nissle delta PAL::CmR; p15A-ptet-RBS-PhoA-FLAG-human hyaluronidase-V5-His tags; SYN2997: Nissle delta PAL::CmR; p15A-ptet-RBS-PhoA-FLAG-human hyaluronidase-V5-His; SYN3369: Nissle delta PAL::CmR; p15A-ptet-RBS-PhoA-FLAG-leech hyaluronidase-V5-His). Sequences for use in the secretion constructs include PhoA-human hyaluronidase-tagged with FLAG, V5, and His (SEQ ID NO: 1199); PhoA-human hyaluronidase (SEQ ID NO: 1200); PhoA-mouse hyaluronidase-tagged with FLAG, V5 and His tags (SEQ ID NO: 1201); PhoA-leech hyaluronidase-tagged with FLAG, V5 and His tags (SEQ ID NO: 1202); and PhoA-leech hyaluronidase (SEQ ID NO: 1203). Sequences for the corresponding polypeptide include PhoA-human hyaluronidase-tagged with FLAG, V5, and His (SEQ ID NO: 1204); human hyaluronidase (SEQ ID NO: 1205); PhoA-human hyaluronidase (SEQ ID NO: 1206); PhoA-mouse hyaluronidase-tagged with FLAG, V5 and His tags (SEQ ID NO: 1207); and leech hyaluronidase (SEQ ID NO: 1208).

E. coli Nissle strains SYN1557, SYN2997 (secreting mouse hyaluronidase), SYN2998 (secreting human hyaluronidase) and SYN3369 (secreting leech hyaluronidase), were grown overnight in LB medium. Cultures were diluted 1:100 in LB and grown shaking (200 rpm) to an optical density of 0.8 at which time culture was cooled down to room temperature and anhydrous tetracycline (ATC) was added to cultures at a concentration of 100 ng/mL to induce expression hyaluronidase.

After 18 hours of induction, cells were spun down, and supernatant was collected. To generate cell free medium, the clarified supernatant was further filtered through a 0.22 micron filter to remove any remaining bacteria and placed on ice.

Supernatants were then analyzed by western blot. Proteins in 25 μL supernatant were transferred onto PVDF membranes and hyaluronidase was detected using anti-V5-HRP antibody (Biolegend). Results are shown in FIG. 28B. A single band was detected around 50 kDa for both secreted mouse and human hyaluronidases and around 57 kDa for leech hyaluronidase.

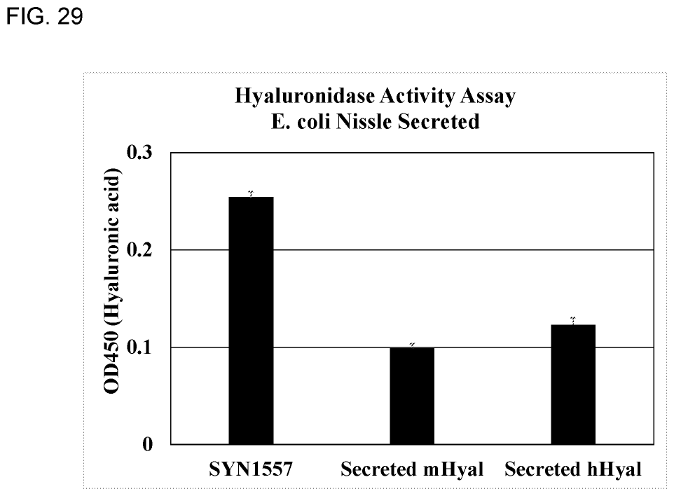

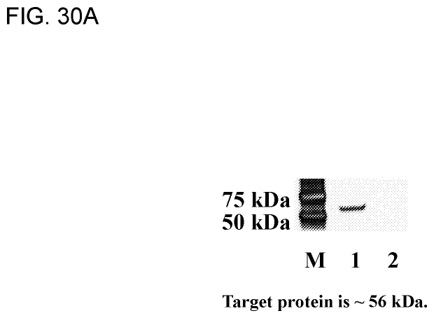

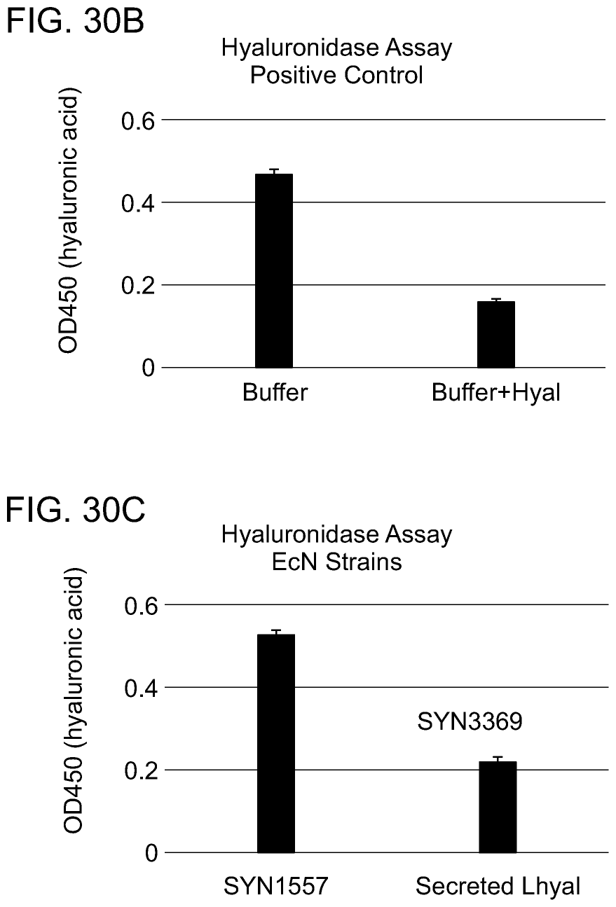

To determine whether the hyaluronidase secreted by the genetically engineered E. coli Nissle is active, a hyaluronidase activity assay on ability to cleave hyaluronan was performed, using biotinylated hyaluronate coated plate, according to manufacturer's instructions. Briefly, hyaluronidase will cleaved the polymer and result in the loss of biotin on plate which can then be detected by streptavidin-HPR and substrate. Results are shown in FIG. 29 and indicate that hyaluronidase secreted by the recombinant bacteria is able to degrade hyaluronan. The parental strain SYN1557 was used as a negative control. Results obtained for secreted leech hyaluronidase are shown in FIGS. 30A-C.

### Example 24. Anti-Cancer Molecules

Exemplary sequences for use in constructing single-chain antibodies for Flagellar Type III secretion include (1) anti CTLA-4 scFv (heavy chain—linker—light chain transcribed from the native FliC promoter and 5′UTR with an optimized ribosome binding site (SEQ ID NO: 765; SEQ ID NO: 766)); (2) anti CTLA-4 scFv (light chain—linker—heavy chain transcribed from the native FliC promoter and 5′UTR with an optimized ribosome binding site (SEQ ID NO: 767; SEQ ID NO: 768)); (3) anti PD-1 scFv (heavy chain—linker—light chain transcribed from the native FliC promoter and 5′UTR with an optimized ribosome binding site (SEQ ID NO: 769; SEQ ID NO: 770)); (4) anti PD-1 scFv (light chain—linker—heavy chain transcribed from the native FliC promoter and 5′UTR with an optimized ribosome binding site (SEQ ID NO: 771; SEQ ID NO: 772)); (5) anti PDL-1 scFv (heavy chain—linker—light chain transcribed from the native FliC promoter and 5′UTR with an optimized ribosome binding site (SEQ ID NO: 773; SEQ ID NO: 774)); (6) anti PDL-1 scFv (light chain—linker—heavy chain transcribed from the native FliC promoter and 5′UTR with an optimized ribosome binding site (SEQ ID NO: 775; SEQ ID NO: 776)).

In some embodiments, recombinant bacteria comprise a nucleic acid sequence that is at least about 80%, 85%, 90%, 95%, or 99% homologous to the DNA sequence or comprises a DNA sequence that encodes a polypeptide that is at least about 80%, 85%, 90%, 95%, or 99% homologous to SEQ ID Nos: 765-776.

Exemplary sequences for use in constructing single-chain antibodies for Type V auto-secretor (pic protein) secretion include (1) anti CTLA-4 scFv (heavy—linker—light transcribed from the native ptet (tetracycline responsive) promoter and with an optimized ribosome binding site expressed as a fusion protein with the native Nissle auto-secreter E. coli_01635; (SEQ ID NO: 777; SEQ ID NO: 778)); (2) anti CTLA-4 scFv (light—linker—heavy transcribed from the native ptet promoter and with an optimized ribosome binding site (SEQ ID NO: 779; SEQ ID NO: 780); (3) anti PD-1 scFv (heavy—linker—light transcribed from the native ptet promoter and with an optimized ribosome binding site; (SEQ ID NO: 781; SEQ ID NO: 782)); (4) anti PD-1 scFv (light—linker—heavy transcribed from the native ptet promoter and with an optimized ribosome binding site (SEQ ID NO:783; SEQ ID NO: 784)); (5) Anti PDL-1 scFv (heavy—linker—light transcribed from the native ptet promoter and with an optimized ribosome binding site (SEQ ID NO: 785; SEQ ID NO: 786)); (6) anti PDL-1 scFv (light—linker—heavy transcribed from the native ptet promoter and with an optimized ribosome binding site (SEQ ID NO: 787; SEQ ID NO: 788)); (7) anti CTLA-4 scFv (heavy—linker—light transcribed from the native ptet promoter and with an optimized ribosome binding site (SEQ ID NO: 777; SEQ ID NO: 778)); (8) anti CTLA-4 scFv (light—linker—heavy transcribed from the native ptet promoter and with an optimized ribosome binding site (SEQ ID NO: 779; SEQ ID NO: 780)); (9) anti PD-1 scFv (heavy—linker—light transcribed from the native ptet promoter and with an optimized ribosome binding site (SEQ ID NO: 781; SEQ ID NO: 782)); (10) anti PD-1 scFv (light—linker—heavy transcribed from the native ptet promoter and with an optimized ribosome binding site (SEQ ID NO: 783; SEQ ID NO: 784)); (11) anti PDL-1 scFv (heavy—linker—light transcribed from the native ptet promoter and with an optimized ribosome binding site (SEQ ID NO: 785; SEQ ID NO: 786)); (12) anti PDL-1 scFv (light—linker—heavy transcribed from the native ptet promoter and with an optimized ribosome binding site (SEQ ID NO: 787; SEQ ID NO: 788)).

In some embodiments, recombinant bacteria comprise a nucleic acid sequence that is at least about 80%, 85%, 90%, 95%, or 99% homologous to the DNA sequence or comprises a DNA sequence that encodes a polypeptide that is at least about 80%, 85%, 90%, 95%, or 99% homologous to SEQ ID NOS: 777-788.

Exemplary sequences for use in constructing single-chain antibodies for Type I hemolysin secretion include (1) anti CTLA-4 scFv (heavy chain—linker—light chain transcribed from the native ptet (tetracycline responsive) promoter and with an optimized ribosome binding site expressed as fusion protein with the 53 amino acids of the C termini of alpha-hemolysin (hlyA) of E. coli CFT073 (SEQ ID NO: 789; SEQ ID NO: 790)); (2) anti CTLA-4 scFv (light chain—linker— heavy chain transcribed from the native ptet promoter and with an optimized ribosome binding site expressed (SEQ ID NO: 791; SEQ ID NO: 792)); (3) anti PD-1 scFv (heavy chain—linker—light chain transcribed from the native ptet promoter and with an optimized ribosome binding site (SEQ ID NO: 793; SEQ ID NO: 794)); (4) anti PD-1 scFv (light chain—linker—heavy chain transcribed from the native ptet promoter and with an optimized ribosome binding site (SEQ ID NO: 795; SEQ ID NO: 796)); (5) anti PDL-1 scFv (heavy chain—linker—light chain transcribed from the native ptet promoter and with an optimized ribosome binding site (SEQ ID NO: 797; SEQ ID NO: 798)); (6) anti PD-1 scFv (light chain—linker—heavy chain transcribed from the native ptet promoter and with an optimized ribosome binding site (SEQ ID NO: 799; SEQ ID NO: 800)). Selected sequences for use in generating constructs for single chain antibody production and secretion include fliC promoter (SEQ ID NO: 801); fliC 5′ untranslated region (SEQ ID NO: 802); Tetracycline responsive promoter (SEQ ID NO: 803; SEQ ID NO: 804); Optimized ribosome binding sites (SEQ ID NOs: 805-817); terminator sequences (SEQ ID NOS: 818-820); N terminal secretion tag for Type V auto-secretor secretion (SEQ ID NO: 821); C terminal secretion tag for Type V auto-secretor secretion (SEQ ID NO: 822); Anti-CTLA-4 single chain antibody coding region (Heavy Chain—linker—Light Chain) (SEQ ID NO: 823); Anti-CTLA-4 single chain antibody coding region (Light Chain—linker—Heavy Chain) (SEQ ID NO: 824); Anti-PD-1 single chain antibody coding region (Heavy Chain—linker—Light Chain) (SEQ ID NO: 825); Anti-PD-1 single chain antibody coding region (Light Chain—linker —Heavy Chain) (SEQ ID NO: 826); Anti-PD-L1 single chain antibody coding region (Light Chain—linker—Heavy Chain) (SEQ ID NO: 827); Anti-PD-L1 single chain antibody coding region (Heavy Chain—linker—Light Chain) (SEQ ID NO: 828); Anti-CTLA-4 single chain antibody coding region (Heavy Chain—linker—Light Chain) with N terminal and C terminal Secretion Tag for Type V auto-secretor (SEQ ID NO: 829); Anti-CTLA-4 single chain antibody coding region (Light Chain—linker—Heavy Chain) with N terminal and C terminal Secretion Tag for Type V auto-secretor (SEQ ID NO: 830); Anti-PD-1 single chain antibody coding region (Heavy Chain—linker—Light Chain) with N terminal and C terminal Secretion Tag for Type V auto-secretor (SEQ ID NO: 831); Anti-PD-1 single chain antibody coding region (Light Chain—linker—Heavy Chain) with N terminal and C terminal Secretion Tag for Type V auto-secretor (SEQ ID NO: 832); Anti-PD-L1 single chain antibody coding region (Light Chain—linker—Heavy Chain) with N terminal and C terminal Secretion Tag for Type V auto-secretor (SEQ ID NO: 833); Anti-PD-L1 single chain antibody coding region (Heavy Chain—linker—Light Chain) with N terminal and C terminal Secretion Tag for Type V auto-secretor (SEQ ID NO: 834); Anti-CTLA-4 single chain antibody coding region (Heavy Chain—linker—Light Chain) for type I hemolysin secretion, including HlyA tag (SEQ ID NO: 835); Anti-CTLA-4 single chain antibody coding region (Light Chain—linker—Heavy Chain) for type I hemolysin secretion, including HlyA tag (SEQ ID NO: 836); Anti-PD-1 single chain antibody coding region (Heavy Chain—linker—Light Chain) for type I hemolysin secretion, including HlyA tag (SEQ ID NO: 837); Anti-PD-1 single chain antibody coding region (Light Chain—linker—Heavy Chain) for type I hemolysin secretion, including HlyA tag (SEQ ID NO: 838); Anti-PD-L1 single chain antibody coding region (Light Chain—linker—Heavy Chain) for type I hemolysin secretion, including HlyA tag (SEQ ID NO: 839); Anti-PD-L1 single chain antibody coding region (Heavy Chain—linker—Light Chain) for type I hemolysin secretion, including HlyA tag (SEQ ID NO: 840); C terminal HlyA secretion Tag (SEQ ID NO: 841); HlyB coding sequence (SEQ ID NO: 842); and HlyD coding sequence (SEQ ID NO: 843). Selected polypeptide sequences for single chain antibody production and secretion include Anti-CTLA-4 Heavy (SEQ ID NO: 844); Anti-CTLA-4 Light (SEQ ID NO: 845); Anti-PD-1 Heavy (SEQ ID NO: 846); Anti-PD-1 Light (SEQ ID NO: 847); Anti-PD-L1 Light (SEQ ID NO: 848); Anti-PD-L1 Heavy (SEQ ID NO: 849); Linker (SEQ ID NO: 850); N Terminal Secretion Tag For Type V Auto-secretor Secretion (SEQ ID NO: 851); C Terminal Secretion Tag For Type V Auto-secretor Secretion (SEQ ID NO: 852); C-Terminal HlyA Secretion Tag (SEQ ID NO: 853); HlyB (SEQ ID NO: 854); HlyD (SEQ ID NO: 855).

Single-chain antibodies or antibody fragments may be generated using the heavy and light chain variable regions and/or sequences disclosed herein. For example, PCR products corresponding to the heavy and light chain variable regions may be amplified, and spliced together with an intervening flexible peptide linker sequence. Non-limiting examples of polypeptide linkers that may be incorporated between the heavy and light chain variable region sequences include NH₂—

(SEQ ID NO: 1052)
GGGGSGGGGSGGGGS—COOH
and
(SEQ ID NO: 1051)
NH₂-SSADDAKKDAAKKDDAKKDDAKKDAS—COOH
(Griffin et al., 2002).

Modifications to the sequences disclosed herein, such as modifications to the complementarity determining regions and/or framework regions, may be designed in order to improve binding affinity for the target epitope (e.g., to lower_{K}D) and increase suitability for expression of a single-chain antibody from a bacterial cell.

The gene encoding the single-chain anti-CTLA-4 antibody or single-chain anti-PD-1 antibody is expressed under the control of each of the following promoters: a constitutive promoter, a tetracycline-inducible promoter with the tet repressor (TetR) expressed constitutively on a plasmid, or a FNR promoter selected from SEQ ID NOs: 1-12. As discussed herein, other promoters may be used.

The construct encoding the single-chain anti-CTLA-4 antibody or single-chain anti-PD-1 antibody is expressed on a high-copy plasmid, a low-copy plasmid, or a chromosome. For chromosomal expression, the insertion site may be anywhere in the genome, e.g., in a gene required for survival and/or growth (to create an auxotroph); in an active area of the genome, such as near the site of genome replication; and/or in between divergent promoters in order to reduce the risk of unintended transcription.

### Example 25. An Engineered IL-15-Producing E. coli Nissle Strain

Strain Construction and Biochemical Analysis:

To generate an engineered E. coli Nissle strain capable of secreting biologically active interleukin 15 (IL-15) a fusion protein was constructed, in which IL-15 is fused to the minimal region of IL-15Rα required for the formation of a functional receptor, known as the Sushi domain. IL-15 and IL-15Rα form functional complexes, which stimulate cell signaling, and activation and proliferation of neighboring lymphocytes expressing IL-2R13 and yc in a process called trans-presentation (see, e.g., Ochoa et al., Oncoimmunology. 2013; 2(4): e23410). The biological activity of IL-15 is greatly improved by two different modifications: an asparagine to aspartic acid substitution at amino acid 72 of IL-15 (Zhu X, Marcus W D, Xu W, Lee H I, Han K, Egan J O, Yovandich J L, Rhode P R, Wong H C. Novel human interleukin-15 agonists. J Immunol. 2009; 183:3598-3607) and/or direct fusion with the sushi domain of IL-15Rα by mimicking trans-presentation of IL-15 by cell-associated IL-15Rα (Mortier et al., i Biol Chem 2006: 281:1612-9).

To produce the modified recombinant IL-15-Sushi fusion protein, an IL-15 monomer with an N72D mutation was fused to the C-terminus of the sushi domain (78 amino acids) linked by a 20 amino acid linker. To the N-termini of the IL-15R sushi domain, a FLAG-tag and Factor Xa cleavage site was included to facilitate detection, purification and removal of the tag. To promote translocation to the periplasm, the IL15 fusion protein was cloned into a 10-member plasmid library. The coding sequence for the secreted fusion protein was codon optimized for expression in E. coli and ordered from IDT Technologies as a double-stranded DNA fragment. Once the DNA was received it was digested and ligated into the 10-member plasmid library using standard cloning procedures. Each member of the plasmid library contains a low-copy plasmid backbone and a Ptet promoter that drives expression of variable optimized ribosome-binding sites and secretion tags. Once the IL-15 fusion was cloned C-terminally of the secretion tags, the plasmids were transformed into SYN1557 (deltaPAL, diffusible outer membrane (DOM) phenotype) to create the IL-15-Sushi secretion strains SYN3516-SYN3525. Non-limiting examples of construct polypeptide sequences include human IL-15Rα sushi domain (SEQ ID NO: 1209); construct comprising PpiA secretion tag—FLAG-tag—Factor Xa site—Sushi—linker—human IL-15 (SEQ ID NO: 1210); Sushi—linker—human IL-15 (SEQ ID NO: 1211); secretion tag PpiA (SEQ ID NO: 1098); Human IL-15 (SEQ ID NO: 1212); and Linker (SEQ ID NO: 1213). Non-limiting IL-15 construct nucleotide sequences include codon optimized human IL-15Rα sushi domain (SEQ ID NO: 1214); codon optimized construct comprising PpiA secretion tag—FLAG tag—Factor Xa—human IL-15—linker—Sushi (SEQ ID NO: 1215); codon optimized human IL-15—linker—Sushi (SEQ ID NO: 1216); codon optimized secretion tag PpiA (SEQ ID NO: 1108); codon optimized human IL-15 (SEQ ID NO: 1217); codon optimized linker (SEQ ID NO: 1218); and codon optimized FLAG tag and Factor Xa site (SEQ ID NO: 1219). Table 43 provides a description of the IL-15-Sushi strains. Table 42 provides a listing of strains generated using WT IL-15.

TABLE 42
Strain descriptions

| ID | Genotype | Construct |
|---|---|---|
| SYN3424 | PAL (PAL::Cm) | pBR322.Ptet.PpiA |
| | | (ECOLIN_18620)-IL-15 |
| SYN3423 | PAL (PAL::Cm) | pBR322.Ptet.phoA-IL-15 |
| SYN3422 | PAL (PAL::Cm) | pBR322.Ptet.PelB-IL-15 |
| SYN3421 | PAL (PAL::Cm) | pBR322.Ptet.OppA-IL-15 |
| SYN3420 | PAL (PAL::Cm) | pBR322.Ptet.MalE-IL-15 |
| SYN3419 | PAL (PAL::Cm) | pBR322.Ptet.HdeB-IL-15 |
| SYN3418 | PAL (PAL::Cm) | pBR322.Ptet.GspD-IL-15 |
| SYN3417 | PAL (PAL::Cm) | pBR322.Ptet.Gltl- IL-15 |
| SYN3416 | PAL (PAL::Cm) | pBR322.Ptet.DsbA- IL-15 |
| SYN3415 | PAL (PAL::Cm) | pBR322.Ptet.Adhesin- IL-15 |

TABLE 43
Strain descriptions

| ID | Genotype | Construct |
|---|---|---|
| SYN3525 | | pBR322.Ptet PpiA |
| | | (ECOLIN_18620)-IL-15-Sushi |
| SYN3524 | PAL (PAL::Cm) | pBR322.Ptet.phoA-IL-15-Sushi |
| SYN3523 | PAL (PAL::Cm) | pBR322.Ptet.PelB-IL-15-Sushi |
| SYN3522 | PAL (PAL::Cm) | pBR322.Ptet.OppA-IL-15-Sushi |
| SYN3521 | PAL (PAL::Cm) | pBR322.Ptet.MalE-IL-15-Sushi |
| SYN3520 | PAL (PAL::Cm) | pBR322.Ptet.HdeB-IL-15-Sushi |
| SYN3519 | PAL (PAL::Cm) | pBR322.Ptet.GspD-IL-15-Sushi |
| SYN3518 | PAL (PAL::Cm) | pBR322.Ptet.Gltl- IL-15-Sushi |
| SYN3517 | PAL (PAL::Cm) | pBR322.Ptet.DsbA- IL-15-Sushi |
| SYN3516 | PAL (PAL::Cm) | pBR322.Ptet.Adhesin- IL-15-Sushi |

Production of and In Vitro Quantification of IL-15 by SYN3525

To assay for production of IL-15 and/or to quantify bioactivity, cultures were grown and induced, then supernatants were harvested and quantified by ELISA. Prior to assay date, both the negative control strain (SYN1557) and the IL-15-sushi domain producing strains (SYN3516—SYN3525) were struck onto LB agar plates and grown at 37° C. with the appropriate antibiotics. A 2 mL starter culture of 2YT broth was inoculated with a single colony for every 50 mL of induced culture to be grown, allowed to grow at 37° C. for 2 hours, spun down at 8000×g for 10 minutes, and cells were resuspended in 2YT media of the original volume with the anhydrotetracycline (aTc) inducer (100 ng/mL). These cultures were placed at 30° C. with shaking for 4 hours to induce IL-15-sushi fusion protein production. Next, cultures were removed from the incubator and centrifuged at 20,000×g for 10 minutes to pellet all cells and the supernatants were passed through a 0.22 μm filter to yield sterilized supernatants. These supernatants were used in cell-based assays.

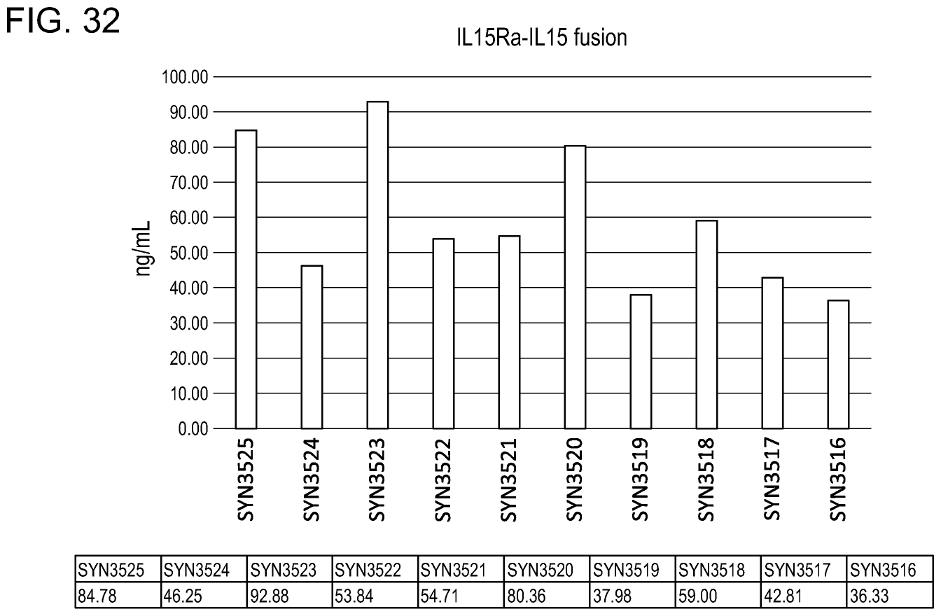

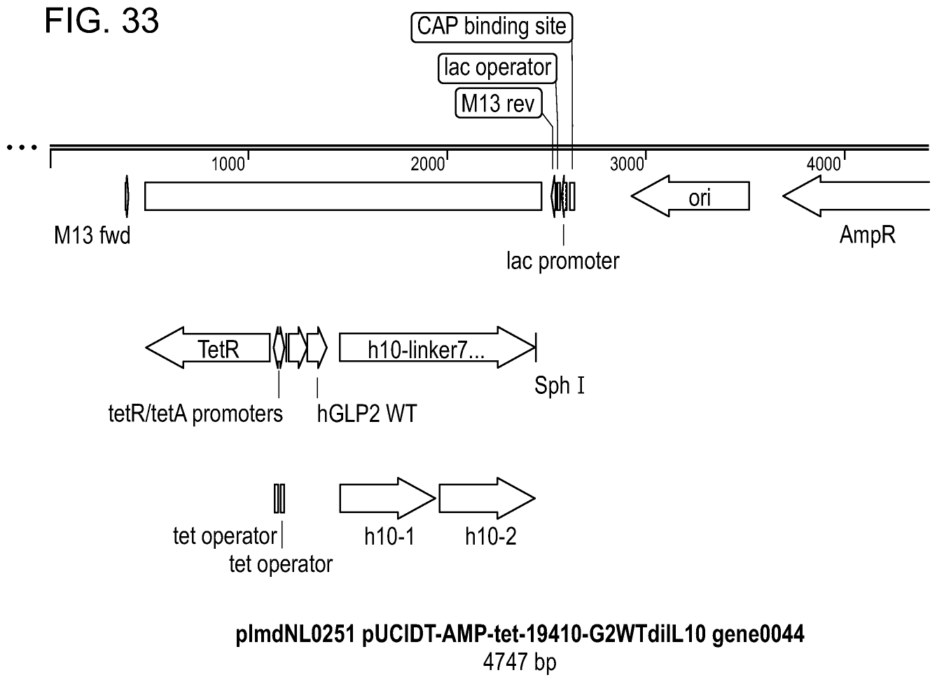

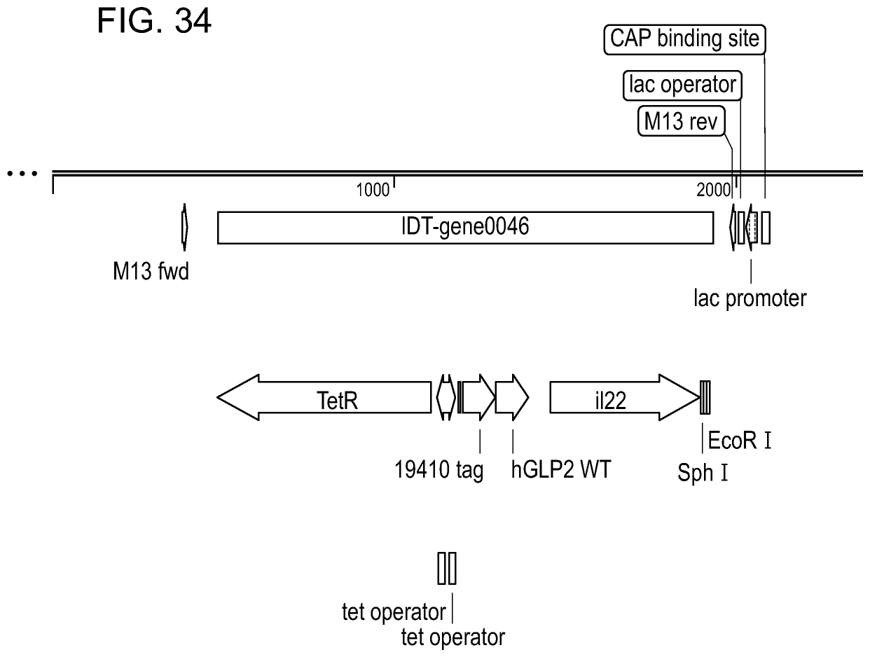

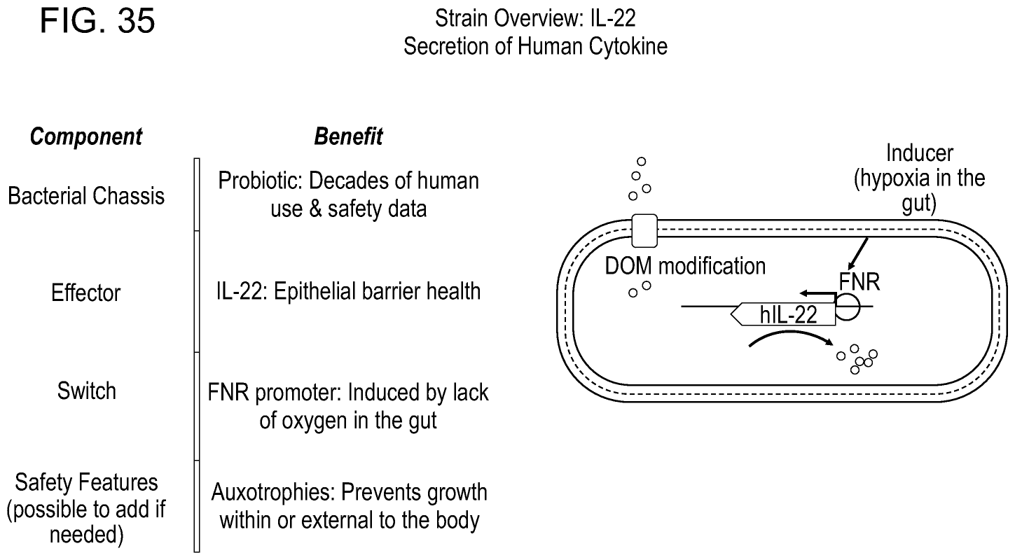

To evaluate the production of IL-15 fusions by the plasmid library, cultures of SYN1557 and the IL-15—producing library were induced and harvested in duplicate. These supernatants were serially-diluted and quantified using an IL-15 ELISA Kit (Human IL-15 Quantikine ELISA Kit—D1500, R&D Systems, Minneapolis, MN). The data from screening our secretion library displayed in FIG. 32 shows maximal production from SYN3525, which contains the PpiA secretion signal from E. coli as the leader peptide. SYN1557 supernatants showed no cross-reactivity in the ELISA (data not shown).

To further evaluate the production of IL-15 from SYN3525, the strain was grown and induced in baffled flasks to generate maximum yield. From the filtered supernatants of SYN3525, samples of SYN1557 and SYN3525 were diluted in triplicate and run on an ELISA Kit (Human IL-15 Quantikine ELISA Kit—D1500, R&D Systems, Minneapolis, MN). The results of these analyses are shown in Table 44. The results showed that under maximal induction conditions the SYN3525 supernatant contained between 733-795 ng/mL of material that reacted positively in the IL-15 ELISA assay. In contrast, the SYN1557 supernatants had undetectable levels (not shown).

TABLE 44
SYN3525 supernatant results from three different ELISA runs.

| SYN3525 | IL-15 (ng/ml) |
|---|---|
| Run 1 | 795.23 |
| Run 2 | 733.75 |
| Run 3 | 792.80 |
| AVERAGE | 773.93 |
| SEM (n = 3) | 20.10 |

### Example 26. GLP-2-IL-22 Fusion Proteins

GLP-2-IL-22 fusion protein constructs were generated according to the methods described above. Non-limiting examples of GLP-2-IL-22 fusion sequences include GLP-2-IL-22 fusion (SEQ ID NO: 1240; SEQ ID NO: 1243); 19410-GLP-2-IL-22 fusion (SEQ ID NO: 1241; SEQ ID NO: 1244); and Tort-GLP-2-IL-22 fusion (SEQ ID NO: 1242; SEQ ID NO: 1245).

### Example 27. GLP-2-diIL-10 Fusion Proteins

GLP-2-diIL-10 fusion protein constructs were generated according to the methods described above. Non-limiting examples of GLP-2-diIL-10 fusion sequences include GLP-2-diIL-10 fusion (SEQ ID NO: 1246; SEQ ID NO: 1249); 19410-GLP-2-diIL-10 fusion (SEQ ID NO: 1247; SEQ ID NO: 1250) and Tort-GLP-2-diIL-10 fusion (SEQ ID NO:1248; SEQ ID NO: 1251).

### Example 28. Construction of a Dimerized IL-12 for Secretion

Strain Engineering:

To generate an engineered E. coli Nissle strain capable of secreting biologically active Interleukin 12 (IL-12) heterodimer, a construct was generated in which two interleukin-12 monomer subunits (IL-12A (p35) and IL-12B (p40)) were covalently linked by a linker.

To facilitate the dimerization of recombinant human IL-12 protein produced from E. coli Nissle, a 15 amino acid linker of ‘GGGGSGGGGSGGGGS’(SEQ ID NO: 1063) was inserted between two monomer subunits (IL-12A (p35) and IL-12B (p40) to produce a forced dimer human IL-12 (diIL-12) fusion protein, and the sequence was codon-optimized.

To promote translocation to the periplasm, secretion tags were added to the N-terminus of the diIL-12 fusion protein. The DNA sequence containing the elements of the inducible Ptet promoter, ribosome binding site, diIL-12 coding sequence and other necessary linkers were synthesized by IDT Technologies and subsequently cloned into a high copy number plasmid vector. The plasmid was transformed into SYN1555, SYN1556, SYN1557 or SYN1625 (comprising the nlpl, tolA, PAL, or lpp deletion respectively to create the diffusible membrane phenotype) to create the dimerized human IL-12 (diIL-12) secretion strains. Non-limiting IL-12 construct polynucleotide sequences include construct comprising secretion tag 19410—human IL-12 (p35)—Linker (15aa)— human IL-12 (p40) (SEQ ID NO: 1252); construct comprising secretion tag dsba—human IL-12 (p35)—Linker (15aa)—human IL-12 (p40) (SEQ ID NO: 1253); construct comprising secretion tag phoA—human IL-12 (p35)—Linker (15aa)—human IL-12 (p40) (SEQ ID NO: 1254); construct comprising secretion tag tolB—human IL-12 (p35)—Linker (15aa)—human IL-12 (p40) (SEQ ID NO: 1255); construct comprising secretion tag malE—human IL-12 (p35)—Linker (15aa)—human IL-12 (p40) (SEQ ID NO: 1256); construct comprising secretion tag mglB—human IL-12 (p35)—Linker (15aa)—human IL-12 (p40) (SEQ ID NO: 1257); construct comprising secretion tag ompF—human IL-12 (p35)—Linker (15aa)—human IL-12 (p40) (SEQ ID NO: 1258); construct comprising secretion tag ompA—human IL-12 (p35)—Linker (15aa)—human IL-12 (p40) (SEQ ID NO: 1259); construct comprising secretion tag tort—human IL-12 (p35)—Linker (15aa)—human IL-12 (p40) (SEQ ID NO: 1260); construct comprising secretion tag lamB—human IL-12 (p35)—Linker (15aa)—human IL-12 (p40) (SEQ ID NO: 1261); construct comprising secretion tag pelB—human IL-12 (p35)—Linker (15aa)—human IL-12 (p40) (SEQ ID NO: 1262); 19410 Secretion Tag (SEQ ID NO: 1117); dsba Secretion Tag (SEQ ID NO: 1118); phoA Secretion Tag(SEQ ID NO: 1119); tolB Secretion Tag(SEQ ID NO: 1120); malE Secretion Tag(SEQ ID NO: 1121); mglB Secretion Tag(SEQ ID NO: 1122); ompF Secretion Tag(SEQ ID NO: 1123); ompA Secretion Tag(SEQ ID NO: 1115); tort Secretion Tag(SEQ ID NO: 1124); lamB Secretion Tag(SEQ ID NO: 1125); pelB Secretion Tag(SEQ ID NO: 1126); and human IL-12 (p35)—Linker (15aa)—human IL-12 (p40) (SEQ ID NO: 1263).

Non-limiting IL-12 construct polypeptide sequences include construct comprising secretion tag 19410—human IL-12 (p35)—Linker (15aa)—human IL-12 (p40) (includes C terminal V5 and HIS tag and FLAG tag located at C terminus of secretion tag) (SEQ ID NO: 1264); construct comprising secretion tag dsba—human IL-12 (p35)—Linker (15aa)—human IL-12 (p40) (includes C terminal V5 and HIS tag and FLAG tag located at C terminus of secretion tag) (SEQ ID NO: 1265); construct comprising secretion tag phoA—human IL-12 (p35)—Linker (15aa)—human IL-12 (p40) (includes C terminal V5 and HIS tag and FLAG tag located at C terminus of secretion tag) (SEQ ID NO: 1266); construct comprising secretion tag tolB—human IL-12 (p35)—Linker (15aa)—human IL-12 (p40) (includes C terminal V5 and HIS tag and FLAG tag located at C terminus of secretion tag) (SEQ ID NO: 1267); construct comprising secretion tag malE—human IL-12 (p35)—Linker (15aa)—human IL-12 (p40) (includes C terminal V5 and HIS tag and FLAG tag located at C terminus of secretion tag) (SEQ ID NO: 1268); construct comprising secretion tag mg1B—human IL-12 (p35)—Linker (15aa)—human IL-12 (p40) (includes C terminal V5 and HIS tag and FLAG tag located at C terminus of secretion tag) (SEQ ID NO: 1269); construct comprising secretion tag ompF— human IL-12 (p35)—Linker (15aa)—human IL-12 (p40)(includes C terminal V5 and HIS tag and FLAG tag located at C terminus of secretion tag) (SEQ ID NO: 1270); construct comprising secretion tag ompA—human IL-12 (p35)—Linker (15aa)—human IL-12 (p40)(includes C terminal V5 and HIS tag and FLAG tag located at C terminus of secretion tag) (SEQ ID NO: 1271); construct comprising secretion tag tort—human IL-12 (p35)—Linker (15aa)—human IL-12 (p40)(includes C terminal V5 and HIS tag and FLAG tag located at C terminus of secretion tag) (SEQ ID NO: 1272); construct comprising secretion tag lamB—human IL-12 (p35)—Linker (15aa)—human IL-12 (p40)(includes C terminal V5 and HIS tag and FLAG tag located at C terminus of secretion tag) (SEQ ID NO: 1273); construct comprising secretion tag pelB—human IL-12 (p35)—Linker (15aa)—human IL-12 (p40)(includes C terminal V5 and HIS tag and FLAG tag located at C terminus of secretion tag) (SEQ ID NO: 12074); 19410 Secretion Tag (SEQ ID NO: 1090); dsba Secretion Tag (SEQ ID NO: 1092); phoA Secretion Tag (SEQ ID NO: 1097); tolB Secretion Tag (SEQ ID NO: 1109); malE Secretion Tag (SEQ ID NO: 1094); mglB Secretion Tag (SEQ ID NO: 1127); ompF Secretion Tag (SEQ ID NO: 1128); ompA Secretion Tag (SEQ ID NO: 1111); tort Secretion Tag (SEQ ID NO: 1110); lamB Secretion Tag (SEQ ID NO: 1129); pelB Secretion Tag (SEQ ID NO: 1096) and human IL-12 (p35)— Linker (15aa)—human IL-12 (p40) (SEQ ID NO: 1275).

Production of dilL-12 by SYN3466 to SYN3505 for In vitro Assays

To assay for production of IL-12 and/or to quantify bioactivity, cultures were grown and induced, then supernatants were harvested and quantified via ELISA, as follows using shaker plate. Both the negative control strain (SYN1555, SYN1556, SYN1557 or SYN1625) and the diIL-12-producing strain (SYN3466 to SYN3505) were struck onto LB agar plates and grown at 37° C. with chloramphenicol, or chloramphenicol and carbenicillin, respectively. After overnight growth, an individual colony was picked and used to inoculate a 4 mL starter culture of 2YT broth for future shaker plate to be grown. The starter cultures were inoculated with the same antibiotics used in the agar plates. When the starter culture reached saturation, the culture was back-diluted 1:25 into a new shaker plate of 2YT with appropriate antibiotics. This starter culture was allowed to grow at 37° C. for 2 hours to an OD600=˜0.8-1. The culture was then induced by adding aTc inducer (100 microg/mL). These induced cultures were incubated at 30° C. with shaking for 4 hours to promote IL-12 production.

When the production phase was complete, the shaker plate of cultures was removed from the incubator and centrifuged at 20000×g for 10 minutes to pellet all cells. The supernatants were kept for ELISA analysis.

Quantification of dilL-12 in Strain supernatants by ELISA

To evaluate the amounts of dilL-12 in the supernatants, samples were assayed using a Human IL-12 p70 Quantikine ELISA Kit from R&D systems. The results of these analyses are shown in Table 45. The results showed that the supernatants contained between 17 and 309 pg/mL of material that reacted positively in the IL-12 ELISA assay. In contrast, the SYN1557 supernatants had undetectable levels.

TABLE 45
Strain supernatant results from ELISA analysis (pg/mL).

| Signal Peptides | | | | | | |
|---|---|---|---|---|---|---|
| Host strain | Vector | 19410 | pelB | tort | LamB | OmpF |
| Nissle delta nlpI::CmR | 15.1 | 130.7 | 61.4 | 255.6 | 81.6 | 17.0 |
| Nissle delta tolA::CmR | 12.8 | 47.7 | 93.0 | 205.3 | 189.8 | 220.2 |
| Nissle delta PAL::CmR | 10.2 | 142.3 | 31.2 | 266.7 | 167.0 | 62.8 |
| Nissle delta lpp::CmR | 10.5 | 233.7 | 57.9 | 278.6 | 107.2 | 128.4 |

| Signal Peptides | | | | | | |
|---|---|---|---|---|---|---|
| Host strain | OmpF | mglB | malE | tolB | phoA | dsbA |
| Nissle delta nlpI::CmR | 17.0 | 194.4 | 224.7 | 234.2 | 241.9 | 99.3 |
| Nissle delta tolA::CmR | 220.2 | 227.0 | 194.4 | 187.0 | 309.1 | 146.0 |
| Nissle delta PAL::CmR | 62.8 | 218.4 | 256.5 | 250.0 | 203.7 | 237.0 |
| Nissle delta lpp::CmR | 128.4 | 203.3 | 242.3 | 197.2 | 250.7 | 101.9 |

### Example 29. An engineered IL-15-producing E. coli Nissle strain

Strain Engineering

To generate an engineered E. coli Nissle strain capable of secreting biologically active interleukin 15 (IL-15) a fusion protein was constructed, in which IL-15 is fused to the minimal region of IL-15Rα required for the formation of a functional receptor, which is known as Sushi domain IL-15 and IL-15Rα form functional complexes, which stimulate cell signaling, and activation and proliferation of neighboring lymphocytes expressing IL-2R13 and yc in a process called trans-presentation (see, e.g., Ochoa et al., High-density lipoproteins delivering interleukin-15; Oncoimmunology. 2013 Apr. 1; 2(4): e23410). The biological activity of IL-15 is greatly improved by direct fusion with the sushi domain of IL-15Rα by mimicing trans-presentation of IL-15 by cell-associated IL-15Rα (Mortier et al., Soluble interleukin-15 receptor alpha (IL-15R alpha)-sushi as a selective and potent agonist of IL-15 action through IL-15R betagamma. Hyperagonist IL-15×IL ISR alpha fusion proteins, I Diol Chem 2006; 281:1612-9).

To produce the recombinant IL-15-Sushi fusion protein, IL-15 monomer was fused to the C-terminus of the sushi domain linked by a 20 amino acid linker. To promote translocation to the periplasm, 19410, tort, or pelB secretion tag was added to the N-terminus of IL-15-Sushi fusion protein. The DNA sequence containing the Ptet promoter, RBS, coding sequence for IL-15-Sushi and other necessary linkers were synthesized by IDT Technologies and subsequently cloned into a high copy number plasmid vector provided by IDT Technologies under the control of a tet-inducible promoter. The plasmids were transformed into SYN1557 (delta PAL, diffusible outer membrane (DOM) phenotype) or SYN94 (no PAL deletion E. coli Nissle strain) to create the IL-15-Sushi secretion strains SYN3458 to SYN3463.

Non-limiting IL-15 construct nucleotide and polypeptide sequences include human IL-15Rα sushi domain (SEQ ID NOS: 1209 and 1214); construct comprising 19410 secretion tag— Sushi—linker—human IL-15 (SEQ ID NOS: 1282 and 1276); construct comprising tort secretion tag—Sushi—linker—human IL-15 (SEQ ID NOS: 1283 and 1277); construct comprising pelB secretion tag—Sushi—linker—human IL-15 (SEQ ID NOS: 1284 and 1278); Sushi—linker—human IL-15 (SEQ ID NOS: 1285 and 1279); human IL-15 (SEQ ID NOS: 1134 and 1217); linker (SEQ ID NO: 1280). Production of IL-15 by SYN3458 to SYN3463 for in vitro Assays

To assay for production of IL-15 and/or to quantify bioactivity, cultures were grown and induced, then supernatants were harvested and quantified by ELISA. Prior to assay date, both the negative control strain (SYN1557) and the IL-15-sushi domain producing strain SYN3458 to SYN3463 were struck onto LB agar plates and grown at 37° C. with the appropriate antibiotics. A 2 mL starter culture of 2YT broth was inoculated with a single colony for every 50 mL of induced culture to be grown, allowed to grow at 37° C. for 2 hours, spun down at 8000×g for 10 minutes, and cells were resuspended in 2YT media of the original volume with the anhydrotetracycline (aTc) inducer (100 ug/mL). These cultures were placed at 30° C. with shaking for 4 hours to induce IL-15-sushi fusion protein production. Next, cultures were removed from the incubator and centrifuged at 20,000×g for 10 minutes to pellet all cells and the supernatants were passed through a 0.22 μm filter to yield sterilized supernatants. These supernatants were used in cell-based assays.

Quantification of IL-15 in SYN3458 to SYN3463 Supernatants by ELISA

To evaluate the production of IL-15 in the filtered supernatants, samples of SYN1557 and SYN3458 to SYN3463 were diluted in triplicate and run on an Human IL-15 Quantikine ELISA Kit (Ra&D Systems). The results of these analyses are shown in Table 46. The results showed that the SYN3458 to SYN3463 supernatant contained between 4 and 275 ng/mL of material that reacted positively in the IL-15 ELISA assay. In contrast, the SYN94 and SYN1557 supernatants had undetectable levels (not shown).

TABLE 46
Supernatant results from three different ELISA runs.

| final | host | | |
|---|---|---|---|
| strain | strain | plasmid | ng/ml |
| SYN3460 | SYN1557 | ptet-pelBss-hIL15-SUSHI-fusion | 275 |
| SYN3461 | SYN1557 | ptet-19410ss-hIL15-SUSHI-fusion | 166 |
| SYN3458 | SYN1557 | ptet-tortss-hIL15-SUSHI-fusion | 59 |
| SYN3459 | SYN94 | ptet-tortss-hIL15-SUSHI-fusion | 78 |
| SYN3462 | SYN94 | ptet-pelBss-hIL15-SUSHI-fusion | 4 |
| SYN3463 | SYN94 | ptet-19410ss-hIL15-SUSHI-fusion | 72 |

### Example 30. CXCL10 Secretion

To produce the recombinant CXCL10 chemokine, a synthetic construct was devised. The secreted/soluble form of the CXCL10 protein was codon optimized for expression in E. coli and ordered from IDT Technologies as a double-stranded DNA fragment. To promote translocation to the periplasm, the CXCL10 soluble protein was cloned into a 10-member plasmid library. Once the DNA was received it was digested and ligated into the 10-member plasmid library using standard cloning procedures. Each member of the plasmid library contains a low-copy plasmid backbone and a Ptet promoter that drives expression of variable optimized ribosome-binding sites and secretion tags. Once the CXCL10 was cloned C-terminally of the secretion tags, the plasmids were transformed into SYN1557 (OPAL, diffusible outer membrane (DOM) phenotype) to create the CXCL10 secretion strains, SYN3404 and SYN3406-SYN3414. Non-limiting examples of construct sequences include CXCL10 (SEQ ID NO: 1291 and 1138) and CXCL10 plus Tag of interest (SEQ ID NO: 1286 and 1150)

TABLE 47
Strain Descriptions

| ID | Genotype | Construct |
|---|---|---|
| SYN3414 | PAL (PAL::Cm) | p15a.Ptet.PpiA-CXCL10 |
| SYN3413 | PAL (PAL::Cm) | p15a.Ptet.phoA-CXCL10 |
| SYN3412 | PAL (PAL::Cm) | p15a.Ptet.PelB-CXCL10 |
| SYN3411 | PAL (PAL::Cm) | p15a.Ptet.OppA-CXCL10 |
| SYN3410 | PAL (PAL::Cm) | p15a.Ptet.MalE-CXCL10 |
| SYN3409 | PAL (PAL::Cm) | p15a.Ptet.HdeB-CXCL10 |
| SYN3408 | PAL (PAL::Cm) | p15a.Ptet.GspD-CXCL10 |
| SYN3407 | PAL (PAL::Cm) | p15a.Ptet.Gltl-CXCL10 |
| SYN3406 | PAL (PAL::Cm) | p15a.Ptet.DsbA-CXCL10 |
| SYN3404 | PAL (PAL::Cm) | p15a.Ptet.Adhesin-CXCL10 |

To assay for production of CXCL10 and/or to quantify bioactivity, cultures were grown and induced, then supernatants were harvested and quantified by ELISA. Prior to assay date, both the negative control strain (SYN1557) and the CXCL10-producing strains (SYN3404—SYN3414) were struck onto LB agar plates and grown at 37° C. with the appropriate antibiotics. A 2 mL starter culture of 2YT broth was inoculated with a single colony for every 50 mL of induced culture to be grown, allowed to grow at 37° C. for 2 hours, spun down at 8000×g for 10 minutes, and cells were resuspended in 2YT media of the original volume with the anhydrotetracycline (aTc) inducer (100 ng/mL). These cultures were placed at 30° C. with shaking for 4 hours to induce CXCL10 protein production. Next, cultures were removed from the incubator and centrifuged at 20,000×g for 10 minutes to pellet all cells and the supernatants were passed through a 0.22 μm filter to yield sterilized supernatants. These supernatants were used in cell-based assays.

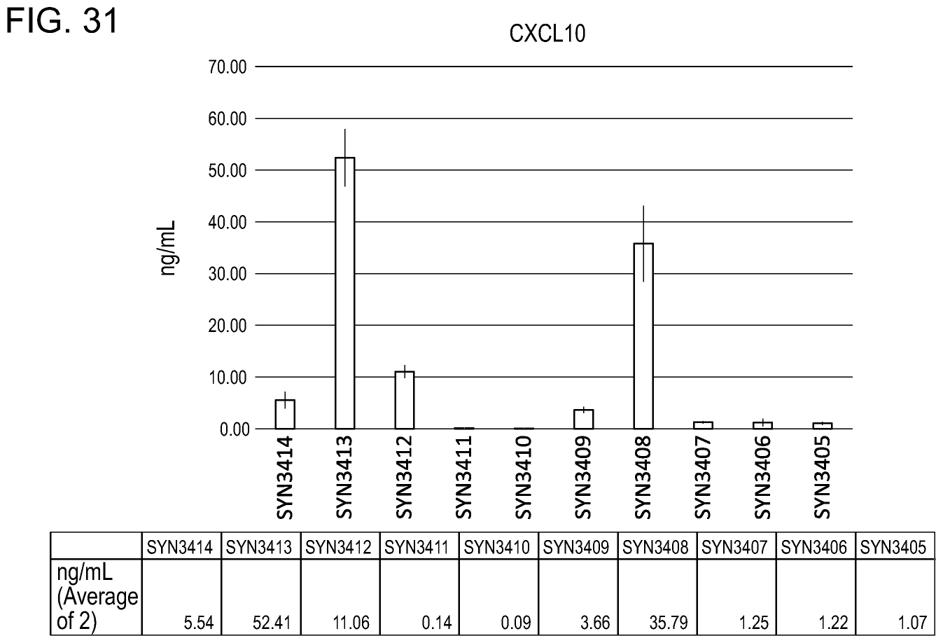

To evaluate the production of CXCL10 by the plasmid library, cultures of SYN1557 and the CXCL10—producing library were induced and harvested in duplicate. These supernatants were serially-diluted and quantified using a CXCL10 ELISA Kit (Human CXCL10/IP-10 Quantikine ELISA Kit—DIP100, R&D Systems, Minneapolis, MN). The data from screening our secretion library displayed in FIG. 31 shows maximal production from SYN3413, which contains the PhoA secretion signal from E. coli as the leader peptide. SYN1557 supernatants showed no cross-reactivity in the ELISA (data not shown).

To further evaluate the production of CXCL10 from SYN3413, the strain was grown and induced in baffled flasks to generate maximum yield. From the filtered supernatants of SYN3413, samples of SYN1557 and SYN3413 were diluted in triplicate and run on an ELISA Kit (Human CXCL10/IP-10 Quantikine ELISA Kit—DIP100, R&D Systems, Minneapolis, MN). The results of these analyses are shown in Table 48. The results showed that under maximal induction conditions the SYN3413 supernatant contained between 199-232 ng/mL of material that reacted positively in the CXCL10 ELISA assay. In contrast, the SYN1557 supernatants had undetectable levels (not shown).

TABLE 48
Concentration of Secreted hCXCL10 from triplicate SYN3414

| SYN3414 | CXCL10 (ng/ml) |
|---|---|
| Run 1 | 199.71 |
| Run 2 | 231.96 |
| Run 3 | 232.16 |
| AVERAGE | 221.28 |
| SEM (n = 3) | 10.78 |

Functional Assay for CXCL10

To determine whether CXCL10 secreted from any of the strains described above is functional, a Chemotaxis Assay is performed, essentially as described in Mikucki et al. (Mikucki, et al., Nat Commun. 2015; 6: 7458, the contents of which is herein incorporated by reference in its entirety).

Briefly, cell trace labeled naïve (or expanded) human CD8+ T cells are transferred into 24-well transwell plate (5 uM pore) with T cells on top (5×105 cells) and rCXCL10/supernatants on bottom, in the presence or absence of PTX or anti-CXCL10. Cells are incubated for 3 hrs, and migrated cells are counted by flow cytometry. Alternatively, phosphoAKT is measured by flow, western or ELISA.

### Example 31. IL-22 Secretion

hIL-22 Production and Quantification In Vitro

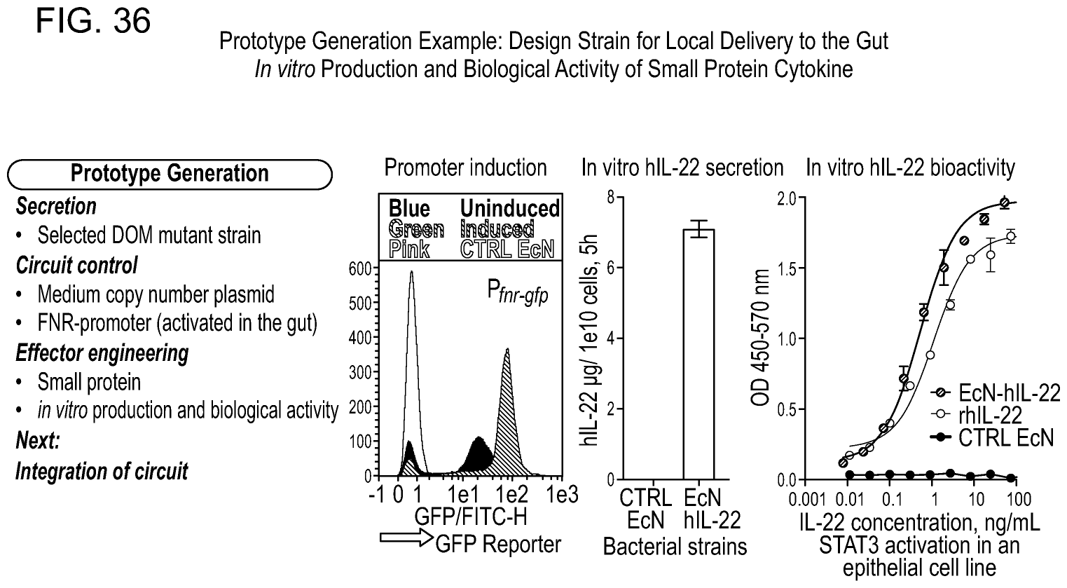

To assay for production of hIL-22, bacteria were inoculated into fresh flasks of 20 ml 2YT medium at OD600=0.25. These flasks were placed at 37° C. and grown for 5 hours shaking. The cultures were harvested by centrifugation at 12.5K×g for 5 minutes. The supernatants of the cultures were removed from the cell pellet and filtered through a 0.22 μm filter to separate any remaining bacteria from the supernatant. To evaluate the production of IL-22 in the filtered supernatants, samples were diluted in triplicate and analyzed by Human IL-22 Quantikine® ELISA Kit (#D2200, Fisher Scientific) according the manufacturer's instructions (FIG. 36).

hIL-22 STAT3 Activation In Vitro

IL-22-induced phosphorylation of STAT3 was determined using the PathScan Phospho-STAT3 (Tyr705) Sandwich ELISA Kit (#7300, Cell Signalling Technology). Briefly, Colo205 cells (#CCL-222, ATCC) were counted and serum starved for 4 hrs with RPMI1640 in ultra-low binding T75 flasks (#3814, Corning). Cells were collected, spun at 1500 rpm, and resuspended at 1.25×10′ cells/mL. 50 uL of cells were then added to a prepared 96-well v-bottom plate (final concentration of 6.25×10 5 cells/well) containing 50 uL of 2× of recombinant IL-22 (rIL22, #782-IL-010, R&D,) or bacterial supernatants (starting with undiluted supernatant) titrated down in 3-fold dilutions. Cells were then incubated for 15 minutes at 37° C. to allow for induction of STAT3 phosphorylation. After 15 minutes, the plate was placed on ice and 175 uL of 1.5 mM sodium vanadate was added to all wells. Plates were then spun at 1500 rpm at 4° C. for 5 minutes. 165 uL of 1× lysis buffer was added to each well, and the plate was incubated on ice and then spun for 10 minutes at 400 rpm to clarify the lysates. Lysates from the different treatment groups were then added to phospho-STAT3 ELISA plates (#7300, Cell Signalling Technology) and ELISAs were performed as per the manufacturer's instructions (FIG. 36).

Characterization of hIL-22-Secreting Strain In Vivo in DSS-Treated Mice

For quantitation of hIL-22 production and Reg3β and Birc5 mRNA expression by SYN4878 (SYN001 strain with PAL-KO-cmR and p15A-Pfnr-phoA-IL22-KanR), naïve C57BL6 mice were randomized into the different treatment groups (10 mice/group) by weight and put on 3% DSS drinking water for 7 days. After day 7, DSS water was removed and substituted with normal water. At day 8, mice were gavaged with 200 uL either of ˜1.25×10¹⁰ CFUs of SYN1557 or hIL-22 secreting stain SYN4878. Six hours after dose, mice were manually restrained and fresh fecal pellets were collected from the mouse rectal area using forceps and placed into individual pre-weighed bead-filled tubes (beadBug, #Z763829, Sigma). Mice were then euthanized by CO₂ asphyxiation and colon tissue were collected for analysis into individual pre-weighed bead-filled tubes. Feces and colon samples were processed and analyzed as described below.

Characterization of hIL-22-Secreting Strain In Vivo in DSS-Treated Mice after 24 h Colonization

For quantitation of hIL-22 production and Reg3β and Birc5 mRNA expression by SYN4878 with streptomycin resistance (SYN6967), naïve C57BL6 mice were randomized into the different treatment groups (10 mice/group) by weight and put on 3% DSS drinking water for 7 days. After day 7, DSS water was removed and substituted with Streptomycin water (5 g/L). At day 8, mice were gavaged with 200 uL either of 2×10⁹ CFUs of SYN5103 (control) or hIL-22 secreting stain SYN6967. Twenty-four hours after dose, mice were manually restrained and fresh fecal pellets were collected from the mouse rectal area using forceps and placed into individual pre-weighed bead-filled tubes (beadBug, #Z763829, Sigma). Mice were then euthanized by CO₂ asphyxiation and colon tissue were collected for analysis into individual pre-weighed bead-filled tubes or in 500 uL of RNALater stabilization solution (#AM7021, Fisher Scientific). Feces, colon tissue and RNA samples were processed and analyzed as described below.

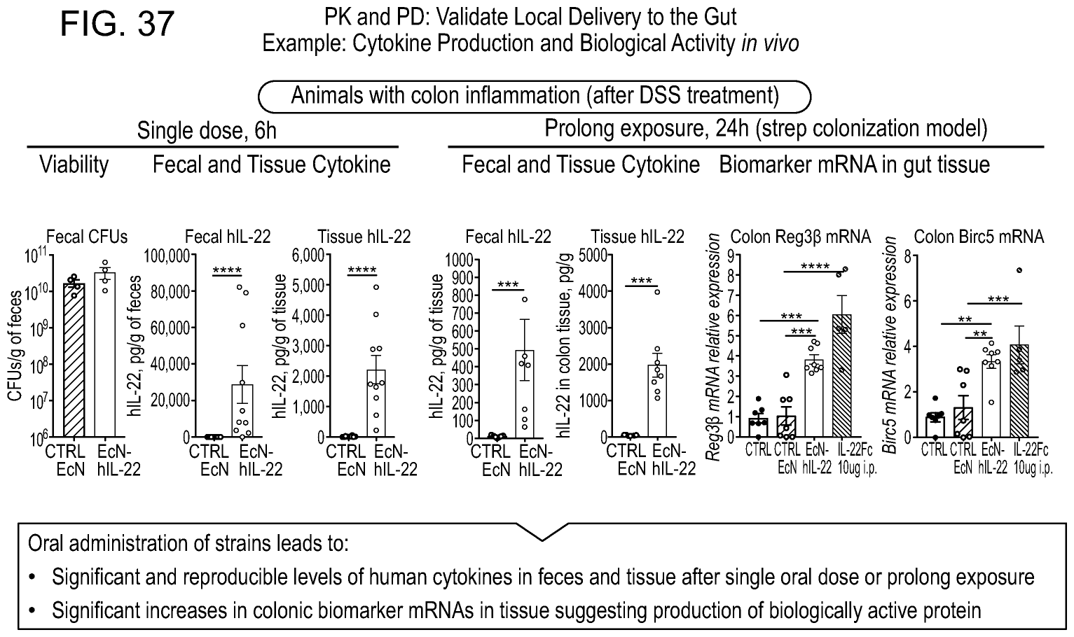

The data demonstrated that oral administration of strains leads to significant and reproducible levels of human cytokines in feces and tissue after single oral dose or prolong exposure (FIG. 37). Significant increases in colonic biomarker mRNAs in tissue further suggest a production of biologically active protein.

Kinetics of SYN4878 hIL-22 Production in DSS-Pretreated Mice

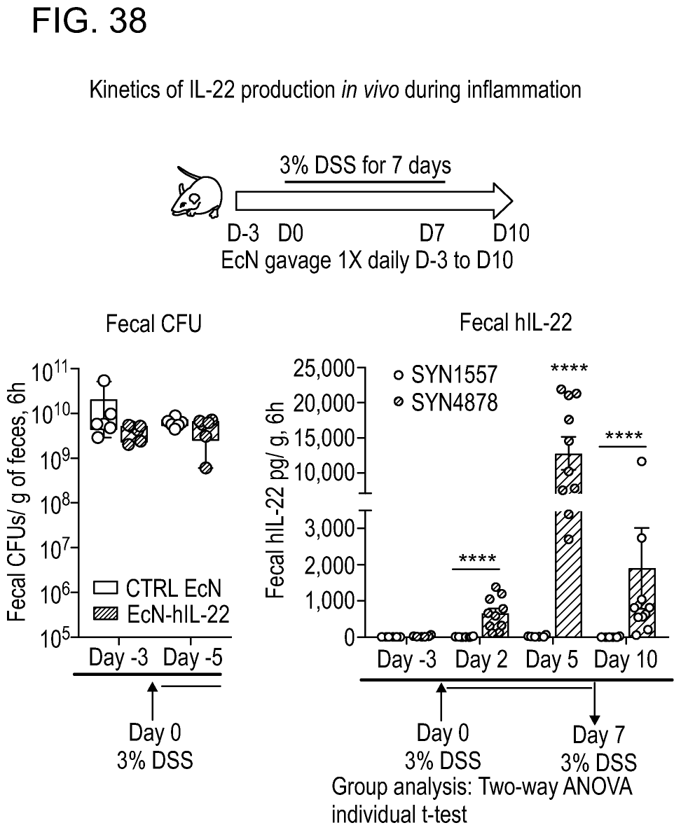

For kinetics of hIL-22 production by SYN4878, naïve C57BL6 mice were randomized into the different treatment groups (10 mice/group) by weight and put on 3% DSS drinking water for 7 days starting from day 0. After day 7, DSS water was removed and substituted with normal water. At day −3, 2, 5 and 10, mice were gavaged with 200 uL either of ˜1.25×10¹⁰ CFUs of SYN1557 or hIL-22 secreting stain SYN4878. Six hours after dose, mice were manually restrained and fresh fecal pellets were collected from the mouse rectal area using forceps and placed into individual pre-weighed bead-filled tubes (beadBug, #Z763829, Sigma). Feces were processed and analyzed as described below. As shown in FIG. 38, a significant level of IL-22 protein was detected in fecal samples starting on day 2, and the level continued to rise on day 5.

Bacterial Enumeration by CFU Counting

To measure CFUs, homogenized samples were plated in a 10×series of 8 dilutions, e.g. the first sample dilution was made as 20 ul of homogenate into 180 ul of sterile PBS, then 10 uL of sample further diluted into 90 uL of sterile PBS, and this was repeated 7 times in a 96 well plate. Then, 10 uL of each dilution was pipetted onto appropriate LB plate with following antibiotics for the strains: SYN1557—Chloramphenicol 30 ug/ml, SYN4878: Kanamycin A 50 ug/ml. The plate was tilted to allow the sample dilutions to streak down the plate. Plates were allowed to dry and then incubated overnight at 37° C. The following day colonies were manually counted from a streak containing >30 individual colonies. The multiplying factor for the first streak of 1:10 diluted homogenate sample was considered as 1×10³ CFU/ml and 10-fold increases were added for each dilution after that. With an assumption that the tube content density ρ=1 g/ml, total number of live bacteria (CFUs) per feces was calculated as N=plate CFU/ml×[0.5 (PBS volume)+(dry weight of the tube content)].

Animal Sample Collection and Processing

For all studies, at the study-specific specified time points defined for each study (see above) animal samples were collected and processed as follows:

For fecal CFU harvest, prior to euthanization mice were manually restrained and fresh fecal pellets were collected from the rectal area using forceps, placed into individual pre-weighed bead-filled tubes (beadBug, #Z763829, Sigma) containing 0.5 mL of cold, sterile PBS. Samples were stored on ice until post-collection tube weight was noted. Then tube content was homogenized with the FastPrep 24 benchtop homogenizer at the speed of 4 m/s for 40 seconds and directly used for enumeration of CFUs.

For fecal or colon tissue preparation for hIL-22 ELISA, feces or colon tissue were harvested and placed into individual pre-weighed bead-filled tubes (beadBug, #Z763829, Sigma) containing 0.5 mL of cold, sterile PBS+15 ul of Halt Protease Inhibitor Cocktail (100×) with 5 uL of EDTA (100×) (#78429, Thermo Fisher Scientific)+0.1% Tween-20 were added to each tube. Tube content was homogenized with the FastPrep 24 benchtop homogenizer at the speed of 4 m/s for 20 seconds The tube content was spun down (15000 rpm, 20 min at 4° C.) and supernatants were aliquoted in 96 well plate for the hIL-22 ELISA analysis.

For colon tissue collection for RNA isolation, colon tissue was harvested, organ content was gently squeezed out and fat tissue was carefully removed. 2 cm of tissue were collected for analysis in 500 uL of RNALater stabilization solution (#AM7021, Fisher Scientific). Tissue was stored in RNALater at 4° C. for the short period of time until RNA was isolated and analyzed as described below.

hIL-22 ELISA Analysis of Fecal Samples

For ELISA analysis of hIL-22 in fecal samples, processed fecal supernatants were loaded onto the hIL-22 ELISA assay plate (Human IL-22 Quantikine® ELISA Kit, #D2200, Fisher Scientific). ELISA assays were run according to the manufacturer's instructions.

RNA Isolation and cDNA Preparation

For RNA isolation with RNeasy Mini Kit (#74106, Qiagen), tissue samples were removed from RNALater and placed into individual bead-filled tubes (beadBug, #Z763829, Sigma) containing 0.6 mL of buffer RLT with 1% of β-mercaptoethanol. Tissues were homogenized twice with FastPrep 24 benchtop homogenizer at the speed of 4.5 m/s for 30 seconds. Tissue lysate was centrifuged for 3 min at full speed and supernatant was used for further RNA purification according to the manufacturer's protocol.

For high throughput RNA isolation from kinetic studies, mRNA was isolated with TurboCapture 96 mRNA Kit (#72251, Qiagen). Tissue samples were removed from RNALater and placed into individual bead-filled tubes (beadBug, #Z763829, Sigma) containing 200 to 300 uL of TCL buffer with 1% of β-mercaptoethanol. Tissue was homogenized twice with a FastPrep 24 benchtop homogenizer at the speed of 4.5 m/s for 30 seconds. Tissue lysate was spun down (5 min, 4° C., 15000 rpm) and 80 ul of the lysate was transferred onto mRNA isolation plate. Plate was used for further RNA purification according to the manufacturer's protocol.

For cDNA preparation, cDNA was synthesized from isolated RNA using High-Capacity cDNA Reverse Transcription Kit with RNase Inhibitor (#4374966, Thermo Fisher Scientific) according to manufacturer's protocol. For 20 ul reaction, 2×RT master mix was prepared as follows: 10×RT Buffer 2 uL, 25×dNTP mix 0.8 uL, 10×Random primers 2 uL, MultiScribe RT (50U/ul) 1 uL, RNAase inhibitor 1 uL, Nuclease-free H₂O 3.2 uL, and mRNA 10 uL. PCR plate was run 10 min at 25° C. and 120 min at 37° C. in T100 Thermal Cycler (Bio-Rad).

qPCR Analysis of Gene Expression

For the quantification of Reg3β (Mm00440616_g1 Taqman probe) and Birc5 (Mm00599749_g1 Taqman probe) mRNA expression, a 20 uL qPCR reaction was performed using Taqman assay probes and TaqMan® Fast Advanced Master Mix (#4444557, Thermo Fisher Scientific) and containing the following components: 2× Master Mix 10 uL, Taqman gene probe (20×) 1 uL (FAM-labeled, #4331182, Thermo Fisher Scientific), Taqman mouse β-actin (endogenous control)* probe (20×) 1 uL (VIC-labeled, #4352341E, Thermo Fisher Scientific), nuclease-free H₂O 4 uL, cDNA 4 uL. Reaction was run in MicroAmp™ Fast Optical 96-Well Reaction Plate (#4346907, Thermo Fisher Scientific) 2 min at 50C, 20 sec at 95C and 40 cycles (1 sec—95 C, 20 sec—60 C) in StepOnePlus Real-Time PCR System, Applied Biosystems. Gene expression was analyzed by using comparative C_{T} Method (ΔΔ_{C}T Method).

Statistical Analysis

Prism7.0c software (GraphPad Software, San Diego, CA) was used for all statistical analysis. In vivo kinetics was analyzed using One- or Two-way ANOVA followed-up by Tukey's post-analysis.

### Example 32. IL-2 Secretion

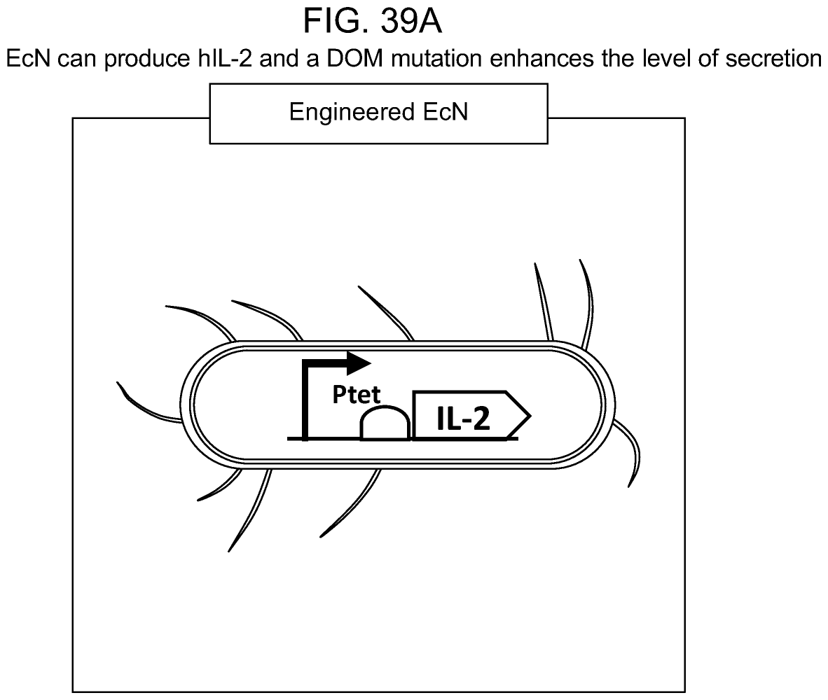

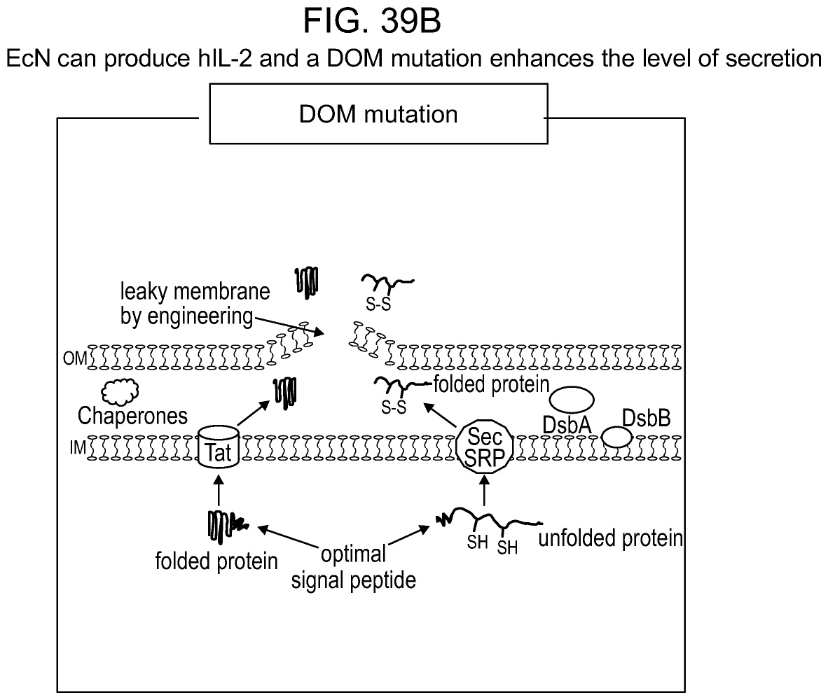

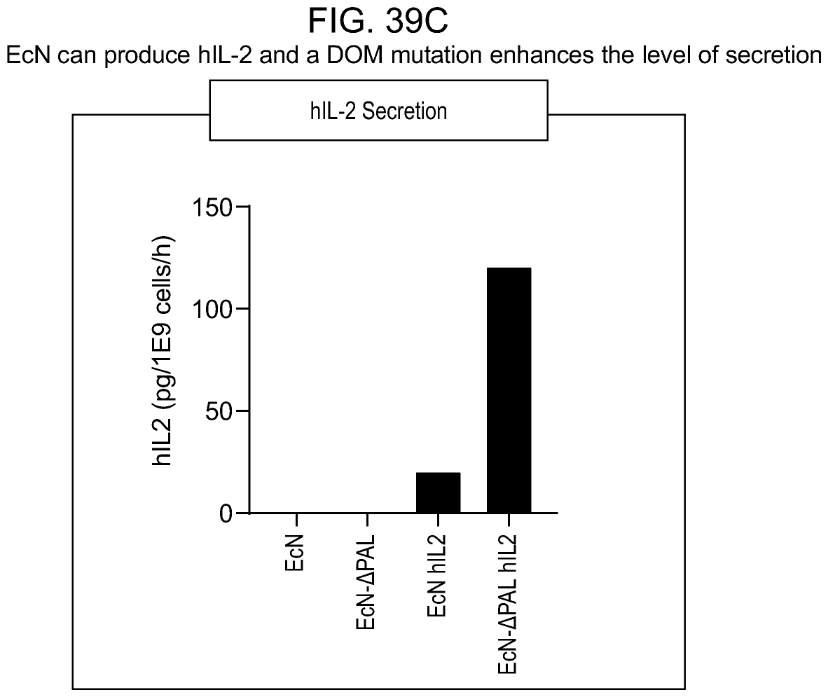

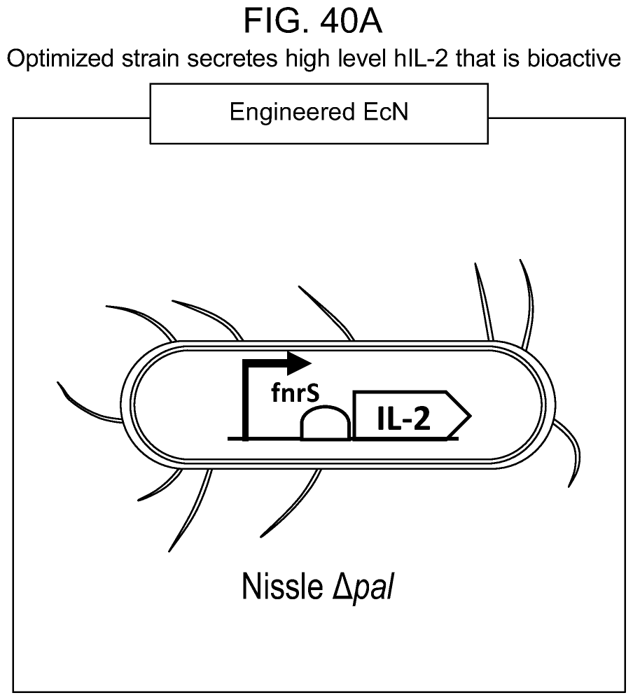

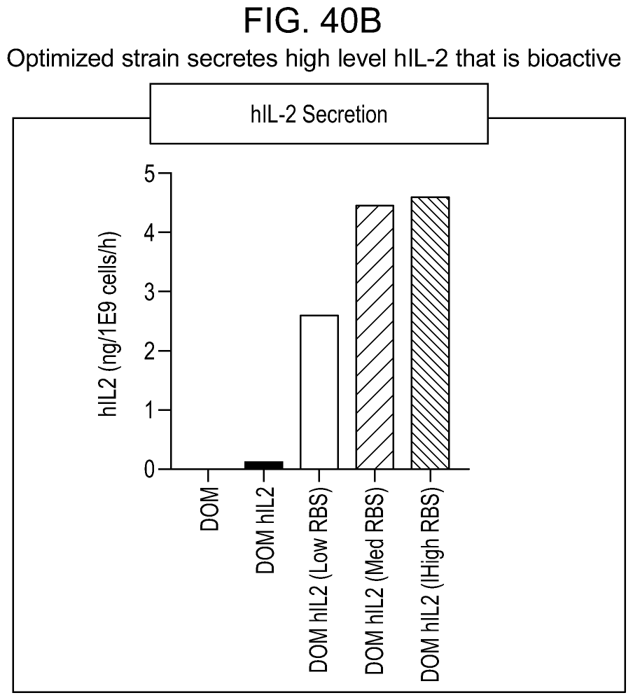

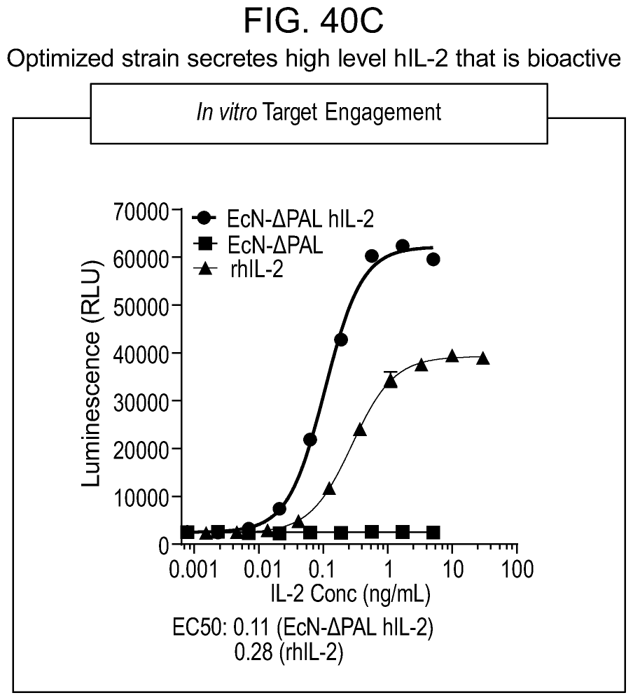

To determine whether the hIL-2 expressed by engineered bacteria is secreted, the concentration of IL-2 in the bacterial supernatant from engineered strains comprising hIL-2 secretion constructs/strains was measured. The strains carrying secretion constructs were in a wild type Nissle or a PAL deletion (PAL::Cm) background. All strains comprise either a plasmid expressing hIL-2 with a HdeB secretion tag from a tetracycline-inducible promoter (FIG. 39C) or a fnrS promoter, (FIG. 40B). For hIL-2 secretion constructs utilizing an fnrS promoter, a 3-member ribosome binding site library was constructed of low, medium, and high strength ribosome binding sites to determine its effect on resulting hIL-2 secretion.

E. coli Nissle strains were grown overnight in 2YT medium. Cultures were diluted 1:20 in 2YT in baffled flasks and grown shaking (250 rpm) for 2 hours. For strains with tetracycline-inducible promoters, cultures were concentrated to an optical density of 2.5 in 20 mL cultures, at which time anhydrous tetracycline (ATC) was added to cultures at a concentration of 100 ng/mL to induce expression of hIL-2. For strains utilizing an fnrS promoter, cultures were concentrated to an optical density of 2.5 in 20 mL cultures in a 125 mL non-baffled flasks to induce expression of hIL-2. After 4 hours of induction, cells were spun down, and supernatant was collected. To generate cell free medium, the clarified supernatant was further filtered through a 0.22 micron filter to remove any remaining bacteria and stored at −80C.

The concentration of hIL-2 in the cell-free medium was measured by hIL-2 ELISA (RnD Systems, Minneapolis, MN), according to manufacturer's instructions. All samples were run in duplicate, and a standard curve was used to calculate secreted levels of hIL-2. Standard curves were generated using recombinant hIL-2. Wild type or PAL::Cm Nissle supernatants were included in the ELISA as negative controls, and no signals were observed. FIGS. 39C and 40B summarize levels of hIL-2 measured in the respective supernatants. The data show that hIL-2 is secreted at various levels from the different bacterial strains.

The bioactivity of PAL::Cm Nissle secreted hIL-2 in the cell-free medium was measured by IL2 Bioassay (Promega Corp, Madison, WI), according to manufacturer's instruction. All samples were run in triplicate. Supernatant of PAL::Cm Nissle was included as a negative control and a recombinant hIL-2 (PeproTech Cranbury, NJ) was included as a positive control. FIG. 40C demonstrates that hIL-2 secreted is bioactive.

### Example 33. hIL-15Rα-hIL-15 Secretion

To determine whether the hIL-15Rα-hIL15 expressed by engineered bacteria is secreted, the concentration of hIL-15 in the bacterial supernatant from engineered strains comprising a hIL-15Rα-hIL15 secretion construct was measured from a wild type Nissle or a PAL::Cm Nissle background. Both strains comprise a plasmid expressing hIL-15Rα-hIL15 with a PpiA secretion tag from a tetracycline-inducible promoter.

E. coli Nissle strains were grown overnight in 2YT medium. Cultures were diluted 1:20 in 2YT and grown shaking (250 rpm) for 2 hours. Cultures were concentrated to an optical density of 2.5 at which time anhydrous tetracycline (ATC) was added to cultures at a concentration of 100 ng/mL to induce expression of hIL-15Rα-hIL15. After 4 hours of induction, cells were spun down, and supernatant was collected. To generate cell free medium, the clarified supernatant was further filtered through a 0.22 micron filter to remove any remaining bacteria and stored at −80C.

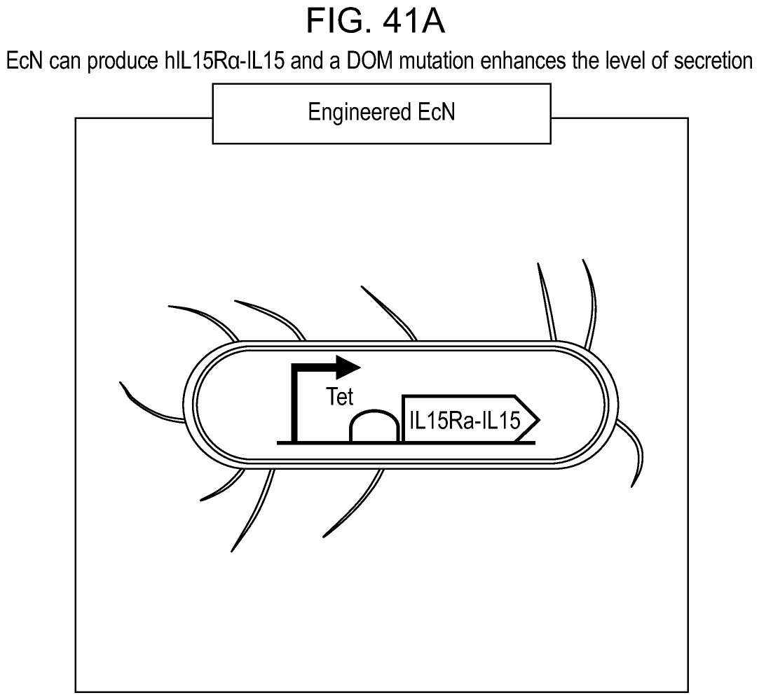

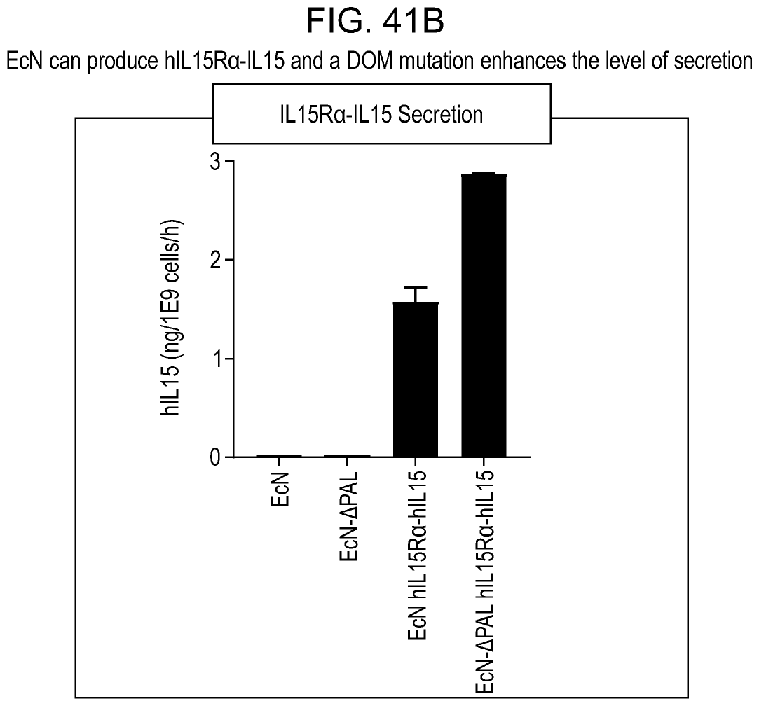

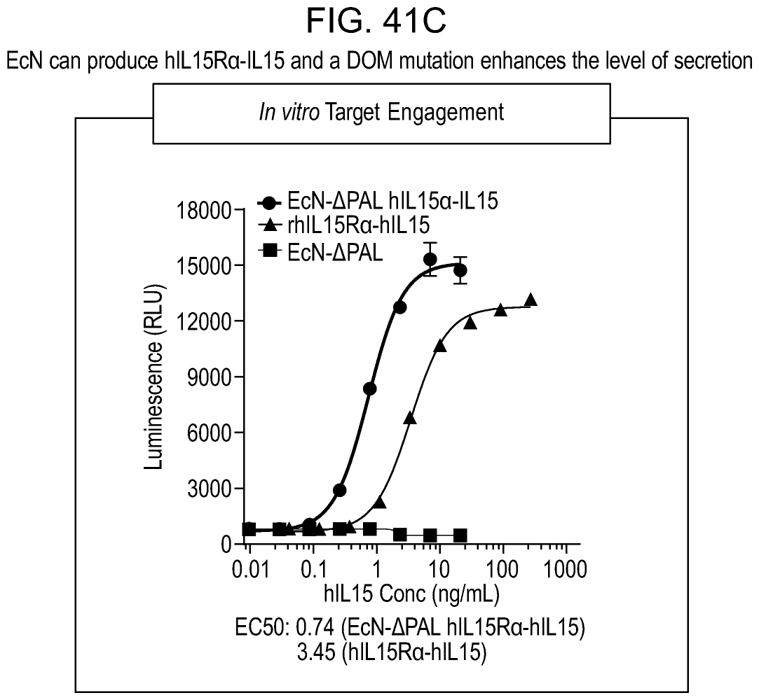

The concentration hIL-15Rα-IL15 in the cell-free medium was measured by hIL-15 ELISA (RnD Systems, Minneapolis, MN), according to manufacturer's instructions. All samples were run in duplicate, and a standard curve was used to calculate secreted levels of hIL-15. Standard curves were generated using recombinant hIL-15. Wild type Nissle or PAL:Cm Nissle was included in the ELISA as a negative control, and no signal was observed. FIG. 41B summarizes levels of hIL-15Rα-IL15 measured in the respective supernatants.

The bioactivity of EcN secreted hIL-15Rα-hIL-15 in the cell-free medium was measured by IL-2Rbg Bioassay Kit (Promega Corp, Madison, WI), according to manufacturer's instruction. All samples were run in triplicate. Supernatant of PAL:Cm Nissle was included as a negative control and a recombinant hIL-15Rα-hIL15 (Bon Opus Biosciences, Millburn, NJ) was included as a positive control. FIG. 41C summarizes potency of hIL-15Rα-hIL15 measured in the supernatants. The data show that hIL-15Rα-hIL15 secreted is bioactive.

### Example 34: 11-22 Secretion

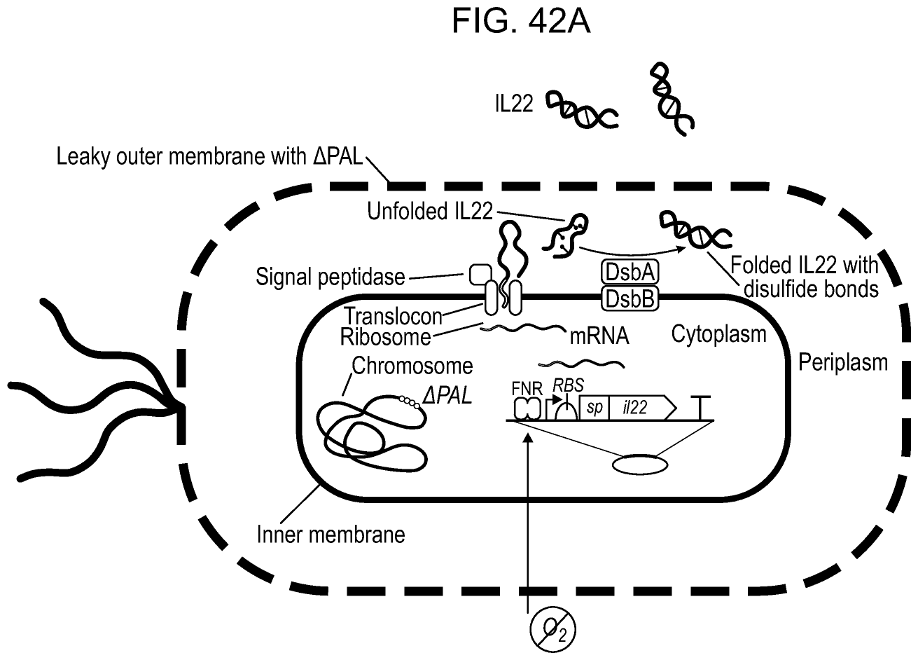

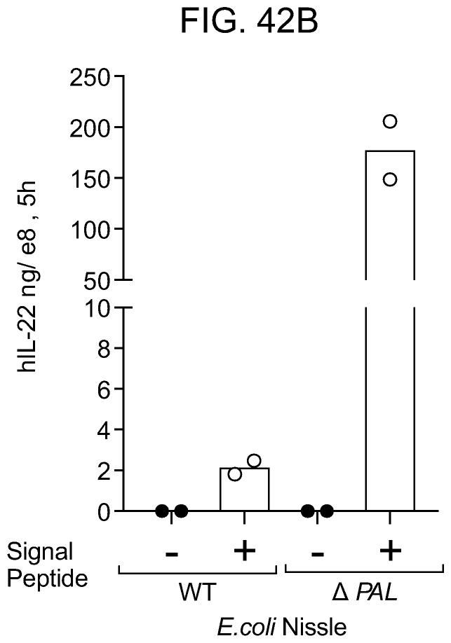

Secreted IL-22 was measured from ΔPAL and wild type bacteria with and without expressing IL-22 (FIG. 42B). IL-22 in the supernatant was measured at ng/1E8 over 5 hours.

### Exemplary Sequences

| Description | Sequence |
|---|---|
| hdeB-hIL2 | ATGGGATATAAGATGAACATTTCGTCACTGCGTAAGGC |
| SEQ ID NO: | GTTCATTTTTATGGGCGCGGTTGCCGCGTTATCATTAG |
| | TGAATGCGCAGAGCGCATTGGCTGCTCCCACATCCAGT |
| | TCAACAAAGAAAACACAGCTTCAGCTTGAGCATTTACT |
| | TCTTGATTTGCAGATGATTCTTAATGGTATTAACAACT |
| | ATAAAAATCCAAAACTTACGCGCATGTTGACGTTCAAA |
| | TTTTATATGCCGAAAAAAGCTACTGAATTGAAACATTT |
| | GCAATGTCTGGAAGAGGAATTGAAACCACTTGAGGAGG |
| | TCCTTAATTTAGCACAGAGCAAGAACTTCCATTTACGC |
| | CCGCGCGATTTAATTTCTAATATTAATGTTATCGTGTT |
| | AGAGCTTAAGGGGTCTGAAACTACGTTCATGTGCGAAT |
| | ATGCCGATGAAACGGCTACTATCGTCGAGTTCTTGAAT |
| | CGCTGGATTACGTTCTGTCAGAGCATTATCTCGACCCT |
| | GACCTAA |
| hdeB-hIL2 | MGYKMNISSLRKAFIFMGAVAALSLVNAQSALAAPTSS |
| SEQ ID NO: | STKKTQLQLEHLLLDLQMILNGINNYKNPKLTRMLTFK |
| | FYMPKKATELKHLQCLEEELKPLEEVLNLAQSKNFHLR |
| | PRDLISNINVIVLELKGSETTFMCEYADETATIVEFLN |
| | RWITFCQSIISTLT |

1. A recombinant bacterium comprising a gene sequence encoding one or more effector polypeptides and one or more secretion tags, wherein the gene sequence is operably linked to a directly or indirectly inducible promoter that is not associated with the gene sequence in nature and wherein the encoded effector polypeptide is secreted in a biologically active form.

2. A recombinant bacterium comprising a gene sequence encoding one or more effector polypeptides and one or more secretion tags, wherein the gene sequence is operably linked to a directly or indirectly inducible promoter that is not associated with the gene sequence in nature, wherein the effector polypeptide is secreted in a biologically active form, and wherein the bacterium comprises one or more mutations or deletions in an outer membrane protein selected from a group consisting of lpp, nlP, tolA, and PAL.

3. The recombinant bacterium of claim 1 or 2, wherein the effector is selected from a group consisting of GLP-2, IL-22, IL-10, IL-27, IL-19, IL-20, IL-24, IL-15, IL-2, GMCSF, TNF-alpha, IFN-gamma, CXCL10, CXCL9 and hyaluronidase.

4. The recombinant bacterium of any one of claims 1-3, wherein the secretion tag is selected from a group consisting of PhoA, OmpF, ompA, cvaC, TorA, fdnG, dmsA, PelB, tolB, torT, dsbA, GltI, GspD, HdeB, MalE, mglB, OppA, PpiA, lamb, ECOLIN_05715, ECOLIN_16495, ECOLIN_19410, and ECOLIN_19880 secretion signals.

5. The recombinant bacterium of any one of claims 1-4, wherein the gene sequence further comprises a sequence encoding a polypeptide linker.

6. The recombinant bacterium of claim 4, wherein the secretion tag is linked to the N terminus of the effector polypeptide via a peptide bond or a polypeptide linker.

7. The recombinant bacterium of claim 4, wherein the secretion tag is linked to the C terminus of the effector polypeptide via a peptide bond or a polypeptide linker.

8. The recombinant bacterium of any one of claims 1-7, wherein the secretion tag is cleaved after secretion of the effector polypeptide into the extracellular environment.

9. The recombinant bacterium of any one of claims 1-8, wherein the secretion tag is a PhoA secretion tag.

10. The recombinant bacterium of any one of claims 1-8, wherein the secretion tag is a ECOLIN 19410 secretion tag.

11. The recombinant bacterium of any one of claims 1-8, wherein the secretion tag is a GspD secretion tag.

12. The recombinant bacterium of any one of claims 1-8, wherein the secretion tag is a HdeB secretion tag.

13. The recombinant bacterium of any one of claims 1-8, wherein the secretion tag is a torT secretion tag.

14. The recombinant bacterium of any one of claims 1 and 3-13, further comprising one or more mutations or deletions in an outer membrane protein selected from a group consisting of lpp, nlP, tolA, and PAL.

15. The recombinant bacterium of any one of claims 1-14, wherein the deleted or mutated outer membrane protein is PAL.

16. The recombinant bacterium of any one of claims 1-14, wherein PAL is mutated.

17. The recombinant bacterium of any one of claims 1-14, wherein PAL is completely or partially deleted.

18. The recombinant bacterium of any one of claims 1-17, wherein the gene sequence further encodes a stabilizing polypeptide.

19. The recombinant bacterium of any one of claims 1-18, wherein the effector polypeptide is linked to the stabilizing polypeptide via a peptide linker or a peptide bond.

20. The recombinant bacterium of any one of claims 1-19, wherein the C terminus of the effector polypeptide is linked to the N terminus of the stabilizing polypeptide via a peptide linker or a peptide bond.

21. The recombinant bacterium of any one of claims 1-19, wherein the N terminus of the effector polypeptide is linked to the C terminus of the stabilizing polypeptide via a peptide linker or a peptide bond.

22. The recombinant bacterium of any one of claims 1-21, wherein the stabilizing polypeptide comprises an immunoglobulin Fc polypeptide.

23. The recombinant bacterium of claim 22, wherein the immunoglobulin Fc polypeptide comprises at least a portion of an immunoglobulin heavy chain CH2 constant region.

24. The recombinant bacterium of claim 22, wherein the immunoglobulin Fc polypeptide comprises at least a portion of an immunoglobulin heavy chain CH3 constant region.

25. The recombinant bacterium of claim 22, wherein the immunoglobulin Fc polypeptide comprises at least a portion of an immunoglobulin heavy chain CH1 constant region.

26. The recombinant bacterium of claim 22, wherein the immunoglobulin Fc polypeptide comprises at least a portion of an immunoglobulin variable hinge region.

27. The recombinant bacterium of claim 22, wherein the immunoglobulin Fc polypeptide comprises at least a portion of an immunoglobulin variable hinge region, an immunoglobulin heavy chain CH2 constant region and an immunoglobulin heavy chain CH3 constant region.

28. The recombinant bacterium of any one of claims 22-27, wherein the immunoglobulin Fc polypeptide is a human IgA or human IgG Fc polypeptide.

29. The recombinant bacterium of any one of claim 28, wherein the immunoglobulin Fc polypeptide is a human IgG Fc polypeptide.

30. The recombinant bacterium of any one of claim 28, wherein the immunoglobulin Fc polypeptide is a human IgA Fc polypeptide.

31. The recombinant bacterium of any one of claims 5-30, wherein the linker comprises a glycine rich peptide.

32. The recombinant bacterium of claim 31, wherein the glycine rich peptide comprises the sequence [GlyGlyGlyGlySer]n where n is 1, 2, 3, 4, 5 or 6 (SEQ ID NO: 1053).

33. The recombinant bacterium of claim 31, wherein the glycine rich peptide comprises the sequence GGGGSGGGGSGGGGSGGGGS (SEQ ID NO: 1280).

34. The recombinant bacterium of any one of claims 1-33, wherein the one or more effector polypeptides require multimerization for the effector polypeptide to be active in vivo.

35. The recombinant bacterium of any one of claims 1-34, wherein the one or more effector polypeptides require dimerization for the effector polypeptide to be active in vivo.

36. The recombinant bacterium of any one of claims 1-35, wherein the effector polypeptide is a homodimer.

37. The recombinant bacterium of any one of claims 1-35, wherein the effector polypeptide is a heterodimer.

38. The recombinant bacterium of any one of claims 1-37, wherein the gene sequence encoding the one or more effector polypeptides encodes a first monomer polypeptide and a second monomer polypeptide and wherein the first and second monomer polypeptides are linked to each other via a peptide linker or a peptide bond.

39. The recombinant bacterium of any one of claims 5-28, wherein the linker comprises
(SEQ ID NO: 1054)
GGGGSGGGS.

40. The recombinant bacterium of any one of claims 18-39, wherein the stabilizing polypeptide has the ability to perform an effector function.

41. The recombinant bacterium of any of claims 18-40, wherein the stabilizing polypeptide is able to perform an anti-inflammatory effector function.

42. The recombinant bacterium of any of claims 18-40, wherein the stabilizing polypeptide is able to perform an pro-inflammatory effector function.

43. The recombinant bacterium of any of claims 18-42, wherein the stabilizing polypeptide is a cytokine.

44. The recombinant bacterium of any of claims 18-43, wherein the stabilizing polypeptide is a multimer.

45. The recombinant bacterium of any of claims 18-44, wherein the stabilizing polypeptide is a dimer.

46. The recombinant bacterium of any one of claims 18-45, wherein the gene sequence encoding the stabilizing polypeptide comprises a first monomer and a second monomer, wherein the first and second monomer are linked to one another via a peptide bond or a peptide linker.

47. The recombinant bacterium of any one of claims 1-46, wherein the promoter is induced by exogenous environmental conditions found in a mammalian gut.

48. The recombinant bacterium of claim 47, wherein the promoter is induced under low-oxygen or anaerobic conditions.

49. The recombinant bacterium of claim 48, wherein the promoter is a FNR-responsive promoter, an ANR-responsive promoter, or a DNR-responsive promoter.

50. The recombinant bacterium of claim 49, wherein the promoter is a FNR-responsive promoter.

51. The recombinant bacterium of any of claims 1-50, wherein the promoter is induced by the presence of reactive nitrogen species.

52. The recombinant bacterium of claim 51, wherein the promoter is a NsrR-responsive promoter, NorR-responsive promoter, or a DNR-responsive promoter.

53. The recombinant bacterium of any one of claims 1-52, wherein the promoter is induced by the presence of reactive oxygen species.

54. The recombinant bacterium of any of claim 53, wherein the promoter is a OxyR-responsive promoter, PerR-responsive promoter, OhrR-responsive promoter, SoxR-responsive promoter, or a RosR-responsive promoter.

55. The recombinant bacterium of any one of claims 1-54, wherein the gene sequence is located on a chromosome in the bacterium.

56. The recombinant bacterium of any one of claims 1-54, wherein the gene sequence is located on a plasmid in the bacterium.

57. The recombinant bacterium of any one of claims 1-56, wherein the bacterium is a probiotic bacterium.

58. The recombinant bacterium of any one of claims 1-57, wherein the bacterium is a tumor targeting bacterium.

59. The recombinant bacterium of claim 58, wherein the bacterium is selected from the group consisting of Bacteroides, Bifidobacterium, Clostridium, Escherichia, Lactobacillus, and Lactococcus.

60. The recombinant bacterium of claim 59, wherein the bacterium is selected from Clostridium novyi NT, and Clostridium butyricum, and Bifidobacterium longum.

61. The recombinant bacterium of claim any of claims 1-60, wherein the bacterium is Escherichia coli strain Nissle.

62. The recombinant bacterium of any one of claims 1-61, wherein the bacterium is an auxotroph in a gene that is complemented when the bacterium is present in a mammalian gut.

63. The recombinant bacterium of claim 62, wherein the bacterium is an auxotroph in diaminopimelic acid or an enzyme in the thymine biosynthetic pathway.

64. The recombinant bacterium of any one of claims 1-63, wherein the recombinant bacterium is capable of producing about 25 pg/1E9 cells/hr to about 4500 pg/1E9 cells/hr of IL-2 in vitro.

65. The recombinant bacterium of any one of claims 1-64, wherein the recombinant bacterium is capable of producing about 1.5 ng/1E9 cells/hr to about 3 ng/1E9 cells/hr of IL15 in vitro.

66. A recombinant bacterium capable of secreting IL-22 comprising
1) a nucleic acid sequence encoding IL-22 and one or more secretion tags, wherein the nucleic acid sequence is operably linked to an FNR-responsive promoter;
2) one or more mutations or deletions in an outer membrane protein PAL.

67. The recombinant bacterium of claim 66, wherein the secretion tag is selected from a group consisting of PhoA, OmpF, ompA, cvaC, TorA, fdnG, dmsA, PelB, tolB, torT, dsbA, GltI, GspD, HdeB, MalE, mglB, OppA, PpiA, lamb, ECOLIN_05715, ECOLIN_16495, ECOLIN_19410, and ECOLIN_19880 secretion signals.

68. The recombinant bacterium of claim 66 or 67, wherein the bacterium is capable of producing about 40 ng/1E8 cells/hr of IL-22 in vitro.

69. A pharmaceutically acceptable composition comprising the recombinant bacterium of any one of claims 1-68; and a pharmaceutically acceptable carrier.

70. The composition of claim 69 formulated for oral or rectal administration.

71. A method of treating or preventing a disorder comprising the step of administering to a patient in need thereof, the composition of any one of claims 1-70.

72. The method of claim 71, wherein the disorder is selected from a group consisting of autoimmune disorders, cancer, metabolic diseases, diseases relating to inborn errors of metabolism, and neurological or neurodegenerative diseases.

73. The method of claim 72, wherein the autoimmune disorder is selected from the group consisting of acute disseminated encephalomyelitis (ADEM), acute necrotizing hemorrhagic leukoencephalitis, Addison's disease, agammaglobulinemia, alopecia areata, amyloidosis, ankylosing spondylitis, anti-GBM/anti-TBM nephritis, antiphospholipid syndrome (APS), autoimmune angioedema, autoimmune aplastic anemia, autoimmune dysautonomia, autoimmune hemolytic anemia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenic purpura (ATP), autoimmune thyroid disease, autoimmune urticarial, Axonal & neuronal neuropathies, Balo disease, Behcet's disease, Bullous pemphigoid, Cardiomyopathy, Castleman disease, Celiac disease, Chagas disease, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogan syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibrosing alveolitis, Giant cell arteritis (temporal arteritis), Giant cell myocarditis, Glomerulonephritis, Goodpasture's syndrome, Granulomatosis with Polyangiitis (GPA), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile idiopathic arthritis, Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (Systemic Lupus Erythematosus), chronic Lyme disease, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatic, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, subacute bacterial endocarditis (SBE), Susac's syndrome, sympathetic ophthalmia, Takayasu's arteritis, temporal arteritis/giant cell arteritis, thrombocytopenic purpura (TTP), Tolosa-Hunt syndrome, transverse myelitis, type 1 diabetes, asthma, ulcerative colitis, undifferentiated connective tissue disease (UCTD), uveitis, vasculitis, vesiculobullous dermatosis, vitiligo, and Wegener's granulomatosis.

74. The method of claim 72, wherein the cancer is selected from adrenal cancer, adrenocortical carcinoma, anal cancer, appendix cancer, bile duct cancer, bladder cancer, bone cancer (e.g., Ewing sarcoma tumors, osteosarcoma, malignant fibrous histiocytoma), brain cancer (e.g., astrocytomas, brain stem glioma, craniopharyngioma, ependymoma), bronchial tumors, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, gestational trophoblastic disease, heart cancer, Kaposi sarcoma, kidney cancer, largyngeal cancer, hypopharyngeal cancer, leukemia (e.g., acute lymphoblastic leukemia, acute myeloid leukemia, chronic lymphocytic leukemia, chronic myelogenous leukemia), liver cancer, lung cancer, lymphoma (e.g., AIDS-related lymphoma, Burkitt lymphoma, cutaneous T cell lymphoma, Hodgkin lymphoma, Non-Hodgkin lymphoma, primary central nervous system lymphoma), malignant mesothelioma, multiple myeloma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma, skin cancer (e.g., basal cell carcinoma, melanoma), small intestine cancer, stomach cancer, teratoid tumor, testicular cancer, throat cancer, thymus cancer, thyroid cancer, unusual childhood cancers, urethral cancer, uterine cancer, uterine sarcoma, vaginal cancer, vulvar cancer, Waldenström macrogloblulinemia, and Wilms tumor.

75. The method of claim 72, wherein the metabolic disorder or condition is selected from the group consisting of: type 1 diabetes; type 2 diabetes; metabolic syndrome; Bardet-Biedel syndrome; Prader-Willi syndrome; non-alcoholic fatty liver disease; tuberous sclerosis; Albright hereditary osteodystrophy; brain-derived neurotrophic factor (BDNF) deficiency; Single-minded 1 (SIM1) deficiency; leptin deficiency; leptin receptor deficiency; pro-opiomelanocortin (POMC) defects; proprotein convertase subtilisin/kexin type 1 (PCSK1) deficiency; Src homology 2B1 (SH2B1) deficiency; pro-hormone convertase 1/3 deficiency; melanocortin-4-receptor (MC4R) deficiency; Wilms tumor, aniridia, genitourinary anomalies, and mental retardation (WAGR) syndrome; pseudohypoparathyroidism type 1A; Fragile X syndrome; Borjeson-Forsmann-Lehmann syndrome; Alstrom syndrome; Cohen syndrome; and ulnar-mammary syndrome.
